(19) United States
(12) Patent Application Publication
(10) Pub. No.: US 2026/0061042 A1
(43) Pub. Date: Mar. 5, 2026

(54) POLYNUCLEOTIDE COMPOSITIONS AND USES THEREOF

(71) Applicant: Pfizer Inc., New York, NY (US)

(72) Inventors: Ye Che, Niantic, CT (US); Robert George Konrad Donald, South Orange, NJ (US); Patricia Michele McMonagle, Wayne, NJ (US); Natalie Clare Silmon de Monerri, New York, NY (US)

(21) Appl. No.: 19/086,236

(22) Filed: Mar. 21, 2025

Related U.S. Application Data
(60) Provisional application No. 63/569,959, filed on Mar. 26, 2024.
(60) Provisional application No. 63/770,673, filed on Mar. 12, 2025.

(51) Int. Cl. A61K 39/108; A61K 39/00; A61P 37/04; C07K 14/245

(52) U.S. Cl. CPC A61K 39/0258; A61P 37/04; C07K 14/245; A61K 2039/53; A61K 2039/6018

(57) ABSTRACT

The invention relates to polypeptides and RNA molecules encoding an E. coli fimbrial antigen PapG mutant. The present disclosure further relates to compositions comprising the RNA molecules formulated in a lipid nanoparticle (RNA-LNP). The present disclosure further relates to the use of the polypeptides, RNA molecules, RNA-LNPs and compositions thereof for the prevention of E. coli infection, including urinary tract infection.

### CROSS REFERENCE TO RELATED APPLICATIONS

This application claims the benefit of and U.S. Provisional Application No. 63/770,673, filed on Mar. 12, 2025 and U.S. Provisional Application No. 63/569,959, filed Mar. 26, 2024. The entire content of each of the foregoing applications is hereby incorporated herein by reference.

### REFERENCE TO SEQUENCE LISTING

This application is being filed electronically via Patent Center and includes an electronically submitted sequence listing in .xml format. The .xml file contains a sequence listing entitled “PC073022A_UpdatedSEQListing_ST26.xml” created on Jun. 24, 2025 and having a size of 202 KB. The sequence listing contained in this .xml file is part of the specification and is hereby incorporated herein by reference in its entirety.

### FIELD OF THE INVENTION

The invention relates to compositions and methods for the preparation, manufacture and therapeutic use of ribonucleic acid vaccines comprising polynucleotide molecules encoding one or more Escherichia coli (E. coli) fimbrial antigens, such as P fimbrial antigen (PapG).

### BACKGROUND OF THE INVENTION

Urinary tract infections (UTI) affect 1 in 5 women at least once during their lifetime and are responsible for significant mobidity and mortality, resulting in a substantial burden on healthcare systems. While several different bacteria can cause UTI, the most common cause (90-95% of cases) is the Gram-negative bacteria Escherichia coli (E. coli). Most E. coli UTI are caused by uropathogenic E. coli (UPEC) that colonise the gastrointestinal tract and migrate from the faecal flora to the urogenital tract, where they adhere to host uroepithelial cells, thus establishing a reservoir for ascending infections of the urinary tract. Adhesion is facilitated by fimbrial adhesins including PapG, a fimbrial adhesin which is present in only ˜30% of UPEC strains but is thought to play a role during development of pyelonephritis, specifically through binding of glycosylated receptors on renal epithelial cells [5]. Acute pyelonephritis is a serious infection of the kidneys resulting from the ascension of uropathogenic E. coli (UPEC) from untreated acute cystitis in the bladder to the kidneys. Acute pyelonephritis is accompanied by urosepsis in about 30% of adults [6] [7]. In children, acute pyelonephritis can delay renal growth and cause renal damage [8]. P fimbriae, which are encoded in the pap (pyelonephritis-associated pili) gene cluster, are expressed in UPEC strains that cause pyelonephritis. P fimbriae consist of rigid stalks composed of approximately one thousand copies of subunit protein PapA connected to a flexible tip of minor subunit proteins PapE and PapF. At the distal end of the pilus is the receptor-binding adhesin, PapG [9].

PapG is similar in structure to FimH, wherein it is composed of an N-terminal lectin domain (LD) and pilin domain (PD). The LD exists in multiple conformational states that differ in affinity for PapG cognate receptors ([10, 11]). In addition, like FimH, the folding of full-length PapG adhesin is mediated by a chaperone, PapD, and the protein is stabilized via the PD through a mechanism termed ‘donor-strand complementation’ [10, 12, 13]. In this mechanism, the chaperone donates a β-strand to complete the immunoglobulin-like fold of the PD [14]. Donor strand complementation is also the mechanism by which fimbrial subunits are assembled into a pilus. The C-terminal PD of the PapG adhesin is linked to the N-terminus of PapF on the pilus via donor stand exchange [13, 15]. The structural dependency of fimbrial adhesins on donor strand complementation renders the full-length proteins unstable when expressed in isolation [16].

Three main alleles of PapG exist (PapG-I, PapG-II and PapG-III) with each allele having varying affinities to different Gal(1-4) Gal-containing receptors [17]. Glycolipid binding studies and hemagglutination experiments have shown that PapGI adhesins preferentially bind globotriaosylceramide (GbO₃), and PapG-II adhesins preferentially bind globoside (GbO₄). Both receptors are abundant on human uroepithelial cells [18]. PapG-III has been found to bind Forssman antigen (GbO₅) which is present on canine uroepithelial cells, and is associated with urinary tract infections in cats and dogs and with human cystitis [19]. Little is known about the clinical association of PapG-I. Most UPEC that express P fimbriae express a PapG-II adhesin which is abundantly expressed in E. coli and is associated with human pyelonephritis and bacteremia [21, 22].

PapG-II is an attractive vaccine target for several reasons. The PapG-II adhesin was proven to be essential in the pathogenesis of experimental E. coli kidney infections in cynomolgus monkeys; when PapG-II was deleted from a pyelonephritic E. coli strain, the strain failed to cause pyelonephritis [23]. Cynomolgus monkeys vaccinated with purified PapDG protein (full-length PapG in complex with the chaperone, PapD) had high IgG serum titers to PapDG as well as full-length isolated P fimbriae, and monkeys were protected against pyelonephritis [24]. In addition, mice immunized with P fimbriae (Gal-Gal pili) were protected against E. coli pyelonephritis [25, 26]. In this report, PapG refers to PapG-II. PapG contains multiple disulfide bonds, and for this reason recombinant proteins (either LD alone or in complex with a chaperone) must be produced in the E. coli periplasm ([27, 28]).

The osmotic shock method used to extract proteins from the E. coli periplasm is not scalable for manufacturing and presents a major hurdle for development of vaccines composed of fimbrial antigens. In earlier work, we established a mammalian expression platform to produce FimH proteins (PCT Intl. Appln No. PCT/IB2020/06008, filed Oct. 28, 2020; Pfizer docket PC072517).

Accordingly, there is a need for improved immunogenic compositions comprising PapG antigens with reduced affinity for their cognate ligands and improved biochemical properties that result in improved functional immunogenicity relative to wild type PapG.

### SUMMARY OF THE INVENTION

The present disclosure provides the unmet need for improved immunogenic compositions against E. coli infection, among other things, as provided herein. This disclosure provides a mammalian expression platform to produce PapG proteins and the engineering of single-chain donor strand-complemented full-length versions of PapG that can be produced without the need for a chaperone. Furthermore, this disclosure provides the design and evaluation of PapG_{LD} variants that were predicted to be unable to bind their cognate ligands, which was evaluated in a series of biochemical and biophysical analyses. This disclosure also describes mRNA constructs encoding PapG (LD or full-length) and demonstrates expression of PapG on the surface of mammalian cells or secreted into the extracellular milieu in modRNA transcript transfected cells. Lastly, a serological assay to measure immune responses to PapG is described, based on its ability to agglutinate human red blood cells. In one aspect, the disclosure provides, a mutant of a wild-type PapG polypeptide, wherein the mutant comprises at least one amino acid mutation relative to the amino acid sequence of the wild type PapG polypeptide, wherein the mutation is selected from the group consisting of: G18, G75, G86, S89, N96, G104, W107, G122, G147, G168, R170, K172, N24S, and N286, or any combination thereof, and wherein the amino acid positions are numbered according to SEQ ID NO:120.

In one aspect, the disclosure provides immunogenic compositions and methods for preventing, treating or ameliorating an infection, disease or condition in a subject comprising the administration of RNA molecules, e.g., immunogenic RNA polynucleotide encoding an amino acid sequence, e.g., an immunogenic antigen, comprising an E. coli PapG protein (“PapG”), an immunogenic variant thereof, or an immunogenic fragment of the PapG protein or the immunogenic variant thereof, e.g., an antigenic peptide or protein. Thus, the immunogenic antigen comprises an epitope of a PapG protein for inducing an immune response against PapG, in the subject. RNA polynucleotide encoding an immunogenic antigen is administered to provide (following expression of the polynucleotide by appropriate target cells) antigen for induction, e.g., stimulation, priming, and/or expansion, of an immune response, e.g., antibodies and/or immune effector cells. In one aspect, the immune response to be induced according to the present disclosure is both B cell-mediated immune response, e.g., an antibody-mediated immune response as well as T-cell-mediated immune response. In one aspect, the immune response is an anti-PapG immune response.

The immunogenic compositions described herein comprise RNA molecules comprising RNA (as the active principle) that may be translated into one or more proteins in a recipient's cells. In addition to wild type, codon-optimized or mutant sequences encoding the antigen sequence, the RNA molecules may contain one or more structural elements optimized for maximal efficacy of the RNA with respect to stability and translational efficiency (5′ cap, 5′ UTR, subgenomic promoter, 3′ UTR, poly-A-tail). In one aspect, the RNA molecules contain all of these elements. The RNA molecules described herein may be complexed with lipids and/or proteins to generate RNA-particles (e.g., lipid nanoparticles (LNPs)) for administration. In one aspect, the RNA molecules described herein are complexed with lipids to generate RNA-lipid nanoparticles (e.g. RNA-LNPs) for administration. In one aspect, the RNA molecules described herein are complexed with proteins for administration. In one aspect, the RNA molecules described herein are complexed with lipids and proteins for administration. If a combination of different RNA molecules is used, the RNA molecules may be complexed together or complexed separately with lipids and/or proteins to generate RNA-particles for administration.

The present disclosure provides for RNA molecules and RNA-LNPs that include at least one open reading frame (ORF) encoding a PapG antigen. In some aspects, the PapG antigen is a PapG polypeptide. In some aspects, the PapG polypeptide is a full-length, truncated, fragment or variant thereof. In some aspects, the PapG polypeptide comprises at least one mutation. In another aspect of the RNA molecule, the PapG antigenic polypeptide comprises amino acids having an amino acid sequence as set forth in SEQ ID NO: 11 to 41. In a further aspect, the PapG antigenic polypeptide comprises PapG_{DSF} (SEQ ID NO: 69), PapG_{DSF} mutant, PapG_{LD} mutant, or an immunogenic fragment thereof. In one aspect, the mutant PapG polypeptide has at least 80% identity to the amino acid sequence set forth in any one of SEQ ID NO: 11 to 41. In a further aspect, the PapG polypeptide has at least 90%, 95, 96%, 97%, 98% or 99% identity to the amino acid sequence selected from SEQ ID NO: 11 to 41.

All methods described herein can be performed in any suitable order unless otherwise indicated herein or otherwise clearly contradicted by context. The use of any and all examples, or exemplary language (e.g., “such as”), provided herein is intended merely to further illustrate the disclosure and does not pose a limitation on the scope of the claims. No language in the specification should be construed as indicating any non-claimed element essential to the practice of the disclosure.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

It is contemplated that any aspect discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE FIGURES

FIG. 1 describes expression of WT PapG_{LD}, alanine mutant and N-glycosylation mutant N96S in Expi293 cells. WT PapG_{LD} was expressed from a vector using a mouse IgGK signal peptide for secretion, by transfection of ExpiCHO (CHO) cells. Expression in cell pellets (P) and culture supernatants(S) was evaluated by Western blot using an anti-His tag antibody. FimH construct 2083, encoding WT FimHLD was used as positive control for the transfection and Western blot. All constructs included the mouse IgGK as a signal peptide to direct the proteins for secretion. Constructs encoded WT PapG_{LD}, WT PapG_{LD} with an N-terminal Alanine residue (+Ala), WT PapG_{LD} with an N-terminal Alanine residue and N-glycosylation site mutation N96S.

FIGS. 2A and 2B describe the design of full-length donor strand complemented PapG_{DSF}. To engineer a single chain full-length donor strand complemented PapG protein, the full-length PapG-II protein is stabilized by a 15-residue complementary donor strand peptide from the N-terminus of PapF (having polypeptide sequence ADVQINIRGNVYIPP (including Ala as the 1st residue); purple cartoon) connected via a 6-residue peptide GGSSGG linker (blue ribbon) to the C-terminus of PapG-II to form a complete immunoglobin fold. The K172A (red sphere) mutation close to the globoside GbO₄ binding site prevents the binding of the tetrasaccharide epitope (discussed in Section B.3 herein). The lectin domain of PapG-II is represented in grey, and the pilin domain is shown in green (FIG. 2A). Schematic of PapG_{DSF} which was cloned into a vector with mouse IgGK secretion signal at the N-terminus and His-tag at the C-terminus; various lengths of linker peptides were explored, 5-8 amino acids (aa) (FIG. 2B).

FIG. 3 describes expression of PapG_{DSF} with different GS linker lengths in ExpiCHO cells. Anti-His antibody Western blot on ExpiCHO cells transfected with plasmids encoding mouse IgGK signal peptide followed by PapG_{DSF} variants wherein the Glycine-Serine (GS) linker between the pilin domain and PapF peptide were of different lengths (5-8 residues, denoted under the Western blot image). PapG_{DSF} variants were tagged with an 8×His tag at their C-termini. A construct encoding His-tagged FimH lectin domain was used as a positive control for the transfection and Western blot.

FIG. 4 describes residues in PapG-IILD selected for mutation. Residues (red spheres) that are in direct contact with the tetrasaccharide epitope of the globoside GbO₄ were chosen for mutation to prevent the binding of the tetrasaccharide epitope. Glycine residues (blue spheres) that have positive protein backbone F-angles (underlined) were chosen for mutation to potentially favor a ligand-free PapG-II conformation. Protein backbone F, Y-angles are also placed next to the Glycine residue labels.

FIGS. 5A and 5B describe Western blot and Octet analysis of relative expression of PapG_{LD} mutants. Small scale transfections of plasmids encoding PapG_{LD} mutants were performed in a 96-well deep well plate format (96DWB) in ExpiCHO cells. Neat supernatant was analyzed by Western blot using an anti-His antibody (FIG. 5A). Expression in supernatants following transfection was quantified by Octet simple quantitation (FIG. 5B).

FIG. 6 describes the design of mRNA constructs encoding secreted PapG. mRNA vectors encoding PapG (LD or DSF) including mouse IgGK signal for secretion. Sequences were cloned into mRNA expression vector BMD2.

FIGS. 7A and 7B describe Western blot and Octet analysis of culture supernatants transfected with modRNA transcripts encoding secreted PapG_{LD} N96S or PapG_{DSF} N96S N286S. Expi293 cells were transfected with modRNA transcripts from BMD2 constructs encoding aglycoslyated PapG_{LD} and partially aglycosylated PapG_{DSF}. After 24 hours, neat supernatants and cell pellets from 500 ng transfection dose were analyzed by Western blot using anti-PapG polyclonal antibodies (wk6)—for detection and purified PapG proteins as a reference. When preparing cell culture samples for Western Blot analysis, cell pellets were resuspended in an equivalent 50 μL volume of spent media as the supernatant sample. 20 ml sample volumes were loaded per well (PapG_{DSF} contains unmodified N242, which is glycosylated, as observed by the double band) (FIG. 7A). Protein expression in the supernatants was measured by Octet simple quantitation using ProG/ECO 726 for capture (FIG. 7B).

FIG. 8 describes the design of mRNA constructs encoding membrane anchored PapG. mRNA vectors encoding PapG (LD or DSF) included mouse IgGK signal for secretion and were targeted to the plasma membrane with a range of transmembrane targeting sequences (DAF-GPI, Decay Accelerating Factor Glycerolphosphatidylinositol, Thyl Glycerolphospharidylinositol phosphate anchor, or viral transmembrane sequence). Sequences were cloned into mRNA constructs BMD2, BMD562, BMD563 or BMD576 which varied in their 5′UTR sequence (3′UTR and polyA tail were identical across mRNA backbones).

FIG. 9 describes expression of PapG on the surface of muscle skeletal cells. Representative confocal fluorescence microscopy image of skeletal muscle cells transfected with modRNA from BMD2 constructs encoding PapG_{LD} cloned in-frame with a sequence encoding DAF-GPI membrane targeting sequence. Nuclei were stained with DAPI (blue) and PapG was detected with anti-PapG rabbit anti-sera (shown in green). 40× magnification.

FIG. 10 describes expression of PapG_{LD} N96S on the surface of modRNA transfected Expi293 cells with different membrane anchoring sequences. Expi293 cells were transfected with RNA transcripts from BMD2 constructs encoding aglycosylated PapG_{LD} with DAF-GPI, Thyl-GPI, or HSV gD TMD. Cell surface expression was detected by anti-PapG polyclonal antibodies and evaluated by flow cytometry 24 hours after transfection.

FIG. 11 depicts expression of PapG_{LD} N96S-DAF-GPI on the surface of modRNA transfected Expi293 cells, with four different 5′UTR/3′UTR backbones. Expi293 cells were transfected with RNA transcripts from BMD2, BMD562, BMD563 or BMD576 constructs encoding aglycosylated PapG_{LD} with DAF-GPI. Cell surface expression was detected by anti-PapG polyclonal antibodies and evaluated by flow cytometry 24 hours after transfection.

FIG. 12 depicts expression of PapG_{LD} N96S-DAF-GPI on the surface of modRNA transfected Expi293 cells, containing GbO₄ non-binding mutations. Expi293 cells were transfected with modRNA transcripts from BMD2 constructs encoding aglycosylated PapG_{LD} with DAF-GPI containing mutations that were found to prevent binding to GbO₄ in Luminex binding assays (R170A, K172A, G104A, W107A) compared to WT. Cell surface expression was detected by anti-PapG polyclonal antibodies and evaluated by flow cytometry 24 hours after transfection.

FIGS. 13A and 13B depict expression of PapG_{LD} N96S and PapG N96S N242S N286S-DSF in BMD2 and BMD576 on the surface of modRNA transfected Expi293 cells, containing GbO₄ non-binding mutation K172A. Expi293 cells were transfected with RNA transcripts from BMD2 (FIG. 13A) and BMD576 (FIG. 13B) constructs encoding aglycosylated PapG_{LD} and PapG_{DSF} with DAF-GPI with and without K172A, a mutation which was found to prevent binding to GbO₄ in Luminex binding assays. Cell surface expression was detected by anti-PapG polyclonal antibodies and evaluated by flow cytometry 24 hours after transfection.

FIG. 14 depicts expression of PapG_{DSF} N96S N242S N286S in different 5′UTR/3′UTR backbones on the surface of modRNA transfected Expi293 cells. Expi293 cells were transfected with modRNA transcripts from BMD2, BMD562 and BMD576 constructs encoding aglycosylated PapG_{DSF} with the DAF-GPI and from a BMD576 construct encoding aglycosylated PapG_{DSF} with Thyl-GPI. Cell surface expression was detected by anti-PapG polyclonal antibodies and evaluated by flow cytometry 24 hours after transfection.

FIG. 15 depicts an overview of hemagglutination assay to evaluate PapG-dependent agglutination of human red blood cells. Assay to probe for E. coli (strain CFT073)-driven and PapG-dependent agglutination of human red blood cells (RBCs). A schematic and a proof-of-concept experiment are depicted in the upper and lower panel, respectively. The lower panel shows wells containing red blood cells and bacteria in a 96-well plate. WT or knockout bacteria were titrated across the plate from left to right, and no bacteria were added to the wells on the right. CFT073 WT strain, CFT073 ΔΔ papG is a double PapG gene knockout.

FIGS. 16A and 16B depict that Hemagglutination can be prevented by PapG ligand or antisera. Pre-incubation of E. coli CFT073 WT strain (2.4×10⁶ CFU) with a synthetic PapG ligand GbO₃ (FIG. 16A) or serum from rabbits vaccinated with three doses of PapG_{LD} (FIG. 16B) efficiently blocks agglutination of RBCs. Serum collected prior to vaccination does not interfere with HA.

FIG. 17 shows that modRNA LNPs expressing membrane-targeted PapG induce potent HA titers in mice after two doses.

FIG. 18 depicts a polypeptide sequence alignment of PapGII version 1 sequence from CFT073 (SEQ ID: 120) compared to PapG sequences from PapGII version 2 sequence from CFT073 (SEQ ID NO: 121) and 44 clinical UPEC isolates which show 56-99% identity.

### DETAILED DESCRIPTION OF THE INVENTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. A mutant of a wild-type PapG polypeptide, wherein the mutant comprises at least one amino acid mutation relative to the amino acid sequence of the wild type PapG polypeptide, wherein the mutation is selected from the group consisting of: G18, G75, G86, S89, N96, G104, W107, G122, G147, G168, R170, K172, N24S, and N286, or any combination thereof, and wherein the amino acid positions are numbered according to SEQ ID NO: 120.
E2. The mutant PapG polypeptide according to embodiment E1, comprising at least one mutation selected from the group consisting of: G18A, G75A, G86A, S89T, N96S, G104A, W107A, G122A, G147A, G168A, R170A, K172A, N242S, and N286S, or any combination thereof.
E3. The mutant PapG polypeptide according to embodiment E2, comprising the mutation N96S.
E4. The mutant PapG polypeptide according to embodiment E2, comprising the mutations N96S and G86A.
E5. The mutant PapG polypeptide according to embodiment E2, comprising the mutations N96S and S89T.
E6. The mutant PapG polypeptide according to embodiment E2, comprising the mutations N96S and G104A.
E7. The mutant PapG polypeptide according to embodiment E2, comprising the mutations N96S and G168A.
E8. The mutant PapG polypeptide according to embodiment E2, comprising the mutations N96S and G18A.
E9. The mutant PapG polypeptide according to embodiment E2, comprising the mutations N96S and G75A.
E10. The mutant PapG polypeptide according to embodiment E2, comprising the mutations N96S and G122A.
E11. The mutant PapG polypeptide according to embodiment E2, comprising the mutations N96S and G147A.
E12. The mutant PapG polypeptide according to embodiment E2, comprising the mutations N96S and W107A.
E13. The mutant PapG polypeptide according to embodiment E2, comprising the mutations N96S and R170A.
E14. The mutant PapG polypeptide according to embodiment E2, comprising the mutations N96S and K172A.
E15. The mutant PapG polypeptide according to embodiment E2, comprising the mutations N96S and N286S.
E16. The mutant PapG polypeptide according to embodiment E2, comprising the mutations N96S, N242S and N286S.
E17. The mutant PapG polypeptide according to embodiment E2, comprising the mutations N96S, N242S and K172A.
E18. The mutant PapG polypeptide according to embodiment E2, comprising the mutations N96S, N242S, N286S and K172A.
E19. The mutant PapG polypeptide according to embodiment E2, comprising the amino acid sequence set forth in any one of SEQ ID NO: 11-41.
E20. The mutant PapG polypeptide of embodiment E19, wherein the mutant PapG polypeptide comprises an amino acid sequence having at least 80% identity to the amino acid sequence set forth in any one of SEQ ID NO: 11-41.
E21. The mutant PapG polypeptide of embodiment E20, wherein the PapG polypeptide has at least 90%, 95, 96%, 97%, 98% or 99% identity to the amino acid sequence set forth in any one of SEQ ID NO: 11-41.
E22. The mutant PapG polypeptide according to any of embodiments E1-E21, wherein the polypeptide is isolated.
E23. A pharmaceutical composition comprising (i) the mutant PapG polypeptide according to any one of embodiments E1-E21 and (ii) a pharmaceutically acceptable carrier.
E24. An immunogenic composition comprising the mutant PapG polypeptide according to any one of embodiments E1-E21.
E25. An immunogenic composition according to embodiment E24, further comprising at least one additional antigen.
E26. An immunogenic composition according to embodiment E25, wherein the at least one additional antigen is a saccharide, or a polysaccharide, or a glycoconjugate, or a protein.
E27. An immunogenic composition according to embodiment E24, further comprising at least one adjuvant.
E28. A nucleic acid molecule comprising a nucleotide sequence that encodes an amino acid sequence of the mutant PapG polypeptide according to any one of embodiments E1-E21.
E29. the mutant PapG polypeptide according to any of embodiments E1-E22, wherein the polypeptide is immunogenic.
E30. A recombinant mammalian cell, comprising a polynucleotide encoding the mutant PapG polypeptide according to any one of embodiments E1-E21.
E31. A culture comprising the recombinant cell of embodiment E30, wherein said culture is at least 5 liters in size.
E32. A method for producing the mutant PapG polypeptide according to any one of embodiments E1-E21, comprising culturing a recombinant mammalian cell according to embodiment E30 under suitable conditions, thereby expressing the polypeptide; and harvesting the polypeptide.
E33. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of a composition according to any one of embodiments E23-E27.
E34. A method according to embodiment E33, wherein the subject is at risk of developing a urinary tract infection.
E35. A method according to embodiment E33, wherein the subject is at risk of developing bacteremia.
E36. A method according to embodiment E33, wherein the subject is at risk of developing sepsis, urosepsis, cystitis and/or pyelonephritis.
E37. A method of eliciting an immune response against E. coli in a mammal, comprising administering to the mammal an effective amount of a composition according to any one of embodiments E23-E29.
E38. A method according to embodiment E37, wherein the immune response comprises opsonophagocytic and/or neutralizing antibodies against E. coli.
E39. A method according to embodiment E37, wherein the immune response protects the mammal from an E. coli infection.
E40. A method of preventing, treating or ameliorating a bacterial infection, disease or condition in a subject, comprising administering to the subject an immunologically effective amount of a composition according to any one of embodiments E23-E28.
E41. A ribonucleic acid polynucleotide (RNA) molecule comprising at least one open reading frame (ORF) encoding a PapG antigenic polypeptide.
E42. The RNA molecule of embodiment E41, wherein the PapG antigenic polypeptide is a full-length, truncated, fragment or variant thereof.
E43. The RNA molecule of any one of embodiments E41 to E42, wherein the PapG antigenic polypeptide comprises at least one mutation relative to the amino acid sequence of a wild type PapG polypeptide.
E44. The RNA molecule of any one of embodiments E41 to E43, wherein the mutation is selected from the group consisting of: G18, G75, G86, S89, N96, G104, W107, G122, G147, G168, R170, K172, N24S, and N286, or any combination thereof, and wherein the amino acid positions are numbered according to SEQ ID NO: 120.
E45. The RNA molecule of embodiment E44, comprising at least one mutation selected from the group consisting of: G18A, G75A, G86A, S89T, N96S, G104A, W107A, G122A, G147A, G168A, R170A, K172A, N242S, and N286S, or any combination thereof.
E46. The RNA molecule of any one of embodiments E41-E45, wherein the PapG antigenic polypeptide comprises PapG_{DSF} (SEQ ID NO: 69), PapG_{DSF} mutant, PapG_{LD} mutant, or an immunogenic fragment thereof.
E47. The RNA molecule of any one of embodiments E41 to E46, wherein the PapG antigenic polypeptide comprises amino acids having an amino acid sequence as set forth in SEQ ID NO: 11 to 41.
E48. The RNA molecule of embodiment E47, wherein the PapG antigenic polypeptide comprises amino acids having an amino acid sequence as set forth in SEQ ID NO: 27 to 41.
E49. The RNA molecule of any one of embodiments E41 to E48, wherein the PapG polypeptide comprises an amino acid sequence having at least 80% identity to the amino acid sequence set forth in any one of SEQ ID NO: 11 to 41.
E50. The RNA molecule of any one of embodiments E41 to E49, wherein the PapG polypeptide comprises an amino acid sequence having at least 90%, 95, 96%, 97%, 98% or 99% identity to the amino acid sequence set forth in any one of SEQ ID NO: 11 to 41.
E51. The RNA molecule of any one of embodiments E41-E50, wherein the RNA is fused to a C-terminal membrane targeting domain.
E52. The RNA molecule of embodiment E51, wherein the RNA molecule and the C-terminal membrane targeting domain are separated by a linker.
E53. The RNA molecule of embodiment E52, wherein the linker has the amino acid sequence GGSSGGGGSSGSGSSSG (SEQ ID NO: 71), SSGGGGSSGSGSSSG (SEQ ID NO: 72), SSGGGGSSGSGSS (SEQ ID NO: 73), or SSGGG (SEQ ID NO: 74).
E54. The RNA molecule of any of embodiments E51-E53, wherein the C-terminal membrane targeting domain is derived from a viral glycoprotein.
E55. The RNA molecule of embodiment E54, wherein the viral glycoprotein is HSV gD (TMD).
E56. The RNA molecule of any of embodiments E51-E53, wherein the C-terminal membrane targeting domain is human Thyl-GPI or human DAF-GPI.
E57. The RNA molecule of any of embodiments E41-E56, wherein the open reading frame is codon-optimized.
E58. The RNA molecule of embodiment E57, wherein the PapG antigenic polypeptide comprises amino acids having an amino acid sequence as set forth in SEQ ID NO: 11-41.
E59. The RNA molecule of any one of embodiments E41 to E58, further comprising a 5′ untranslated region (5′ UTR).
E60. The RNA molecule of embodiment E59, wherein the 5′ UTR comprises nucleotides having SEQ ID NO: 1, 3, 5 or 7.
E61. The RNA molecule any one of embodiments E41 to E60, further comprising a 3′ untranslated region (3′ UTR).
E62. The RNA molecule of embodiment E61, wherein the 3′ UTR comprises nucleotides having SEQ ID NO: 2, 4, 6, 8 or 10.
E63. The RNA molecule of any one of embodiments E41 to E62, further comprising a 3′ poly-A tail.
E64. The RNA of embodiment E63, wherein the poly A tail comprises a sequence having SEQ ID NO: 75.
E65. The RNA molecule of any one of embodiments E41 to E64, wherein the RNA molecule comprises a 5′ UTR and a 3′ UTR.
E66. The RNA molecule of any one of embodiments E41 to E65, wherein the RNA molecule comprises a 5′ UTR, 3′ UTR, and poly-A tail.
E67. The RNA molecule of any one of embodiments E41 to E66, wherein the nucleic acid comprises nucleotides having a sequence as set forth in any one of SEQ ID NO: 42-66.
E68. The RNA molecule of any one of embodiments E41 to E67, wherein the RNA molecule further comprises a 5′ cap moiety.
E69. The RNA molecule of embodiment E68, wherein the 5′ cap moiety is m⁷G (5′)ppp(5′)(2′OMeA)pG.
E70. The RNA molecule of any of embodiments E41 to E69, wherein the RNA molecule comprises stabilized RNA.
E71. The RNA molecule of any one of embodiments E41 to E70, wherein the RNA comprises at least one modified nucleotide.
E72. The RNA molecule of embodiment E71, wherein the modified nucleotide is pseudouridine, 1-methyl-3′-pseudouridylyl, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, or 5-methoxyuridine OR 2′-O-methyl uridine.
E73. The RNA molecule of embodiment E72, wherein the modified nucleotide is 1-methyl-3′-pseudouridylyl.
E74. The RNA molecule of any one of embodiments E41 to E73, wherein the RNA is mRNA.
E75. A composition comprising the RNA molecule of any one of embodiments E41 to E74, wherein the RNA molecule is formulated in a lipid nanoparticle (RNA-LNP).
E76. The composition of embodiment E75, wherein lipid nanoparticle comprises at least one of a cationic lipid, a PEG-lipid, a neutral lipid, and a steroid or steroid analog.
E77. The composition of embodiment E76, wherein the lipid nanoparticle comprises a cationic lipid.
E78. The composition of embodiment E77, wherein the cationic lipid is (4-hydroxybutyl) azanediyl)bis(hexane-6,1-diyl)bis(2-hexyldecanoate) (ALC-0315).
E79. The composition of any one of embodiments E41 to E78, wherein the lipid nanoparticle comprises a PEG-lipid.
E80. The composition of embodiment E79, wherein the PEG-lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]—N,N-ditetradecylacetamide, glycol-lipids including PEG-c-DOMG, PEG-c-DMA, PEG-s-DMG,N-[(methoxy polyethylene glycol) 2000) carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy) propyl-1-O-((o-methoxy(polyethoxy)ethyl) butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl) carbamate or 2,3-di(tetradecanoxy)propyl-N-(u>-methoxy(polyethoxy)ethyl) carbamate.
E81. The composition of embodiment E80, wherein the PEG-lipid is 2-[(polyethylene glycol)-2000]—N,N-ditetradecylacetamide (ALC-0159).
E82. The composition of any one of embodiments E75-E81, wherein the lipid nanoparticle comprises a neutral lipid.
E83. The composition of embodiment E82, wherein the neutral lipid is (DSPC), dioleoylphosphatidylcholine (DOPC), distearoylphosphatidylcholine dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).
E84. The composition of embodiment E83, wherein the neutral lipid is 1,2-distearoyl-sn-glycero-3-phosphocholine (DSPC).
E85. The composition of any one of embodiments E75 to E84, wherein the lipid nanoparticle comprises a steroid or steroid analog.
E86. The composition of embodiment E85, wherein the steroid or steroid analog is cholesterol.
E87. The composition of any one of embodiments E75 to E86, wherein lipid nanoparticle wherein has a mean diameter of about 1 to about 500 nm.
E88. The composition of any one of embodiments E75 to E87, wherein the composition is a vaccine.
E89. The composition of any one of embodiments E75 to E88, wherein the lipid nanoparticle size is at least 40 nm.
E90. The composition of any one of embodiments E75 to E89, wherein the lipid nanoparticle size is at most 180 nm.
E91. The composition of any one of embodiments E75 to E90, wherein the composition is a vaccine.
E92. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of the RNA molecule, RNA-LNP and/or vaccine of any one of embodiments E41 to E91.
E93. The method of embodiment E92, wherein the subject is at risk of developing a urinary tract infection.
E94. The method of embodiment E92, wherein the subject is at risk of developing bacteremia.
E95. The method of embodiment E92, wherein the subject is at risk of developing sepsis or urosepsis.
E96. The method of embodiment E92, wherein the subject is at risk of developing cystitis.
E97. The method of embodiment E92, wherein the subject is at risk of developing pyelonephritis.
E98. Use of the RNA molecule, RNA-LNP and/or composition of any one of embodiments E41 to E91 in the manufacture of a medicament for use in (i) inducing an immune response in a subject against extra-intestinal pathogenic E. coli, or (ii) inducing the production of opsonophagocytic and/or neutralizing antibodies in a subject that are specific to extra-intestinal pathogenic E. coli.
E99. The use of embodiment E98, wherein the infection, disease or condition is a urinary tract infection.
E100. The use of embodiment E98, wherein the subject is at risk of developing bacteremia.
E101. The use of embodiment E98, wherein the subject is at risk of developing sepsis or urosepsis.
E102. The use of embodiment E98, wherein the subject is at risk of developing cystitis.
E103. The use of embodiment E102, wherein the subject is at risk of developing pyelonephritis.
E104. The method or use of any one of embodiments E92 to E103, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older, from about 18 years of age to 49 years of age, or from 50 years of age to 64 years of age.
E105. The method or use of embodiment E104, wherein the subject is about 50 years of age or older.
E106. The method or use of embodiment E104, wherein the subject is a pregnant woman.
E107. The method or use of any one of embodiments E104 to E106, wherein the RNA molecule or composition is administered as a vaccine.
E108. The method or use of any one of embodiments E104 to E107, wherein the RNA molecule or composition is administered by intradermal or intramuscular injection.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

### I. Examples of Definitions

Unless otherwise defined herein, scientific and technical terms used in connection with the present invention have the meanings that are commonly understood by those of ordinary skill in the art.

Throughout this application, the term “about” is used according to its plain and ordinary meaning in the area of cell and molecular biology to indicate a deviation of +10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) identity to a wild-type PapG over the entire length of the sequence; or a nucleotide sequence complementary to said isolated polynucleotide. In some aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

In certain aspects, the isolated polypeptide will comprise an amino acid sequence encoding a polypeptide that has at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) identity to a wild-type PapG over the entire length of the sequence. In some aspects, the isolated polypeptide will comprise an amino acid sequence encoding a polypeptide that has at least 90% identity to an amino acid sequence described herein, over the entire length of the sequence. In some aspects, the isolated polypeptide will comprise an amino acid sequence encoding a polypeptide that has at least 95% identity to an amino acid sequence described herein, over the entire length of the sequence.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1)production of an RNA template from a DNA sequence (e.g., by transcription); (2)processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a 3-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine (denoted by the symbol m¹⁴′) in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease. In specific aspects, the pathogenic are peptide or protein antigens derived from E. coli PapG. Conditions and/or diseases that may be treated with RNA disclosed herein include, but are not limited to, those caused and/or impacted by bacterial infection. Such bacteria include, but are not limited to, E. coli.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified)protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. The term “mutant” of a wild-type E. coli PapG protein, “mutant” of a E. coli PapG protein, “E. coli PapG protein mutant,” or “modified E. coli PapG protein” refers to a polypeptide that displays introduced mutations relative to a wild-type PapG protein and is immunogenic against the wild-type PapG protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a bacteria). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more prophylactic/therapeutic regimens (e.g., two or more prophylactic/therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity). In one aspect of this invention, a combination therapy comprises one or more fimbrial antigens, for example FimH, PapG and/or FmIH or mutants thereof, in either protein subunit or RNA compositions. In one embodiment, the fimbrial antigen includes a mutant of a wildtype FimH protein and/or RNA as described in PCT International Publication No. WO2022137078, WO2023111907, and U.S. Provisional Application No. 63/508,060, file Jun. 14, 2023, which are herein incorporated by reference in their entireties for all purposes. In another embodiment, the combination therapy further comprises an E. coli or K.pneumoniae O-antigen conjugate as described in PCT International Publication No. WO2020/039359, WO2021/084429, WO2021/165928, and WO2022090893, which are herein incorporated by reference in their entireties for all purposes. In a further embodiment, the combination therapy further comprises an adjuvant including, but not limited to, Lina-2, which is described in any one of the patent publications/applications listed above, and also PCT International Publication No. WO2023111907, and PCT Internation AppIn No. PCT/IB2023/062031, which which are herein incorporated by reference in their entireties for all purposes.

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

II. E. coli Fimbrial Antigen PapG

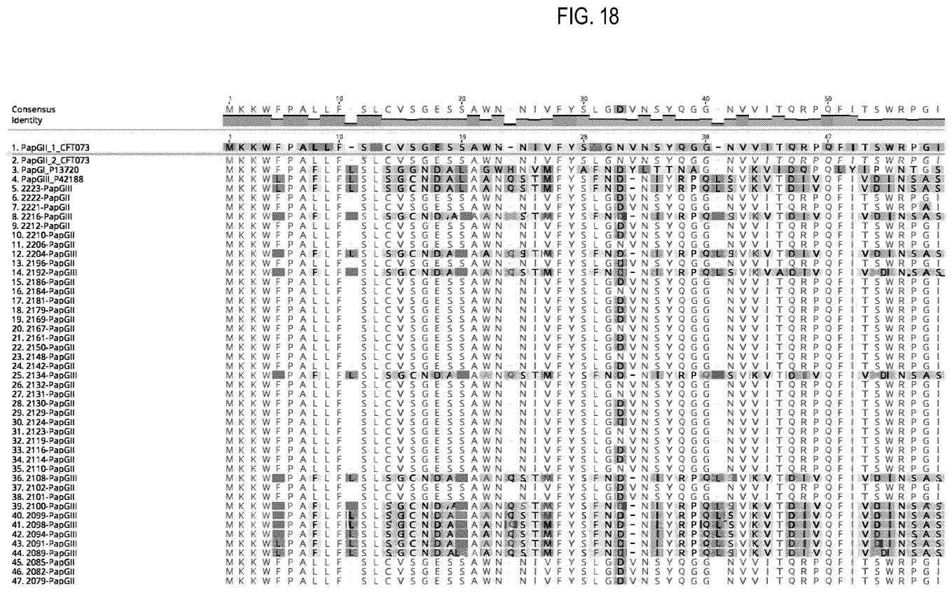

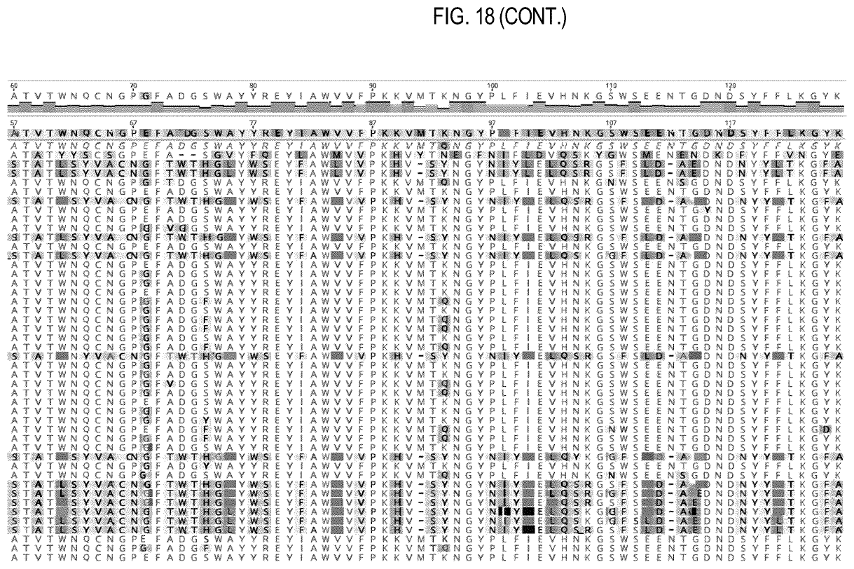

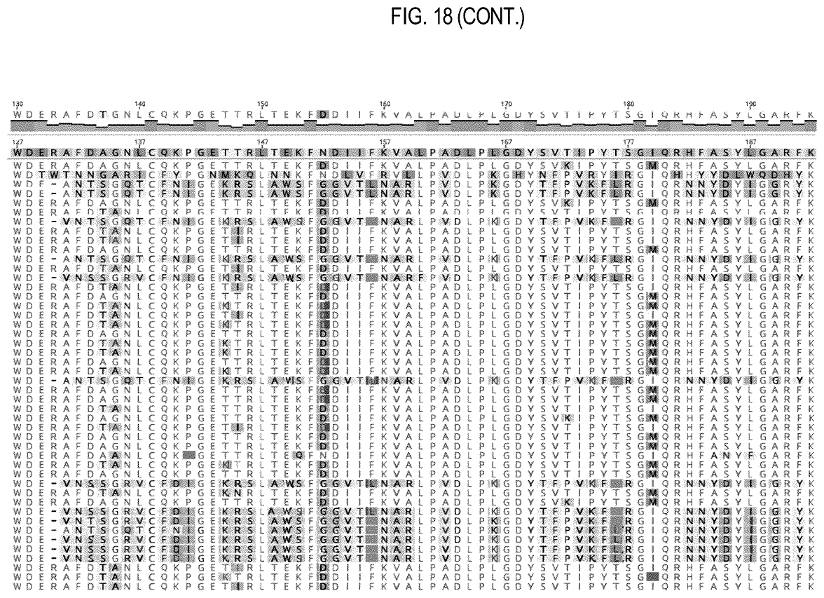

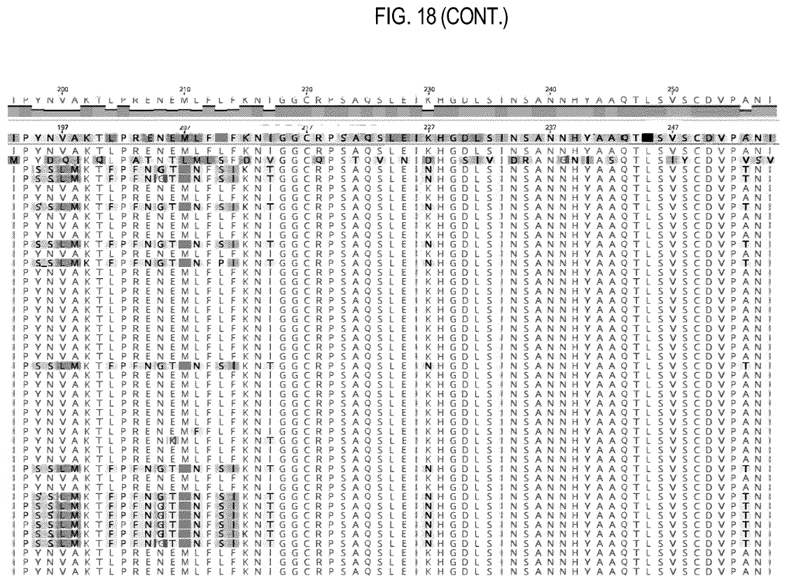

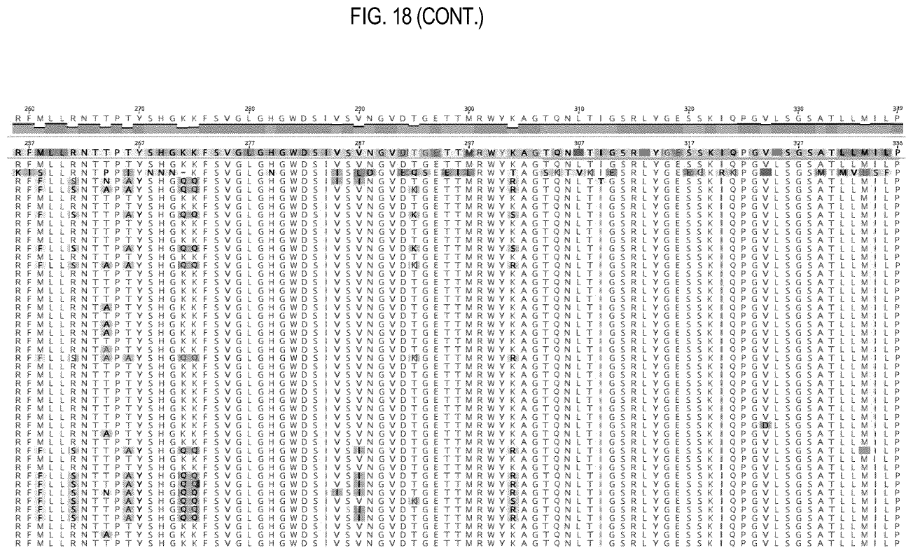

The PapGII polypeptide sequence from the CFT073 strain was compared to PapG sequences from 44 clinical UPEC isolates and showed 56-99% identity (see SEQ ID Nos: 123-165 in Table 13). The polypeptide sequence alignment of these sequences is shown in FIG. 18. Percent identity between PapG II version 1 (SEQ ID NO: 120) and PapG II version 2 (SEQ ID NO: 121) is 99%. Percent identity between PapG I (SEQ ID NO: 119) and PapG II version 2 (SEQ ID NO: 121) is 80%. Percent identity between PapG II version 2 (SEQ ID NO: 121) and PapG III (SEQ ID NO: 122) is 80%. Percent identity between PapG I (SEQ ID NO: 119) and PapG III (SEQ ID NO: 122) is 79%. In one aspect, the mutant PapG polypeptide described herein comprises an amino acid sequence having at least, at most, exactly, or between any two of 99%, 98%, 97%, 96%, 95%, 90%, 85%, 80% or 79% identity to SEQ ID NO: 120. In another aspect, the mutant PapG polypeptide described herein comprises an amino acid sequence having at least, at most, exactly, or between any two of 99%, 98%, 97%, 96%, 95%, 94%, 93%, 92%, 91%, 90%, 89%, 88%, 87%, 86%, 85%, 84%, 83%, 82%, 81%, 80% or 79% identity to SEQ ID NO: 120.

As used herein, the terms “PapG antigenic polypeptide” or “PapG polypeptide” may be used interchangeably and include any PapG polypeptide or immunogenic mutant thereof, including but not limited to, the PapG polypeptides set forth in SEQ ID NOs: 11-41 and SEQ ID Nos: 119-122. In one aspect, the mutant PapG polypeptide described herein comprises an amino acid sequence having at least, at most, exactly, or between any two of 99%, 98%, 97%, 96%, 95%, 90%, 85%, 80% or 79% identity to any one of SEQ ID NOs: 11-41. In one aspect, the mutant PapG polypeptide described herein comprises an amino acid sequence having at least, at most, exactly, or between any two of 99%, 98%, 97%, 96%, 95%, 90%, 85%, 80% or 79% identity to any one of SEQ ID NOs: 11-41. In another aspect, the mutant PapG polypeptide described herein comprises an amino acid sequence having at least 80% identity to any one of SEQ ID NOs: 11-41.

As used herein, the term “E. coli polypeptide” includes any E. coli polypeptide. In a preferred embodiment, the E. coli polypeptide is a fimbrial antigen. In a preferred embodiment, the E. coli fimbrial antigen is PapG.

Embodiments of the present disclosure provide RNA (e.g., mRNA) vaccines that comprise polynucleotides encoding an E. coli PapG antigenic polypeptide. E. coli PapG RNA vaccines, as provided herein may be used to induce a balanced immune response, comprising both cellular and humoral immunity.

Some embodiments provide E. coli vaccines comprising one or more RNA polynucleotides having an open reading frame encoding a PapG antigenic polypeptide and a pharmaceutically acceptable carrier or excipient, formulated within a cationic lipid nanoparticle. In some embodiments, the PapG antigenic polypeptide is selected from PapG_{DSF}, a PapG_{DSF} mutant or a PapG_{LD} mutant as described in Tables 10-12.

Some embodiments provide methods of preventing or treating E. coli infection comprising administering to a subject any of the vaccines described herein. In some embodiments, the antigen specific immune response comprises a T cell response. In some embodiments, the antigen specific immune response comprises a B cell response. In some embodiments, the antigen specific immune response comprises both a T cell response and a B cell response. In some embodiments, the method of producing an antigen specific immune response involves a single administration of the vaccine. In some embodiments, the vaccine is administered to the subject by intradermal, intramuscular injection, subcutaneous injection, intranasal inoculation, or oral administration.

In some embodiments, the RNA (e.g., mRNA) polynucleotides or portions thereof may encode one or more polypeptides or fragments thereof of E. coli PapG as an antigen.

### III. RNA Molecule

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) OFRs, which may be a sequence of codons that is translatable into a polypeptide or protein of interest.

A number of mRNA vaccine platforms are available in the prior art. The basic structure of in vitro transcribed (IVT) mRNA closely resembles “mature” eukaryotic mRNA and consists of (i) a protein-encoding open reading frame (ORF), flanked by (ii) 5′ and 3′ untranslated regions (UTRs), and at the end sides (iii) a 7-methyl guanosine 5′ cap structure and (iv) a 3′ poly(A) tail. The non-coding structural features play important roles in the pharmacology of mRNA and can be individually optimized to modulate the mRNA stability, translation efficiency, and immunogenicity.

By incorporating modified nucleosides, mRNA transcripts referred to as “nucleoside-modified mRNA” or “modRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antibacterial pathways that are induced by type IFNs and are programmed to degrade and inhibit invading mRNA. For instance, the replacement of uridine with pseudouridine (denoted by the symbol 4) or N1-methylpseudouridine (denoted by the symbol m¹⁴′) in in vitro transcribed (IVT) mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleosides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the invention relates to an immunogenic composition comprising an mRNA molecule that encodes one or more polypeptides or fragments thereof of E. coli PapG as an antigen. In some embodiments, the mRNA molecule comprises a nucleoside-modified mRNA.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence. In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA.

In some aspects, the RNA molecule includes from about 20 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides.

mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, the mRNA of the invention further includes a poly-A region or a Kozak sequence (e.g., in the 5′-UTR). In some cases, mRNA of the invention may contain one or more intronic nucleotide sequences capable of being excised from the polynucleotide. In some embodiments, mRNA of the invention may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine).

In some embodiments, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a payload (e.g., an E. coli PapG protein); a 3′ untranslated region (3′ UTR); and a Poly-A sequence.

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 6.0:1, about 6.5:1, or about 7.0:1.

a. Modified Nucleobases

In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

mRNA of the invention may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the invention may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity. mRNA of the invention may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof. mRNA of the invention may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may be any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, e.g., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thiopseudouridine (s⁴ψ), 2-thiopseudouridine (s²ψ), 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nmVu), 5-methylaminomethyl-uracil (mnm⁵U), 5-methylaminomethyl-2-thio-uracil (mnmVu), 5-methylaminomethyl-2-seleno-uracil (mnm⁵se2U), 5-carbamoylmethyl-uracil (ncm⁵U), 5-carboxymethylaminomethyl-uracil (cmnm⁵U), 5-carboxymethylaminomethyl-2-thio-uracil (cmnmVu), 5-propynyl-uracil, 1-propynyl-pseudouracil, 5-taurinomethyl-uracil (xm⁵U), 1-taurinomethyl-pseudouridine, 5-taurinomethyl-2-thio-uracil (xm⁵s²U), 1-taurinomethyl-4-thio-pseudouridine, 5-methyl-uracil (m⁵U, e.g., having the nucleobase deoxythymine), 1-methyl-pseudouridine (mV), 5-methyl-2-thio-uracil (m⁵s²U), 1-methyl-4-thio-pseudouridine (ms⁴ψ), 4-thio-1-methyl-pseudouridine, 3-methyl-pseudouridine (m \|/), 2-thio-1-methyl-pseudouridine, 1-methyl-1-deaza-pseudouridine, 2-thio-I-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine (m¹ψ), 3-(3-amino-3-carboxypropyl) uracil (acpU), I-methyl-3-(3-amino-3-carboxypropyl) pseudouridine (acp ψ), 5-(isopentenylaminomethyl) uracil (inm⁵U), 5-(isopentenylaminomethyl)-2-thio-uracil (inm⁵s²U), 5,2′-O-dimethyl-uridine (m⁵Um), 2-thio-2′-O_methyl-uridine (s²Um), 5-methoxycarbonylmethyl-2′-O-methyl-uridine (mem Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (mUm), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)] uracil. Pseudouridine is one example of a modified nucleoside that is an isomer of uridine, where the uracil is attached to the pentose ring via a carbon-carbon bond instead of a nitrogen-carbon glycosidic bond.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm⁵C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methy 1-pseudoisocytidine, 4-thio-1-methyl-1-deaza-pseudoisocytidine, 1-methyl-1-deaza-pseudoisocytidine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4, N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (ml A), 2-methyl-adenine (m2A), N6-methyl-adenine (m6A), 2-methylthio-N6-methyl-adenine (ms²m6A), N6-isopentenyl-adenine (16A), 2-methylthio-N6-isopentenyl-adenine (ms²i6A), N6-(cis-hydroxyisopentenyl) adenine (io6A), 2-methylthio-N6-(cis-hydroxyisopentenyl) adenine (ms²i06A), N6-glycinylcarbamoyl-adenine (g6A), N6-threonylcarbamoyl-adenine (16A), N6-methyl-N6-threonylcarbamoyl-adenine (m6t6A), 2-methylthio-N6-threonylcarbamoyl-adenine (ms²g6A), N6,N6-dimethyl-adenine (m62A), N6-hydroxynorvalylcarbamoyl-adenine (hn6A), 2-methylthio-N6-hydroxynorvalylcarbamoyl-adenine (ms²hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (02yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQI), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2, N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2, N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (mlGm), N2,7-dimethyl-2′-O-methyl-guanosine (m2,7Gm), 2′-O-methyl-inosine (Im), 1,2′-O-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a] 1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

In some aspects, the RNA molecule comprises a nucleic acid sequence having at least one uridine replaced by pseudouridine, 1-methyl-3′-pseudouridylyl or N1-methylpseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by pseudouridine, 1-methyl-3′-pseudouridylyl or N1-methylpseudouridine. In some aspects, the RNA molecule comprises a nucleic acid sequence having all uridines replaced by pseudouridine, 1-methyl-3′-pseudouridylyl or N1-methylpseudouridine.

### B. 5′ CAP

The mRNA may include a 5 ‘-cap structure. The 5’-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5 ‘end of the polynucleotide may optionally also be 2’—O-methylated. 5′-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as α-methyl-phosphonate and seleno-phosphate nucleotides. Additional alterations include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5 ‘-cap structures can be used to generate the 5’-cap of an mRNA molecule.

Cap analogs, which herein are also referred to as synthetic cap analogs, chemical caps, chemical cap analogs, or structural or functional cap analogs, differ from natural (e.g., endogenous, wild-type, or physiological) 5 ‘-caps in their chemical structure, while retaining cap function. Cap analogs may be chemically (e.g., non-enzymatically) or enzymatically synthesized and/linked to a polynucleotide. For example, the Anti-Reverse Cap Analog (ARCA) cap contains two guanosines linked by a 5’-5 ‘-triphosphate group, wherein one guanosine contains an N7-methyl group as well as a 3’—O-methyl group (e.g., N7, ‘—O-dimethyl-guanosine-5’-triphosphate-5 ‘-guanosine, m7G-3′mppp-G, which may equivalently be designated 3’ 0-Me-m7G (5′)ppp(5′) G). The 3′-0 atom of the other, unaltered, guanosine becomes linked to the 5 ‘-terminal nucleotide of the capped polynucleotide (e.g., an mRNA). The N7- and 3’—O-methylated guanosine provides the terminal moiety of the capped polynucleotide (e.g., mRNA). Another exemplary cap is mCAP, which is similar to ARCA but has a 2′-O-methyl group on guanosine (e.g., N7,2′-O-dimethyl-guanosine-5 ‘-triphosphate-5’-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G (5)ppp(5 ‘) G and a N7-(4-chlorophenoxyethyl)-m3’-OG (5)ppp(5′) G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5 ‘-cap structures useful in the polynucleotides of the present disclosure are those which, among other things, have enhanced binding of cap binding proteins, increased half-life, reduced susceptibility to 5’-endonucleases, and/or reduced 5′-decapping, as compared to synthetic 5 ‘-cap structures known in the art (or to a wild-type, natural or physiological 5’-cap structure). For example, recombinant Vaccinia Virus Capping Enzyme and recombinant 2′-O-methyltransferase enzyme can create a canonical 5 ‘-5’-triphosphate linkage between the 5 ‘-terminal nucleotide of a polynucleotide and a guanosine cap nucleotide wherein the cap guanosine contains an N7-methylation and the 5’-terminal nucleotide of the polynucleotide contains a 2′-O-methyl. Such a structure is termed the Capl structure. This cap results in a higher translational-competency, cellular stability, and a reduced activation of cellular pro-inflammatory cytokines, as compared, e.g., to other 5′ cap analog structures known in the art. Other exemplary cap structures include 7 mG(5 ‘)ppp(5’)N,pN2p (Cap 0), 7 mG(5 ‘)ppp(5’)NImpNp (Cap 1), 7 mG(5 ‘)-ppp(5’)NImpN2mp(Cap 2), and m(7)Gpppm(3)(6,6,2′)Apm(2′)Apm(2′)Cpm(2)(3,2′)Up(Cap 4).

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction.

5 ‘-terminal caps may include endogenous caps or cap analogs. A 5’-terminal cap may include a guanosine analog. Useful guanosine analogs include inosine, NI-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5 ‘-cap. A modification on the 5’-cap may increase the stability of polynucleotide, increase the half-life of the polynucleotide, and could increase the polynucleotide translational efficiency. The modified 5 ‘-cap may include, but is not limited to, one or more of the following modifications: modification at the 2’- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

### C. Untranslated Regions (UTRs)

The 5′ UTR is a regulatory region situated at the 5′ end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a β-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

To alter one or more properties of an mRNA, 5 ‘UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5’ UTR may have on the mRNA. Variants of the 5′UTRs may be utilized wherein one or more nucleotides are added or removed to the termini, including A, T, C or G. 5′UTRs may also be codon-optimized, or altered in any manner described herein.

In some aspects, the RNA molecule includes a 5′ untranslated region (5′-UTR). In some aspects, the 5′ UTR comprises a sequence selected from any of SEQ ID NO: 1, 3, 5, 7 or 9. In some aspects, the 5′ UTR comprises a sequence having at least 90%, 91%, 92%, 93%, 94%, 95, 96%, 97%, 98% or 99% or higher identity to any of SEQ ID NO: 1, 3, 5, 7 or 9. In some aspects, the 5′ UTR comprises a sequence selected from any of SEQ ID NO: 3, 5 or 7. In some aspects, the 5′ UTR comprises a sequence consisting of any of SEQ ID NO: 3, 5 or 7.

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a Xhol site.

In some aspects, the RNA molecules and RNA-LNPs include a 3′ untranslated region (3′-UTR). In some aspects, the 3′ UTR comprises a sequence selected from any of SEQ ID NO: 2, 4, 6, 8, or 10. In some aspects, the 3′ UTR comprises a sequence having at least 90%, 91%, 92%, 93%, 94%, 95, 96%, 97%, 98% or 99% or higher identity to any of SEQ ID NO: 2, 4, 6, 8 or 10. In some aspects, the 3′ UTR comprises a sequence selected from any of SEQ ID NO: 4, 6, 8 or 10. In some aspects, the 3′ UTR comprises a sequence consisting of any of SEQ ID NO: 4, 6, 8 or 10.

mRNAs may include a stem loop such as, but not limited to, a histone stem loop. The stem loop may be a nucleotide sequence that is about 25 or about 26 nucleotides in length. The histone stem loop may be located 3 ‘-relative to the coding region (e.g., at the 3’-terminus of the coding region). As a non-limiting example, the stem loop may be located at the 3 ‘-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3’-UTR) in a second terminal region. In some cases, a mRNA which includes the histone stem loop may be stabilized by the addition of a 3 ‘-stabilizing region (e.g., a 3’-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3 ‘-region of the polynucleotide that can prevent and/or inhibit the addition of oligio (U). In yet other cases, a mRNA, which includes the histone stem loop may be stabilized by the addition of an oligonucleotide that terminates in a 3’-deoxynucleoside, 2′,3 ‘-dideoxynucleoside 3’—O-methylnucleosides, 3-O-ethylnucleosides, 3 ‘-arabinosides, and other alternative nucleosides known in the art and/or described herein. In some instances, the mRNA of the present disclosure may include a histone stem loop, a poly-A region, and/or a 5’-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5 ‘-cap structure. The 5’-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

### D. Open Reading Frame (ORF)

The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, which are known in the art. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

The present disclosure provides for an RNA molecule comprising at least one open reading frame encoding an E. coli PapG polypeptide as described herein. In some aspects, an RNA molecule comprising at least one open reading frame encoding an E. coli PapG protein as described herein.

### E. Genes of Interest

The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. The sequence for a particular gene of interest is readily identified by one of skill in the art using public and private databases, e.g., GENBANK®.

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

The term “antigen” may refer to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g. an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from E. coli fimbrial antigen PapG.

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

In some aspects, the RNA molecule encodes a PapG protein comprising the sequence of any one of SEQ ID NOs: 11-41, or a fragment or variant thereof.

In some aspects, the RNA molecule encodes an E. coli PapG protein synthesized from the nucleic acid sequence comprising any one of SEQ ID NOs: 42-66, or fragment or variant thereof.

### F. Poly-A Tail

In some aspects, RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 30, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10, 20, 30, 40, 50, 60, 70, 80, 90 or 100% greater in length than the additional feature. The poly-A region may also be designed as a fraction of the alternative polynucleotide to which it belongs. In this context, the poly-A region may be 10, 20, 30, 40, 50, 60, 70, 80, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3 ‘-deoxyadenosine (cordycepin), 3’-deoxyuridine, 3′-deoxycytosine, 3′-deoxyguanosine, 3′-deoxy thymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligio (U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

In one aspect, an RNA disclosed herein comprises a poly-A tail comprising a sequence having at least, at most, exactly, or between any two of 99%, 98%, 97%, 96%, 95%, 90%, 85%, or 80% identity to SEQ ID NO: 75. In one aspects, the poly-A tail comprises a sequence of SEQ ID NO: 75.

### IV. RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include, e.g., but are not limited to mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, RNase inhibitor, pyrophosphatase, and/or T7 RNA polymerase. In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor may contain at least, at most, exactly, or between any two of 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration at least, at most, exactly, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg2+, Na+, Ca2+), RNA fragments, residual nucleotides, free phosphates, or any combinations thereof.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for the ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to at least, at most, exactly, or between any two of 30 kDa, 40 kDa, 50 kDa, 60 kDa, 70 kDa, 80 kDa, 90 kDa, or more. In some aspects, a TFF membrane may have a MWCO equal to at least, at most, exactly, or between any two of 100 kDa, 150 kDa, 200 kDa, 250 kDa, 300 kDa, 350 kDa, 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to at least, at most, exactly, or between any two of 2, 3, 4, 5, 6, 7, 8, 9, 10, or more. In some aspects, the second diafiltration step is conducted with diavolumes equal to at least, at most, exactly, or between any two of 5, 6, 7, 8, 9, 10, 11, 12, 13, 14, 15, 16, 17, 18, 19, 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to at least, at most, exactly, or between any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size of at least, at most, exactly, or between any two of 0.2 um, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

### V. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs may be designed to protect RNA molecules (e.g., mRNA) from extracellular RNases and/or may be engineered for systemic delivery of the RNA to target cells. In some aspects, such LNPs may be particularly useful to deliver RNA molecules (e.g., mRNA, modRNA) when RNA molecules are intravenously administered to a subject in need thereof. In some aspects, such LNPs may be particularly useful to deliver RNA molecules (e.g., mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/ml to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., an E. coli PapG protein) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that may be used to deliver an active agent or therapeutic agent, such as a nucleic acid (e.g., mRNA, modRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA, modRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA, modRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, provided RNA molecules (e.g., mRNA, modRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” “size” or “mean size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have, have at least, or have at least, at most, exactly, or between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein may be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than it is achieved in the absence of interactions between nucleic acids and at least one of the lipid components.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (e.g. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

a. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order;

alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

### B. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, the LNPs encompass, or encapsulate, the nucleic acid molecules.

i. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may be at least, at most, exactly, or between any two of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle. Examples of cationic lipids include, but are not limited to: ((4-hydroxybutyl) azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl) cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N, N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N, N-dimethyl-I-propanamium trifluoroacetate (DOSPA), 1,2-dilinoleyloxy-N, N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-I-(cis, cis-9′, 12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DM A), N-(2-Hydroxyethyl)-N, N-dimethyl-2,3-bis(tetradecyloxy)-1-propanaminium bromide (DMRIE), (+)—N-(3-aminopropyl)-N,N-dimethyl-2,3-bis(cis-9-tetradecenyloxy)-1-propanaminium bromide (GAP-DMORIE), (+)—N-(3-aminopropyl)-N,N-dimethyl-2,3-bis(dodecyloxy)-1-propanaminium bromide (GAP-DLRIE), (+)—N-(3-aminopropyl)-N,N-dimethyl-2,3-bis(tetradecyloxy)-I-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2-({8-[(3b)-cholest-5-en-3-yloxy]octyl}oxy)-N,N-dimethyl-3-[(9Z, 12Z)-octadeca-9, 12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di [oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N, N-dimethyl-2,3-bis(tetradecyloxy)propan-1-aminium bromide (DMORIE), di((Z)-non-2-en-I-yl) 8,8′-((((2 (dimethylamino)ethyl)thio) carbonyl) azanediyl) dioctanoate (ATX), N,N-dimethyl-2,3-bis(dodecyloxy)propan-1-amine (DLDMA), N, N-dimethyl-2,3-bis(tetradecyloxy)propan-1-amine (DMDMA), Di((Z)-non-2-en-I-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis (2 hydroxydodecyl)amino]ethyl]piperazin-|-yl]ethyl]amino]dodecan-2-ol (lipidoid 02-200); or heptadecan-9-yl 8-((2-hydroxyethyl) (6-oxo-6-(undecyloxy) hexyl)amino) octanoate (SM-102).

In some aspects, the lipid nanoparticles comprise one or more cationic lipids. In one aspect, the lipid nanoparticles comprise (4-hydroxybutyl) azanediyl)bis(hexane-6,1-diyl)bis(2-hexyldecanoate) (ALC-0315), having the formula:

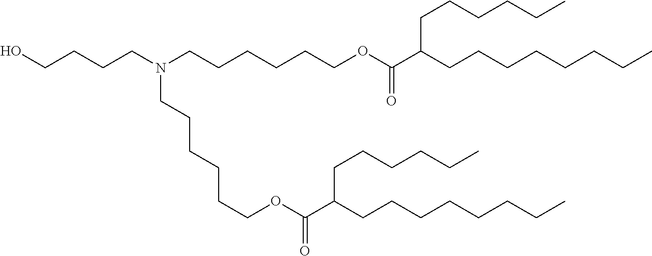 [SMILES: CCCCCCCCC(CCCCCC)C(=O)OCCCCCCN(CCCCO)CCCCCCOC(=O)C(CCCCCC)CCCCCCCC]

Cationic lipids are disclosed in, e.g., U.S. Pat. No. 10,166,298, the full disclosures of which are herein incorporated by reference in their entirety for all purposes. Representative cationic lipids include:

| No. | Structure |
|---|---|
| 1 | 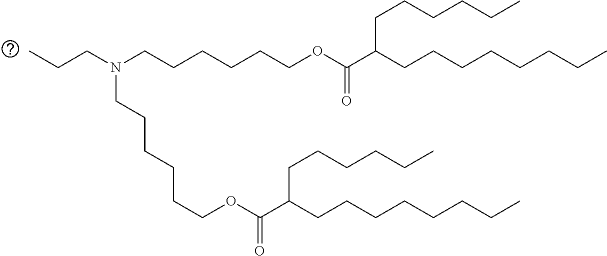 [SMILES: CCCCCCCCC(CCCCCC)C(=O)OCCCCCCN(CCC)CCCCCCOC(=O)C(CCCCCC)CCCCCCCC] |
| 2 | 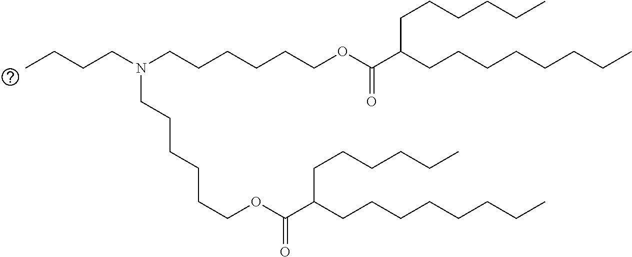 [SMILES: CCCCCCCCC(CCCCCC)C(=O)OCCCCCCN(CCCC)CCCCCCOC(=O)C(CCCCCC)CCCCCCCC] |
| 3 | 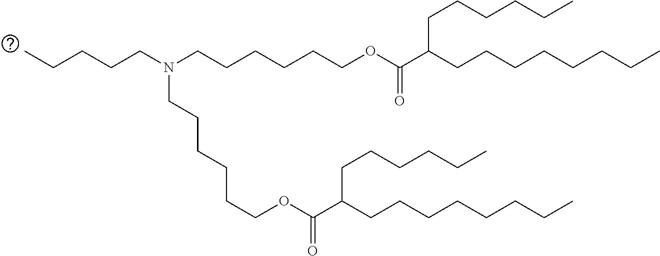 [SMILES: CCCCCCCCC(CCCCCC)C(=O)OCCCCCCN(CCCCC)CCCCCCOC(=O)C(CCCCCC)CCCCCCCC] |
| 4 | 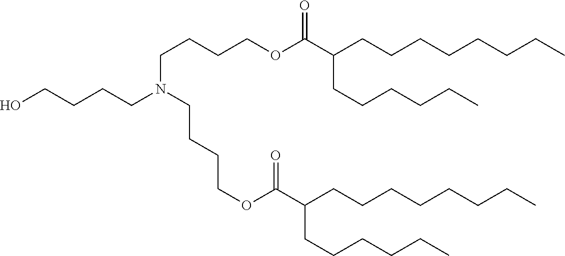 [SMILES: CCCCCCCCC(CCCCCC)C(=O)OCCCCN(CCCCO)CCCCOC(=O)C(CCCCCC)CCCCCCCC] |
| 5 | 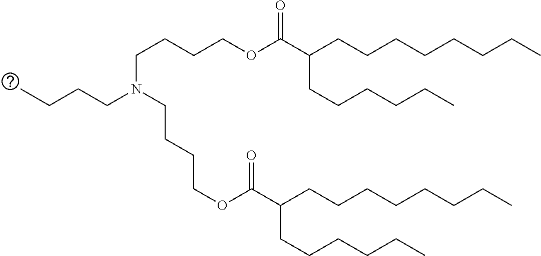 [SMILES: CCCCCCCCC(CCCCCC)C(=O)OCCCCN(CCCC)CCCCOC(=O)C(CCCCCC)CCCCCCCC] |
| 6 | 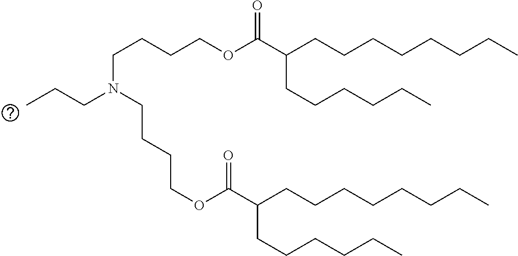 [SMILES: CCCCCCCCC(CCCCCC)C(=O)OCCCCN(CCC)CCCCOC(=O)C(CCCCCC)CCCCCCCC] |
| 7 | 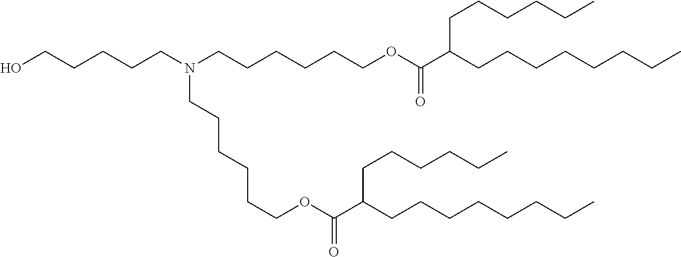 [SMILES: CCCCCCCCC(CCCCCC)C(=O)OCCCCCCN(CCCCCO)CCCCCCOC(=O)C(CCCCCC)CCCCCCCC] |
| 8 | 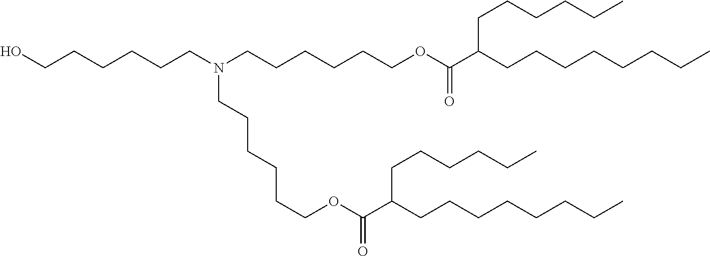 [SMILES: CCCCCCCCC(CCCCCC)C(=O)OCCCCCCN(CCCCCCO)CCCCCCOC(=O)C(CCCCCC)CCCCCCCC] |
| 9 | 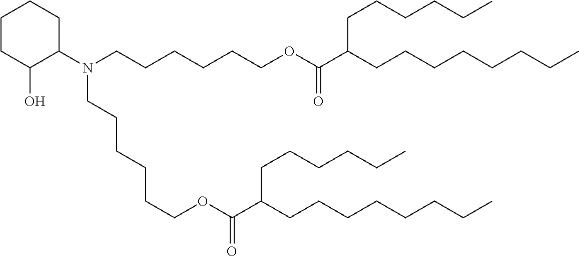 [SMILES: CCCCCCCCC(CCCCCC)C(=O)OCCCCCCN(CCCCCCOC(=O)C(CCCCCC)CCCCCCCC)C1CCCCC1O] |
| 10 | 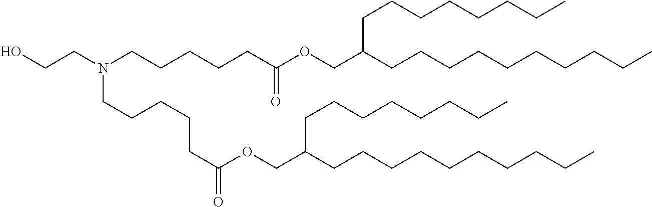 [SMILES: CCCCCCCCCCC(CCCCCCCC)COC(=O)CCCCCN(CCO)CCCCCC(=O)OCC(CCCCCCCC)CCCCCCCCCC] |
| 11 | 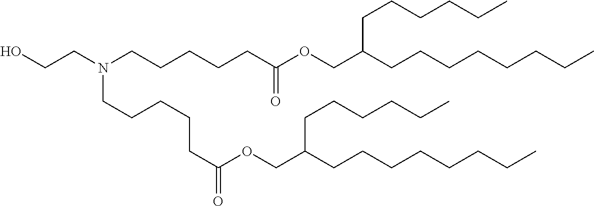 [SMILES: CCCCCCCCC(CCCCCC)COC(=O)CCCCCN(CCO)CCCCCC(=O)OCC(CCCCCC)CCCCCCCC] |
| 12 | 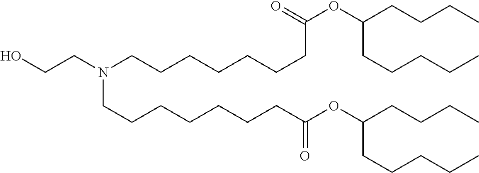 [SMILES: CCCCCC(CCCC)OC(=O)CCCCCCCN(CCO)CCCCCCCC(=O)OC(CCCC)CCCCC] |
| 13 | 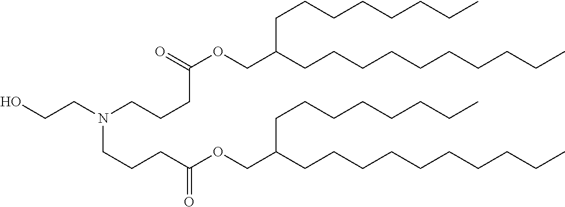 [SMILES: CCCCCCCCCCC(CCCCCCCC)COC(=O)CCCN(CCO)CCCC(=O)OCC(CCCCCCCC)CCCCCCCCCC] |
| 14 | 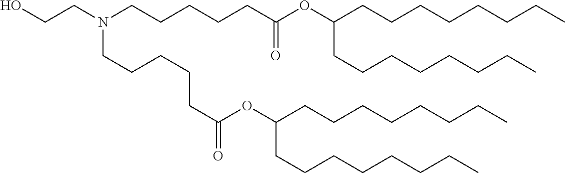 [SMILES: CCCCCCCCC(CCCCCCCC)OC(=O)CCCCCN(CCO)CCCCCC(=O)OC(CCCCCCCC)CCCCCCCC] |
| 15 | 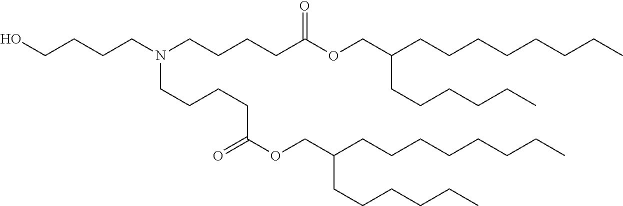 [SMILES: CCCCCCCCC(CCCCCC)COC(=O)CCCCN(CCCCO)CCCCC(=O)OCC(CCCCCC)CCCCCCCC] |
| 16 | 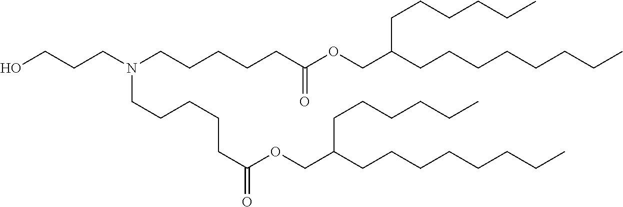 [SMILES: CCCCCCCCC(CCCCCC)COC(=O)CCCCCN(CCCO)CCCCCC(=O)OCC(CCCCCC)CCCCCCCC] |
| 17 | 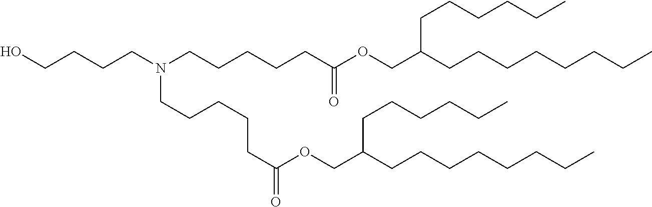 [SMILES: CCCCCCCCC(CCCCCC)COC(=O)CCCCCN(CCCCO)CCCCCC(=O)OCC(CCCCCC)CCCCCCCC] |
| 18 | 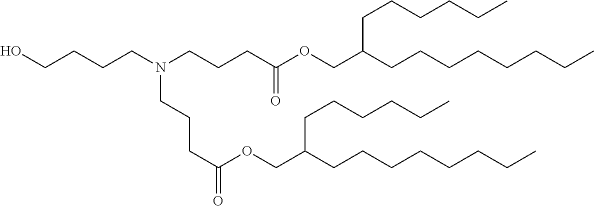 [SMILES: CCCCCCCCC(CCCCCC)COC(=O)CCCN(CCCCO)CCCC(=O)OCC(CCCCCC)CCCCCCCC] |
| 19 | 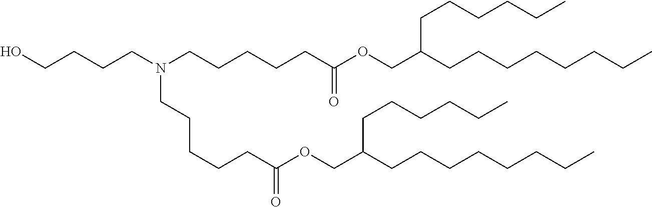 [SMILES: CCCCCCCCC(CCCCCC)COC(=O)CCCCCN(CCCCO)CCCCCC(=O)OCC(CCCCCC)CCCCCCCC] |
| 20 | 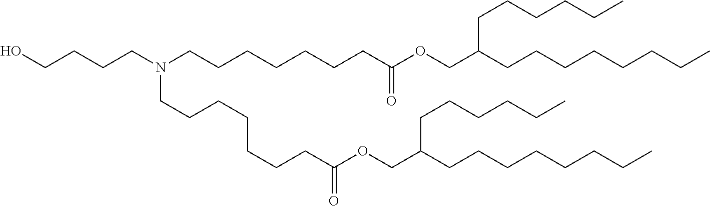 [SMILES: CCCCCCCCC(CCCCCC)COC(=O)CCCCCCCN(CCCCO)CCCCCCCC(=O)OCC(CCCCCC)CCCCCCCC] |
| 21 | 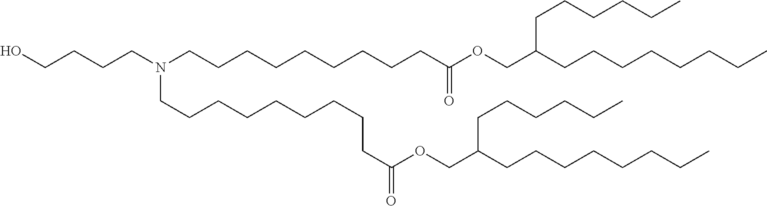 [SMILES: CCCCCCCCC(CCCCCC)COC(=O)CCCCCCCCCN(CCCCO)CCCCCCCCCC(=O)OCC(CCCCCC)CCCCCCCC] |
| 22 | 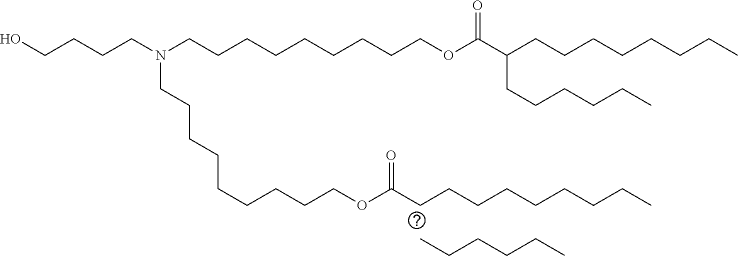 |
| 23 | 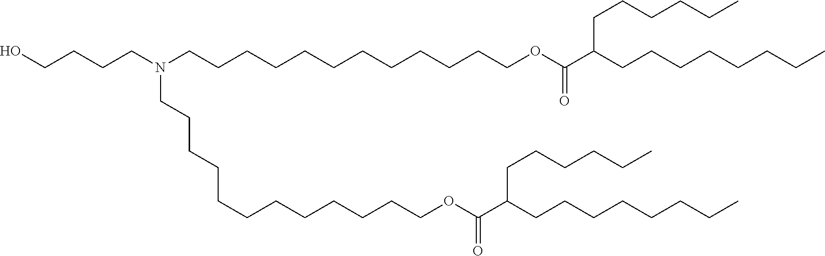 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 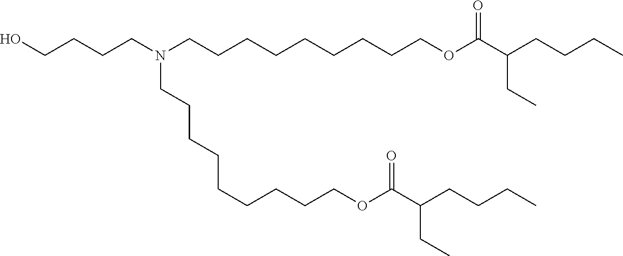 [SMILES: CCCCC(CC)C(=O)OCCCCCCCCCN(CCCCO)CCCCCCCCCOC(=O)C(CC)CCCC] |
| 25 | 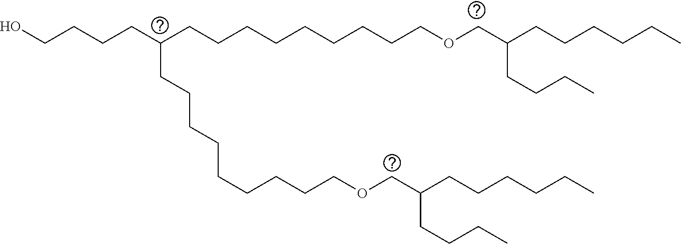 [SMILES: CCCCCCC(CCCC)COCCCCCCCCCC(CCCCO)CCCCCCCCCOCC(CCCC)CCCCCC] |
| 26 | 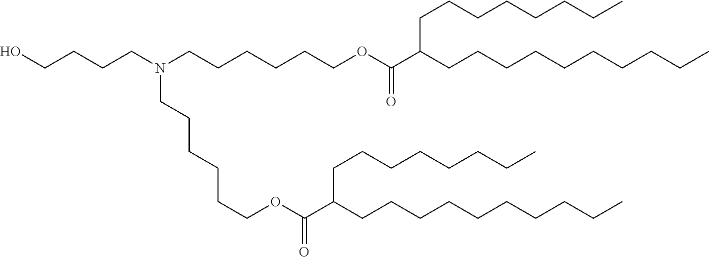 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 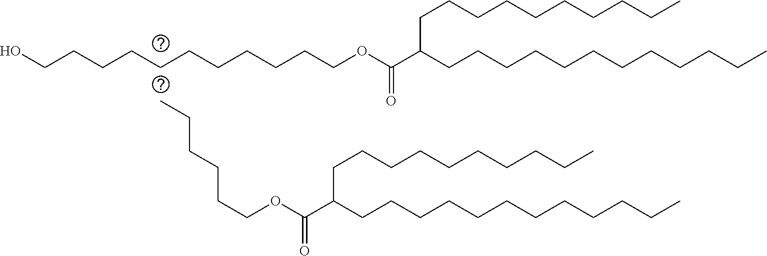 |
| 28 | 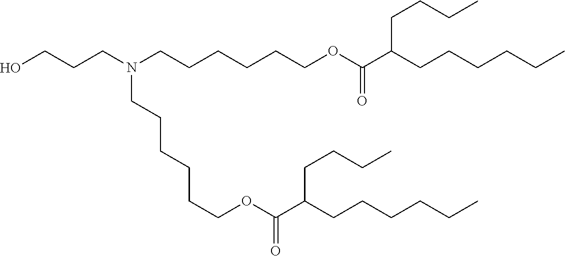 [SMILES: CCCCCCC(CCCC)C(=O)OCCCCCCN(CCCO)CCCCCCOC(=O)C(CCCC)CCCCCC] |
| 29 | 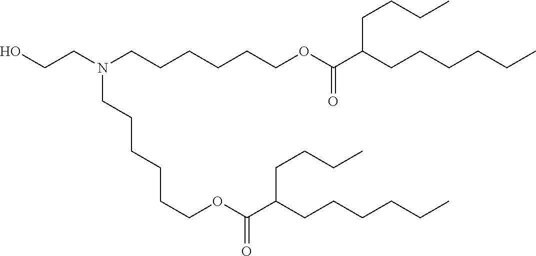 [SMILES: CCCCCCC(CCCC)C(=O)OCCCCCCN(CCO)CCCCCCOC(=O)C(CCCC)CCCCCC] |
| 30 | 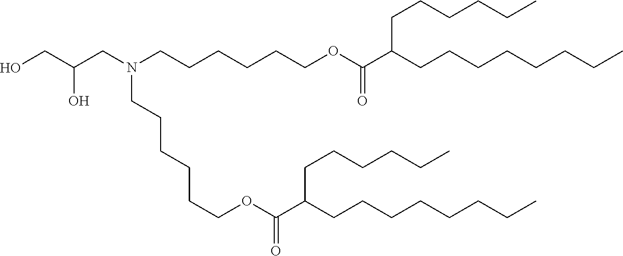 [SMILES: CCCCCCCCC(CCCCCC)C(=O)OCCCCCCN(CCCCCCOC(=O)C(CCCCCC)CCCCCCCC)CC(O)CO] |
| 31 | 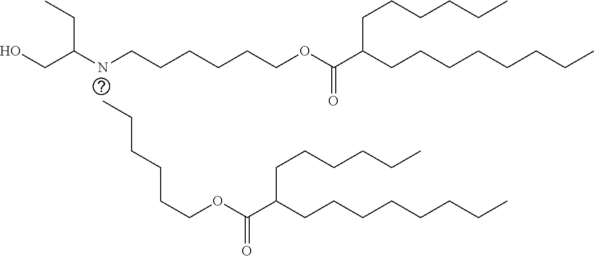 |
| 32 | 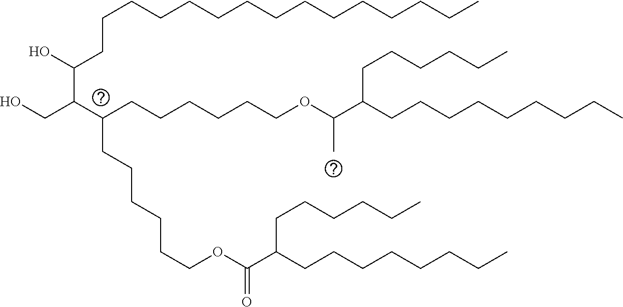 [SMILES: CCCCCCCCCCCCCCCC(O)C(CO)C(CCCCCCOC(=O)C(CCCCCC)CCCCCCCC)CCCCCCOC(C)C(CCCCCC)CCCCCCCCC] |
| 33 | 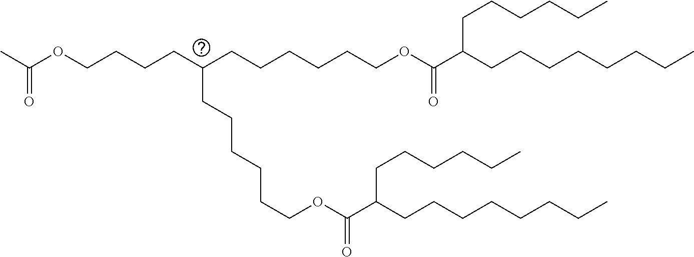 [SMILES: CCCCCCCCC(CCCCCC)C(=O)OCCCCCCC(CCCCCCOC(=O)C(CCCCCC)CCCCCCCC)CCCCOC(C)=O] |
| 34 | 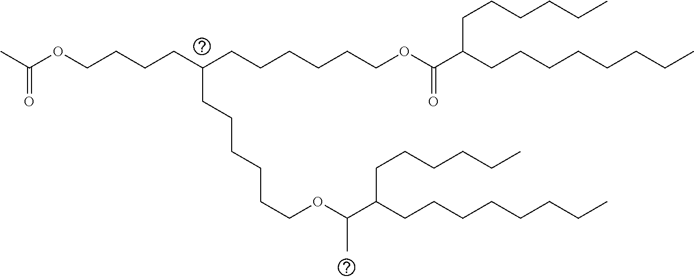 [SMILES: CCCCCCCCC(CCCCCC)C(=O)OCCCCCCC(CCCCCCOC(C)C(CCCCCC)CCCCCCCC)CCCCOC(C)=O] |
| 35 | 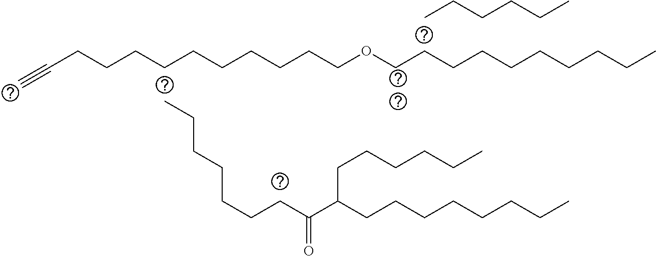 |
| 36 | 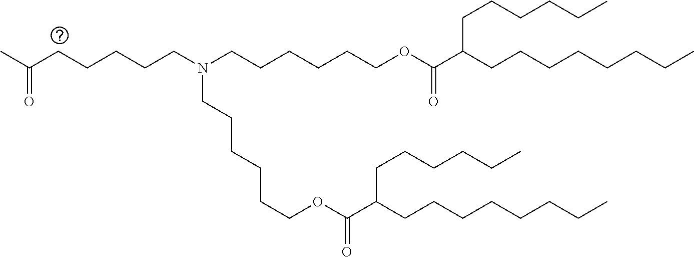 [SMILES: CCCCCCCCC(CCCCCC)C(=O)OCCCCCCN(CCCCCCOC(=O)C(CCCCCC)CCCCCCCC)CCCCCC(C)=O] |

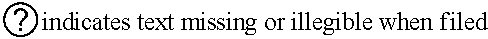

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between any two of about 40, 41, 42, 43, 44, 45, 46, 47, 48, 49, 50, 51, 52, 53, 54, 55, 56, 57, 58, 59 or 60 mole percent, respectively.

In some aspects of the disclosure the LNP comprises a combination or mixture of any the lipids described above.

### II. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]—N, N-ditetradecylacetamide, and the like.

In certain aspects, the LNP comprises an additional, stabilizing-lipid which is a polyethylene glycol-lipid (pegylated lipid). A polymer conjugated lipid (e.g. PEG-lipid) refers to a molecule comprising both a lipid portion and a polymer portion. An example of a polymer conjugated lipid is a PEG-lipid. A PEG-lipid refers to a molecule comprising both a lipid portion and a polyethylene glycol portion. PEG-lipids include, but are not limited to, PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one aspect, the polyethylene glycol-lipid is N-[(methoxy polyethylene glycol) 2000) carbamyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy) propyl-1-O-((o-methoxy(polyethoxy)ethyl) butanedioate (PEG-S-DMG), a PEGylated ceramide (PEG-cer), or a PEG dialkoxypropylcarbamate such as co-methoxy(polyethoxy)ethyl-N-(2,3di(tetradecanoxy) propyl) carbamate or 2,3-di(tetradecanoxy) propyl-N-(u>-methoxy(polyethoxy)ethyl) carbamate. PEG-lipids are disclosed in, e.g., U.S. Pat. No. 9,737,619, the full disclosures of which are herein incorporated by reference in their entirety for all purposes.

In some aspects, the lipid nanoparticles comprise a polymer conjugated lipid. In one aspect, the lipid nanoparticle comprises 2-[(polyethylene glycol)-2000]—N, N-ditetradecylacetamide (ALC-0159), having the formula:

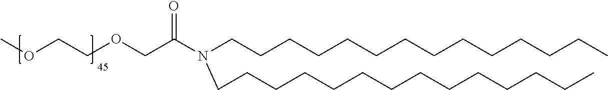

In another aspect, a polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and polyglycerol.

In various aspects, the molar ratio of the cationic lipid to the pegylated lipid or polymer lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid or polymer lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle.

### III. Additional Lipids

In certain aspects, the LNP comprises one or more additional lipids or lipid-like materials that stabilize the formation of particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DpyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC), having the formula:

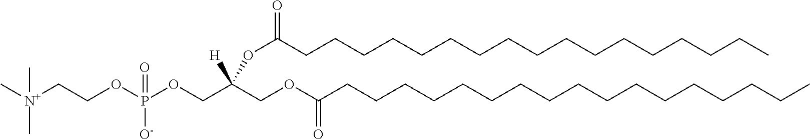 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

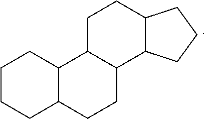 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In one aspect, the cholesterol has the formula:

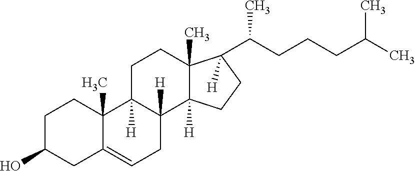

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from about 2:1 to about 8:1, or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle. In some aspects, the non-cationic lipid, e.g., neutral lipid (e.g., one or more phospholipids and/or cholesterol), may be at least, at most, exactly, or between any two of 0 mol %, 10 mol %, 20 mol %, 30 mol %, 40 mol %, 50 mol %, 60 mol %, 70 mol %, 80 mol %, or 90 mol % of the total lipid present in the particle.

### C. Other Materials

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJR 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

### VI. Characterization and Analysis of RNA Molecule

The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-a. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a m7 (3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest (e.g., an antigen) is at least 2-fold to at least 10-fold. In some aspects, elevated expression of a gene of interest (e.g., an antigen) is at least 2-fold. In some aspects, elevated expression of a gene of interest (e.g., an antigen) is at least 3-fold. In some aspects, elevated expression of a gene of interest (e.g., an antigen) is at least 4-fold. In some aspects, elevated expression of a gene of interest (e.g., an antigen) is at least 6-fold. In some aspects, elevated expression of a gene of interest (e.g., an antigen) is at least 8-fold. In some aspects, elevated expression of a gene of interest (e.g., an antigen) is at least 10-fold.

In some aspects, elevated expression of a gene of interest (e.g., an antigen) is about 2-fold to about 50-fold. In some aspects, elevated expression of a gene of interest (e.g., an antigen) is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest (e.g., an antigen) is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest (e.g., an antigen) persists for at least, at most, exactly, or between any two of 24 hours, 36 hours, 48 hours, 60 hours, 72 hours, 84 hours, 96 hours, 108 hours, or 120 hours, or any range or value derivable therein.

### VII. Immune Response

As discussed herein, the disclosure concerns evoking or inducing an immune response in a subject against a PapG protein, e.g., a wild type or variant PapG protein. In one aspect, the immune response may protect against or treat a subject having, suspected of having, or at risk of developing an infection or related disease, particularly those related to E. coli PapG. One use of the immunogenic compositions of the disclosure is to prevent E. coli infections by inoculating or vaccination of a subject.

In some aspects of the disclosure, nanoparticles, RNA molecules encoding E. coli PapG protein, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against an antigen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an of an antigen, e.g. an E. coli PapG protein encoded by an RNA molecule of the present disclosure.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as bacteria, including but not limited to E. coli.

Passive immunity may be imparted to a patient or subject by administering to the patient immunoglobulins (lg) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against E. coli or other organism. A subject thus treated would donate plasma from which hyperimmune globulin would then be obtained, via conventional plasma-fractionation methodology, and administered to another subject in order to impart resistance against or to treat E. coli infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In some aspects, an RNA molecules and/or RNA-LNPs disclosed herein may be administered in a pharmaceutical composition or a medicament and may be administered in the form of any suitable pharmaceutical composition. In some aspects, a pharmaceutical composition is for therapeutic or prophylactic treatments. In one aspect, the disclosure relates to a composition for administration to a host. In some aspects, the host is a human. In other aspects, the host is a non-human.

In some aspects, an RNA molecules and/or RNA-LNPs disclosed herein may be administered in a pharmaceutical composition which may be formulated into preparations in solid, semi-solid, liquid, lyophilized, frozen, or gaseous forms. In some aspects, an RNA molecule and/or RNA-LNPs disclosed herein may be administered in a pharmaceutical composition which may comprise a pharmaceutically acceptable carrier and may optionally comprise one or more adjuvants, stabilizers, salts, buffers, preservatives, and optionally other therapeutic agents. In some aspects, a pharmaceutical composition disclosed herein comprises one or more pharmaceutically acceptable carriers, diluents and/or excipients. In some aspects, pharmaceutical compositions do not include an adjuvant (e.g., they are adjuvant free).

Suitable preservatives for use in a pharmaceutical compositions of the present disclosure include, without limitation, benzalkonium chloride, chlorobutanol, paraben and thimerosal. The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some aspects, the pharmaceutical composition of the present disclosure includes sodium chloride.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents may be selected with regard to the intended route of administration and standard pharmaceutical practice.

In some aspects, the composition comprises an RNA molecule comprising an open reading frame encoding an immunogenic polypeptide. In some aspects, the immunogenic polypeptide comprises an E. coli antigen. In some aspects, the E. coli antigen is an E. coli PapG protein.

In some aspects, the composition comprises an RNA molecule comprising an open reading frame encoding a full-length E. coli PapG protein. In some aspects, the encoded immunogenic polypeptide is a truncated E. coli PapG protein. In some aspects, the encoded immunogenic polypeptide is a variant of an E. coli PapG protein. In some aspects, the encoded immunogenic polypeptide is a fragment of an E. coli PapG protein.

a. Immunogenic Compositions Including LNPS

In some aspects, a pharmaceutical composition comprises an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding a PapG polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., PapG RNA-LNPs).

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further include a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 5% to about 10% w/v. For example, the total concentration of the stabilizing agent may be equal to at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is equal to at least, at most, exactly, or between any two of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent: 1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. In one aspect, the buffer is Tris buffer. In another aspect, the buffer is a HEPES buffer. In a further aspect, the buffer is a PBS buffer. For example, the buffer concentration may be equal to at least, at most, exactly, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may be at least, at most, exactly, or between any two of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. The concentration of the salts in the composition may be about 70 mM to about 140 mM. For example, the salt concentration may be equal to at least, at most, exactly, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is equal to at least, at most, exactly, or between any two of 1 mg/ml, 2 mg/ml, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may be at a pH equal to at least, at most, exactly, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, or cyclodextrins.

Examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition may range from about 10% to about 90% by weight of the total composition, about 25% to about 75%, about 30% to about 60% by weight, or about 12% to about 60%.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, a pharmaceutical composition comprises a PapG RNA molecule encoding a PapG polypeptide as disclosed herein that is complexed with, encapsulated in, and/or formulated with one or more lipids to form PapG RNA-LNPs. In some aspects, the PapG RNA-LNP composition is a liquid. In some aspects, the PapG RNA-LNP composition is frozen. In some aspects, the PapG RNA-LNP composition is lyophilized. In some aspects, a PapG RNA-LNP composition comprises a PapG RNA polynucleotide molecule encoding a PapG polypeptide as disclosed herein, encapsulated in LNPs with a lipid composition of a cationic lipid, a PEGylated lipid (e.g. PEG-lipid), and one or more structural lipids (e.g., a neutral lipid).

In some aspects, a PapG RNA-LNP composition comprises an cationic lipid. The cationic lipid may comprise any one or more cationic lipids disclosed herein. In specific aspects, the cationic lipid comprises ((4-hydroxybutyl) azanediyl)bis(hexane-6,1-diyl)bis(2-hexyldecanoate) (ALC-0315). In some aspects, the cationic lipid (e.g., ALC-0315) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/ug/mg per mL. In some aspects, the cationic lipid (e.g., ALC-0315) is included in the composition at a concentration of at least, at most, between any two of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the cationic lipid (e.g., ALC-0315) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315) is included in the composition at a concentration of 0.8 to 0.95 mg/mL. In specific aspects, the cationic lipid (e.g., ALC-0315) is included in the composition at a concentration of 0.8 to 0.9 mg/mL. In specific aspects, the cationic lipid (e.g., ALC-0315) is included in the composition at a concentration of 0.85 to 0.9 mg/mL. In specific aspects, the cationic lipid (e.g., ALC-0315) is included in the composition at a concentration of 0.8, 0.81, 0.82, 0.83, 0.84, 0.85, 0.86, 0.87, 0.88, 0.89, 0.9, 0.91, 0.92, 0.93, 0.94, or 0.95 mg/mL. Concentrations for lyophilized compositions are determined post-reconstitution.

In some aspects, a PapG RNA-LNP composition further comprises a PEGylated lipid (e.g., PEG-lipid). The PEGylated lipid may comprise any one or more PEGylated lipids disclosed herein. In specific aspects, the PEGylated lipid comprises 2-[(polyethylene glycol)-2000]—N,N-ditetradecylacetamide (ALC-0159). In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, or between 0.2 and 0.25 mg/mL.

In specific aspects, the PEGylated lipid (e.g., ALC-0159) is included in the composition at a concentration of 0.05 to 0.15 mg/mL. In specific aspects, the PEGylated lipid (e.g., ALC-0159) is included in the composition at a concentration of 0.10 to 0.15 mg/mL. In specific aspects, the PEGylated lipid (e.g., ALC-0159) is included in the composition at a concentration of 0.05, 0.06, 0.07, 0.08, 0.09, 0.1, 0.11, 0.12, 0.13, 0.14 or 0.15 mg/mL. Concentrations for lyophilized compositions are determined post-reconstitution.

In some aspects, a PapG RNA-LNP composition further comprises one or more structural lipids. The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-Distearoyl-sn-glycero-3-phosphocholine (DSPC) and cholesterol. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the one or more structural lipids (e.g., DSPC and cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC and cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the one or more structural lipids include DSPC, and the DSPC is included in the composition at a concentration of 0.1 to 0.25 mg/mL. In specific aspects, the one or more structural lipids include DSPC, and the DSPC is included in the composition at a concentration of 0.15 to 0.25 mg/mL. In specific aspects, the one or more structural lipids include DSPC, and the DSPC is included in the composition at a concentration of 0.1, 0.11, 0.12, 0.13, 0.14, 0.15, 0.16, 0.17, 0.18, 0.19, 0.2, 0.21, 0.22, 0.23, 0.24 or 0.25 mg/mL.

In specific aspects, the one or more structural lipids include cholesterol, and the cholesterol is included in the composition at a concentration of 0.3 to 0.45 mg/mL. In specific aspects, the one or more structural lipids include cholesterol, and the cholesterol is included in the composition at a concentration of 0.3 to 0.4. In specific aspects, the one or more structural lipids include cholesterol, and the cholesterol is included in the composition at a concentration of 0.35 to 0.45. In specific aspects, the one or more structural lipids include cholesterol, and the cholesterol is included in the composition at a concentration of 0.30, 0.31, 0.32, 0.33, 0.34, 0.35, 0.36, 0.37, 0.38, 0.39, 0.40, 0.41, 0.42, 0.43, 0.44, or 0.45 mg/mL. Concentrations for lyophilized compositions are determined post-reconstitution.

In some aspects, the PapG RNA-LNP composition further comprises one or more buffers and stabilizing agents, and optionally, salts. Thus, in some aspects, the PapG RNA-LNP composition comprises an cationic lipid, a PEGylated lipid, one or more structural lipids, one or more buffers, a stabilizing agent, and optionally, a salt.

In some aspects, a PapG RNA-LNP composition comprises one or more buffers. The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. Concentrations for lyophilized compositions are determined post-reconstitution.

In some aspects, the PapG RNA-LNP composition is a liquid composition comprising a Tris buffer. In some aspects, the Tris buffer comprises a first buffer. In some aspects, the first buffer is tromethamine. In some aspects, the first buffer (e.g., tromethamine) is included in the liquid composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.1, at least.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the first buffer (e.g., tromethamine) is included in the liquid composition at a concentration of 0.1 to 0.3 mg/mL. In specific aspects, the first buffer (e.g., tromethamine) is included in the liquid composition at a concentration of 0.15 to 0.25 mg/mL. In specific aspects, the first buffer (e.g., tromethamine) is included in the liquid composition at a concentration of 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.3 mg/mL.

In some aspects, the PapG RNA-LNP composition is a liquid composition comprising a Tris buffer comprising a second buffer. In some aspects, the second buffer comprises Tris HCl. In some aspects, the second buffer (e.g., Tris HCl) is included in the liquid composition at a concentration of at least, at most, between any two of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of 1.25 to 1.40 mg/mL. In specific aspects, the second buffer (e.g., Tris HCl) is included in the liquid composition at a concentration of 1.30 to 1.40 mg/mL. In specific aspects, the second buffer (e.g., Tris HCl) is included in the liquid composition at a concentration of 1.25, 1.26, 1.27, 1.28, 1.29, 1.30, 1.31, 1.32, 1.33, 1.34, or 1.35, 1.36, 1.37, 1.38, 1.39, or 1.40 mg/mL.

In some aspects, the PapG RNA-LNP composition is a lyophilized composition comprising a Tris buffer. In some aspects, the Tris buffer comprises a first buffer. In some aspects, the first buffer is tromethamine. In some aspects, the first buffer (e.g., tromethamine) is included in the lyophilized composition at a concentration, after reconstitution, of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of 0.01 and 0.15 mg/mL. In specific aspects, the first buffer (e.g., tromethamine) is included in the lyophilized composition at a concentration, after reconstitution, of 0.01 and 0.10 mg/ml. In specific aspects, the first buffer (e.g., tromethamine) is included in the lyophilized composition at a concentration, after reconstitution, of 0.05 and 0.15 mg/mL. In specific aspects, the first buffer (e.g., tromethamine) is included in the lyophilized composition at a concentration, after reconstitution, of 0.01, 0.02, 0.03, 0.04, 0.05, 0.06, 0.07, 0.08, 0.09, 0.1, 0.11, 0.12, 0.13, 0.14, or 0.15 mg/mL.

In some aspects, the PapG RNA-LNP composition is a lyophilized composition comprising a Tris buffer comprising a second buffer. In some aspects, the second buffer comprises Tris HCl. In some aspects, the second buffer (e.g., Tris HCl) is included in the lyophilized composition at a concentration, after reconstitution, of at least, at most, between any two of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of 0.5 and 0.65 mg/mL. In specific aspects, the second buffer (e.g., Tris HCl) is included in the lyophilized composition at a concentration, after reconstitution, of 0.5 and 0.6 mg/mL. In specific aspects, the second buffer (e.g., Tris HCl) is included in the lyophilized composition at a concentration, after reconstitution, of 0.55 and 0.65 mg/mL. In specific aspects, the second buffer (e.g., Tris HCl) is included in the lyophilized composition at a concentration, after reconstitution, of 0.5, 0.51, 0.52, 0.53, 0.54, 0.55, 0.56, 0.57, 0.58, 0.59, 0.6, 0.61, 0.62, 0.63, 0.64, or 0.65 mg/mL.

In some aspects, a PapG RNA-LNP composition comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is included in the composition at a concentration of at least, at most, between any two of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/μg/mg per mL.

In some aspects, the PapG RNA-LNP composition is a liquid composition, and the stabilizing agent (e.g., sucrose) is included in the liquid composition at a concentration of at least, at most, between any two of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125 or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of 95 to 110 mg/mL. In specific aspects, the stabilizing agent (e.g., sucrose) is included in the liquid composition at a concentration of 95 to 105 mg/mL. In specific aspects, the stabilizing agent (e.g., sucrose) is included in the liquid composition at a concentration of 100 to 110 mg/mL. In specific aspects, the stabilizing agent (e.g., sucrose) is included in the liquid composition at a concentration of 95, 96, 97, 98, 99, 100, 101, 102, 103, 104, 105, 106, 107, 108, 109, or 110 mg/mL.

In some aspects, the PapG RNA-LNP composition is a lyophilized composition, and the stabilizing agent (e.g., sucrose) is included in the lyophilized composition at a concentration, after reconstitution, of at least, at most, between any two of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL. In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75 or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70 or between 70 to 80 mg/mL.

In specific aspects, the stabilizing agent (e.g., sucrose) is included in the lyophilized composition at a concentration, after reconstitution, of 35 to 50 mg/mL. In specific aspects, the stabilizing agent (e.g., sucrose) is included in the lyophilized composition at a concentration, after reconstitution, of 35 to 45 mg/mL. In specific aspects, the stabilizing agent (e.g., sucrose) is included in the lyophilized composition at a concentration, after reconstitution, of 40 to 50 mg/mL. In specific aspects, the stabilizing agent (e.g., sucrose) is included in the lyophilized composition at a concentration, after reconstitution, of 35, 36, 37, 38, 39, 40, 41, 42, 43, 44, 45, 46, 47, 48, 49 or 50 mg/mL.

In some aspects, the PapG RNA-LNP composition is a lyophilized composition, and the lyophilized PapG RNA-LNP composition further comprises a salt. The salt may comprise any one or more salts disclosed herein. In specific aspects, the salt comprises sodium chloride (NaCl). In some aspects, the salt (e.g., NaCl) is included in the lyophilized composition at a concentration, after reconstitution, of at least, at most, between any two of, or exactly 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/μg/mg per mL. In some aspects, the salt (e.g., NaCl) is included in the lyophilized composition at a concentration, after reconstitution, of in at least, at most, between any two of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some aspects, the salt (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt (e.g., NaCl) is included in the lyophilized composition at a concentration, after reconstitution, of between 5 and 15 mg/mL. In some aspects, the salt (e.g., NaCl) is included in the lyophilized composition at a concentration, after reconstitution, of between 5 and 10 mg/mL. In specific aspects, the salt (e.g., NaCl) is included in the lyophilized composition at a concentration, after reconstitution, of 5, 6, 7, 8, 9, 10, 11, 12, 13, 14, or 15 mg/mL.

In some aspects, lyophilized compositions are reconstituted in a suitable carrier or diluent. The carrier or diluent may comprise any one or more carriers or diluents disclosed herein. In specific aspects, the carrier or diluent comprises saline, e.g., physiological saline. The saline may comprise 0.9% saline for injection. In some aspects, the lyophilized compositions are reconstituted in at least, at most, between any two of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of saline.

In specific aspects, the lyophilized compositions are reconstituted in 0.6 to 0.75 ml of saline. In specific aspects, the lyophilized compositions are reconstituted in 0.65 to 0.75 ml of saline. In specific aspects, the lyophilized compositions are reconstituted in 0.6, 0.61, 0.62, 0.63, 0.64, 0.65, 0.66, 0.67, 0.68, 0.69, 0.7, 0.71, 0.72, 0.73, 0,74 or 0.75 mL of saline.

The pH of the PapG RNA-LNP composition may be at least, at most, exactly, or between any two of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the PapG RNA-LNP composition is at a pH of at least 6.5, at least 7.0, at least 7.5, at least 8.0, or at least 8.5. In specific aspects, the PapG RNA-LNP composition is at a pH between 6.0 and 7.5, between 6.5 and 7.5, between 7.0 and 8.0, between and 7.5 and 8.5. In specific aspects, the PapG RNA-LNP composition is between 7.0 and 8.0. In specific aspects, the PapG RNA-LNP composition is at pH 7.0, 7.1, 7.2, 7.3, 7.4, 7.5, 7.6, 7.7, 7.8, 7.9 or 8.0.

In specific aspects, a PapG RNA-LNP composition comprises a PapG RNA polynucleotide encoding a PapG polypeptide as disclosed herein, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL.

In specific aspects, a PapG RNA-LNP composition comprises a PapG RNA polynucleotide encoding a PapG polypeptide as disclosed herein, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL.

In specific aspects, the PapG RNA-LNP composition is a liquid PapG RNA-LNP composition, and the liquid PapG RNA-LNP composition further comprises a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL. In specific aspects, the PapG RNA-LNP composition is a liquid PapG RNA-LNP composition, and the liquid PapG RNA-LNP composition further comprises a Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

Thus, in specific aspects, a liquid PapG RNA-LNP composition comprises an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.1 to 0.3 mg/mL, a second buffer at a concentration of 1.25 to 1.4 mg/mL, and a stabilizing agent at a concentration of 95 to 110 mg/mL.

Thus, in specific aspects, a liquid PapG RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the PapG RNA-LNP composition is a lyophilized PapG RNA-LNP composition, and the lyophilized PapG RNA-LNP composition further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL.

In specific aspects, the PapG RNA-LNP composition is a lyophilized PapG RNA-LNP composition, and the lyophilized PapG RNA-LNP composition further comprises a Tris buffer composition comprising tromethamine at a concentration of 0.01 and 0.15 mg/mL, Tris HCl at a concentration of 0.5 and 0.65 mg/mL, sucrose at a concentration of 35 to 50 mg/mL, and a sodium chloride (NaCl) at a concentration of 5 to 15 mg/mL.

Thus, in specific aspects, a lyophilized PapG RNA-LNP composition comprises a cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, a second structural lipid at a concentration of 0.3 to 0.45 mg/mL, and further comprises a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 ml of carrier or diluent.

Thus, in some aspects, a lyophilized PapG RNA-LNP composition comprises ALC-0315 at a concentration of 0.8 to 0.95 mg/mL, ALC-0159 at a concentration of 0.05 to 0.15 mg/mL, DSPC at a concentration of 0.1 to 0.25 mg/mL, cholesterol at a concentration of 0.3 to 0.45 mg/mL, and further comprises tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 mL of saline.

Concentrations in the lyophilized PapG RNA-LNP composition are determined post-reconstitution.

The PapG RNA-LNP compositions further comprise PapG RNA described herein encapsulated in LNPs, see section D. ADMINISTRATION.

In specific aspects, a PapG RNA-LNP composition is a liquid PapG RNA-LNP composition comprising a PapG RNA polynucleotide encoding a PapG polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a buffer composition comprising a first buffer at a concentration of 0.15 to 0.3 mg/mL, a second buffer at a concentration of 1.25 to 1.4 mg/mL, and a stabilizing agent at a concentration of 95 to 110 mg/mL.

In specific aspects, a liquid PapG RNA-LNP composition comprises a PapG RNA polynucleotide encoding a PapG polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising a

Tris buffer composition comprising tromethamine at a concentration of 0.1 to 0.3 mg/mL, Tris HCl at a concentration of 1.25 to 1.4 mg/mL, and sucrose at a concentration of 95 to 110 mg/mL.

In specific aspects, the PapG RNA-LNP composition is a lyophilized PapG RNA-LNP composition comprising a PapG RNA polynucleotide encoding a PapG polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of an cationic lipid at a concentration of 0.8 to 0.95 mg/mL, a PEGylated lipid at a concentration of 0.05 to 0.15 mg/mL, a first structural lipid at a concentration of 0.1 to 0.25 mg/mL, and a second structural lipid at a concentration of 0.3 to 0.45 mg/mL, and further comprising a first buffer at a concentration of 0.01 and 0.15 mg/mL, a second buffer at a concentration of 0.5 and 0.65 mg/mL, a stabilizing agent at a concentration of 35 to 50 mg/mL, and a salt at a concentration of 5 to 15 mg/mL. In specific aspects, the lyophilized compositions are reconstituted in 0.6 to 0.75 ml of carrier or diluent. Concentrations in the lyophilized PapG RNA-LNP composition are determined post-reconstitution.

In specific aspects, a lyophilized PapG RNA-LNP composition comprises a PapG RNA polynucleotide encoding a PapG polypeptide as disclosed herein at a concentration of at least, at most, exactly, or between any two of 0.01, 0.15, 0.30, 0.45, 0.60, 0.75, or 0.90 mg/mL, encapsulated in LNPs with a lipid composition of ALC-0315 at a concentration of 0.8 to 0.95 mg/mL, ALC-0159 at a concentration of 0.05 to 0.15 mg/mL, DSPC at a concentration of 0.1 to 0.25 mg/mL, and cholesterol at a concentration of 0.3 to 0.45 mg/mL, and further comprising tromethamine at a concentration of 0.01 and 0.15 mg/mL, Tris HCl at a concentration of 0.5 and 0.65 mg/mL, sucrose at a concentration of 35 to 50 mg/mL, and NaCl at a concentration of 5 to 15 mg/mL. In specific aspects, the lyophilized compositions are reconstituted in 0.6 to 0.75 ml of saline. Concentrations in the lyophilized PapG RNA-LNP composition are determined post-reconstitution.

### B. Vaccines

In some aspects, a pharmaceutical composition described herein is an immunogenic composition for inducing an immune response. For example, in some aspects, an immunogenic composition is a vaccine. In some aspects, the compositions described herein include at least one isolated nucleic acid or polypeptide molecule as described herein. In specific aspects, the immunogenic compositions comprise nucleic acids, and the immunogenic compositions are nucleic acid vaccines. In some aspects, the immunogenic compositions comprise RNA (e.g. mRNA), and vaccines are RNA vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that may be treated with the nucleic acid and/or peptide or polypeptide compositions include, but are not limited to, those caused and/or impacted by infection, cancer, rare diseases, and other diseases or conditions caused by overproduction, underproduction, or improper production of protein or nucleic acids.

In some aspects, the composition is substantially free of one or more impurities or contaminants and, for instance, includes nucleic acid or polypeptide molecules that are equal to at least, at most, exactly, or between any two of 90%, 91%, 92%, 93%, 94%, 95%, 96%, 97%, 98%, or 99% pure; at least 98% pure, or at least 99% pure.

The present disclosure includes methods for preventing, treating or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. Immunogenic compositions, proposed to be suitable for use as a vaccine, may be prepared from RNA molecules encoding polypeptide(s), such as the E. coli PapG polypeptides described herein. In certain aspects, immunogenic compositions are lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference in their entireties. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference in their entireties.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%. In some aspects, suppositories may be formed from mixtures containing the active ingredient in the range of about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient.

The polypeptide-encoding nucleic acid constructs and polypeptides may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6 or more administrations. The vaccinations may be at 1, 2, 3, 4, 5, 6, 7, 8, to 5, 6, 7, 8, 9, 10, 11, 12 twelve week intervals, including all ranges there between. In some aspects, vaccinations may be at 1, 2, 3, 4, 5, 6, 7, 8, 9, 10, 11, 12 month intervals, including all ranges there between. Periodic boosters at intervals of 1-5 years may be desirable to maintain protective levels of the antibodies.

i. CARRIERS

A pharmaceutically acceptable carrier may include the liquid or non-liquid basis of a composition. If a composition is provided in liquid form, the carrier may be water, such as pyrogen-free water; isotonic saline or buffered (aqueous) solutions, e.g. phosphate, citrate buffered solutions. Water or a buffer, such as an aqueous buffer, may be used, containing a sodium salt, a calcium salt, and and/or a potassium salt. The sodium, calcium and/or potassium salts may occur in the form of their halogenides, e.g. chlorides, iodides, or bromides, in the form of their hydroxides, carbonates, hydrogen carbonates, or sulfates, etc. Examples of sodium salts include, but are not limited to, NaCl, Nal, NaBr, Na₂CO₃, NaHCO₃, Na₂SO₄, Na₂HPO₄, Na₂HPO₄: 2H₂O, examples of potassium salts include, but are not limited to, KCl, KI, KBr, K₂CO₃, KHCO₃, K₂SO₄, KH₂PO₄, and examples of calcium salts include, but are not limited to, CaCl₂), Cal₂, CaBr₂, CaCO₃, CaSO₄, Ca(OH)₂. Examples of further carriers may include sugars, such as, for example, lactose, glucose, trehalose and sucrose; starches, such as, for example, com starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. Examples of further carriers may include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

### II. Adjuvants

Suitable adjuvants include all acceptable immunostimulatory compounds, such as cytokines, toxins, or synthetic compositions. A number of adjuvants may be used to enhance an antibody response. Adjuvants include, but are not limited to, oil-in-water emulsions, water-in-oil emulsions, mineral salts, polynucleotides, and natural substances. Specific adjuvants that may be used include Freund's adjuvant, oil such as MONTANIDER ISA51, IL1, IL2, IL3, IL4, IL5, IL6, IL7, IL8, IL9, IL10, IL12, alpha-interferon, PTNGg, GM-CSF, GMCSP, BCG, LT-a, aluminum salts, such as aluminum hydroxide or other aluminum compound, MDP compounds, such as thur-MDP and nor-MDP, CGP (MTP-PE), lipid A, monophosphoryl lipid A (MPL), lipopeptides (e.g., Pam³Cys). RIBI, which contains three components extracted from bacteria, MPL, trehalose dimycolate (TDM), and cell wall skeleton (CWS) in a 2% squalene/Tween 80 emulsion. MHC antigens may even be used.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (CARBOPOL®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (FLUOSOL-DA®) used as a block substitute may also be employed to produce an adjuvant effect.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as y-interferon, IL-2, or IL-12 or genes encoding proteins involved in immune helper functions, such as B-7.

Administration of the compositions described herein may be carried out via any of the accepted modes of administration of agents for serving similar utilities. In some aspects, a pharmaceutical composition described herein may be administered intravenously, intraarterially, subcutaneously, intradermally or intramuscularly. In specific aspects, the PapG RNA molecules and/or RNA-LNP compositions are administered intramuscularly. In certain aspects, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In one aspect, the pharmaceutical composition is formulated for intramuscular administration. In another aspect, the pharmaceutical composition is formulated for systemic administration, e.g., for intravenous administration.

Pharmaceutical compositions may be formulated into preparations in solid, semi-solid, liquid, lyophilized, frozen, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid and may be frozen or lyophilized. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration. In some aspects, when intended for oral administration, the pharmaceutical composition is in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, PRIMOJEL®, corn starch and the like; lubricants such as magnesium stearate or STEROTEX®; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. In some aspects, when intended for oral administration, compositions contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or excluded.

A liquid pharmaceutical composition, whether they be solutions, suspensions, or other like form, may include or exclude one or more of the following adjuvants: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation may be enclosed in ampoules, disposable syringes, or multiple dose vials made of glass or plastic. In one aspect, physiological saline is the adjuvant. In one aspect, an injectable pharmaceutical composition is sterile. A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection may be prepared by combining the nucleic acid or polypeptide with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The pharmaceutical compositions according to the present disclosure, or their pharmaceutically acceptable salts, are generally applied in a “therapeutically effective amount” or a “prophylactically effective amount” and in “a pharmaceutically acceptable preparation.” The term “pharmaceutically acceptable” refers to the non-toxicity of a material which does not interact with the action of the active component of the pharmaceutical composition. The terms “therapeutically effective amount” and “prophylactically effective amount” refer to the amount which achieves a desired reaction or a desired effect alone or together with further doses. In the case of the treatment of a particular disease, in one aspect, the desired reaction relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the individual parameters of the patient, including the age, body weight, general health, gender, and diet of the patient; the mode, time, and/or duration of administration; the rate of excretion; the drug combination; the severity of the particular disorder or condition; and the subject undergoing therapy. Accordingly, the doses administered of the compositions described herein may depend on various of such parameters. In the case that a reaction in a patient is insufficient with an initial dose, higher doses (or effectively higher doses achieved by a different, more localized route of administration) may be used. In some aspects, compositions (e.g., PapG RNA-LNP compositions) may be administered at dosage levels sufficient to deliver 0.0001 ng/μg/mg per kg to 100 ng/μg/mg per kg, 0.001 ng/μg/mg per kg to 0.05 ng/μg/mg per kg, 0.005 ng/μg/mg per kg to 0.05 ng/μg/mg per kg, 0.001 ng/μg/mg per kg to 0.005 ng/μg/mg per kg, 0.05 ng/μg/mg per kg to 0.5 ng/μg/mg per kg, 0.01 ng/μg/mg per kg to 50 ng/μg/mg per kg, 0.1 ng/μg/mg per kg to 40 ng/μg/mg per kg, 0.5 ng/μg/mg per kg to 30 ng/μg/mg per kg, 0.01 ng/μg/mg per kg to 10 ng/μg/mg per kg, 0.1 ng/μg/mg per kg to 10 ng/μg/mg per kg, or 1 ng/μg/mg per kg to 25 ng/μg/mg per kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). In some aspects, compositions (e.g., PapG RNA-LNP compositions) may be administered at dosage levels sufficient to deliver at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per kg, of subject body weight per day, one or more times a day, per week, per month, etc. to obtain the desired therapeutic, diagnostic, prophylactic, or imaging effect.

In some aspects, compositions (e.g., PapG RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per day, one or more times a day, per week, per month, etc. to obtain the desired therapeutic, diagnostic, prophylactic, or imaging effect.

In specific aspects, compositions (e.g., PapG RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL PapG RNA encapsulated in LNP.

In exemplary aspects, compositions (e.g., PapG RNA-LNP compositions) may be administered at dose levels of at least, at most, exactly, or between any two of 0.01, 0.15, 0.30, 0.45, 0.60, 0.75, or 0.90 mg/mL PapG RNA encapsulated in LNP. In exemplary aspects, compositions (e.g., PapG RNA-LNP compositions) may be administered at dose levels of at least, at most, exactly, or between any two of 0.01, 0.15, 0.30, 0.45, 0.60, 0.75, or 0.90 mg PapG RNA encapsulated in LNP.

In specific aspects, compositions (e.g., PapG RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL PapG RNA encapsulated in LNP.

In exemplary aspects, compositions (e.g., PapG RNA-LNP compositions) may be administered at dose levels of at least, at most, exactly, or between any two of 1, 15, 30, 45, 60, 75, or 90 μg/mL PapG RNA encapsulated in LNP. In exemplary aspects, compositions (e.g., PapG RNA-LNP compositions) may be administered at dose levels of at least, at most, exactly, or between any two of 1, 15, 30, 45, 60, 75, or 90 ug PapG RNA encapsulated in LNP.

The desired dosage may be delivered multiple times a day (e.g., 1, 2, 3, 4, 5, or more times a day), every other day, every third day, every week, every two weeks, every three weeks, every four weeks, every 2 months, every three months, every 6 months, etc. In certain aspects, the desired dosage may be delivered using a single-dose administration. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. The time of administration between the initial administration of the composition and a subsequent administration of the composition may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some aspects, compositions (e.g., PapG RNA-LNP compositions) may be administered in a single dose. In some aspects, compositions (e.g., PapG RNA-LNP compositions) may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 1 month later, Day 0 and 2 months later, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later), with each administration at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg PapG RNA encapsulated in LNP. Higher and lower dosages and frequency of administration are encompassed by the present disclosure. For example, compositions (e.g., PapG RNA-LNP compositions) may be administered three or four times. Periodic boosters at intervals of 1-5 years may be desirable to maintain protective levels of the antibodies.

In some aspects, the compositions (e.g., PapG RNA-LNP compositions) are administered to a subject as a single dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of PapG RNA encapsulated in LNP. In some aspects, the compositions (e.g., PapG RNA-LNP compositions) are administered the subject as a single dose of at least, at most, exactly, or between any two of 1 μg, 15 ug, 30 ug, 45 ug, 60 ug, 75 ug, or 90 μg of PapG RNA encapsulated in LNP.

In some aspects, the compositions (e.g., PapG RNA-LNP compositions) are administered to a subject as two doses of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of PapG RNA encapsulated in LNP. In some aspects, the compositions (e.g., PapG RNA-LNP compositions) are administered the subject as two doses of at least, at most, exactly, or between any two of 1 μg, 15 ug, 30 ug, 45 ug, 60 ug, 75 ug, or 90 μg of PapG RNA encapsulated in LNP.

In specific aspects, compositions (e.g., PapG RNA-LNP compositions) may be administered twice (e.g., Day 0 and Day 28, Day 0 and Day 60, Day 0 and Day 180, Day 0 and 2 months later, Day 0 and 6 months later), with each administration at a total dose of or at dosage levels sufficient to deliver a total dose of at least, at most, exactly, or between any two of 1 μg, 15 μg, 30 μg, 45 μg, 60 μg, 75 μg, or 90 μg PapG RNA encapsulated in LNP.

### IX. Methods of Use

Provided herein are compositions (e.g., pharmaceutical compositions comprising PapG RNA molecules and/or PapG RNA-LNPs), methods, kits and reagents for prevention and/or treatment of E. coli infection in humans and other mammals.

The RNA (e.g., mRNA) vaccines may be utilized in various settings depending on the prevalence of the infection or the degree or level of unmet medical need. The RNA vaccines may be utilized to treat and/or prevent E. coli infection of various genotypes, strains, and isolates. The RNA vaccines typically have superior properties in that they produce much larger antibody titers and produce responses earlier than commercially available anti-bacterial therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

There may be situations in which persons are at risk for infection with more than one E. coli antigen. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one E. coli antigen, a combination vaccine can be administered that includes RNA (e.g., mRNA) encoding at least one antigenic polypeptide protein (or antigenic portion thereof) of a first E. coli antigen, e.g. PapG or a fragment thereof, or organism and further includes RNA encoding at least one antigenic polypeptide protein (or antigenic portion thereof) of a second antigen. RNA (e.g., mRNA) can be co-formulated, for example, in a single lipid nanoparticle (LNP) or can be formulated in separate LNPs for co-administration.

PapG RNA compositions (e.g., PapG RNA-LNP compositions) may be used as prophylactic agents. They may be used in medicine to prevent and/or treat infectious disease. In exemplary aspects, the PapG RNA compositions are used to provide prophylactic protection from urinary tract infections (UTI). The PapG vaccines of the present disclosure may be particularly useful for prevention and/or treatment of immunocompromised and elderly patients to prevent or to reduce the severity and/or duration of E. coli infection.

In some aspects, the PapG RNA compositions (e.g., PapG RNA-LNP compositions) of the disclosure are administered to a subject (e.g., a mammalian subject, such as a human subject), and the RNA polynucleotides are translated in vivo to produce an antigenic polypeptide.

In some aspects, the PapG RNA compositions of the disclosure may be used to prime immune effector cells, for example, to activate peripheral blood mononuclear cells (PBMCs) ex vivo, which are then infused (re-infused) into a subject.

In some aspects, after administration of a PapG RNA molecule described herein, e.g., formulated as RNA-LNPs, at least a portion of the RNA is delivered to a target cell. In some aspects, at least a portion of the RNA is delivered to the cytosol of the target cell. In some aspects, the RNA is translated by the target cell to produce the polypeptide or protein it encodes. In some aspects, the target cell is a spleen cell. In some aspects, the target cell is an antigen presenting cell such as a professional antigen presenting cell in the spleen. In some aspects, the target cell is a dendritic cell or macrophage. RNA molecules such as RNA-LNPs described herein may be used for delivering RNA to such target cell. Accordingly, the present disclosure also relates to a method for delivering RNA to a target cell in a subject comprising the administration of the RNA-particles described herein to the subject.

In some aspects, the RNA is delivered to the cytosol of the target cell. In some aspects, the RNA is translated by the target cell to produce the polypeptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

In some aspects, nucleic acid compositions described herein, e.g., compositions comprising a PapG RNA-LNP are characterized by (e.g., when administered to a subject) sustained expression of an encoded polypeptide. For example, in some aspects, such compositions are characterized in that, when administered to a human, they achieve detectable polypeptide expression in a biological sample (e.g., serum) from such human and, in some aspects, such expression persists for a period of time that is at least at least 36 hours or longer, including, e.g., at least 48 hours, at least 60 hours, at least 72 hours, at least 96 hours, at least 120 hours, at least 148 hours, or longer.

In some aspects, the disclosure relates to a method of inducing an immune response in a subject. The method includes administering to the subject an effective amount of an RNA molecule, RNA-LNP and/or composition as described herein.

In another aspect, the disclosure relates to a method of vaccinating a subject. The method includes administering to the subject in need thereof an effective amount of an RNA molecule, RNA-LNP and/or composition described herein.

In another aspect, the disclosure relates to a method of treating or preventing a bacterial disease. The method includes administering to the subject an effective amount of an RNA molecule RNA-LNP and/or composition as described herein.

In another aspect, the disclosure relates to a method of treating or preventing or reducing the severity of an E. coli infection and/or illness caused by E. coli. The method includes administering to the subject an effective amount of an RNA molecule, RNA-LNP and/or composition as described herein.

In another aspect, the disclosure relates to a method of treating or preventing or reducing the severity of an infection in a subject by, for example, inducing an immune response to the infectious agent, e.g. E. coli, in the subject. In some aspects, the method includes administering a priming composition that includes an effective amount of an RNA molecule, RNA-LNP and/or composition described herein, and administering a booster composition including an effective amount of an RNA molecule, RNA-LNP and/or composition. In some aspects, the composition elicits an immune response including an antibody response. In some aspects, the composition elicits an immune response including a T cell response.

In another aspect, the disclosure relates to a method of treating or preventing or reducing the severity of an E. coli infection and/or illness caused by E. coli in a subject by, for example, inducing an immune response to E. coli PapG in the subject. In some aspects, the method includes administering a priming composition that includes an effective amount of an RNA molecule, RNA-LNP and/or composition described herein, and administering a booster composition including an effective amount of an RNA molecule RNA-LNP and/or composition as described herein. In some aspects, the composition elicits an immune response including an antibody response. In some aspects, the composition elicits an immune response including a T cell response.

The methods disclosed herein may involve administering to the subject a PapG RNA-LNP composition comprising at least one PapG RNA molecule having an open reading frame encoding at least one PapG antigenic polypeptide, thereby inducing in the subject an immune response specific to E. coli PapG antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine against E. coli. An “anti-antigenic polypeptide antibody” is a serum antibody the binds specifically to the antigenic polypeptide. In some aspects, the anti-antigenic polypeptide antibody titer in the subject is increased at least, at most, between any two of, or exactly 1, 2, 3, 4, 5, 6, 7, 8, 9, or 10 log following administration of the PapG RNA-LNP composition relative to anti-antigenic polypeptide antibody titer in a subject administered a prophylactically effective dose of a traditional composition against PapG.

The methods disclosed herein may involve administering to the subject a PapG RNA-LNP composition comprising at least one PapG RNA molecule having an open reading frame encoding at least one PapG antigenic polypeptide, thereby inducing in the subject an immune response specific to PapG antigenic polypeptide, wherein the immune response in the subject is equivalent to an immune response in a subject administered with a traditional composition against the PapG at least, at most, in between any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

In some aspects, the RNA molecule, RNA-LNP and/or composition is used as a vaccine. In some aspects, the RNA molecule, RNA-LNP and/or composition may be used in various therapeutic or prophylactic methods for preventing, treating or ameliorating of urinary tract infection, urosepsis, pyelonephritis or cystitis.

PapG RNA compositions may be administered prophylactically to healthy subjects or early in infection during the incubation phase or during active infection after onset of symptoms. In some aspects, the subject is immunocompetent. In some aspects, the subject is immunocompromised.

In some aspects, the RNA molecule, RNA-LNP and/or composition is administered in a single dose. In some aspects, a second, third or fourth dose may be given. In some aspects, the RNA molecule, RNA-LNP and/or composition is administered in multiple doses.

In some aspects, the RNA molecule, RNA-LNP and/or composition is administered intramuscularly (IM) or intradermally (ID).

The present disclosure further provides a kit comprising the RNA molecule, RNA-LNP, and/or composition.

In some aspects, the RNA molecule, RNA-LNP and/or composition described herein is administered to a subject that is less than about 1 years old, or about 1 years old to about 10 years old, or about 10 years old to about 20 years old, or about 20 years old to about 50 years old, or about 60 years old to about 70 years old, or older.

In some aspects the subject is at least, at most, exactly, or between any two of less than 1 year of age, greater than 1 year of age, greater than 5 years of age, greater than 10 years of age, greater than 20 years of age, greater than 30 years of age, greater than 40 years of age, greater than 50 years of age, greater than 60 years of age, greater than 70 years of age, or older. In some aspects, the subject is greater than 50 years of age.

In some aspects the subject is at least, at most, exactly, or between any two of about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject may be about 50 years of age or older.

In some aspects the subject is at least, at most, exactly, or between any two of 1 year of age or older, 5 years of age or older, 10 years of age or older, 20 years of age or older, 30 years of age or older, 40 years of age or older, 50 years of age or older, 60 years of age or older, 70 years of age or older, or older. In some aspects the subject may be 50 years of age or older. In an embodiment, the mRNA vaccines of the invention comprise lipids. The lipids and modRNA can together form nanoparticles. The lipids can encapsulate the mRNA in the form of a lipid nanoparticle (LNP) to aid cell entry and stability of the RNA/lipid nanoparticles.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

A LNP may include one or more components described herein. In some embodiments, the LNP formulation of the disclosure includes at least one lipid nanoparticle component. Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

In some embodiments, for example, a polymer may be included in and/or used to encapsulate or partially encapsulate a LNP. A polymer may be biodegradable and/or biocompatible. A polymer may be selected from, but is not limited to, polyamines, polyethers, polyamides, polyesters, poly carbamates, polyureas, polycarbonates, polystyrenes, polyimides, polyethylenes, polyethyleneimines, polysulfones, polyurethanes, polyacetylenes, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho) esters, poly(butyric acid), poly(valeric acid), poly(lactide-co-caprolactone), trimethylene carbonate, poly(N-acryloylmorpholine) (PACM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN® 60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21 st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80 PC, −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

Lipid nanoparticles may include a lipid component and one or more additional components, such as a therapeutic and/or prophylactic, such as a nucleic acid. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combination of elements. The efficacy and tolerability of a LNP formulation may be affected by the stability of the formulation.

The lipid component of a LNP may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (e.g. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

The RNA (e.g., mRNA) vaccines may be utilized in various settings depending on the prevalence of the infection or the degree or level of unmet medical need. The RNA vaccines may be utilized to treat and/or prevent E. coli infection of various genotypes, strains, and isolates. The RNA vaccines typically have superior properties in that they produce much larger antibody titers and produce responses earlier than commercially available anti-viral or anti-bacterial therapeutic treatments. While not wishing to be bound by theory, it is believed that the RNA vaccines, as mRNA polynucleotides, are better designed to produce the appropriate protein conformation upon translation as the RNA vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

There may be situations in which persons are at risk for infection with more than one E. coli antigen. RNA (e.g., mRNA) therapeutic vaccines are particularly amenable to combination vaccination approaches due to a number of factors including, but not limited to, speed of manufacture, ability to rapidly tailor vaccines to accommodate perceived geographical threat, and the like. Moreover, because the vaccines utilize the human body to produce the antigenic protein, the vaccines are amenable to the production of larger, more complex antigenic proteins, allowing for proper folding, surface expression, antigen presentation, etc. in the human subject. To protect against more than one E. coli antigen, a combination vaccine can be administered that includes RNA (e.g., mRNA) encoding at least one antigenic polypeptide protein (or antigenic portion thereof) of a first E. coli antigen, e.g. PapG or a fragment thereof, or organism and further includes RNA encoding at least one antigenic polypeptide protein (or antigenic portion thereof) of a second antigen. RNA (e.g., mRNA) can be co-formulated, for example, in a single lipid nanoparticle (LNP) or can be formulated in separate LNPs for co-administration.

Some embodiments of the present disclosure provide E. coli vaccines (or compositions or immunogenic compositions) that include at least one RNA polynucleotide having an open reading frame encoding at least one E. coli PapG antigenic polypeptide or an immunogenic fragment thereof (e.g., an immunogenic fragment capable of inducing an immune response to E. coli).

Some embodiments of the present disclosure provide E. coli vaccines that include at least one RNA polynucleotide having an open reading frame encoding at least one E. coli PapG polypeptide or an immunogenic fragment of the novel PapG polypeptide sequences described above (e.g., an immunogenic fragment capable of inducing an immune response to E. coli). In some embodiments, an E. coli vaccine comprises at least one RNA (e.g., mRNA) polynucleotide having an open reading frame encoding at least one PapG polypeptide comprising a modified sequence that is at least 75% (e.g., any number between 75% and 100%, inclusive, e.g., 70%, 80%, 85%, 90%, 95%, 99%, and 100%) identity to an amino acid sequence of the novel PapG sequences described above. The modified sequence can be at least 75% (e.g., any number between 75% and 100%, inclusive, e.g., 70%, 80%, 85%, 90%, 95%, 99%, and 100%) identical to an amino acid sequence of the novel PapG sequences described above.

Some embodiments of the present disclosure provide an isolated nucleic acid comprising a sequence encoding the novel E. coli PapG polypeptide sequences described above; an expression vector comprising the nucleic acid; and a host cell comprising the nucleic acid. The present disclosure also provides a method of producing a polypeptide of any of the novel E. coli PapG sequences described above. A method may include culturing the host cell in a medium under conditions permitting nucleic acid expression of the novel E. coli PapG sequences described above, and purifying from the cultured cell or the medium of the cell a novel E. coli PapG polypeptide.

In some embodiments, a RNA (e.g., mRNA) vaccine further comprising an adjuvant.

In some embodiments, at least one RNA polynucleotide encodes at least one E. coli PapG polypeptide that does not attach to cells.

In some embodiments, at least one RNA polynucleotide encodes at least one E. coli PapG polypeptide that does not allow binding of the bacteria to a cell, wherein the cell is a bladder epithelial cell. Some embodiments of the present disclosure provide a vaccine that includes at least one ribonucleic acid (RNA) (e.g., mRNA) polynucleotide having an open reading frame encoding at least one E. coli PapG polypeptide, at least one 5′ terminal cap and at least one chemical modification, formulated within a lipid nanoparticle.

In some embodiments, a 5′ terminal cap is m⁷G (5′)ppp(5′)(2′OMeA)pG.

In some embodiments, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino) butanoyl)oxy) heptadecanedioate (L319), (12Z, 15Z)—N,N-dimethyl-2-nonylhenicosa-12, 15-dien-1-amine (L608), and N,N-dimethyl-1-[(1S,2R)-2-octylcyclopropyl] heptadecan-8-amine (L530).

Some embodiments of the present disclosure provide a vaccine that includes at least one RNA (e.g., mRNA) polynucleotide having an open reading frame encoding at least one E. coli PapG polypeptide, wherein at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil. In some embodiments, a chemical modification is a 1-methyl-3′-pseudouridylyl. In some embodiments, 100% of the uracil in the open reading frame have a 1-methyl-3′-pseudouridylyl in the 5-position of the uracil.

In some embodiments, an open reading frame of an RNA (e.g., mRNA) polynucleotide encodes at least one E. coli polypeptide. In a preferred embodiment, the E. coli polypeptide is a fimbrial antigen. In a preferred embodiment, the E. coli fimbrial antigen is PapG. In some embodiments, the open reading frame encodes at least two, at least five, or at least ten E. coli polypeptides. In some embodiments, the open reading frame encodes at least 100 E. coli polypeptides. In some embodiments, the open reading frame encodes 1-100 E. coli polypeptides. In some embodiments, a vaccine comprises at least two RNA (e.g., mRNA) polynucleotides, each having an open reading frame encoding at least one E. coli PapG polypeptide. In some embodiments, the vaccine comprises at least five or at least ten RNA (e.g., mRNA) polynucleotides, each having an open reading frame encoding at least one E. coli PapG polypeptide or an immunogenic fragment thereof. In some embodiments, the vaccine comprises at least 100 RNA (e.g., mRNA) polynucleotides, each having an open reading frame encoding at least one E. coli PapG polypeptide. In some embodiments, the vaccine comprises 2-100 RNA (e.g., mRNA) polynucleotides, each having an open reading frame encoding at least one E. coli PapG polypeptide.

In a further aspect, the invention provides a multivalent vaccine, wherein the multivalent vaccine comprises at least two RNA (e.g., mRNA) polynucleotides, each having an open reading frame encoding at least one E. coli PapG polypeptide. In some embodiments, the multivalent vaccine comprises at least five or at least ten RNA (e.g., mRNA) polynucleotides, each having an open reading frame encoding at least one E. coli PapG polypeptide or an immunogenic fragment thereof. In some embodiments, the multivalent vaccine comprises at least 100 RNA (e.g., mRNA) polynucleotides, each having an open reading frame encoding at least one E. coli

PapG polypeptide. In some embodiments, the multivalent vaccine comprises 2-100 RNA (e.g., mRNA) polynucleotides, each having an open reading frame encoding at least one E. coli PapG polypeptide. In a further embodiment, the multivalent vaccine comprises RNA (e.g., mRNA) polynucleotides, each having an open reading frame encoding at least one additional polypeptide including, but not limited to, E. coli FmIH, E. coli PapG, K. pneu. MrkA, E. faecalis EbpA, or immunogenic fragments thereof.

Also provided herein is an E. coli RNA (e.g., mRNA) vaccine of any one of the foregoing paragraphs formulated in a nanoparticle (e.g., a lipid nanoparticle).

In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol.

In some embodiments, the nanoparticle has a polydispersity value of less than 0.4 (e.g., less than 0.3, 0.2 or 0.1).

In some embodiments, the nanoparticle has a net neutral charge at a neutral pH value. Some embodiments of the present disclosure provide methods of inducing an antigen specific immune response in a subject, comprising administering to the subject any of the RNA (e.g., mRNA) vaccine as provided herein in an amount effective to produce an antigen-specific immune response. In some embodiments, the RNA (e.g., mRNA) vaccine is an E. coli vaccine. In some embodiments, the RNA (e.g., mRNA) vaccine is a combination vaccine comprising a combination of E. coli vaccines (a broad-spectrum E. coli vaccine).

In some embodiments, an antigen-specific immune response comprises a T cell response or a B cell response.

In some embodiments, a method of producing an antigen-specific immune response comprises administering to a subject a single dose (no booster dose) of an E. coli RNA (e.g., mRNA) vaccine of the present disclosure.

In some embodiments, a method further comprises administering to the subject a second (booster) dose of an E. coli RNA (e.g., mRNA) vaccine. Additional doses (boosters) of an E. coli RNA (e.g., mRNA) vaccine may be administered.

In some embodiments, the subjects exhibit a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, an antigen can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an E. coli RNA (e.g., mRNA) vaccine is administered to a subject by intradermal injection, intramuscular injection, or by intranasal administration. In some embodiments, an E. coli RNA (e.g., mRNA) vaccine is administered to a subject by intramuscular injection.

Some embodiments, of the present disclosure provide methods of inducing an antigen specific immune response in a subject, including administering to a subject an E. coli RNA (e.g., mRNA) vaccine in an effective amount to produce an antigen specific immune response in a subject. Antigen-specific immune responses in a subject may be determined, in some embodiments, by assaying for antibody titer (for titer of an antibody that binds to an E. coli PapG polypeptide) following administration to the subject of any of the E. coli RNA (e.g., mRNA) vaccines of the present disclosure. In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered a RNA (e.g., mRNA) vaccine of the present disclosure. In some embodiments, the control is an anti-antigenic polypeptide antibody titer produced in a subject who has been administered an E. coli PapG polypeptide or fragment thereof, or wherein the control is an anti-antigenic polypeptide antibody titer produced in a subject who has been administered a recombinant or purified E. coli PapG vaccine. In some embodiments, the control is an anti-antigenic polypeptide antibody titer produced in a subject who has not been administered a recombinant or purified E. coli PapG vaccine.

A RNA (e.g., mRNA) vaccine of the present disclosure is administered to a subject in an effective amount (an amount effective to induce an immune response). In some embodiments, the effective amount is a dose equivalent to an at least 2-fold, at least 4-fold, at least 10-fold, at least 100-fold, at least 1000-fold reduction in the standard of care dose of a recombinant E. coli vaccine, wherein the anti-antigenic polypeptide antibody titer produced in the subject is equivalent to an anti-antigenic polypeptide antibody titer produced in a control subject administered the standard of care dose of a recombinant E. coli protein vaccine. In some embodiments, the effective amount is a dose equivalent to 2- to 1000-fold reduction in the standard of care dose of a recombinant E. coli protein vaccine, wherein the anti-antigenic polypeptide antibody titer produced in the subject is equivalent to an anti-antigenic polypeptide antibody titer produced in a control subject administered the standard of care dose of a recombinant E. coli protein vaccine.

In some embodiments, the RNA (e.g., mRNA) vaccine is formulated in an effective amount to produce an antigen specific immune response in a subject.

In some embodiments, the effective amount is a total dose ≤25 μg. In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two or more times. In some embodiments, the effective amount is a dose of 100 μg administered to the subject a total of two or more times. In some embodiments, the effective amount is a dose of 400 μg administered to the subject a total of two or more times. In some embodiments, the effective amount is a dose of 500 μg administered to the subject a total of two or more times.

In some embodiments, the efficacy (or effectiveness) of a RNA (e.g., mRNA) vaccine is greater than 60%. In some embodiments, the RNA (e.g., mRNA) polynucleotide of the vaccine encodes at least one E. coli PapG polypeptide.

Vaccine efficacy may be assessed using standard analyses. For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARU×100; and Efficacy=(1−RR)×100.

Likewise, vaccine effectiveness may be assessed using standard analyses. Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In some embodiments, the efficacy (or effectiveness) of a RNA (e.g., mRNA) vaccine is at least 65%, at least 70%, at least 75%, at least 80%, at least 85%, or at least 90%.

In some embodiments, the vaccine immunizes the subject against E. coli for up to 2 years. In some embodiments, the vaccine immunizes the subject against E. coli for more than 2 years, more than 3 years, more than 4 years, or for 5-10 years.

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, an RNA (e.g., mRNA) vaccine may be administered later in life, for example, at the age of about 6 months to about 5 years, or older.

In some embodiments, the subject is an adolescent between the ages of about 11-19 years (e.g., about 11, 12, 13, 14, 15, 16, 17, 18, or 19 years old).

In some embodiments, the subject is an adult between the ages of about 20 years and about 59 years (e.g., about 20, 25, 30, 35, 40, 45, 50, 55 or 59 years old).

In some embodiments, the subject is an older adult subject about 60 years old, about 70 years old, or older (e.g., about 60, 65, 70, 75, 80, 85 or 90 years old).

In some embodiments, the subject has been exposed to E. coli; the subject is infected with E. coli; or subject is at risk of infection by E. coli.

In some embodiments, the subject is immunocompromised (has an impaired immune system, e.g., has an immune disorder or autoimmune disorder).

In some embodiments the nucleic acid vaccines described herein are chemically modified. In other embodiments the nucleic acid vaccines are unmodified.

Yet other aspects provide compositions for and methods of vaccinating a subject comprising administering to the subject a nucleic acid vaccine comprising one or more RNA polynucleotides having an open reading frame encoding a first antigenic polypeptide, wherein the RNA polynucleotide does not include a stabilization element, and wherein an adjuvant is not coformulated or co-administered with the vaccine.

In other aspects the invention is a composition for or method of vaccinating a subject comprising administering to the subject a nucleic acid vaccine comprising one or more RNA polynucleotides having an open reading frame encoding a first antigenic polypeptide wherein a dosage of between 10 μg/kg and 400 μg/kg of the nucleic acid vaccine is administered to the subject. In some embodiments the dosage of the RNA polynucleotide is ≤1 μg, 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300μ, 50-350μ, 100-350μ, 200-350μ, 300-350μ, 320-400μ, 40-380μ, 40-100μ g, 100-400 μg, 200-400 μg, or 300-400 μg per dose. In some embodiments, the nucleic acid vaccine is administered to the subject by intradermal or intramuscular injection.

In some embodiments, a dosage of 25 micrograms of the RNA polynucleotide is included in the nucleic acid vaccine administered to the subject. In some embodiments, a dosage of 100 micrograms of the RNA polynucleotide is included in the nucleic acid vaccine administered to the subject. In some embodiments, a dosage of 50 micrograms of the RNA polynucleotide is included in the nucleic acid vaccine administered to the subject. In some embodiments, a dosage of 75 micrograms of the RNA polynucleotide is included in the nucleic acid vaccine administered to the subject. In some embodiments, a dosage of 150 micrograms of the RNA polynucleotide is included in the nucleic acid vaccine administered to the subject. In some embodiments, a dosage of 400 micrograms of the RNA polynucleotide is included in the nucleic acid vaccine administered to the subject. In some embodiments, a dosage of 200 micrograms of the RNA polynucleotide is included in the nucleic acid vaccine administered to the subject. In some embodiments, the RNA polynucleotide accumulates at a 100-fold higher level in the local lymph node in comparison with the distal lymph node. In other embodiments the nucleic acid vaccine is chemically modified and in other embodiments the nucleic acid vaccine is not chemically modified.

Aspects of the invention provide a nucleic acid vaccine comprising one or more RNA polynucleotides having an open reading frame encoding a first antigenic polypeptide, wherein the RNA polynucleotide does not include a stabilization element, and a pharmaceutically acceptable carrier or excipient, wherein an adjuvant is not included in the vaccine. In some embodiments, the stabilization element is a histone stem-loop. In some embodiments, the stabilization element is a nucleic acid sequence having increased GC content relative to wild type sequence.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for seroprotection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of bacteria binding to the plate.

Also provided are nucleic acid vaccines comprising one or more RNA polynucleotides having an open reading frame encoding a first antigenic polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce neutralizing antibodies within one week of a single administration. In some embodiments, the adjuvant is selected from a cationic peptide and an immunostimulatory nucleic acid. In some embodiments, the cationic peptide is protamine.

Aspects provide nucleic acid vaccines comprising one or more RNA polynucleotides having an open reading frame comprising at least one chemical modification or optionally no modified nucleotides, the open reading frame encoding a first antigenic polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no modified nucleotides, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 μg and 400 μg of one or more RNA polynucleotides having an open reading frame comprising at least one chemical modification or optionally no modified nucleotides, the open reading frame encoding a first antigenic polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a bacteria or virus in an individual or population of individuals comprising administering to said individual or population an antigenic memory booster nucleic acid vaccine comprising (a) at least one RNA polynucleotide, said polynucleotide comprising at least one chemical modification or optionally no modified nucleotides and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide, wherein the vaccine has at least 10-fold less RNA polynucleotide than is required for an unmodified mRNA vaccine to produce an equivalent antibody titer. In some embodiments, the RNA polynucleotide is present in a dosage of 25-100 micrograms.

Other aspects provide nucleic acid vaccines comprising an LNP-formulated RNA polynucleotide having an open reading frame comprising no nucleotide modifications (unmodified), the open reading frame encoding a first antigenic polypeptide, wherein the vaccine has at least 10-fold less RNA polynucleotide than is required for an unmodified mRNA vaccine not formulated in a LNP to produce an equivalent antibody titer. In some embodiments, the RNA polynucleotide is present in a dosage of 25-100 micrograms.

Both chemically modified and unmodified RNA vaccines are useful according to the invention. Prior art reports that it is preferable to use chemically unmodified mRNA formulated in a carrier for the production of vaccines. Both the chemically modified and unmodified RNA vaccines of the invention produce better immune responses than mRNA vaccines formulated in a different lipid carrier.

In other aspects the invention encompasses a method of treating an older adult subject age 60 years or older comprising administering to the subject a nucleic acid vaccine comprising one or more RNA polynucleotides having an open reading frame encoding an E. coli antigenic polypeptide in an effective amount to vaccinate the subject.

In other aspects the invention encompasses a method of treating a young subject age 17 years or younger comprising administering to the subject a nucleic acid vaccine comprising one or more RNA polynucleotides having an open reading frame encoding an E. coli antigenic polypeptide in an effective amount to vaccinate the subject.

In other aspects the invention encompasses a method of treating an adult subject between the ages of about 20 years and about 50 years old comprising administering to the subject a nucleic acid vaccine comprising one or more RNA polynucleotides having an open reading frame encoding an E. coli antigenic polypeptide in an effective amount to vaccinate the subject.

In some aspects, the invention is a method of vaccinating a subject with a combination vaccine including at least two nucleic acid sequences encoding antigens wherein the dosage for the vaccine is a combined therapeutic dosage wherein the dosage of each individual nucleic acid encoding an antigen is a subtherapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject.

In preferred aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in a subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA) or Luminex. In exemplary embodiments, antibody titer is determined or measured by neutralization assay, e.g., by E. coli binding inhibition assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose). In exemplary aspects of the invention, antigen-specific antibodies are measured in units of ug/ml or are measured in units of IU/L (International Units per liter) or mlU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mlU/ml,>20 mlU/ml,>50 mlU/ml,>100 mlU/ml,>200 mlU/ml,>500 mlU/ml or >1000 mlU/ml antigen-specific antibodies. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose). In exemplary embodiments, antibody level or concentration is determined or measured by enzyme-linked immunosorbent assay (ELISA) or Luminex. In exemplary embodiments, antibody level or concentration is determined or measured by neutralization assay, e.g., by E. coli binding inhibition assay.

### X. Nanoparticles

In another aspect, disclosed herein is an immunogenic complex that includes 1) a nanostructure; and 2) at least one fimbrial polypeptide antigen or fragment thereof. Preferably, the fimbrial polypeptide or fragment thereof is derived from E. coli PapG. In a preferred embodiment, the fimbrial polypeptide is selected from any one of the fimbrial polypeptides described above. For example, the fimbrial polypeptide may comprise any one amino acid sequence selected from SEQ ID NOs: 11-25 or 26-41.

In some embodiments, the antigen is fused or conjugated to the nanostructure exterior to stimulate development of adaptive immune responses to the displayed epitopes. In some embodiments, the immunogenic complex further includes an adjuvant or other immunomodulatory compounds attached to the exterior and/or encapsulated in the cage interior to help tailor the type of immune response generated for each pathogen.

In some embodiments, the nanostructure includes a single assembly including a plurality of identical first nanostructure-related polypeptides.

In alternative embodiments, the nanostructure includes a plurality assembly, including a plurality of identical first nanostructure-related polypeptides and a plurality of second assemblies, each second assembly comprising a plurality of identical second nanostructure-related polypeptides.

Various nanostructure platforms can be employed in generating the immunogenic compositions described herein. In some embodiments, the nanostructures employed are formed by multiple copies of a single subunit. In some embodiments, the nanostructures employed are formed by multiple copies of multiple different subunits.

The nanostructures are typically ball-like shaped, and/or have rotational symmetry (e.g., with 3-fold and 5-fold axis), e.g., with an icosahedral structure exemplified herein.

In some embodiments, the antigen is presented on self-assembling nanoparticles such as self-assembling nanostructures derived from ferritin (FR), E2p, Qβ, and 13-01. E2p is a redesigned variant of dihydrolipoyl acyltransferase from Bacillus stearothermophilus. 13-01 is an engineered protein that may self-assemble into hyperstable nanoparticles. Sequences of the subunits of these proteins are known in the art. In a first apsect, disclosed herein is a nanostructure-related polypeptide comprising an amino acid sequence that is at least 75% identical over its length, and identical at least at one identified interface position, to the amino acid sequence of a nanostructure-related polypeptide selected from the group consisting of SEQ ID NOS: 76-118. The nanostructure-related polypeptides can be used, for example, to prepare the nanostructures. The nanostructure-related polypeptides were designed for their ability to self-assemble in pairs to form nanostructures, such as icosahedral nanostructures.

In some embodiments, the nanostructure includes (a) a plurality of first assemblies, each first assembly comprising a plurality of identical first nanostructure-related polypeptides, wherein the first nanostructure-related polypeptides comprise the amino acid sequence of a nanostructure-related polypeptide selected from the group consisting of SEQ ID NOS: 76-118; and (b) a plurality of second assemblies, each second assembly comprising a plurality of identical second nanostructure-related polypeptides, wherein the second nanostructure-related polypeptides comprise the amino acid sequence of a nanostructure-related polypeptide selected from the group consisting of SEQ ID NOS: 76-118, and wherein the second nanostructure-related polypeptide differs from the first nanostructure-related polypeptide; wherein the plurality of first assemblies non-covalently interact with the plurality of second assemblies to form a nanostructure;

The nanostructures include symmetrically repeated, non-natural, non-covalent polypeptide-polypeptide interfaces that orient a first assembly and a second assembly into a nanostructure, such as one with an icosahedral symmetry.

SEQ ID NOS: 76-118 set forth in Table 14 provide the amino acid sequence of exemplary nanostructure-related polypeptides. The number of interface residues for the exemplary nanostructure-related polypeptides of SEQ ID NO:76-118 range from 4-13 residues. In various embodiments, the nanostructure-related polypeptides comprise an amino acid sequence that is at least 75%, 80%, 85%, 90%, 91%, 92%, 93%, 94%, 95%, 96%, 97%, 98%, or 99% identical over its length, and identical at least at 1, 2, 3, 4, 5, 6, 7, 8, 9, 10, 11, 12, or 13 identified interface positions (depending on the number of interface residues for a given nanostructure-related polypeptide), to the amino acid sequence of a nanostructure-related polypeptide selected from the group consisting of SEQ ID NOS: 76-118. In other embodiments, the nanostructure-related polypeptides comprise an amino acid sequence that is at least 75%, 80%, 85%, 90%, 91%, 92%, 93%, 94%, 95%, 96%, 97%, 98%, or 99% identical over its length, and identical at least at 20%, 25%, 33%, 40%, 50%, 60%, 70%, 75%, 80%, 90%, or 100% of the identified interface positions, to the amino acid sequence of a nanostructure-related polypeptide selected from the group consisting of SEQ ID NOS: 76-118. In further embodiments, the nanostructure-related polypeptides include a nanostructure-related polypeptide having the amino acid sequence of a nanostructure-related polypeptide selected from the group consisting of SEQ ID NOS: 76-118.

In one non-limiting embodiment, the nanostructure-related polypeptides can be modified to facilitate covalent linkage to a “cargo” of interest. In one non-limiting example, the nanostructure-related polypeptides can be modified, such as by introduction of various cysteine residues at defined positions to facilitate linkage to one or more antigens of interest, such that a nanostructure of the nanostructure-related polypeptides would provide a scaffold to provide a large number of antigens for delivery as a vaccine to generate an improved immune response.

In some embodiments, some or all native cysteine residues that are present in the nanostructure-related polypeptides but not intended to be used for conjugation may be mutated to other amino acids to facilitate conjugation at defined positions. In another non-limiting embodiment, the nanostructure-related polypeptides may be modified by linkage (covalent or non-covalent) with a moiety to help facilitate “endosomal escape.” For applications that involve delivering molecules of interest to a target cell, such as targeted delivery, a critical step can be escape from the endosome-a membrane-bound organelle that is the entry point of the delivery vehicle into the cell. Endosomes mature into lysosomes, which degrade their contents. Thus, if the delivery vehicle does not somehow “escape” from the endosome before it becomes a lysosome, it will be degraded and will not perform its function. There are a variety of lipids or organic polymers that disrupt the endosome and allow escape into the cytosol. Thus, in this embodiment, the nanostructure-related polypeptides can be modified, for example, by introducing cysteine residues that will allow chemical conjugation of such a lipid or organic polymer to the monomer or resulting assemly surface. In another non-limiting example, the nanostructure-related polypeptides can be modified, for example, by introducing cysteine residues that will allow chemical conjugation of fluorophores or other imaging agents that allow visualization of the nanostructures in vitro or in vivo.

Surface amino acid residues on the nanostructure-related polypeptides can be mutated in order to improve the stability or solubility of the protein subunits or the assembled nanostructures. As will be known to one of skill in the art, if the nanostructure-related polypeptide has significant sequence homology to an existing protein family, a multiple sequence alignment of other proteins from that family can be used to guide the selection of amino acid mutations at non-conserved positions that can increase protein stability and/or solubility, a process referred to as consensus protein design (9).

Surface amino acid residues on the nanostructure-related polypeptides can be mutated to positively charged (Arg, Lys) or negatively charged (Asp, Glu) amino acids in order to endow the protein surface with an overall positive or overall negative charge. In one non-limiting embodiment, surface amino acid residues on the nanostructure-related polypeptides can be mutated to endow the interior surface of the self-assembling nanostructure with a high net charge.

Such a nanostructure can then be used to package or encapsulate a cargo molecule with the opposite net charge due to the electrostatic interaction between the nanostructure interior surface and the cargo molecule. In one non-limiting embodiment, surface amino acid residues on the nanostructure-related polypeptides can be mutated primarily to Arginine or Lysine residues in order to endow the interior surface of the self-assembling nanostructure with a net positive charge. Solutions containing the nanostructure-related polypeptides can then be mixed in the presence of a nucleic acid cargo molecule such as a dsDNA, ssDNA, dsRNA, ssRNA, cDNA, miRNA., siRNA, shRNA, piRNA, or other nucleic acid in order to encapsulate the nucleic acid inside the self-assembling nanostructure. Such a nanostructure could be used, for example, to protect, deliver, or concentrate nucleic acids.

In one embodiment, the nanostructure has icosahedral symmetry. In this embodiment, the nanostructure may comprise 60 copies of the first nanostructure-related polypeptide and 60 copies of the second nanostructure-related polypeptide. In one such embodiment, the number of identical first nanostructure-related polypeptides in each first assembly is different than the number of identical second nanostructure-related polypeptides in each second assembly. For example, in one embodiment, the nanostructure comprises twelve first assemblies and twenty second assemblies; in this embodiment, each first assembly may; for example, comprise five copies of the identical first nanostructure-related polypeptide, and each second assembly may, for example, comprise three copies of the identical second nanostructure-related polypeptide. In another embodiment, the nanostructure comprises twelve first assemblies and thirty second assemblies; in this embodiment, each first assembly may, for example, comprise five copies of the identical first nanostructure-related polypeptide, and each second assembly may, for example, comprise two copies of the identical second nanostructure-related polypeptide. In a further embodiment, the nanostructure comprises twenty first assemblies and thirty second assemblies; in this embodiment, each first assembly may, for example, comprise three copies of the identical first nanostructure-related polypeptide, and each second assembly may, for example, comprise two copies of the identical second nanostructure-related polypeptide. All of these embodiments are capable of forming synthetic nanomaterials with regular icosahedral symmetry.

### EXAMPLES

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for. The following Examples illustrate some embodiments of the invention.

The aim of the studies set forth in the following Example(s) was to a) develop a mammalian expression platform for production of E. coli PapG proteins, b) produce full-length single chain PapG proteins, c) engineer PapG proteins to remove ligand binding ability and potentially increase immunogenicity, d) to ultimately identify lead PapG modRNA candidates for further development. Mutants designed to eliminate binding to cognate ligands were expressed in mammalian cells and subjected to a series of biochemical evaluations which enabled prioritization of candidates with little to no ligand binding, while maintaining acceptable thermal stability of the protein. Wild type (WT) and mutant versions that were either secreted or membrane associated were encoded on mRNA constructs and expression was evaluated.

### Example 1

### Design and Expression of Escherichia coli PapG modRNA Constructs

This Example describes a mammalian expression platform to produce PapG proteins and the engineering of single-chain donor strand-complemented full-length versions of PapG that can be produced without the need for a chaperone. Furthermore, this Example describes the design and evaluation of PapG_{LD} variants that were predicted to be unable to bind their cognate ligands, which was evaluated in a series of biochemical and biophysical analyses. This Example further describes mRNA constructs encoding PapG (LD or full-length) and demonstrates expression of PapG on the surface of mammalian cells or secreted into the extracellular milieu in modRNA transcript transfected cells. Expression of the fimbrial antigen, FimH, in mammalian cells has been described in International Patent Publication NO. WO2021084429A1, which is hereby incorporated by reference in its entirety. Lastly, a serological assay to measure immune responses to PapG is described, based on its ability to agglutinate human red blood cells.

a. Materials and Methods

### 1. Design of Antigens

Identities of the PapG antigens produced in this study are described below. The genes encoding the pap operon are located on pathogenicity islands, large horizontally transferable genetic elements [29]. Note that in E. coli strain CFT073, there are two copies of the pap operon, each encoding all of the Pap components including PapG [30]. Mobley et al. demonstrated that each pap operon could be mutated independently, and the strain retained P-fimbria media binding in agglutination assays [31]. However, if both operons were mutated, binding was abolished [31]. The PapG amino acid sequence encoded by each of these loci (version 1 (V1) and version 2 (V2), located in islands close to pheV and pheU tRNA gene loci respectively) differ at four amino acid positions (version 1: K73, N132, T152, 1159 and version 2: Q73, D132, K152, M159). While both versions of the wild type PapG_{LD} (V1 and V2 respectively) were expressed and evaluated in Luminex assays, all stabilizing mutations and full-length proteins described herein are based on version 1 (encoded by the operon located close to the pheV chromosomal locus in CFT073). PapG sequences are listed herein.

Asparagine-linked-glycans (N-glycans) are common post-translational modifications on proteins in mammalian cells and can occur on bacterial proteins produced in mammalian cells that are unmodified when produced in bacteria. N-linked glycosylation was predicted using the NetNGlyc N-glycosylation prediction server, https://services.healthtech.dtu.dk/services/NetNGlyc-1.0/[33]. Based on analysis of the natural variants in the PapG amino acid sequence at the predicted glycosylated Asparagine in clinical E. coli isolates, predicted glycosylation sites were mutated to Serine to prevent glycosylation (see Table 1, Table 2, Table 3 and Table 4). The resulting proteins were confirmed to be aglycosylated by mass spectrometry by comparing intact mass results with and without PNGase F treatment to remove N-glycosylation Additional mutations were introduced which are either located in the ligand binding site or could potentially stabilize PapG in an open confirmation (see Table 2, Table 3 and Table 4).

TABLE 1
WT and aglycosylated PapG_{LD} proteins

| Variant | Purpose |
|---|---|
| PapG_{LD} V1 | WT PapG_{LD} (V1, first PapG operon), His-tagged |
| PapG_{LD} N96S V1 | WT PapG_{LD} (V1, first PapG operon), removal of |
| | non-native glycosylation at N96, His-tagged |
| PapG_{LD} N96S V2 | WT PapG_{LD} (V2, second PapG operon), removal |
| | of non-native glycosylation at N96, His-tagged |

TABLE 2
Mutations located in loops involved in ligand binding in PapG_{LD}

| Variant | Purpose |
|---|---|
| PapG_{LD} N96S G86A | Removal of non-native glycosylation at N96, |
| | G86S ligand binding site mutation aimed to |
| | block globoside binding, His-tagged |
| PapG_{LD} N96S S89T | Removal of non-native glycosylation at N96, |
| | S89T ligand binding site mutation aimed to |
| | block globoside binding at S89T, His-tagged |
| PapG_{LD} N96S G104A | Removal of non-native glycosylation at N96, |
| | G104A ligand binding site mutation aimed to |
| | block globoside binding, His-tagged |
| PapG_{LD} N96S G168A | Removal of non-native glycosylation at N96, |
| | G168A ligand binding site mutation aimed to |
| | block globoside binding, His-tagged |
| PapG_{LD} N96S W107A | Removal of non-native glycosylation at N96, |
| | W107A ligand binding site mutation aimed to |
| | block globoside binding, His-tagged |
| PapG_{LD} N96S R170A | Removal of non-native glycosylation at N96, |
| | R170A ligand binding site mutation aimed to |
| | block globoside binding, His-tagged |
| PapG_{LD} N96S K172A | Removal of non-native glycosylation at N96, |
| | K172A ligand binding site mutation aimed to |
| | block globoside binding, His-tagged |

TABLE 3
Putative conformation stabilizing mutations in PapG_{LD}

| Variant | Purpose |
|---|---|
| PapG_{LD} N96S G18A | Removal of non-native glycosylation at N96, |
| | G14A mutation of potential allosteric site to |
| | stabilize PapGLD in an open conformation |
| | with low affinity for cognate globoside receptor, |
| | His-tagged |
| PapG_{LD} N96S G75A | Removal of non-native glycosylation at N96, |
| | G75A mutation of potential allosteric site to |
| | stabilize PapGLD in an open conformation |
| | with low affinity for cognate globoside receptor, |
| | His-tagged |
| PapG_{LD} N96S G122A | Removal of non-native glycosylation at N96, |
| | G122A mutation of potential allosteric site to |
| | stabilize PapGLD in an open conformation |
| | with low affinity for cognate globoside receptor, |
| | His-tagged |
| PapG_{LD} N96S G147A | Removal of non-native glycosylation at N96, |
| | G147A mutation of potential allosteric site to |
| | stabilize PapGLD in an open conformation with |
| | low affinity for cognate globoside receptor, |
| | His-tagged |

TABLE 4
Full-length PapG_{DSF}

| Variant | Purpose |
|---|---|
| PapG_{DSF} N96S N286S | Full-length PapG with predicted |
| | mutations designed to remove non- |
| | native glycosylation at N96S and |
| | N286S , contained N242 |
| | glycosylation site, His-tagged |
| PapG_{DSF} N96S N242S N286S | Full-length PapG with mutations |
| | designed to remove non-native |
| | glycosylation, fully aglycosylated |
| | at N96S, N242S and N286S, His- |
| | tagged |
| PapG_{DSF} N96S N242S N286S | Full-length PapG with mutations |
| K172A | designed to remove non-native |
| | glycosylation at N96S, N242S and |
| | N286S, including the K172A ligand |
| | binding site mutation, His-tagged |

### 2. Cloning of DNA Constructs for Expression in Mammalian Cells

PapG sequences were codon optimized for human expression by IDT, Azenta or GeneArt software (varied). DNA fragments containing PapG-II sequences (from CFT073 E. coli reference strain) with C-terminal 8×His tags were synthetized. The DNA fragments were assembled into linearized pcDNA3.1 (+) mammalian expression vector (Invitrogen V79020) containing a sequence encoding a mouse IgGK signal peptide for secretion into the cell culture supernatant with NEBuilder HiFi DNA Assembly Master Mix (NEB C2621) according to manufacturer's instructions. Specific mutations were introduced into the PapG pcDNA3.1 constructs by site-directed mutagenesis according to the New England Biolabs protocol using the Q5 Site-Directed Mutagenesis Kit (NEB E0554).

### 3. Protein Expression in Mammalian Cells and Protein Purification

Endotoxin-free pcDNA3.1 plasmid DNA (purified from E. coli using a MidiPrep kit (Qiagen)) encoding PapG proteins was transfected into ExpiCHO cells (ThermoFisher Scientific) according to manufacturer's instructions. After 5-8 days when approximately 80% viability was reached, cell culture supernatants were harvested by filtration with diatomaceous earth (Sartorius-Sartoclear Dynamics Lab Filter Aid-SDLKG-10.0). Supernatants were adjusted to pH 8.0 with 1M Tris pH 8.0. For 250 ml supernatant, 4 ml 50% complete® His-Tag Purification Resin (Roche-05893682001) slurry equilibrated in Nickel buffer A (25 mM Tris pH 8.0, 300 mM NaCl, 20 mM imidazole) was added to the supernatants and were incubated overnight at 4° C. with rotation. After incubation, the supernatants were poured into BioRad gravity columns and washed with 20-25 column volumes of Nickel buffer A. The proteins were eluted with four column volumes of Nickel Buffer B (25 mM Tris pH 8, 300 mM NaCl, 500 mM Imidazole). The proteins were applied to a HiTrap Phenyl HP column (Cytiva-17519501) under high sodium citrate conditions (see Table 5, HIC buffer A) The material bound to the column was eluted with a linear gradient of decreasing sodium citrate using 25 mM Tris pH 8.0, 10% glycerol (see Table 5, HIC buffer B). Fractions were analyzed by SDS-PAGE and Coomassie staining. Selected fractions were pooled and dialyzed against 1X PBS pH 7.4.

TABLE 5
Buffers used for HiTrap Phenyl 152epharose
purification of PapG proteins, applied to all variants

| Protein ID | HIC buffer A | HIC buffer B |
|---|---|---|
| PapG_{LD} | 400 mM Na-Citrate, | 25 mM Tris pH 8.0, |
| | 25 mM Tris pH 8.0 | 10% glycerol |
| PapG_{DSF} | 350 mM Na-Citrate, | 25 mM Tris pH 8.0, |
| | 25 mM Tris pH 8.0 | 10% glycerol |

### 4. Mouse Immunizations

Anti-PapG monoclonal antibodies were produced from two groups of CD1 mice with five animals each. CD1 mice were immunized thrice subcutaneously with purified PapG_{LD} N96S 8×His protein with LiNA-2 adjuvant (50 or 100 μg) in the scruff of the neck (0.1 mL each) at weeks 0, 4, and 8; and bled at weeks 0, 6, 10, and 15. Animals with high titers were boosted intraperitoneally (IP) (0.1 mL each) at weeks 16 and 36, and spleens were harvested 3 days after the IP boost.

### 5. Rabbit Immunizations

For production of anti-PapG rabbit sera, one group of New Zealand White rabbits with five animals was immunized with 100 μg doses of mammalian produced PapG_{LD} N96S WT, with a 1:1 ratio of Complete Freund's Adjuvant (week 0) or IFA (week 4 and 8). Animals were vaccinated SC at four sites (0.25 mL each, total of 1.0 mL) at weeks 0,4, and 8; and bled at weeks 0,4,6,8, and 10.

### 6. PapG Monoclonal Antibody Production

For PapG mAb production, spleen cells from mice with high titers were harvested and fused with the myeloma P3X63-Ag8.653 cell line using Electrofusion (BTX-ECM 2001). Fused cells were cultured in 96-well plates at 37° C., 8% CO₂ in DMEM containing HAT supplement (21060-017 Gibco). After 10 days in culture, hybridomas were screened by enzyme-linked immunosorbent assay (ELISA) using Maxisorp high binding 384 well plates (464717, Thermo Fisher Scientific) coated with 20 ng of PapG_{LD} N96S protein. The positive hybridomas secreting anti-PapG antibody were subcloned and clonality was confirmed by DNA sequencing.

### 7. Galactoside and Globoside Ligands Used in Assays.

Below is an overview of PapG ligands used in biochemical assays in this report (Table 6).

TABLE 6
Galactoside and globoside ligands used in assays

| Target | Assay | Compound name |
|---|---|---|
| PapG | Thermal shift assay | Globotriaosylceramide |
| | | (globoside; GbO3) |
| PapG | Luminex ligand binding | GalNAc-Gal-Gal-Glc-ceramide |
| | assay | (globoside; GbO4) |
| PapG | Luminex ligand binding | Gal-Glc-phenoxy-octyl-amine |
| | assay | (GbO2Mpo) |

### 8. Thermal Shift

A 96-well thermal stability assay using SYPRO orange (Invitrogen S6650) was developed to determine the melting temperatures of purified PapG proteins in APO (unbound) form and in the presence of GbO₃ (TCI-M1767), mimicking the natural ligand of PapG (GbO₄). PapG proteins were added to assay buffer (consisting of 4% DMSO, 1:1000 dilution of SYPRO orange (Invitrogen S6650) in PBS pH 7.4) at a final concentration of 2 mM. Melt curves were analyzed in a QuantStudio 6 Real-time PCR system (ThermoFisher) using a dissociation protocol from 25° C. to 99° C., at 0.05° C./second. ROX was specified as the reporter. The data were analyzed with Protein Thermal Shift Software v1.0 (Applied Biosystems) using a Maxwell-Boltzmann distribution to determine melting temperature (Tm) of the protein and the shift in melting temperature (ΔTm) in the presence and absence of GbO₃.

9. PapG Ligand Binding Assay with Globoside GbO₄

To determine the ability of PapG_{LD} mutants to bind the porcine derived globotetraosyl ceramide ligand GalNAc-Gal-Gal-Glc-ceramide (GbO₄), a Luminex ligand binding assay was developed. Surfactant Protein-A (SP-A) was coupled to the surface of MagPlex beads by EDC/NHS, to capture the lipid tail of the globotetraosyl ceramide. The Globotetraosyl ceramide/SP-A beads were incubated with an eight-point threefold titration of PapG in a 96 well flat bottom white plate with final volume of 100 microliters of 50 mM HEPES PH 7.4, 10 mM NaCl, 0.0005% Tween 20 shaking at 300 rpm, 4° C. overnight. After 20-24 hours the ligand binding plates were read on a FlexMap 3D plate reader for measurement of well-bead region specific MFI. Ligand binding equilibrium dissociation constants (Kd) were calculated assuming one site specific binding and interpolated using non-linear curve fitting (GraphPad Prism) as the ligand concentration needed to achieve half-maximal binding. The Luminex derived apparent Kd values are based on approximately fifty bead counts per assay sample.

10. PapG Ligand Binding Assay with Globoside Compound GbO₂Mpo

To determine Luminex ligand binding affinities of PapG mutants for the synthetic globoside ligand Gal-Glc-phenoxy-octyl-amine (GbO₂Mpo), a Luminex ligand binding assay was developed based on the direct coupling of the amine reactive globoside ligand to the EDC/NHS activated carboxylic surface of MagPlex beads. Synthesis of GbO₂Mpo ligand with an octyl amine linker (for coupling to beads) was performed via methods described previously [34, 35]. The GbO₂Mpo beads were incubated with an 8-point, threefold titration of PapG in a 96-well flat bottom white plate with final volume of 100 μl of 50 mM HEPES pH 7.4, 10 mM NaCl, 0.0005% Tween 20 shaking at 300 rpm, 4° C. overnight. After 20-24 hours the ligand binding plates were read on a FlexMap 3D plate reader for measurement of well-bead region specific MFI.

11. mRNA Vector Cloning for Mammalian Cell Expression

mRNA constructs BMD2, BMD562, BMD563 and BMD576 are described hereinabove. Common features of these vectors include: T7 promoter for in vitro transcription (IVT), a vector-specific 5′UTR, a 3′UTR from Human Beta Globin (HBB) and a 3′ 80nt polyA adenylation site. Synthetic PapG open reading frames codon-optimized for mammalian cell expression were used.

### 12. PapG 96-Well Expression Assay

ExpiCHO cells (ThermoFisher Scientific) were diluted to 6×10⁶ viable cells/mL with fresh ExpiCHO Expression Medium and 0.5 mL of was pipetted into each well of a round bottom 96-deep well block (Axygen™ Storage Microplate PDW20CS). Transfections of pcDNA3.1 plasmids encoding PapG_{LD} variants were performed according to ExpiCHO manufacturer's instructions in 0.5 mL. Plates were sealed with a gas-permeable seal and incubated in a humidified chamber at 37° C. with 8% CO₂ on an orbital shaker at 1500 rpm. CHO enhancer and feed were added 18-22 hours post transfection, according to manufacturer's instructions. Cells and supernatants were harvested by centrifugation on day 4 post transfection, for analysis by Western blot and Octet (described herein).

### 13. In Vitro Transcription (IVT)

Five (5) μg of BspQI linearized plasmid DNA was used as template in each 100 μL in vitro transcription reaction. Linearized DNA templates were purified prior to in vitro transcription with Proteinase K/SDS, and phenol/chloroform extraction followed by EtOH precipitation. The CleanCap® AG kit (TriLink) was used which simultaneously caps newly transcribed mRNA molecules with m7G (5′)ppp(5′)(2′OMeA)pG. Following 2 hours at 37° C., each reaction was treated with DNaseI for 30 minutes and mRNA was precipitated by addition of LiCl solution. After LiCl precipitation, centrifuged pellets were washed with 70% ethanol and resuspended in nuclease free water. Size and integrity of the transcribed RNAs were confirmed using an Advanced Analytical Fragment Analyzer and RNA markers. Between 250-450 μg of purified mRNA was generated in each reaction.

### 14. In Vitro Expression (IVEs)

Twenty-five (25) μL volumes of 5-fold serial dilutions of RNA (from 50 ng/well) were combined with an equal volume of OptMEM/lipofectamine mix (MessengerMax, Invitrogen) for 5 minutes RT in a 96-well deep-well (2.2 mL) plate (FisherScientific AB-0661). Expi293 suspension cells (ThermoFisher) were diluted in in 0.45 mL of Opti-MEM growth media to a final concentration of 1×106/well and were added to each sample well. The plate was incubated with shaking for 24 hours at 37° C. 8% CO₂ and 80% humidity overnight. The next day 150 UL of transfected cells were transferred to a 96-well plate (Corning 3894) to perform surface staining using a 1:1000 dilution of PD3 rabbit polyclonal anti-sera PapG, followed by PE conjugated antibody. Cells were also stained with Fixable Dye eFluor™ 780 to assess cell viability. Plates were read on an LSRII flow cytometer instrument (BD Biosciences).

PapG secreted into culture media supernatants 24 hour after the transfection of Expi293 cells with PapG_{LD} or PapG_{DSF} modRNA (see protocol above) was quantified by Octet biolayer interferometry. Transfections with 4-fold dilutions of RNA from 500 ng/well were evaluated. ProG biosensors were used to first bind the PapG mouse parental antibody (ECO-726) followed by binding reactions with clarified transfection supernatants. The biosensors were first hydrated with conditioned Expi293 media (200 μL/well) for 10 minutes at RT, followed by capture with saturating concentration of parental Ab (final 1 μg/mL, 200 μL/well) for 10 minutes at room temperature on an orbital shaker. Transfected supernatants were clarified by centrifugation (550×g, 10 minutes), and secreted PapG binding to the biosensors was measured by Octet. PapG concentrations were determined by interpolating values from a parallel titration of purified recombinant PapG protein standard using linear regression analysis. In addition, a 50 μL samples from the wells transfected with 500 ng RNA were taken for both constructs and centrifuged at 550×g for 4 minutes to separate the supernatants from the cell pellets. The cell pellets were resuspended in 50 ml of spent media. The supernatant and cell pellet samples were analyzed by SDS/PAGE followed by Coomassie staining and Western blot using anti-PapG polyclonal antibodies (wk6-pf13813) followed by goat anti-rabbit AP (Southern Biotech4030-04) for detection.

### 15. Immunofluorescence Microscopy

Human skeletal muscle cells (skMC) (Lonza #CC-2561) were seeded at 1×10⁴ cells/well in a polylysine coated 96-well plate (Perkin Elmer #6055508). RNA was diluted to 100 ng/well and 50 ng/well in media, combined with diluted lipofectamine (MessengerMax, Invitrogen) for 5 minutes RT. Next, the mRNA plus lipofectamine mixture was added to each well. The microplate was centrifuged at 500×g for 5 minutes at RT and incubated for 24 hr at 37° C. and 5% CO₂. The next day the monolayers were rinsed in PBS with Ca²⁺/Mg²⁺ and fixed with 4% paraformaldehyde for 20 minutes at room temperature. Plates were washed three times with PBS with Ca²⁺/Mg²⁺ and permeabilized with 0.1% saponin (Saponin permeating solution, 0.5% w/v soln. in PBS, 5X (Alfa Aesar #J63209-AK) diluted in goat serum (10% Normal Goat Serum, Fisher #50062Z) for 15 minutes at room temperature. Plates were washed three times, then cell surfaces were stained with rabbit polyclonal anti-PapG serum diluted 1:2000 in PBS with Ca²⁺/Mg²⁺ and 2% BSA overnight at room temperature. Wells were washed three times with PBS with Ca²⁺/Mg²⁺ and stained with goat anti-rabbit AlexaFluor488-conjugated secondary antibody (Invitrogen #A11008), diluted 1:500 in PBS with Ca²⁺/Mg²⁺2% BSA in PBS with Ca²⁺/Mg²⁺ for 2 hours at 4° C., or 37° C. for 30 minutes. Nuclei were stained with 4′6-Diamidino-2-Phenylindole, Dilactate (DAPI) at 1:2000 for 15 minutes at room temperature, before washing three times in PBS with Ca²⁺/Mg²⁺. Imaging of stained cells was performed using a CellInsight CX7 instrument (ThermoFisher Scientific).

### 16. PapG Hemagglutination (HA) Assay

A PapG HA assay was developed based on procedures described previously [13, 36, 37]. WT E. coli strain CFT073 bacteria were streaked out from a frozen glycerol stock for single colonies on a tryptic soy blood agar plate. To grow bacteria for use in the HA assay, bacteria were cultured overnight in Luria Bertani liquid media at 37° C. and shaking at 230 rpm. The next morning, bacteria were washed once with 1×PBS and normalized to an OD₅₄₀ of 1.0 in PBS. 30 μL of bacteria were then placed in a V-bottom 96-well plate (Corning, 3894) and serially diluted across the plate. Next, 30 μL serially diluted PapG specific ligand (“GbO₃”, TCI) or antisera from PapG immunized rabbits were incubated in the V-bottom plate with the bacteria for approximately 2 hours at 4° C. on an orbital shaker set to 190 rpm. All serial dilutions are 7-point titrations; for GbO₃ ligand, a 1:4 series was used and for rabbit sera, a 1:3. After this incubation, 60 μL of a 1.75% red blood cell (Human Red Blood Cells, Washed, Rockland Immunochemicals R407-0050) solution (diluted from 10% stock using 1×PBS) was added to all the wells in the plate. The plate was incubated for 2 hours at 4° C. and then imaged using a light box and camera or ImageQuant System. To collect quantitative data, assay plates were analyzed using a Clariostar instrument. Absorbance was measured at a wavelength of 540 nm, which was the maximum absorbance observed using an RBC only control well, and a read diameter of 4 mm within each well. Absorbance was normalized using blank controls (1×PBS only).

### B. Results and Discussion

### 1. Development of a Mammalian Cell Expression Platform for Production of PapG Recombinant Proteins

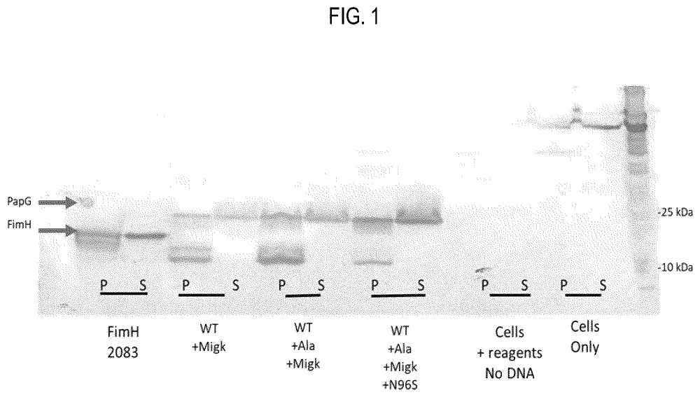

pcDNA3.1 plasmids were constructed wherein mammalian codon-optimized DNA sequences encoding PapG-II lectin domain (PapG_{LD}) (v1) were cloned in frame with sequence encoding a mouse IgGK signal peptide, to enable secretion of recombinant proteins into the culture supernatant of ExpiCHO cells. For PapG, since the ligand binding site of PapG is at the side of the molecule [27], multiple constructs were designed based on the prediction that the native signal peptide would be cleaved at either the N-terminal Tryptophan or Alanine residue (constructs are designated ‘+Ala’ in FIG. 1). Following transfection with these constructs, robust expression of PapG_{LD} proteins was observed in the culture supernatant (FIG. 1). PapG_{LD} was purified and subjected to mass spectrometry analysis which confirmed correct molecular weight and correct processing of the mouse IgGK signal peptide. Based on comparison with the published structure of PapG_{LD} [27], next generation constructs were all designed with the Tryptophan as the N-terminal residue.

When the PapG_{LD} protein sequence was analyzed by the glycosylation prediction software a single site was identified to be glycosylated, Asn96 (N96), which was mutated to N96S and the resulting protein confirmed to be aglycosylated by mass spectrometry; interestingly, this mutant appeared to express at slightly higher levels compared to WT PapG_{LD} (FIG. 1).

### 2. Engineering of Single Chain, Donor Strand Complemented Full-Length PapG Protein (PapG_{DSF})

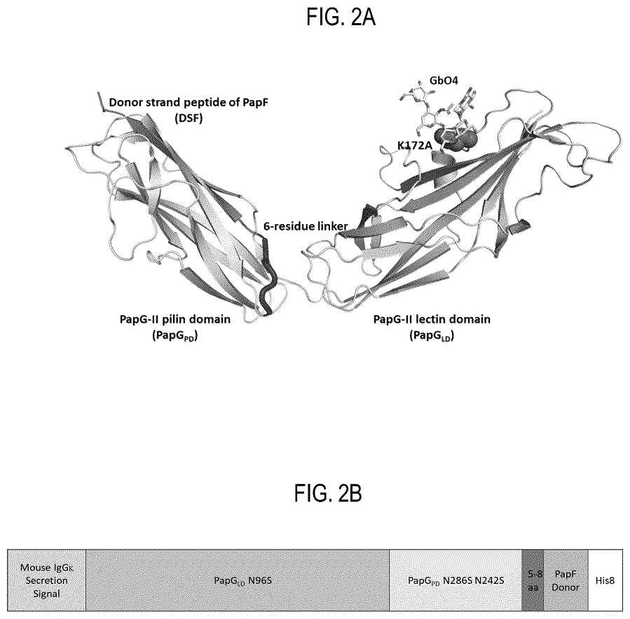

PapG, like many other fimbrial adhesin proteins, is composed of two domains, a lectin binding domain (PapG_{LD}) and a pilin domain (PapG_{PD}). PapG_{PD} forms an incomplete immunoglobin fold, resulting in a groove that provides a binding site for the N-terminal B-strand of PapF, forming a strong intermolecular linkage between PapG and PapF. While PapG_{LD} can be expressed in a soluble, stable form, full-length PapG is unstable unless complexed with the chaperone PapD [15]. Full-length PapG proteins (PapG_{DSF}) was designed wherein the pilin domain was donor strand complemented with a peptide from the N-terminus of PapF attached to the C-terminal residue of the pilin domain. We designed the full-length PapG protein (PapG-II sequence) which was fused to the N-terminal B-strand of PapF via a Glycine-Serine peptide linker to form a single chain fusion protein, PapG_{DSF}. A structural model of a PapG_{DSF} fusion protein based on AlphaFold2 prediction is illustrated in FIG. 2A. DNA sequences of PapG_{DSF} with different peptide linker lengths (5-9 GS residues) were cloned into pcDNA3.1 behind a mouse IgGK signal peptide and expressed in the supernatants of ExpiCHO cells. The PapG_{DSF} protein with the 6-residue linker was selected as the final design because this construct showed slightly higher expression levels then the constructs containing 7 and 8 amino acid linker lengths (FIG. 2B).

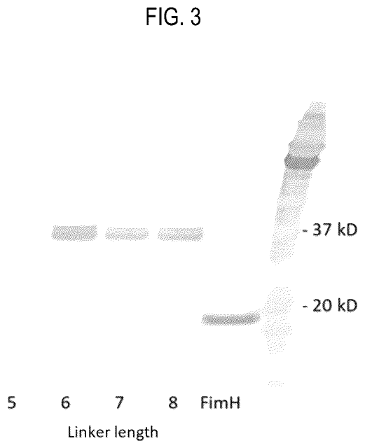

Glycosylation sites were predicted to be N96 and N286 using the software NetNGlyc (https://services.healthtech.dtu.dk/services/NetNGlyc-1.0/). The initial PapG_{DSF} work was performed with the glycosylation site mutant PapG_{DSF} N96S N286S. After expression in ExpiCHO cells and purification, a doublet was observed on the Coomassie gels and Western blots (FIG. 3). An additional glycosylation site (N242) was mapped by mass spectrometry (not shown). This additional glycosylation site was mutated to a Serine. PapG_{DSF} N96S N242S N286S was purified and confirmed to be devoid of N-glycosylation by mass spectrometry.

3. Structure-Based Design of PapG Mutants Unable to Bind their Cognate Receptors.

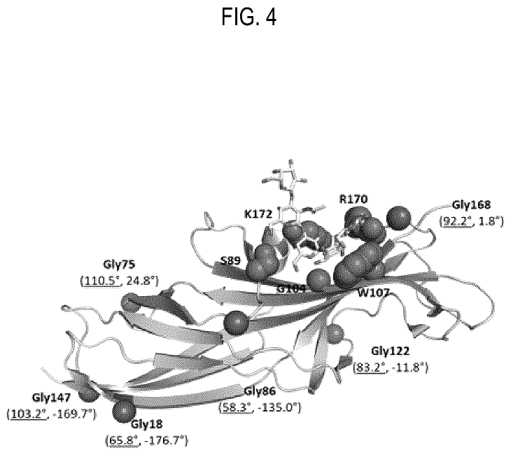

Structure-based design was used to guide the design of mutations in PapG_{LD} that were predicted to remove the ability to bind their cognate receptors, and potentially confer conformational stability. The location of mutations in the PapG lectin domains are shown in the AlphaFold2 generated model in FIG. 4.

Summary of Mutations Engineered into PapG_{LD} N96S:

For PapG_{LD}, residues that are in direct contact with the tetrasaccharide epitope of the globoside GbO₄ were selected for mutation to prevent the binding of the tetrasaccharide epitope based on the PapG-II complex structure. These include sites at S89, G104, W107, R170 and K172, as illustrated in FIG. 4. Glycine residues that have positive protein backbone F-angles in the PapG-II complex structure were also selected for mutation to favor a ligand-free PapG-II conformation. These include sites at G18, G75, G86, G122, G147 and G168, as illustrated in FIG. 4.

### 4. Expression of PapG Mutants in ExpiCHO Cells

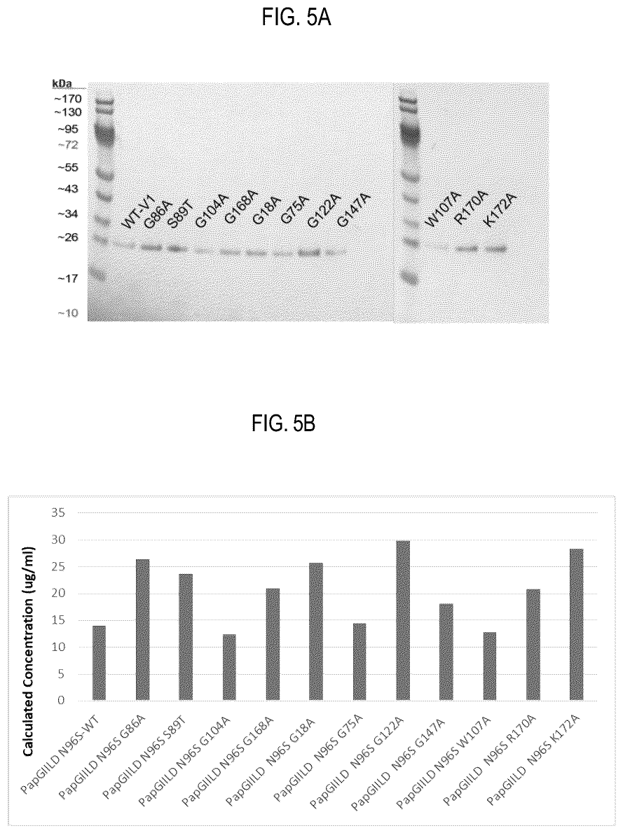

ExpiCHO cells were transfected with plasmids encoding PapG_{LD} variants and supernatants were screened by Western blot and Octet to quantify relative expression (FIG. 5). Several mutations led to increased expression levels of PapG_{LD} relative to their WT versions; and for PapG, introduction of G122A or K172A led to the highest increase in expression levels. Full length (FL) and ligand domain (LD) versions of PapG mutants were purified from culture supernatants from large scale transfections by nickel histidine chelation followed by hydrophobic interaction chromatography.

### 5. Biochemical Characterization of PapG Mutants

Purified proteins were evaluated in ligand binding and thermal stability studies, described below.

### Ligand Binding Affinity of PapG Mutants

Binding of PapG proteins to GbO₂ or GbO₄ ligands was evaluated using a Luminex platform wherein ligands were coupled to MagPlex beads via surfactant protein A (for GbO₂) or directly using EHS/NHS conjugation (for GbO₄). This assay was used to rank order binding affinities of the mutants, reported as Kd values interpolated from protein antigen titration curves (Table 7). G122A and K172A mutants also had reduced binding to GbO₄ ligand and K172A had reduced binding to GbO₂ ligand. While wild type PapG_{DSF} proteins with glycosylation mutations were able to bind both GbO₂ and GbO₄, no binding was detected for PapG_{DSF} N96S N242S N286S K172A.

TABLE 7
PapG Luminex ligand binding at equilibrium

| Mutant | GbO2/Kd | GbO4/Kd |
|---|---|---|
| PapG^{LD} N96S WTv1 | 0.7 | 14.1 |
| PapG^{LD} N96S WTv2 | 1.1 | 29 |
| PapG^{LD} N96SG18A | 1.8 | >10 |
| PapG^{LD} N96S G75A | 2.2 | >10 |
| PapG^{LD} N96S G86A | 1.3 | 15.0 |
| PapG^{LD} N96S S89T | 1.0 | <50 |
| PapG^{LD} N96S G104A | >>300^{Error! Reference source} ^{not found.} | >>300^{Error! Reference source not found.} |
| PapG^{LD} N96S G122A | 1.4 | >300^{Error! Reference source not found.} |
| PapG^{LD} N96S G147A | 4.6 | >300^{Error! Reference source not found.} |
| PapG^{LD} N96S G168A | 2.7 | >100 |
| PapG^{LD} N96S W107A | >>300^{Error! Reference source not found.} | >>300^{Error! Reference source not found.} |
| PapG^{LD} N96S R170A | >>300^{Error! Reference source not found.} | >>300^{Error! Reference source not found.} |
| PapG^{LD} N96S K172A | >>300^{Error! Reference source not found.} | >>300^{Error! Reference source not found.} |
| PapG^{DSF} N96S N286S | 1.6 | 8.8 |
| PapG^{DSF} N96S N242S | 2.2 | 3.0 |
| N286S | | |
| PapG^{DSF} N96S N242S | >>300^{Error! Reference source not found.} | >>300^{Error! Reference source not found.} |
| N286S K172A | | |

“a” = No binding detected at tested concentration

### Thermal Stability of PapG Mutants

The thermal stability of PapG WT and mutant proteins were determined using a SYPRO orange thermal shift-based differential scanning fluorimetry assay, wherein Tₘ designates the temperature at which 50% of the protein is unfolded. Non-covalent ligands often stabilize protein targets upon specific binding, resulting in an increase in protein melting temperature. Tm were therefore determined in the presence and absence of GbO₃ for PapG, and the difference in Tₘ (ΔTm) of protein in the presence of ligand relative to the apo form was calculated.

WT aglycosylated PapG_{LD} proteins (all including the N96S glycosylation mutation) exhibited similar Tₘ compared to aglycosylated PapG_{DSF} and had similar ΔTₘ in the presence of ligand (Table 8). WT PapG_{LD} had a melting temperature of 61.29° C. while the Tₘ of WT PapG_{DSF} was 61.82° C. In the presence of GbO₃, the Tₘ of WT PapG_{LD} and WT PapG_{DSF} increased by 3.84° C. and 3.67° C., respectively. This suggests that WT PapG_{LD} and PapG_{DSF} are both stabilized by GbO₃.

Introduction of ligand binding site mutations into PapG_{LD} impacted the Tₘ of PapG proteins. Mutations G104A, W107A, R170A and K172A are in the binding pocket of PapG and were shown to lower the binding affinity for GbO₄ in Luminex binding assays (Table 7). The Tm of the apo forms of PapG_{LD} G104A and W107A which were 64.54° C. and 62.64° C., respectively, were higher than WT PapG_{LD} (61.29° C.) suggesting that these mutations exert a stabilizing effect on the protein. The apo forms of PapG_{LD} R170A and K172A exhibited lower Tm (55.83° C. and 56.38° C., respectively) than aglycosylated WT PapG_{LD} which may indicate a destabilizing effect. There was very little shift in Tₘ in the presence of GbO₃ for all four of the PapG_{LD} mutant proteins (<0.5° C.) consistent with their reduced affinity for the ligand. This data agrees with the previous findings from the Luminex binding assays.

TABLE 8
Melting temperatures of PapG_{LD} mutants and PapGDsF in apo state and
in the presence of GbO₃.

| | | | | ΔTm | St. Dev. |
|---|---|---|---|---|---|
| | | Tm (Ave.)/ | St. Dev. | (Average)/ | (ΔTm)/ |
| PapG variant* | Replicates | ° C. | (Tm)/° C. | ° C. | ° C. |
| PapG_{LD} N96S_WT | 5 | 61.29 | 0.13 | 3.84 | 0.11 |
| PapG_{LD} N96S_G104A | 3 | 64.54 | 0.05 | 0.22 | 0.03 |
| PapG_{LD} N96S_W107A | 3 | 62.64 | 0.12 | 0.22 | 0.12 |
| PapG_{LD} N96S_R170A | 3 | 55.83 | 0.38 | 0.33 | 0.14 |
| PapG_{LD} N96S_K172A | 3 | 56.38 | 0.21 | 0.42 | 0.23 |
| PapG_{DSF} N96S N242S | 3 | 61.82 | 0.18 | 3.67 | 0.15 |
| N286S WT | | | | | |

*The N96S mutation in PapG_{LD} and the N96S, N242S and N286S mutations in PapG_{DSF} were introduced at sites found to be glycosylated during mammalian expression to eliminate non-native glycosylation.

Based on expression, ligand binding and thermal stability data, PapG K172A was selected for introduction into mRNA constructs for evaluation in in vitro expression experiments and animal immunogenicity studies, which will be compared to constructs encoding WT PapG proteins.

6. Development of an mRNA Platform for PapG Expression

### Expression of Secreted PapG in Supernatants of Mammalian Cells

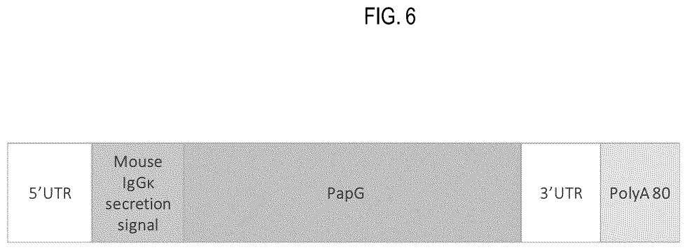

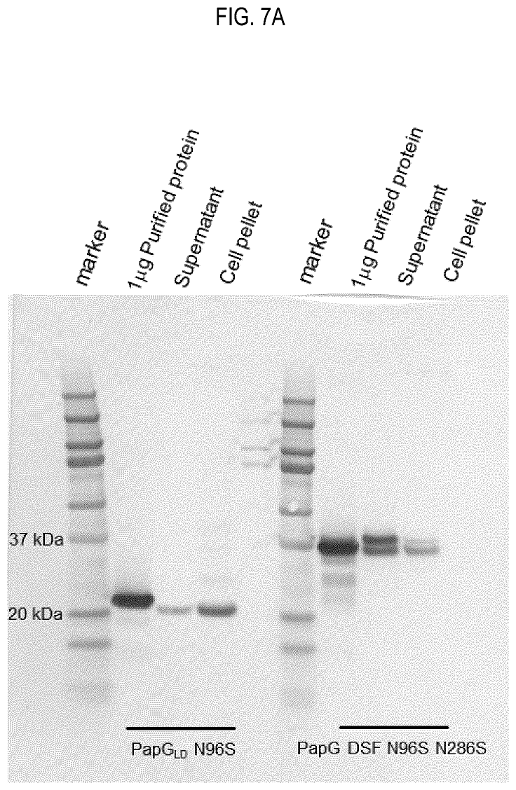

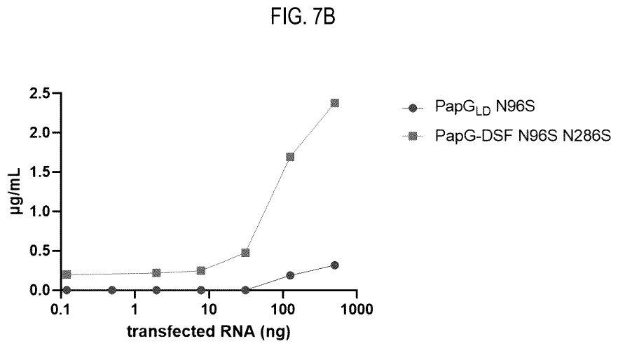

mRNA constructs encoding secreted WT PapG_{LD} or PapG_{DSF} versions (FIG. 6), using a mouse IgGK signal peptide, were cloned and modRNA transcribed from these constructs used to transfect Expi293 cells. PapG expression was evaluated in cell culture supernatants by Octet and Western blot; expression was readily detected (FIG. 7A & 7B). Higher levels of PapG_{DSF} were detected in culture supernatants than PapG_{LD} by either method. This confirmed that PapG can be expressed as a secreted antigen from transfected modRNAs.

### Expression of Membrane Anchored PapG on the Surface of Mammalian Cells

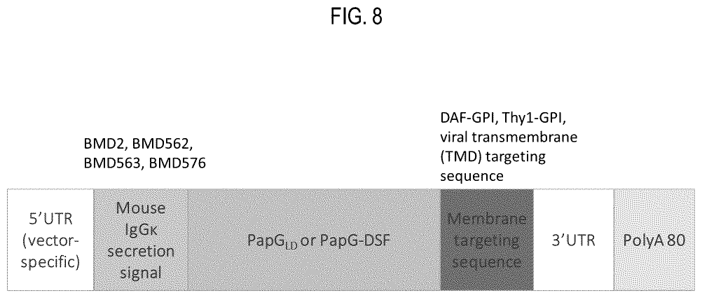

mRNA constructs encoding membrane anchored PapG_{LD} or PapG_{DSF} versions are described in FIG. 8. Several heterologous transmembrane targeting sequences were evaluated for anchoring PapG to the mammalian plasma membrane. Two Glycosylphosphatidylinositol phosphate (GPI) anchors were evaluated, which could potentially be used to express 2 antigens concurrently within the same cell [38].

Membrane Anchors Evaluated:

a. GPI anchors:
i. GPI sequence from human Decay Accelerating Factor (DAF) (also known as CD55): PapG was fused to the C-terminal 37 residues of human DAF protein, which are sufficient for conferring heterologous membrane association on viral glycoprotein ectodomains. The strategy for sequence replacement was informed from the consensus sequence developed from a computational model based on known mammalian GPI

signals [39]. For DAF-GPI, the bulk of the C-terminal GPI-attachment signal is cleaved in the endoplasmic reticulum (ER) concomitantly with addition of the GPI lipid moiety [40-42]. A surface-exposed potentially autoimmune peptide epitope on the C-terminal GPI motif was replaced with a serine-glycine linker to address possible safety concerns.
ii. GPI sequence from human Thyl: PapG was fused to the C-terminal 32 residues of human Thymocyte differentiation antigen 1 (Thyl), a GPI anchored surface glycoprotein for which the bulk of the C-terminal GPI anchor signal is also cleaved off in the ER, as for DAF-GPI [43, 44]. Fusion of different GPI anchor sequences to Horse Radish Peroxidase results in targeting to distinct lipid rafts, as well as differential glycosylation [44]. It was of interest to evaluate a second GPI anchor, if this GPI anchor drives higher expression or induces superior immune responses relative to the DAF-GPI anchor described above. As for DAF-GPI, a consensus sequence was developed from a mammalian GPI signal based computational model [39]. A surface-exposed, potentially autoimmune peptide epitope on the C-terminal GPI motif, was replaced with a serine-glycine linker to address possible safety concerns. According to this prediction algorithm, the exposed GPI peptide is naturally enriched in serine and glycine residues, suggesting a straightforward replacement strategy.
   b. Viral Transmembrane Domains (TMD):

Herpes Simplex Virus gD (HSV gD): PapG was fused to the C-terminal 60 amino acid residues of HSV gD protein including a hydrophobic TMD (HSV gD TMD) [45].

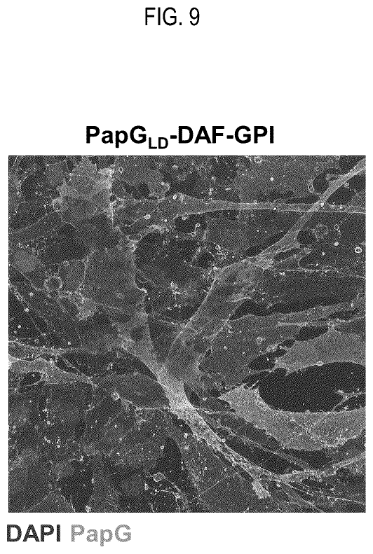

Using the DAF-GPI sequence, modRNA transcribed from the PapG_{LD} WT/BMD2 constructs was transfected into skeletal muscle cells which were stained with rabbit polyclonal anti-PapG antibodies to evaluate surface staining. Surface expression was confirmed by microscopy (FIG. 9).

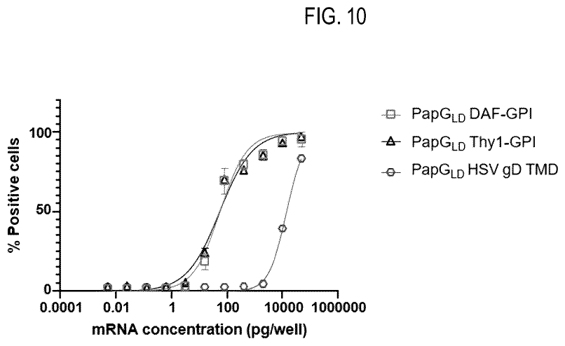

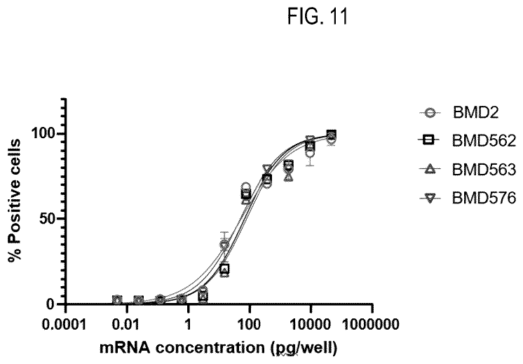

Expression of PapG on the cell surface was further evaluated by in vitro expression assays (IVE). Expi293 cells were transfected with a dilution series of modRNA transcripts encoding PapG_{LD} containing a GPI anchor or transmembrane domain. After 24 hours, cell surface protein expression was detected with PD3 rabbit sera (PapG) followed by R-Phycoerythrin-conjugated IgG and analyzed by flow cytometry (FIG. 10). Cells were transfected with modRNA transcripts encoding aglycosylated PapG_{LD} in the BMD2 backbone with either DAF-GPI, Thyl-GPI, or the TMD of HSV gD protein. The PapG_{LD} proteins with the DAF-GPI and Thyl-GPI anchors had comparable cell surface expression. The cell surface expression of PapG_{LD} with the HSV gD TMD was not as robust as it required a higher modRNA concentration to reach levels of expression comparable to that of the DAF and Thyl-GPI anchored proteins. Next, the cell surface expression levels of PapG_{LD} with the DAF-GPI anchor in the BMD562, BMD563 and BMD576 backbones were compared to BMD2 (FIG. 11). Each of the backbones contain a vector specific 5′UTR region (provided in Table 9). Cell surface expression appeared to be similar among these constructs.

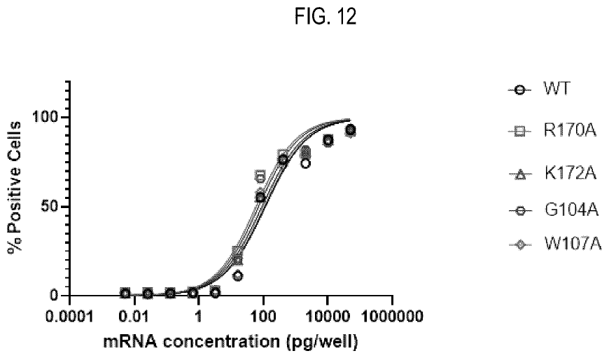

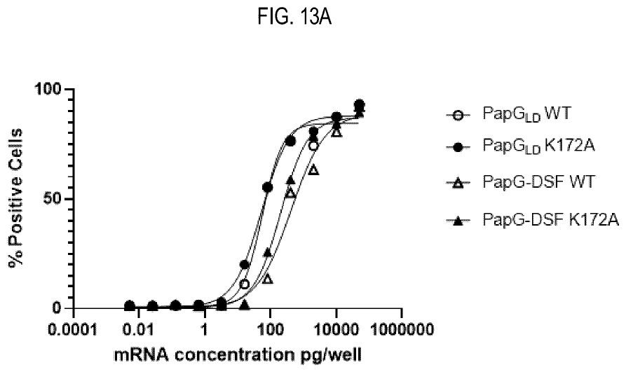

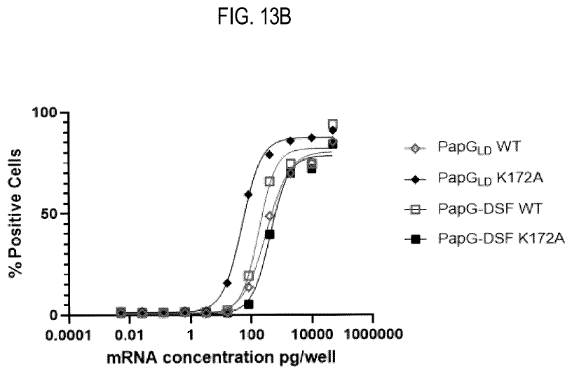

When purified PapG_{LD} proteins with engineered mutations were tested for GbO₄ binding in the Luminex assay, four mutations (R170A, K172A, G104A and W107A) were found to eliminate ligand binding (Table 7). modRNA transcripts encoding PapG_{LD} with DAF-GPI in the BMD2 backbone containing each of these non-binding mutations were generated. Expi293 cells were transfected with the mutant and wild-type modRNA transcripts, and cell surface expression was evaluated by IVE (FIG. 12). Cell surface expression levels of PapG_{LD} with the non-binding mutations were similar to WT. K172A was selected as the candidate to move forward in an animal study (see Example 2 hereinbelow), based on GbO₄ non-binding and higher expression levels (Table 7 and FIG. 5A & 5B). To differentiate backbones and expand to full-length protein, K172A was engineered into a full-length aglycosylated PapG_{DSF}-DAF-GPI BMD2 mRNA construct. modRNA transcripts from K172A and the WT construct were evaluated by IVE assay (FIG. 13A & 13B). Surface expression levels were similar for the WT and the K172A mutant.

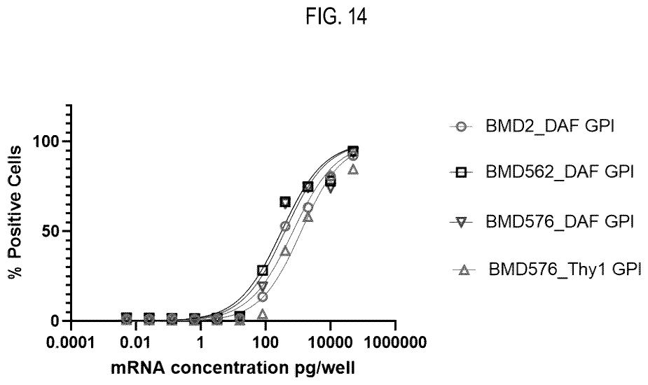

To compare expression between backbones, format (LD or DSF) and conformation (WT or non-binding mutant), mRNA constructs with K172A and wild-type PapG_{LD} and PapG_{DSF} with DAF-GPI and Thyl-GPI anchors in the BMD576 and BMD562 backbones were generated. IVE confirmed expression was similar for all constructs (FIG. 14).

7. Development of a Hemagglutination (HA) Assay as a Functional Assay to Evaluate Sera from PapG Immunized Animals.

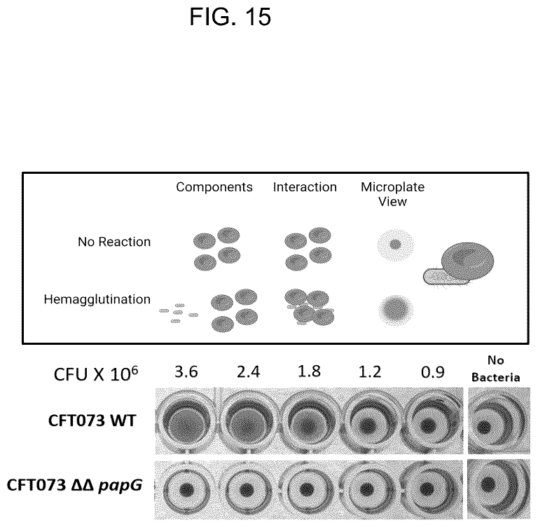

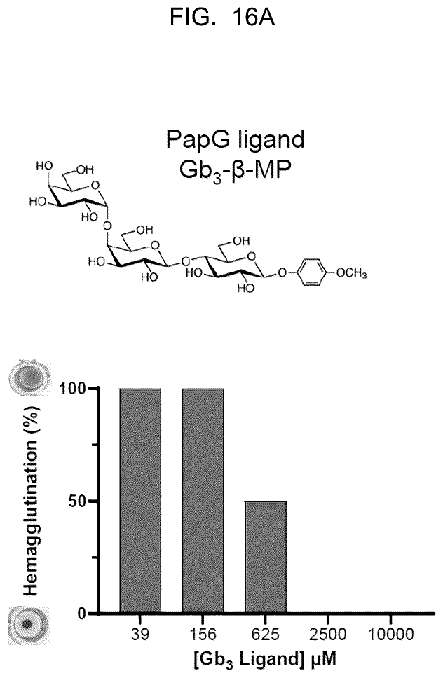

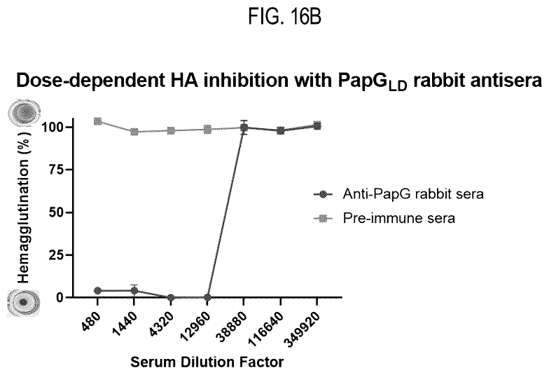

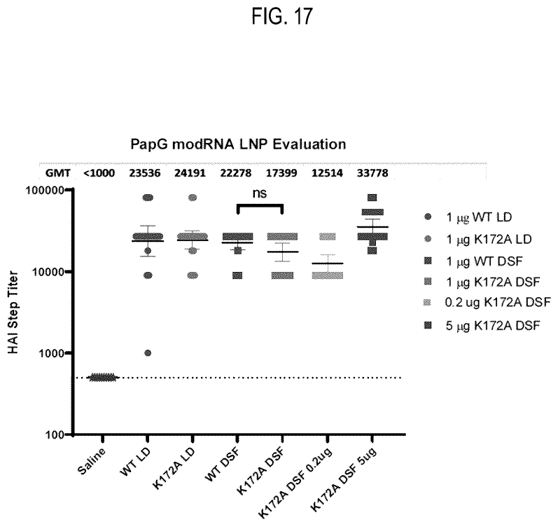

PapG mediates the ability of E. coli to agglutinate human red blood cells (RBC) [13, 36, 37]. An HA assay was developed in which RBC were incubated with decreasing CFU of E. coli strain CFT073; hemagglutination was visible using the WT CFT073 strain and did not occur with a mutant CFT073 strain in which both copies of PapG were deleted (FIG. 15). Incubation of WT CFT073 strain with a PapG ligand, GbO₃, led to a concentration-dependent inhibition of hemagglutination (FIG. 16). Similarly, hemagglutination was inhibited in the presence of polyclonal sera from rabbits immunized with recombinant PapG protein (FIG. 16).

In the above Example(s), a mammalian expression platform was successfully developed for production of high yields of full-length single chain donor strand complemented versions of PapG, which were designed and produced as described herein. Mutants of PapG were identified that have reduced binding to their cognate ligands and exhibit acceptable thermal stability. modRNA constructs encoding secreted and membrane associated WT and mutant versions of LD or full-length forms of PapG were shown to express protein in mammalian cells. Finally, proof of concept for a human red blood cell agglutination assay for the evaluation of PapG neutralizing antibodies was generated by demonstrating specificity with PapG antisera and a competitive ligand that mimics the natural receptor of PapG.

### Example 2

### Immunogenicity of Monovalent PapG modRNA LNPs [Study VAC-2023-PRL-EC-398]

PapG is an adhesin of the type P pilus that mediates attachment of uropathogenic

Escherichia coli to the uroepithelium of human kidney. The PapG lectin binds to the globoside (GbO₄) glycolipid, which is enriched in urinary tract tissue and consists of the tetrasacharide GalNAcβ1-3Galα1-4Galβ1-4Glc linked to ceramide. Here we present the use of a hemagglutination inhibition assay (HAI) to determine vaccine functional responses to protein subunit PapG or mRNA-expressed PapG antigens. In this assay the agglutination of human red blood cells (hRBCs) expressing GbO₄ is caused by live E. coli CFT073 bacteria expressing PapG. The HAI assay measures the ability of PapG-elicited antibodies to prevent hRBC agglutination, exemplifying inhibition of the key bacterial adherence mechanism leading to clinical pyelonephritis. The assay was used to characterize the functional activity of PapG monoclonal antibodies (mAbs) raised against a PapG lectin domain subunit antigen. PapG HAI data from a mouse immunogenicity study are described that evaluated various 5 monovalent PapG modRNA LNPs and a PapG subunit comparator.

In this mouse study immunogenicity of different PapG mRNA constructs were evaluated. The following comparisons were investigated: the PapG lectin domain only vs the full-length PapG_{DSF} (lectin plus pilin domain); wild type PapG vs the PapG K172A ligand binding mutant (does not bind GbO₄-ceramide); the DAF vs Thy GPI surface anchor; the 5′ UTRs BMD562 vs BMD576, and mRNA doses of 0.2,1 and 5 μg. The study design including vaccine dosing details and schedule is in shown in Table 15.

TABLE 15
Study Design

| | Antigen RNA construct | Vaccine | Dose Vol/ | Vax | Bleed |
|---|---|---|---|---|---|
| Grp# | [SEQ ID NO:] | Dose | Route | (Week) | (Week) |
| 1 | Saline | N/A | 50 μl/ IM | 0, 4 | 0, 6 |
| 2 | BMD576_PapG-DSF K172A_Thy1-GPI | 1 μg | 50 μl/ IM | 0, 4 | 0, 6 |
| | [SEQ ID NO: 66] | | | | |
| 3 | BMD576_PapGLD WT_DAF-GPI | 1 μg | 50 μl/ IM | 0, 4 | 0, 6 |
| | [SEQ ID NO: 45] | | | | |
| 4 | BMD576_PapG-DSF WT_DAF-GPI | 1 μg | 50 μl/ IM | 0, 4 | 0, 6 |
| | [SEQ ID NO: 65] | | | | |
| 5 | BMD576_PapGLD K172A_DAF-GPI | 1 μg | 50 μl/ IM | 0,4 | 0, 6 |
| | [SEQ ID NO: 52] | | | | |
| 6 | BMD576_PapG-DSF K172A_DAF-GPI | 0.2 μg | 50 μl/ IM | 0,4 | 0, 6 |
| | [SEQ ID NO: 64] | | | | |
| 7 | BMD576_PapG-DSF K172A_DAF-GPI | 1 μg | 50 μl/ IM | 0, 4 | 0, 6 |
| | [SEQ ID NO: 64] | | | | |
| 8 | BMD576_PapG-DSF K172A_DAF-GPI | 5 μg | 50 μl/ IM | 0, 4 | 0, 6 |
| | [SEQ ID NO: 64] | | | | |
| 9 | BMD562_PapG-DSF K172A_DAF-GPI | 1 μg | 50 μl/ IM | 0, 4 | 0, 6 |
| | [SEQ ID NO: 63] | | | | |

This study was performed to determine the immunogenicity of various PapG modRNA LNPs in mice, and the results showed full-length PapG-DSF K172A construct as the best nonligand binding vaccine antigen. Functional response data from HAI and T24 assays were comparable across the groups tested and are summarized in Table 16. Individual serum titers and group GMT titers for the HAI assay are shown in Table 16. Corresponding T24 assay raw data (serum titration curves) for the serum pools (generating the IC50 titers) are also shown in Table 16. The most important result was confirmation that similar inhibitory responses are generated by both the non-ligand binding mutant K172A PapG antigens (Groups 2, 5, 7, 9) and the wild type PapG antigens (Groups 3, 4). Similar inhibitory antibodies were generated by both lectin domain (Groups 3 and 5) and full-length PapG adhesin constructs (Groups 2, 4, 5, 7, 9). Dose responses were as expected for Groups 6, 7, 8 with lowest step titer/GMT from the 0.2 μg dose (Group 6) and highest step titer/GMT with the 5 μg dose (Group 8). There were no statistical differences in GMTs between modRNA LNPs with the BMD576 or BMD562 mRNA 5′ UTR modRNA transcripts; also, no statistical differences between use of either the DAF GPI or the Thyl GPI lipid anchor. The extraordinarily high inhibitory titers generated by these mRNA constructs in the PapG HAI were unexpected, but confirmed by similarly potent T24 kidney cell neutralization assay data (Table 16).

TABLE 16
Study Results

| | | | | | T24** |
|---|---|---|---|---|---|
| | | Vaccine | Step Titer* | Step Titer* | Pool |
| Grp# | Antigen | Dose | Pool | GMT | IC50 |
| 1 | Saline | N/A | <1,000 | <1,000 | N/I |
| | | | (LOD) | (LOD) | |
| 2 | BMD576_PapG-DSF K172A_Thy1-GPI | 1 μg | 81,000 | 85,681 | 69,700 |
| 3 | BMD576_PapGLD WT_DAF-GPI | 1 μg | 81,000 | 94,822 | 76,000 |
| 4 | BMD576_PapG-DSF WT_DAF-GPI | 1 μg | 81,000 | 89,753 | 70,300 |
| 5 | BMD576_PapGLD K172A_DAF-GPI | 1 μg | 81,000 | 98,165 | 63,800 |
| 6 | BMD576_PapG-DSF K172A_DAF-GPI | 0.2 μg | 54,000 | 50,054 | 63,300 |
| 7 | BMD576_PapG-DSF K172A_DAF-GPI | 1 μg | 27,000 | 69,595 | 76,500 |
| 8 | BMD576_PapG-DSF K172A_DAF-GPI | 5 μg | 81,000 | 141,214 | 288,500 |
| 9 | BMD562_PapG-DSF K172A_DAF-GPI | 1 μg | 81,000 | 65,875 | 115,500 |

N/I = no inhibition;
*HAI Assay Data: Individual Titers and GMT;
**Pooled serum Postdose 2 (PD2 ; wk6) IC50 Titers in T24 Kidney Cell Ligand Binding Inhibition (Neutralization) Assay.

i. modRNA LNP Production

DNA plasmids encoding E. coli PapG proteins were prepared and utilized for in vitro transcription reactions to generate RNA. In vitro transcription of RNA is known in the art and is described herein. DNA templates were cloned into a modRNA cloning entry vector with backbone sequence elements (T7 promoter, 5′ UTR, 3′ UTR, and 3′ poly-A tail) with improved

RNA stability and translational efficiency. The DNA was purified, spectrophotometrically quantified and in vitro-transcribed by T7 RNA polymerase in the presence of a trinucleotide cap1 analogue ((m₂^{7,3′-O})Gppp(m^{2′O})ApG) known as CleanCap® AG; TriLink Biotechnologies) set forth below:

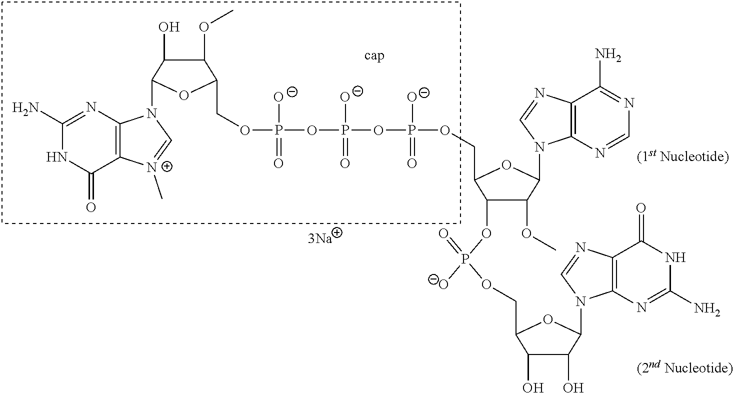 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

and N¹-methylpseudouridine-5′-triphosphate (also known as N¹-methylpseudouridine-5′-triphosphate, N¹meψTP, m¹ψTP, 1-methyl-pseudouridine phosphoramidite or N¹-methyl-pseudouridine-5′-triphosphate; TriLink Biotechnologies), which has the structure set forth below:

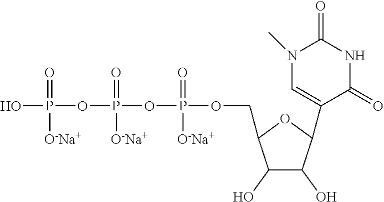

in order to replace the uridine residues and thereby form the modified RNA (modRNA).

The PapG RNA was generated from codon-optimized (CO) DNA for stabilization and superior protein expression. DNA constructs of the present disclosure, and corresponding RNA sequences, comprising a 5′ UTR, an open reading frame encoding a PapG polypeptide, a 3′ UTR and a 3′ poly-A tail are described herein.

The purification of the transcripts was performed by Turbo DNase digestion followed by LiCl precipitation. The modRNA pellet was resuspended in Nuclease Free Water and henceforth identified as Drug Substance (DS) and LNP encapsulated drug product (DP).

RNAs were formulated with a mixture of synthetic lipids ALC-315 and ALC-159, distearoylphosphatidylcholine (DSPC) and cholesterol (ALC315: cholesterol: DSPC: ALC159=46.3:42.7:9.4:1.6).

ii. PapG Luminex Ligand Binding Inhibition Assay (LLIA)

The PapG Luminex Ligand-binding Inhibition Assay uses synthetic or native ligands of PapG tethered onto the carboxylic MagPlex Luminex beads for binding of recombinant PapG protein. Bound PapG was detected with antibody to the C-terminal histidine tag. Structures of the native ligand (GbO₄-ceramide) and synthetic ligand (GbO₄-phenoxy-octyl-amine) used for bead adsorption or co-valent coupling are shown below:

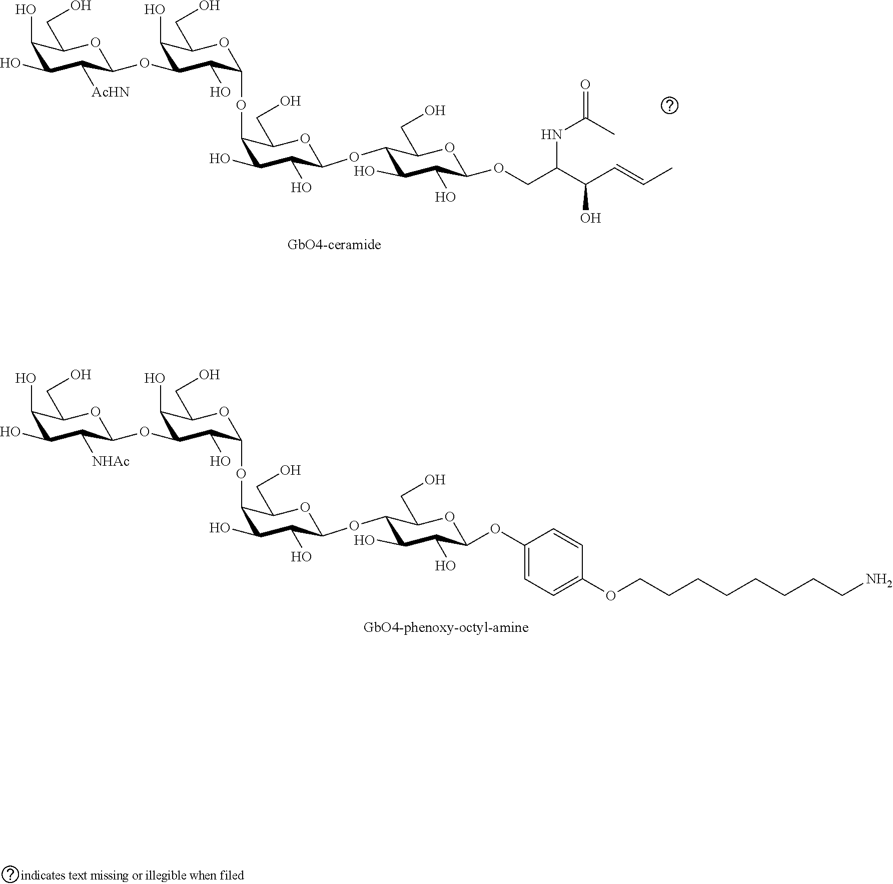

The native PapG ligand globotetraosyl ceramide (GbO₄-ceramide) is adsorbed onto MagPlex beads pre-coupled with a lipid binding protein, which consists of the N-terminal domain of mammalian Surfactant protein A (SP-A). The SP-A protein was randomly coupled to MagPlex beads, with free amines cross-linking with the EDC/NHS-activated carboxyl-ester beads. Coupling resulted in approximately 50-80% of available bead carboxylic sites bound to the SP-A protein. The resulting SP-A beads were adsorbed with a suspension of purified porcine GbO₄-cer. The amount of GbO₄-cer adsorbed is calculated by using the MagPlex beads specifications from the manufacturer (Luminex) whereby each MagPlex bead has 1×108 carboxylic (binding) sites: by dividing the number of carboxylic sites by Avogadro's number 6.0221×1023 particles per mole, the maximum moles of binding per bead is 1.66×10-16, assuming 100% coupling. A one milliliter suspension of MagPlex beads containing 1.25×107 beads can bind a maximum of 2.075×10−9 moles of ligand. For the lipidated native ligand GbO₄-cer to adsorb to the MagPlex beads, the lipid binding SP-A protein is first bound to approximately 50-80% of bead carboxylic sites; for one milliliter of beads, 50% coupling is equivalent to 1×10−9 moles, or for 80% coupling, 1.7×10−9 moles of SP-A loaded on the beads. An additional assumption is that 100% of the SP-A protein on the bead surface can bind to (adsorb) the GbO₄-cer ligand.

The GbO₄-phenoxy synthetic globoside ligand (Z Biotech) has an eight-carbon chain linker and a terminal free amine, allowing directional coupling to MagPlex beads via EDC/NHS activated carboxyl-ester (35-70% of carboxylic sites used). Therefore, assuming 100% coupling efficiency, addition of 7.3×10-10 moles or 1.45×10−9 moles of ligand to 1 mL of EDC/NHS activated MagPlex beads will result in ligand loads of 35% or 70% per bead, respectively.

The PapG LLIA uses a fixed (2 nM) concentration of recombinant wild-type (WT) PapG lectin domain protein or full-length protein to bind 2,500×ligand-coated beads per well, while incubating overnight at 4° C. Antibody serial dilutions of 1:3 were evaluated starting with top concentrations of either 100 nM or 300 nM, for mAbs, or starting at 1:100 dilution for immune sera. After overnight incubation, the beads are washed, and PapG ligand binding is detected by an anti-poly histidine-phycoerythrin labelled mAb, with fluorescence signal detected on a FlexMap 3D instrument. IC50s are determined using GraphPad Prism with a non-linear regression 4-PL curve fit.

iii. PapG Hemagglutination Inhibition Assay (HAI)
1. Preparation of Single Use Frozen E. coli CFT073 Banks

Frozen bacterial stocks for use in the PapG HAI are stored at a concentration of ˜15 OD/mL. For stock preparation, take 6 ×100 μL of frozen parental strain CFT073 glycerol stock to seed six overnight cultures with ˜85 mL Luria's Broth (LB) each in 250 mL vented flasks. Flasks are shaken at 200 rpm and 37° C. overnight. On the same day, prepare 100 mL LB media with 30% glycerol and 0.4% sucrose by mixing 36 mL 80% Glycerol with 0.8 mL 50% Sucrose in 64 mL LB and pre-chill at 4° C. After ˜16 h growth, determine the OD₆₀₀ nm of each flask with the SpectraMax M5e plate reader by adding 100 μL culture to 900 μL LB (1:10 dilution). After reading the OD600, harvest cells by pelleting in a centrifuge at 4,000g, 4° C., for 30 minute. Determine the total OD of each of the six flasks, by multiplying the volume by OD/mL of each flask and divide by 15 OD to give the appropriate pellet resuspension volume. Discard supernatants and resuspend all pellets in the pre-chilled LB cryopreservant containing 30% glycerol and 0.4% sucrose, with a final volume as calculated above. Pool all resuspended pellets and mix gently, then aliquot into 1 mL/cryotube, and freeze at −70° C.

2. Preparing 10% hRBC Working Solution From Stock

Gently mix human red blood cells (hRBC) bottle (Rockland) by rocking ˜20×times, pipet out 10 mL of the hRBCs into a 15 ml conical tube. Centrifuge at 2,000 rpm(˜990×g) for 10 minutes and 4° C. Aspirate and discard the supernatant, then resuspend the pellet with 9 mL 4° C. PBS. The packed hRBCs in 15 mL conical tube should constitute roughly 1 mL of this total volume. Store these 10% hRBCs at 4° C. until use.

iv. PapG Hemagglutination Inhibition (HAI) Assay

The PapG hemagglutination inhibition (HAI) assay uses human red blood cells (hRBCs), which naturally express the PapG ligand GbO₄ (globotetraosyl ceramide) on the cell surface, and the E. coli strain CFT073 expressing the fimbrial adhesin PapG. Upon mixing bacteria with hRBCs, precipitated cells remain suspended (presenting as a cloudy well), while no binding results in the red blood cells falling out of solution towards the center of the well (observed as pellet).

The total final well volume is 100 μL, with serum dilutions comprising 25 μL of this volume, strain CFT073 cells in 25 pL volume, and hRBCs in a 50 μL volume. The assay plate is a V-bottom plate to which 25 μL 4° C. PBS is initially added to all wells. The positive control wells receive an additional 25 μL 4° C. PBS. The no-serum negative control consists of E. coli CFT073 cells plus hRBCs only (resulting agglutinated well appears cloudy). To accommodate pre-diluted sera transferred from the serum Master Dilution plate (MDP) an extra 5 μL 4° C. PBS is added to wells in Row A of the assay plate (columns 1 to 12). The positive control for inhibition of agglutination will ultimately include a serum standard; for now, hRBCs only.

The MDP is prepared in a 0.5 mL deep-well plate by pre-diluting the test sera (and replicates) using 0.1% BSA PBS as diluent, with a typical pre-dilution of one to fifty (1:50). Serum samples in a single row of the MDP are diluted by adding 5 μL of sera (plus adjacent replicates) to 245 μL of 0.1% BSA in PBS and mixed. In this way, six individual serum samples with replicates are accommodated. Each MDP row contains sufficient prediluted sera for a total of 8 assay plates (to enable repeat runs if needed). Transfer and mixing of 7.5 μL from the MDP into 30 μl in the first row of each individual assay plate is performed with a 12-channel pipette. 12.5 microliter volumes are transferred serially by row (into preprepared 25 μL volumes) until row G, when after mixing 12.5 microliters are aspirated and discarded. The top serum pre-dilution is dependent on the desired serum dilution range to be evaluated in the PapG HAI, typically equivalent to a 1:1000 assay plate final dilution.

The assay plate, now with transferred serum serial dilutions, is set aside and stored at 4° C. prior to adding hRBC and bacteria.

Each CFT073 vial is thawed in the biosafety cabinet and contents transferred from the cryotube stock into a 2 mL Eppendorf for two cycles of washing: after centrifugation at 5,000×g for 3.5 minutes, the supernatant is removed and discarded, and the cell pellet is resuspended gently in 2 mL 4° C. PBS; the tube is then centrifuged again at 5,000g for 3 minutes, the supernatant removed and discarded, and the cell pellet gently resuspended in 1 mL 4° C. PBS. The washed bacteria (equivalent to 15 OD600 units of CFT073) are transferred to a 5 ml tube and the volume adjusted to 3 mL with 4° C. PBS. 25 μL of this washed and diluted CFT073 bacterial suspension is added to all wells of the assay plate (with serum dilutions), except for the Positive Control wells.

The plate is then incubated for 1 hour, at 4° C. with shaking (150 RPM). To prepare a 1% RBC stock for addition to the assay plate, add 700 μL of 10% hRBC (previously washed and prepared) to 6.3 mL of 4° C. PBS for each 96-well plate being used. After the pre-incubation of serum and cells in the assay plate, add 50 μL 1% hRBC to all wells in the assay plate and gently mix by pipetting up and down (2×). Then the assay plate is incubated at room temperature for 1, 2, 16 hours (without shaking/disturbing the assay plate). The image of the plate (picture) is the raw data and the step titers for each replicate serum dilution is recorded. Results are recorded after 1 hour, 2 hours and/or overnight ˜16 hours.
1. To record PapG HAI data, take a camera image(s) of the assay plate so that all 96 wells are visible and calls for last dilution with an RBC pellet (no HAI activity) can be made. Place the assay plate on a white background (printer paper) then screw the Sony camera in place on its stand. Block glare from overhead lights by using an umbrella. Take pictures and re-center/focus as adjustments are made. Then connect the camera a via USB cable to a computer and transfer images. To transfer files from camera to PC select the USB/camera folder and drag and drop images into a folder on PC/One Drive. The images can then be deleted from the camera.
2. Step titers are determined by observing and recording each column dilution and calling the last well inhibited. Inhibition is determined by the well that exhibits a similar circular solid red dot in center of well like the positive control wells. The breakpoint dilution between pelleted RBC and cloudy agglutination is recorded as the step titer IC50.
3. Data analysis is performed both in Excel (for collation of step titer (IC50s)) and in GraphPad Prism (group analysis performed using column analysis and ANOVA on log transformed data for Group-to-Group comparisons).
   v. PapG T24 Bladder-Cell Ligand-Binding-Inhibition (Neutralization) Assay

The neutralization assay depends on the binding of P-piliated bacteria to GbO₄ present on the surface of T24 bladder cells. A monolayer of T24 bladder cells is grown in a 96-well plate. Once confluent, the cells are fixed in a 1% paraformaldehyde for 15 minutes, then blocked in a blocking buffer of PBS containing 1× fish gelatin (diluted from 10× commercial stock, Biotium). In a separate plate, antibodies are serially diluted in PBS containing 0.1× fish gelatin and then co-incubated with 1×106 E. coli cells for 1 hour at 37° C. The E. coli/antibody complex is added to the T24 cells and after a 1 hour incubation at 37° C., unbound cells are washed away. Adherent CFT073 cells are detected with a fluorescently labelled anti-06 antigen mAb (mAb ECO-232-16). The IC50 titers of anti-PapG serum pools are then determined in GraphPad Prism using a 4-PL variable slope curve fit.

### Sequences

TABLE 9
5′ UTR and 3′ UTR Sequences

SEQ ID NO: 1 >BMD2 5′UTR
AGGAAAUAAGAGAGGAUAAGACGACUAAGGAGACAUACAGAA
UAAGAGGCA
SEQ ID NO: 2 >BMD2 3′UTR
GCUCGCUUUCUUGCUGUCCAAUUUCUAUUAAAGGUUCCUUUG
UUCCCUAAGUCCAACUACUAAACUGGGGGAUAUUAUGAAGGG
CCUUGAGCAUCUGGAUUCUGCCUAAUAAAAAACAUUUAUUUU
CAUUGCAA
SEQ ID NO: 3 >BMD576 5′UTR
AGGAGGACUGGUCGAACCUGCAUAGUGAUCAUAAGGUCAGCA
UA
SEQ ID NO: 4 >BMD576 3′UTR
GCUCGCUUUCUUGCUGUCCAAUUUCUAUUAAAGGUUCCUUUG
UUCCCUAAGUCCAACUACUAAACUGGGGGAUAUUAUGAAGGG
CCUUGAGCAUCUGGAUUCUGCCUAAUAAAAAACAUUUAUUUU
CAUUGCAA
SEQ ID NO: 5 >BMD563 5′UTR
AGGAAAUAAGAGAGGAUAAGACGACUAAGGAGACAUACAGAA
UAAGAAACAGGCA
SEQ ID NO: 6 >BMD563 3′UTR
GCUCGCUUUCUUGCUGUCCAAUUUCUAUUAAAGGUUCCUUUG
UUCCCUAAGUCCAACUACUAAACUGGGGGAUAUUAUGAAGGG
CCUUGAGCAUCUGGAUUCUGCCUAAUAAAAAACAUUUAUUUU
CAUUGCAA
SEQ ID NO: 7 >BMD562 5′UTR
AGGAAAUAAGAGAAAGAGGAUAAGACGACUAAGGAGACAUAC
AGAAUAAGAGGCA
SEQ ID NO: 8 >BMD562 3′UTR
GCUCGCUUUCUUGCUGUCCAAUUUCUAUUAAAGGUUCCUUUG
UUCCCUAAGUCCAACUACUAAACUGGGGGAUAUUAUGAAGGG
CCUUGAGCAUCUGGAUUCUGCCUAAUAAAAAACAUUUAUUUU
CAUUGCAA
SEQ ID NO: 9 >hHBB 5′UTR
AGGACAUUUGCUUCUGACACAACUGUGUUCACUAGCAACCUC
AAACAGCCACC
SEQ ID NO: 10 >hHBB 3′UTR
GCUCGCUUUCUUGCUGUCCAAUUUCUAUUAAAGGUUCCUUUG
UUCCCUAAGUCCAACUACUAAACUGGGGGAUAUUAUGAAGGG
CCUUGAGCAUCUGGAUUCUGCCUAAUAAAAAACAUUUAUUUU
CAUUGCA

TABLE 10
PapG mutant polypeptide sequences*

SEQ ID NO: 11> PapG_{LD}_V1
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDNDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 12> PapG_{LD} V2
METDTLLLWVLLLWVPGSTGWNNIVFYSLGNVNSYQGGNVVITQRPQF
ITSWRPGIATVTWNQCNGPEFADGSWAYYREYIAWVVFPKKVMTQNGY
PLFIEVHNKGSWSEENTGDNDSYFFLKGYKWDERAFDAGNLCQKPGET
TRLTEKFDDIIFKVALPADLPLGDYSVKIPYTSGMQRHFASYLGARFK
IPYNVAKTLPRENEMLFLFKNIGG
SEQ ID NO: 13> PapG_{LD} N96S
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 14> PapG_{LD} N96S G86A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKASWSEENTGDSDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 15> PapG_{LD} N96S S89T
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWTEENTGDSDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 16> PapG_{LD} N96S G104A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKAYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 17> PapG_{LD}_N96S_G168A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLAARFK
IPYNVAKTLPRENEMLFLFKNIGG
SEQ ID NO: 18> PapG_{LD}_N96S_G18A
METDTLLLWVLLLWVPGSTGWNNIVFYSLGNVNSYQGANVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 19> PapG_{LD}_N96S_G75A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNAY
PLFIEVHNKGSWSEENTGDSDSYFFLKGYKWDERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 20> PapG_{LD}_N96S_G122A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPAET
TRLTEKFNDIIFKVALPADLPLGDYSVTIPYTSGIQRHFASYLGARFK
IPYNVAKTLPRENEMLFLFKNIGG
SEQ ID NO: 21> PapG_{LD}_N96S_G147A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLADYSVTIPYTSGIQRHFASYLGARFK
IPYNVAKTLPRENEMLFLFKNIGG
SEQ ID NO: 22> PapG_{LD}_N96S_W107A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKADERAFDAGNLCQKPGET
TRLTEKFNDIIFKVALPADLPLGDYSVTIPYTSGIQRHFASYLGARFK
IPYNVAKTLPRENEMLFLFKNIGG
SEQ ID NO: 23> PapG_{LD}_N96S_R170A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLGAAFK
IPYNVAKTLPRENEMLFLFKNIGG
SEQ ID NO: 24> PapG_{LD}_N96S_K172A
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLGARFA
IPYNVAKTLPRENEMLFLFKNIGG
SEQ ID NO: 26> PapG_{DSF} N96S N242S N286S K172A*‡
METDTLLLWVLLLWVPGSTGWNNIVFYSLGNVNSYQGGNVVITQRPQF
ITSWRPGIATVTWNQCNGPEFADGSWAYYREYIAWVVFPKKVMTKNGY
PLFIEVHNKGSWSEENTGDSDSYFFLKGYKWDERAFDAGNLCQKPGET
TRLTEKFNDIIFKVALPADLPLGDYSVTIPYTSGIQRHFASYLGARFA
IPYNVAKTLPRENEMLFLFKNIGGCRPSAQSLEIKHGDLSINSANNHY
AAQTLSVSCDVPANIRFMLLRSTTPTYSHGKKFSVGLGHGWDSIVSVN
GVDTGETTMRWYKAGTQSLTIGSRLYGESSKIQPGVLSGSATLLMILP
GGSSGGADVQINIRGNVYIPP
*sequences may also comprise a C-terminal His8 tag sequence (e.g. HHHHHHHH); mouse IgGκ signal peptide sequence is indicated in italics.
‡for the PapG_{DSF} constructs, the underlined amino acids are Gly-Ser linkers separating full-length PapG from the stabilizing donor strand F-peptide (DSF). PapG_{DSF} contains alleles N96S, N242S, N286S (fully aglycosylated)(numbering based on processed polypeptide starting with the proximal phenylalanine residue).

TABLE 11
mRNA constructs: PapG protein and RNA sequences

| SEQ ID: | Construct | Sequence |
|---|---|---|
| 27 | PapG_{LD} N96S DAF | METDTLLLWVLLLWVPGSTGWNNIVFYSLGNVNSYQGGNVVITQR |
| | Ser-Gly GPI | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGGSSGGGGSSG |
| | (underlined); | SGSSSGTTRLLSGHTCFTLTGLLGTLVTMGLLT** |
| | Stop codons | |
| | (asterisks) | |
| 42 | BMD2 PapG_{LD} N96S | AGGAAAUAAGAGAGGAUAAGACGACUAAGGAGACAUACAGA |
| | DAF Ser-Gly GPI_ | AUAAGAGGCAGCCACCAUGGAGACCGACACACUGCUGCUGU |
| | modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGUGGGACGAGAGAGCCUUCG |
| | SEQ ID NO: 27) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGGUUCAAGUGGUAGUGGCAGUUCAAG |
| | | UGGGACAACACGACUGUUGAGCGGGCAUACGUGUUUUACGC |
| | | UGACAGGUCUUCUGGGCACGCUGGUUACUAUGGGCUUGCUU |
| | | ACGUGAUGAGCUCGCUUUCUUGCUGUCCAAUUUCUAUUAAA |
| | | GGUUCCUUUGUUCCCUAAGUCCAACUACUAAACUGGGGGAU |
| | | AUUAUGAAGGGCCUUGAGCAUCUGGAUUCUGCCUAAUAAAA |
| | | AACAUUUAUUUUCAUUGCAAaaaaaaaaaaaaaaaaaaaaaaaaaaaa |
| | | aaaaaaaaaaaaaaaaaaaaaaaaaaaaaaaaaaaaaaaaaaaaaaaaaaaa |
| 43 | BMD562 PapG_{LD} | AGGAAAUAAGAGAAAGAGGAUAAGACGACUAAGGAGACAUA |
| | N96S DAF Ser-Gly | CAGAAUAAGAGGCAGCCACCAUGGAGACCGACACACUGCUG |
| | GPI_modRNA | CUGUGGGUGCUUUUGUUGUGGGUGCCCGGUUCUACCGGAUG |
| | Underline = 5′ cap; | GAACAACAUCGUGUUCUACAGCCUGGGCAACGUGAACAGCUA |
| | bold = 5′ UTR and 3′ | CCAAGGCGGCAACGUGGUGAUCACACAGAGACCUCAGUUCAU |
| | UTR; | CACAAGCUGGAGACCCGGCAUCGCCACCGUGACCUGGAAUCA |
| | italics = KOZAK | GUGCAACGGCCCCGAGUUCGCCGACGGCAGCUGGGCCUACU |
| | sequence; | ACAGAGAGUACAUCGCCUGGGUGGUGUUCCCCAAGAAGGUG |
| | lowercase = polyA | AUGACCAAGAACGGCUACCCCCUGUUCAUCGAGGUGCACAAC |
| | tail | AAGGGCAGCUGGAGCGAGGAGAACACCGGCGACAGCGACAG |
| | (Amino acid | CUACUUCUUCCUGAAGGGCUACAAGUGGGACGAGAGAGCCU |
| | SEQ ID NO: 27) | UCGACGCCGGCAACCUGUGUCAGAAGCCCGGCGAGACCACAA |
| | | GACUGACCGAGAAGUUCAACGACAUCAUCUUCAAGGUGGCCC |
| | | UGCCCGCCGACCUGCCCCUGGGCGACUACAGCGUGACCAUC |
| | | CCCUACACAAGCGGCAUUCAGAGACACUUCGCUAGCUACCUG |
| | | GGCGCUAGAUUCAAGAUCCCCUACAACGUGGCCAAGACCCUG |
| | | CCUAGAGAGAACGAGAUGCUGUUCCUGUUCAAGAACAUCGGC |
| | | GGCAGUUCUGGUGGCGGUGGUUCAAGUGGUAGUGGCAGUUC |
| | | AAGUGGGACAACACGACUGUUGAGCGGGCAUACGUGUUUUA |
| | | CGCUGACAGGUCUUCUGGGCACGCUGGUUACUAUGGGCUUG |
| | | CUUACGUGAUGAGCUCGCUUUCUUGCUGUCCAAUUUCUAUU |
| | | AAAGGUUCCUUUGUUCCCUAAGUCCAACUACUAAACUGGGG |
| | | GAUAUUAUGAAGGGCCUUGAGCAUCUGGAUUCUGCCUAAUA |
| | | AAAAACAUUUAUUUUCAUUGCAAaaaaaaaaaaaaaaaaaaaaaaaa |
| | | aaaaaaaaaaaaaaaaaaaaaaaaaaaaaaaaaaaaaaaaaaaaaaaaaaaaaa |
| | | aa |
| 44 | BMD563 PapG_{LD} | AGGAAAUAAGAGAGGAUAAGACGACUAAGGAGACAUACAGA |
| | N96S DAF Ser-Gly | AUAAGAAACAGGCAGCCACCAUGGAGACCGACACACUGCUG |
| | GPI_modRNA | CUGUGGGUGCUUUUGUUGUGGGUGCCCGGUUCUACCGGAUG |
| | Underline = 5′ cap; | GAACAACAUCGUGUUCUACAGCCUGGGCAACGUGAACAGCUA |
| | bold = 5′ UTR and 3′ | CCAAGGCGGCAACGUGGUGAUCACACAGAGACCUCAGUUCAU |
| | UTR; | CACAAGCUGGAGACCCGGCAUCGCCACCGUGACCUGGAAUCA |
| | italics = KOZAK | GUGCAACGGCCCCGAGUUCGCCGACGGCAGCUGGGCCUACU |
| | sequence; | ACAGAGAGUACAUCGCCUGGGUGGUGUUCCCCAAGAAGGUG |
| | lowercase = polyA | AUGACCAAGAACGGCUACCCCCUGUUCAUCGAGGUGCACAAC |
| | tail | AAGGGCAGCUGGAGCGAGGAGAACACCGGCGACAGCGACAG |
| | (Amino acid | CUACUUCUUCCUGAAGGGCUACAAGUGGGACGAGAGAGCCU |
| | SEQ ID NO: 27) | UCGACGCCGGCAACCUGUGUCAGAAGCCCGGCGAGACCACAA |
| | | GACUGACCGAGAAGUUCAACGACAUCAUCUUCAAGGUGGCCC |
| | | UGCCCGCCGACCUGCCCCUGGGCGACUACAGCGUGACCAUC |
| | | CCCUACACAAGCGGCAUUCAGAGACACUUCGCUAGCUACCUG |
| | | GGCGCUAGAUUCAAGAUCCCCUACAACGUGGCCAAGACCCUG |
| | | CCUAGAGAGAACGAGAUGCUGUUCCUGUUCAAGAACAUCGGC |
| | | GGCAGUUCUGGUGGCGGUGGUUCAAGUGGUAGUGGCAGUUC |
| | | AAGUGGGACAACACGACUGUUGAGCGGGCAUACGUGUUUUA |
| | | CGCUGACAGGUCUUCUGGGCACGCUGGUUACUAUGGGCUUG |
| | | CUUACGUGAUGAGCUCGCUUUCUUGCUGUCCAAUUUCUAUU |
| | | AAAGGUUCCUUUGUUCCCUAAGUCCAACUACUAAACUGGGG |
| | | GAUAUUAUGAAGGGCCUUGAGCAUCUGGAUUCUGCCUAAUA |
| | | AAAAACAUUUAUUUUCAUUGCAAaaaaaaaaaaaaaaaaaaaaaaaa |
| | | aaaaaaaaaaaaaaaaaaaaaaaaaaaaaaaaaaaaaaaaaaaaaaaaaaaaaa |
| | | a |
| 45 | BMD576 PapG_{LD} | AGGAGGACUGGUCGAACCUGCAUAGUGAUCAUAAGGUCAGC |
| | N96S DAF Ser-Gly | AUAGCCACCAUGGAGACCGACACACUGCUGCUGUGGGUGCU |
| | GPI_modRNA | UUUGUUGUGGGUGCCCGGUUCUACCGGAUGGAACAACAUCG |
| | Underline = 5′ cap; | UGUUCUACAGCCUGGGCAACGUGAACAGCUACCAAGGCGGCA |
| | bold = 5′ UTR and 3′ | ACGUGGUGAUCACACAGAGACCUCAGUUCAUCACAAGCUGGA |
| | UTR; | GACCCGGCAUCGCCACCGUGACCUGGAAUCAGUGCAACGGC |
| | italics = KOZAK | CCCGAGUUCGCCGACGGCAGCUGGGCCUACUACAGAGAGUA |
| | sequence; | CAUCGCCUGGGUGGUGUUCCCCAAGAAGGUGAUGACCAAGA |
| | lowercase = polyA | ACGGCUACCCCCUGUUCAUCGAGGUGCACAACAAGGGCAGCU |
| | tail | GGAGCGAGGAGAACACCGGCGACAGCGACAGCUACUUCUUC |
| | (Amino acid | CUGAAGGGCUACAAGUGGGACGAGAGAGCCUUCGACGCCGG |
| | SEQ ID NO: 27) | CAACCUGUGUCAGAAGCCCGGCGAGACCACAAGACUGACCGA |
| | | GAAGUUCAACGACAUCAUCUUCAAGGUGGCCCUGCCCGCCGA |
| | | CCUGCCCCUGGGCGACUACAGCGUGACCAUCCCCUACACAAG |
| | | CGGCAUUCAGAGACACUUCGCUAGCUACCUGGGCGCUAGAU |
| | | UCAAGAUCCCCUACAACGUGGCCAAGACCCUGCCUAGAGAGA |
| | | ACGAGAUGCUGUUCCUGUUCAAGAACAUCGGCGGCAGUUCU |
| | | GGUGGCGGUGGUUCAAGUGGUAGUGGCAGUUCAAGUGGGAC |
| | | AACACGACUGUUGAGCGGGCAUACGUGUUUUACGCUGACAG |
| | | GUCUUCUGGGCACGCUGGUUACUAUGGGCUUGCUUACGUGA |
| | | UGAGCUCGCUUUCUUGCUGUCCAAUUUCUAUUAAAGGUUCC |
| | | UUUGUUCCCUAAGUCCAACUACUAAACUGGGGGAUAUUAUG |
| | | AAGGGCCUUGAGCAUCUGGAUUCUGCCUAAUAAAAAACAUU |
| | | UAUUUUCAUUGCAAaaaaaaaaaaaaaaaaaaaaaaaaaaaaaaaaaaaa |
| | | aaaaaaaaaaaaaaaaaaaaaaaaaaaaaaaaaaaaaaaaaaaa |
| 28 | PapG_{LD} N96S Thy1 | METDTLLLWVLLLWVPGSTGWNNIVFYSLGNVNSYQGGNVVITQR |
| | Ser-Gly GPI | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGGSSGGGGSSG |
| | (underlined); | SGSSCEGISLLAQNTSWLLLLLLSLSLLQATDFMSL** |
| | Stop codons | |
| | (asterisks) | |
| 46 | BMD2 PapG_{LD} N96S | AGGAAAUAAGAGAGGAUAAGACGACUAAGGAGACAUACAGA |
| | Thy1 Ser-Gly GPI_ | AUAAGAGGCAGCCACCAUGGAGACCGACACACUGCUGCUGU |
| | modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGUGGGACGAGAGAGCCUUCG |
| | SEQ ID NO: 28) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGGUUCAAGUGGUAGUGGCAGUUCAUG |
| | | UGAAGGGAUCAGUCUCCUCGCCCAAAACACUUCAUGGCUGUU |
| | | GCUCCUCCUGUUGUCCCUUUCUCUUUUGCAGGCAACAGAUU |
| | | UUAUGAGCUUGUGAUGAGCUCGCUUUCUUGCUGUCCAAUUU |
| | | CUAUUAAAGGUUCCUUUGUUCCCUAAGUCCAACUACUAAAC |
| | | UGGGGGAUAUUAUGAAGGGCCUUGAGCAUCUGGAUUCUGCC |
| | | UAAUAAAAAACAUUUAUUUUCAUUGCAAaaaaaaaaaaaaaaaaaa |
| | | aaaaaaaaaaaaaaaaaaaaaaaaaaaaaaaaaaaaaaaaaaaaaaaaaaaaaa |
| | | aaaaaaaa |
| 29 | PapG_{LD} N96S | METDTLLLWVLLLWVPGSTGWNNIVFYSLGNVNSYQGGNVVITQR |
| | CT60HSVgD | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGGSSGGGATPN |
| | (underlined); | NMGLIAGAVGGSLLAALVICGIVYWMRRHTQKAPKRIRLPHIREDD |
| | Stop codons | QPSSHQPLFY** |
| | (asterisks) | |
| 47 | BMD2 PapG_{LD} | AGGAAAUAAGAGAGGAUAAGACGACUAAGGAGACAUACAGA |
| | N96S CT60HSVgD_ | AUAAGAGGCAGCCACCAUGGAGACCGACACACUGCUGCUGU |
| | modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGUGGGACGAGAGAGCCUUCG |
| | SEQ ID NO: 29) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGCGACCCCCAACAACAUGGGGCUGAU |
| | | AGCCGGUGCCGUAGGCGGCAGCCUGCUGGCCGCCCUGGUGA |
| | | UCUGCGGCAUCGUGUACUGGAUGAGAAGACACACACAGAAGG |
| | | CCCCCAAGAGAAUCAGACUGCCCCACAUCAGAGAGGACGAUC |
| | | AGCCUAGCAGCCAUCAGCCCCUGUUCUACUGAUGAGCUCGCU |
| | | UUCUUGCUGUCCAAUUUCUAUUAAAGGUUCCUUUGUUCCCU |
| | | AAGUCCAACUACUAAACUGGGGGAUAUUAUGAAGGGCCUUG |
| | | AGCAUCUGGAUUCUGCCUAAUAAAAAACAUUUAUUUUCAUU |
| | | GCAAaaaaaaaaaaaaaaaaaaaaaaaaaaaaaaaaaaaaaaaaaaaaaaaaa |
| | | aaaaaaaaaaaaaaaaaaaaaaaaaaaaaaa |
| 13 | PapG_{LD} N96S | METDTLLLWVLLLWVPGSTGWNNIVFYSLGNVNSYQGGNVVITQR |
| | secreted | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGG** |
| | (underlined); | |
| | Stop codons | |
| | (asterisks) | |
| 48 | BMD2 PapG_{LD} N96S | AGGAAAUAAGAGAGGAUAAGACGACUAAGGAGACAUACAGA |
| | secreted_modRNA | AUAAGAGGCAGCCACCAUGGAGACCGACACACUGCUGCUGU |
| | Underline = 5′ cap; | GGGUGCUGCUGCUGUGGGUGCCCGGCUCCACCGGCUGGAAC |
| | bold = 5′ UTR and 3′ | AACAUCGUGUUCUACAGCCUGGGCAACGUGAACAGCUACCAA |
| | UTR; | GGCGGCAACGUGGUGAUCACACAGAGACCUCAGUUCAUCACA |
| | italics = KOZAK | AGCUGGAGACCCGGCAUCGCCACCGUGACCUGGAAUCAGUG |
| | sequence; | CAACGGCCCCGAGUUCGCCGACGGCAGCUGGGCCUACUACA |
| | lowercase = polyA | GAGAGUACAUCGCCUGGGUGGUGUUCCCCAAGAAGGUGAUG |
| | tail | ACCAAGAACGGCUACCCCCUGUUCAUCGAGGUGCACAACAAG |
| | (Amino acid | GGCAGCUGGAGCGAGGAGAACACCGGCGACAGCGACAGCUA |
| | SEQ ID NO: 13) | CUUCUUCCUGAAGGGCUACAAGUGGGACGAGAGAGCCUUCG |
| | | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | UGAUGAGCUCGCUUUCUUGCUGUCCAAUUUCUAUUAAAGGU |
| | | UCCUUUGUUCCCUAAGUCCAACUACUAAACUGGGGGAUAUU |
| | | AUGAAGGGCCUUGAGCAUCUGGAUUCUGCCUAAUAAAAAAC |
| | | AUUUAUUUUCAUUGCAAaaaaaaaaaaaaaaaaaaaaaaaaaaaaaaaa |
| | | aaaaaaaaaaaaaaaaaaaaaaaaaaaaaaaaaaaaaaaaaaaaaaaa |
| 30 | PapG_{LD} N96S | METDTLLLWVLLLWVPGSTGWNNIVFYSLGNVNSYQGGNVVITQR |
| | R170A DAF Ser- | PQFITSWRPGIATVTWNQCNGPEFADGSWAYYREYIAWVVFPKK |
| | Gly GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGAAFKIPYNVAKTLPRENEMLFLFKNIGGSSGGGGSSG |
| | Interdomain linker | SGSSSGTTRLLSGHTCFTLTGLLGTLVTMGLLT** |
| | (underlined); | |
| | Stop codons | |
| | (asterisks) | |
| 49 | BMD2 PapG_{LD} N96S | AGGAAAUAAGAGAGGAUAAGACGACUAAGGAGACAUACAGA |
| | R170A DAF Ser-Gly | AUAAGAGGCAGCCACCAUGGAGACCGACACACUGCUGCUGU |
| | GPI_modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGUGGGACGAGAGAGCCUUCG |
| | SEQ ID NO: 30) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUGCCUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGGUUCAAGUGGUAGUGGCAGUUCAAG |
| | | UGGGACAACACGACUGUUGAGCGGGCAUACGUGUUUUACGC |
| | | UGACAGGUCUUCUGGGCACGCUGGUUACUAUGGGCUUGCUU |
| | | ACGUGAUGAGCUCGCUUUCUUGCUGUCCAAUUUCUAUUAAA |
| | | GGUUCCUUUGUUCCCUAAGUCCAACUACUAAACUGGGGGAU |
| | | AUUAUGAAGGGCCUUGAGCAUCUGGAUUCUGCCUAAUAAAA |
| | | AACAUUUAUUUUCAUUGCAAaaaaaaaaaaaaaaaaaaaaaaaaaaaa |
| | | aaaaaaaaaaaaaaaaaaaaaaaaaaaaaaaaaaaaaaaaaaaaaaaaaaaa |
| 31 | PapG_{LD} N96S | METDTLLLWVLLLWVPGSTGWNNIVFYSLGNVNSYQGGNVVITQR |
| | K172A DAF Ser- | PQFITSWRPGIATVTWNQCNGPEFADGSWAYYREYIAWVVFPKK |
| | Gly GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGARFAIPYNVAKTLPRENEMLFLFKNIGGSSGGGGSSG |
| | Interdomain linker | SGSSSGTTRLLSGHTCFTLTGLLGTLVTMGLLT** |
| | (underlined); | |
| | Stop codons | |
| | (asterisks) | |
| 50 | BMD2 PapG_{LD} N96S | AGGAAAUAAGAGAGGAUAAGACGACUAAGGAGACAUACAGAA |
| | K172A DAF Ser-Gly | UAAGAGGCAGCCACCAUGGAGACCGACACACUGCUGCUGUG |
| | GPI_modRNA | GGUGCUUUUGUUGUGGGUGCCCGGUUCUACCGGAUGGAACA |
| | Underline = 5′ cap; | ACAUCGUGUUCUACAGCCUGGGCAACGUGAACAGCUACCAAG |
| | bold = 5′ UTR and 3′ | GCGGCAACGUGGUGAUCACACAGAGACCUCAGUUCAUCACAA |
| | UTR; | GCUGGAGACCCGGCAUCGCCACCGUGACCUGGAAUCAGUGC |
| | italics = KOZAK | AACGGCCCCGAGUUCGCCGACGGCAGCUGGGCCUACUACAG |
| | sequence; | AGAGUACAUCGCCUGGGUGGUGUUCCCCAAGAAGGUGAUGA |
| | lowercase = polyA | CCAAGAACGGCUACCCCCUGUUCAUCGAGGUGCACAACAAGG |
| | tail | GCAGCUGGAGCGAGGAGAACACCGGCGACAGCGACAGCUAC |
| | (Amino acid | UUCUUCCUGAAGGGCUACAAGUGGGACGAGAGAGCCUUCGA |
| | SEQ ID NO: 31) | CGCCGGCAACCUGUGUCAGAAGCCCGGCGAGACCACAAGACU |
| | | GACCGAGAAGUUCAACGACAUCAUCUUCAAGGUGGCCCUGCC |
| | | CGCCGACCUGCCCCUGGGCGACUACAGCGUGACCAUCCCCU |
| | | ACACAAGCGGCAUUCAGAGACACUUCGCUAGCUACCUGGGCG |
| | | CUAGAUUCGCCAUCCCCUACAACGUGGCCAAGACCCUGCCUA |
| | | GAGAGAACGAGAUGCUGUUCCUGUUCAAGAACAUCGGCGGCA |
| | | GUUCUGGUGGCGGUGGUUCAAGUGGUAGUGGCAGUUCAAGU |
| | | GGGACAACACGACUGUUGAGCGGGCAUACGUGUUUUACGCU |
| | | GACAGGUCUUCUGGGCACGCUGGUUACUAUGGGCUUGCUUA |
| | | CGUGAUGAGCUCGCUUUCUUGCUGUCCAAUUUCUAUUAAAG |
| | | GUUCCUUUGUUCCCUAAGUCCAACUACUAAACUGGGGGAUA |
| | | UUAUGAAGGGCCUUGAGCAUCUGGAUUCUGCCUAAUAAAAA |
| | | ACAUUUAUUUUCAUUGCAAaaaaaaaaaaaaaaaaaaaaaaaaaaaaa |
| | | aaaaaaaaaaaaaaaaaaaaaaaaaaaaaaaaaaaaaaaaaaaaaaaaaaa |
| 51 | BMD562 PapG_{LD} | AGGAAAUAAGAGAAAGAGGAUAAGACGACUAAGGAGACAUA |
| | N96S K172A DAF | CAGAAUAAGAGGCAGCCACCAUGGAGACCGACACACUGCUG |
| | Ser-Gly GPI_ | CUGUGGGUGCUUUUGUUGUGGGUGCCCGGUUCUACCGGAUG |
| | modRNA | GAACAACAUCGUGUUCUACAGCCUGGGCAACGUGAACAGCUA |
| | Underline = 5′ cap; | CCAAGGCGGCAACGUGGUGAUCACACAGAGACCUCAGUUCAU |
| | bold = 5′ UTR and 3′ | CACAAGCUGGAGACCCGGCAUCGCCACCGUGACCUGGAAUCA |
| | UTR; | GUGCAACGGCCCCGAGUUCGCCGACGGCAGCUGGGCCUACU |
| | italics = KOZAK | ACAGAGAGUACAUCGCCUGGGUGGUGUUCCCCAAGAAGGUG |
| | sequence; | AUGACCAAGAACGGCUACCCCCUGUUCAUCGAGGUGCACAAC |
| | lowercase = polyA | AAGGGCAGCUGGAGCGAGGAGAACACCGGCGACAGCGACAG |
| | tail | CUACUUCUUCCUGAAGGGCUACAAGUGGGACGAGAGAGCCU |
| | (Amino acid | UCGACGCCGGCAACCUGUGUCAGAAGCCCGGCGAGACCACAA |
| | SEQ ID NO: 31) | GACUGACCGAGAAGUUCAACGACAUCAUCUUCAAGGUGGCCC |
| | | UGCCCGCCGACCUGCCCCUGGGCGACUACAGCGUGACCAUC |
| | | CCCUACACAAGCGGCAUUCAGAGACACUUCGCUAGCUACCUG |
| | | GGCGCUAGAUUCGCCAUCCCCUACAACGUGGCCAAGACCCUG |
| | | CCUAGAGAGAACGAGAUGCUGUUCCUGUUCAAGAACAUCGGC |
| | | GGCAGUUCUGGUGGCGGUGGUUCAAGUGGUAGUGGCAGUUC |
| | | AAGUGGGACAACACGACUGUUGAGCGGGCAUACGUGUUUUA |
| | | CGCUGACAGGUCUUCUGGGCACGCUGGUUACUAUGGGCUUG |
| | | CUUACGUGAUGAGCUCGCUUUCUUGCUGUCCAAUUUCUAUU |
| | | AAAGGUUCCUUUGUUCCCUAAGUCCAACUACUAAACUGGGG |
| | | GAUAUUAUGAAGGGCCUUGAGCAUCUGGAUUCUGCCUAAUA |
| | | AAAAACAUUUAUUUUCAUUGCAAaaaaaaaaaaaaaaaaaaaaaaaa |
| | | aaaaaaaaaaaaaaaaaaaaaaaaaaaaaaaaaaaaaaaaaaaaaaaaaaaaaa |
| | | aa |
| 52 | BMD576 PapG_{LD} | AGGAGGACUGGUCGAACCUGCAUAGUGAUCAUAAGGUCAGC |
| | N96S K172A DAF | AUAGCCACCAUGGAGACCGACACACUGCUGCUGUGGGUGCU |
| | Ser-Gly GPI_ | UUUGUUGUGGGUGCCCGGUUCUACCGGAUGGAACAACAUCG |
| | modRNA | UGUUCUACAGCCUGGGCAACGUGAACAGCUACCAAGGCGGCA |
| | Underline = 5′ cap; | ACGUGGUGAUCACACAGAGACCUCAGUUCAUCACAAGCUGGA |
| | bold = 5′ UTR and 3′ | GACCCGGCAUCGCCACCGUGACCUGGAAUCAGUGCAACGGC |
| | UTR; | CCCGAGUUCGCCGACGGCAGCUGGGCCUACUACAGAGAGUA |
| | italics = KOZAK | CAUCGCCUGGGUGGUGUUCCCCAAGAAGGUGAUGACCAAGA |
| | sequence; | ACGGCUACCCCCUGUUCAUCGAGGUGCACAACAAGGGCAGCU |
| | lowercase = polyA | GGAGCGAGGAGAACACCGGCGACAGCGACAGCUACUUCUUC |
| | tail | CUGAAGGGCUACAAGUGGGACGAGAGAGCCUUCGACGCCGG |
| | (Amino acid | CAACCUGUGUCAGAAGCCCGGCGAGACCACAAGACUGACCGA |
| | SEQ ID NO: 31) | GAAGUUCAACGACAUCAUCUUCAAGGUGGCCCUGCCCGCCGA |
| | | CCUGCCCCUGGGCGACUACAGCGUGACCAUCCCCUACACAAG |
| | | CGGCAUUCAGAGACACUUCGCUAGCUACCUGGGCGCUAGAU |
| | | UCGCCAUCCCCUACAACGUGGCCAAGACCCUGCCUAGAGAGA |
| | | ACGAGAUGCUGUUCCUGUUCAAGAACAUCGGCGGCAGUUCU |
| | | GGUGGCGGUGGUUCAAGUGGUAGUGGCAGUUCAAGUGGGAC |
| | | AACACGACUGUUGAGCGGGCAUACGUGUUUUACGCUGACAG |
| | | GUCUUCUGGGCACGCUGGUUACUAUGGGCUUGCUUACGUGA |
| | | UGAGCUCGCUUUCUUGCUGUCCAAUUUCUAUUAAAGGUUCC |
| | | UUUGUUCCCUAAGUCCAACUACUAAACUGGGGGAUAUUAUG |
| | | AAGGGCCUUGAGCAUCUGGAUUCUGCCUAAUAAAAAACAUU |
| | | UAUUUUCAUUGCAAaaaaaaaaaaaaaaaaaaaaaaaaaaaaaaaaaaaa |
| | | aaaaaaaaaaaaaaaaaaaaaaaaaaaaaaaaaaaaaaaaaaaa |
| 32 | PapG_{LD} N96S | METDTLLLWVLLLWVPGSTGWNNIVFYSLGNVNSYQGGNVVITQR |
| | G104A DAF Ser- | PQFITSWRPGIATVTWNQCNGPEFADGSWAYYREYIAWVVFPKK |
| | Gly GPI | VMTKNGYPLFIEVHNKGSWSEENTGDSDSYFFLKAYKWDERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGSSGGGGSSG |
| | Interdomain linker | SGSSSGTTRLLSGHTCFTLTGLLGTLVTMGLLT** |
| | (underlined); | |
| | Stop codons | |
| | (asterisks) | |
| 53 | BMD2 PapG_{LD} N96S | AGGAAAUAAGAGAGGAUAAGACGACUAAGGAGACAUACAGA |
| | G104A DAF Ser-Gly | AUAAGAGGCAGCCACCAUGGAGACCGACACACUGCUGCUGU |
| | GPI_modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGCCUACAAGUGGGACGAGAGAGCCUUCG |
| | SEQ ID NO: 32) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGGUUCAAGUGGUAGUGGCAGUUCAAG |
| | | UGGGACAACACGACUGUUGAGCGGGCAUACGUGUUUUACGC |
| | | UGACAGGUCUUCUGGGCACGCUGGUUACUAUGGGCUUGCUU |
| | | ACGUGAUGAGCUCGCUUUCUUGCUGUCCAAUUUCUAUUAAA |
| | | GGUUCCUUUGUUCCCUAAGUCCAACUACUAAACUGGGGGAU |
| | | AUUAUGAAGGGCCUUGAGCAUCUGGAUUCUGCCUAAUAAAA |
| | | AACAUUUAUUUUCAUUGCAAaaaaaaaaaaaaaaaaaaaaaaaaaaaa |
| | | aaaaaaaaaaaaaaaaaaaaaaaaaaaaaaaaaaaaaaaaaaaaaaaaaaaa |
| 33 | PapG_{LD} N96S | METDTLLLWVLLLWVPGSTGWNNIVFYSLGNVNSYQGGNVVITQR |
| | W107A DAF Ser- | PQFITSWRPGIATVTWNQCNGPEFADGSWAYYREYIAWVVFPKK |
| | Gly GPI | VMTKNGYPLFIEVHNKGSWSEENTGDSDSYFFLKGYKADERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGSSGGGGSSG |
| | Interdomain linker | SGSSSGTTRLLSGHTCFTLTGLLGTLVTMGLLT** |
| | (underlined); | |
| | Stop codons | |
| | (asterisks) | |
| 54 | BMD2 PapG_{LD} N96S | AGGAAAUAAGAGAGGAUAAGACGACUAAGGAGACAUACAGA |
| | W107A DAF Ser- | AUAAGAGGCAGCCACCAUGGAGACCGACACACUGCUGCUGU |
| | Gly GPI_modRNA | GGGUGCUUUUGUUGUGGGUGCCCGGUUCUACCGGAUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGGCCGACGAGAGAGCCUUCG |
| | SEQ ID NO: 33) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | AGUUCUGGUGGCGGUGGUUCAAGUGGUAGUGGCAGUUCAAG |
| | | UGGGACAACACGACUGUUGAGCGGGCAUACGUGUUUUACGC |
| | | UGACAGGUCUUCUGGGCACGCUGGUUACUAUGGGCUUGCUU |
| | | ACGUGAUGAGCUCGCUUUCUUGCUGUCCAAUUUCUAUUAAA |
| | | GGUUCCUUUGUUCCCUAAGUCCAACUACUAAACUGGGGGAU |
| | | AUUAUGAAGGGCCUUGAGCAUCUGGAUUCUGCCUAAUAAAA |
| | | AACAUUUAUUUUCAUUGCAAaaaaaaaaaaaaaaaaaaaaaaaaaaaa |
| | | aaaaaaaaaaaaaaaaaaaaaaaaaaaaaaaaaaaaaaaaaaaaaaaaaaaa |
| 34 | PapG_{DSF} N96S | METDTLLLWVLLLWVPGSTGWNNIVFYSLGNVNSYQGGNVVITQR |
| | N286S DAF Ser-Gly | PQFITSWRPGIATVTWNQCNGPEFADGSWAYYREYIAWVVFPKK |
| | GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGCRPSAQSLEI |
| | Interdomain linker | KHGDLSINSANNHYAAQTLSVSCDVPANIRFMLLRNTTPTYSHGKK |
| | (underlined); | FSVGLGHGWDSIVSVNGVDTGETTMRWYKAGTQSLTIGSRLYGE |
| | Stop codons | SSKIQPGVLSGSATLLMILPGGSSGGADVQINIRGNVYIPPSSGGG |
| | (asterisks) | GSSGSGSSSGTTRLLSGHTCFTLTGLLGTLVTMGLLT** |
| 55 | BMD2 PapG_{DSF} | AGGAAAUAAGAGAGGAUAAGACGACUAAGGAGACAUACAGA |
| | N96S N286S DAF | AUAAGAGGCAGCCACCAUGGAGACCGACACCCUCCUGCUGU |
| | Ser-Gly GPI_ | GGGUGCUGUUACUGUGGGUGCCCGGUAGCACCGGCUGGAAC |
| | modRNA | AACAUCGUGUUCUACAGCCUGGGCAACGUGAACAGCUACCAA |
| | Underline = 5′ cap; | GGCGGCAACGUGGUGAUCACACAGAGACCUCAGUUCAUCACA |
| | bold = 5′ UTR and 3′ | AGCUGGAGACCCGGCAUCGCCACCGUGACCUGGAAUCAGUG |
| | UTR; | CAACGGCCCCGAGUUCGCCGACGGCAGCUGGGCCUACUACA |
| | italics = KOZAK | GAGAGUACAUCGCCUGGGUGGUGUUCCCCAAGAAGGUGAUG |
| | sequence; | ACCAAGAACGGCUACCCCCUGUUCAUCGAGGUGCACAACAAG |
| | lowercase = polyA | GGCAGCUGGAGCGAGGAGAACACCGGCGACAGCGACAGCUA |
| | tail | CUUCUUCCUGAAGGGCUACAAGUGGGACGAGAGAGCCUUCG |
| | (Amino acid | ACGCCGGCAACCUGUGUCAGAAGCCGGGGGAGACGACCCGG |
| | SEQ ID NO: 34) | CUUACAGAAAAGUUCAACGACAUCAUCUUCAAGGUGGCCCUG |
| | | CCCGCCGACCUGCCCCUGGGCGACUACAGCGUGACCAUCCC |
| | | CUACACAAGCGGCAUUCAGAGACACUUCGCUAGCUACCUGGG |
| | | CGCUAGAUUCAAGAUCCCCUACAACGUGGCCAAGACCCUGCC |
| | | UAGAGAGAACGAGAUGCUGUUCCUGUUCAAGAACAUCGGCGG |
| | | CUGCAGACCUAGCGCUCAGAGCCUGGAGAUCAAGCACGGCG |
| | | ACCUGAGCAUCAACAGCGCCAACAACCACUACGCCGCUCAGA |
| | | CCCUGAGCGUGAGCUGCGACGUGCCCGCCAACAUCAGAUUC |
| | | AUGCUGCUGAGAAACACCACCCCCACCUACAGCCACGGCAAG |
| | | AAGUUCAGCGUGGGCCUGGGCCACGGCUGGGACAGCAUCGU |
| | | GAGCGUGAACGGCGUGGACACGGGCGAGACCACUAUGCGUU |
| | | GGUACAAGGCCGGCACACAGAGCCUGACCAUCGGCAGCAGAC |
| | | UGUACGGCGAGAGCAGCAAGAUUCAACCGGGCGUGCUUAGU |
| | | GGUUCCGCCACCCUGCUGAUGAUUCUGCCCGGCGGAAGCAG |
| | | CGGCGGCGCCGACGUGCAGAUCAACAUCAGAGGCAACGUGU |
| | | ACAUACCCCCUUCGAGCGGAGGCGGCGGAUCGUCCGGCUCC |
| | | GGGAGCAGCAGCGGCACCACACGGCUGCUGAGCGGCCACAC |
| | | CUGCUUCACCCUGACCGGCCUGCUGGGCACCCUGGUGACCA |
| | | UGGGCCUGCUGACCUGAUGAGCUCGCUUUCUUGCUGUCCAA |
| | | UUUCUAUUAAAGGUUCCUUUGUUCCCUAAGUCCAACUACUA |
| | | AACUGGGGGAUAUUAUGAAGGGCCUUGAGCAUCUGGAUUCU |
| | | GCCUAAUAAAAAACAUUUAUUUUCAUUGCAAaaaaaaaaaaaaaa |
| | | aaaaaaaaaaaaaaaaaaaaaaaaaaaaaaaaaaaaaaaaaaaaaaaaaaaaaa |
| | | aaaaaaaaaaaa |
| 35 | PapG_{DSF} N96S | METDTLLLWVLLLWVPGSTGWNNIVFYSLGNVNSYQGGNVVITQR |
| | N286S Thy1 Ser- | PQFITSWRPGIATVTWNQCNGPEFADGSWAYYREYIAWVVFPKK |
| | Gly GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGCRPSAQSLEI |
| | Interdomain linker | KHGDLSINSANNHYAAQTLSVSCDVPANIRFMLLRNTTPTYSHGKK |
| | (underlined); | FSVGLGHGWDSIVSVNGVDTGETTMRWYKAGTQSLTIGSRLYGE |
| | Stop codons | SSKIQPGVLSGSATLLMILPGGSSGGADVQINIRGNVYIPPSSGGG |
| | (asterisks) | GSSGSGSSCEGISLLAQNTSWLLLLLLSLSLLQATDFMSL** |
| 56 | BMD2 PapG_{DSF} | AGGAAAUAAGAGAGGAUAAGACGACUAAGGAGACAUACAGA |
| | N96S N286S Thy1 | AUAAGAGGCAGCCACCAUGGAGACCGACACCCUCCUGCUGU |
| | Ser-Gly GPI_ | GGGUGCUGUUACUGUGGGUGCCCGGUAGCACCGGCUGGAAC |
| | modRNA | AACAUCGUGUUCUACAGCCUGGGCAACGUGAACAGCUACCAA |
| | Underline = 5′ cap; | GGCGGCAACGUGGUGAUCACACAGAGACCUCAGUUCAUCACA |
| | bold = 5′ UTR and 3′ | AGCUGGAGACCCGGCAUCGCCACCGUGACCUGGAAUCAGUG |
| | UTR; | CAACGGCCCCGAGUUCGCCGACGGCAGCUGGGCCUACUACA |
| | italics = KOZAK | GAGAGUACAUCGCCUGGGUGGUGUUCCCCAAGAAGGUGAUG |
| | sequence; | ACCAAGAACGGCUACCCCCUGUUCAUCGAGGUGCACAACAAG |
| | lowercase = polyA | GGCAGCUGGAGCGAGGAGAACACCGGCGACAGCGACAGCUA |
| | tail | CUUCUUCCUGAAGGGCUACAAGUGGGACGAGAGAGCCUUCG |
| | (Amino acid | ACGCCGGCAACCUGUGUCAGAAGCCGGGGGAGACCACAAGA |
| | SEQ ID NO: 35) | CUUACCGAGAAGUUCAACGACAUCAUCUUCAAGGUGGCCCUG |
| | | CCCGCCGACCUGCCCCUGGGCGACUACAGCGUGACCAUCCC |
| | | CUACACAAGCGGCAUUCAGAGACACUUCGCUAGCUACCUGGG |
| | | CGCUAGAUUCAAGAUCCCCUACAACGUGGCCAAGACCCUGCC |
| | | UAGAGAGAACGAGAUGCUGUUCCUGUUCAAGAACAUCGGCGG |
| | | CUGCAGACCUAGCGCUCAGAGCCUGGAGAUCAAGCACGGCG |
| | | ACCUGAGCAUCAACAGCGCCAACAACCACUACGCCGCUCAGA |
| | | CCCUGAGCGUGAGCUGCGACGUGCCCGCCAACAUCAGAUUC |
| | | AUGCUGCUGAGAAACACCACCCCCACCUACAGCCACGGCAAG |
| | | AAGUUCAGCGUGGGCCUGGGCCACGGCUGGGACAGCAUCGU |
| | | GAGCGUGAACGGCGUGGACACGGGCGAGACCACUAUGCGUU |
| | | GGUACAAGGCCGGCACACAGAGCCUGACCAUCGGCAGCAGAC |
| | | UGUAUGGCGAAAGCUCCAAAAUCCAACCCGGCGUGCUGUCG |
| | | GGGAGCGCCACCCUGCUGAUGAUACUGCCCGGAGGCAGCAG |
| | | CGGCGGCGCCGACGUGCAGAUCAACAUCAGAGGCAACGUGU |
| | | AUAUUCCGCCUAGCAGUGGUGGUGGGGGUAGCUCCGGCUCC |
| | | GGAAGCAGCUGCGAGGGCAUCAGCCUGCUGGCUCAGAACAC |
| | | AAGCUGGCUGCUGCUGCUGCUGCUGAGCCUGAGCCUGCUGC |
| | | AAGCCACCGACUUCAUGAGCCUGUGAUGAGCUCGCUUUCUU |
| | | GCUGUCCAAUUUCUAUUAAAGGUUCCUUUGUUCCCUAAGUC |
| | | CAACUACUAAACUGGGGGAUAUUAUGAAGGGCCUUGAGCAU |
| | | CUGGAUUCUGCCUAAUAAAAAACAUUUAUUUUCAUUGCAAaa |
| | | aaaaaaaaaaaaaaaaaaaaaaaaaaaaaaaaaaaaaaaaaaaaaaaaaaaaaa |
| | | aaaaaaaaaaaaaaaaaaaaaaaa |
| 36 | PapG_{DSF} N96S | METDTLLLWVLLLWVPGSTGWNNIVFYSLGNVNSYQGGNVVITQR |
| | N286S CT60HSVgD | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGGCRPSAQSLEI |
| | (underlined); | KHGDLSINSANNHYAAQTLSVSCDVPANIRFMLLRNTTPTYSHGKK |
| | Stop codons | FSVGLGHGWDSIVSVNGVDTGETTMRWYKAGTQSLTIGSRLYGE |
| | (asterisks) | SSKIQPGVLSGSATLLMILPGGSSGGADVQINIRGNVYIPPSSGGG |
| | | ATPNNMGLIAGAVGGSLLAALVICGIVYWMRRHTQKAPKRIRLPHI |
| | | REDDQPSSHQPLFY** |
| 57 | BMD2 PapG_{DSF} | AGGAAAUAAGAGAGGAUAAGACGACUAAGGAGACAUACAGA |
| | N96S N286S | AUAAGAGGCAGCCACCAUGGAGACCGACACCCUCCUGCUGU |
| | CT60HSVgD_ | GGGUGCUGUUACUGUGGGUGCCCGGUAGCACCGGCUGGAAC |
| | modRNA | AACAUCGUGUUCUACAGCCUGGGCAACGUGAACAGCUACCAA |
| | Underline = 5′ cap; | GGCGGCAACGUGGUGAUCACACAGAGACCUCAGUUCAUCACA |
| | bold = 5′ UTR and 3′ | AGCUGGAGACCCGGCAUCGCCACCGUGACCUGGAAUCAGUG |
| | UTR; | CAACGGCCCCGAGUUCGCCGACGGCAGCUGGGCCUACUACA |
| | italics = KOZAK | GAGAGUACAUCGCCUGGGUGGUGUUCCCCAAGAAGGUGAUG |
| | sequence; | ACCAAGAACGGCUACCCCCUGUUCAUCGAGGUGCACAACAAG |
| | lowercase = polyA | GGCAGCUGGAGCGAGGAGAACACCGGCGACAGCGACAGCUA |
| | tail | CUUCUUCCUGAAGGGCUACAAGUGGGACGAGAGAGCCUUCG |
| | (Amino acid | ACGCCGGCAACCUGUGUCAGAAGCCCGGCGAGACCACAAGAC |
| | SEQ ID NO: 36) | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | UGCAGACCUAGCGCUCAGAGCCUGGAGAUCAAGCACGGCGA |
| | | CCUGAGCAUCAACAGCGCCAACAACCACUACGCCGCUCAGAC |
| | | CCUGAGCGUGAGCUGCGACGUGCCCGCCAACAUCAGAUUCA |
| | | UGCUGCUGAGAAACACCACCCCCACCUACAGCCACGGCAAGA |
| | | AGUUCAGCGUGGGCCUGGGCCACGGCUGGGACAGCAUCGUG |
| | | AGCGUGAACGGCGUGGACACCGGCGAGACUACCAUGAGAUG |
| | | GUACAAGGCCGGCACACAGAGCCUGACCAUCGGCAGCAGACU |
| | | GUAUGGCGAAAGCAGCAAGAUUCAGCCCGGCGUGUUAAGCG |
| | | GCUCUGCGACCCUCCUGAUGAUACUGCCCGGUGGAAGCAGU |
| | | GGGGGCGCCGACGUGCAGAUCAACAUCAGAGGCAAUGUCUA |
| | | CAUCCCCCCUAGCUCUGGUGGCGGCGCAACCCCCAACAACAU |
| | | GGGCCUUAUUGCGGGCGCCGUUGGUGGUUCCCUGCUGGCC |
| | | GCCCUGGUGAUCUGCGGCAUCGUGUACUGGAUGAGAAGACA |
| | | CACACAGAAGGCCCCCAAGAGAAUCAGACUGCCCCACAUCAG |
| | | AGAGGACGAUCAGCCUAGCAGCCAUCAGCCCCUGUUCUACUG |
| | | AUGAGCUCGCUUUCUUGCUGUCCAAUUUCUAUUAAAGGUUC |
| | | CUUUGUUCCCUAAGUCCAACUACUAAACUGGGGGAUAUUAU |
| | | GAAGGGCCUUGAGCAUCUGGAUUCUGCCUAAUAAAAAACAU |
| | | UUAUUUUCAUUGCAAaaaaaaaaaaaaaaaaaaaaaaaaaaaaaaaaaaa |
| | | aaaaaaaaaaaaaaaaaaaaaaaaaaaaaaaaaaaaaaaaaaaaa |
| 37 | PapG_{DSF} N96S | METDTLLLWVLLLWVPGSTGWNNIVFYSLGNVNSYQGGNVVITQR |
| | N286S Secreted | PQFITSWRPGIATVTWNQCNGPEFADGSWAYYREYIAWVVFPKK |
| | Mouse IgK signal | VMTKNGYPLFIEVHNKGSWSEENTGDSDSYFFLKGYKWDERAFD |
| | peptide (italics); | AGNLCQKPGETTRLTEKFNDIIFKVALPADLPLGDYSVTIPYTSGIQ |
| | Interdomain linker | RHFASYLGARFKIPYNVAKTLPRENEMLFLFKNIGGCRPSAQSLEI |
| | (underlined); | KHGDLSINSANNHYAAQTLSVSCDVPANIRFMLLRNTTPTYSHGKK |
| | Stop codons | FSVGLGHGWDSIVSVNGVDTGETTMRWYKAGTQSLTIGSRLYGE |
| | (asterisks) | SSKIQPGVLSGSATLLMILPGGSSGGADVQINIRGNVYIPP** |
| 58 | BMD2 PapG_{DSF} | AGGAAAUAAGAGAGGAUAAGACGACUAAGGAGACAUACAGA |
| | N96S N286S | AUAAGAGGCAGCCACCAUGGAGACCGACACCCUCCUGCUGU |
| | Secreted_modRNA | GGGUGCUGUUACUGUGGGUGCCCGGUAGCACCGGCUGGAAC |
| | Underline = 5′ cap; | AACAUCGUGUUCUACAGCCUGGGCAACGUGAACAGCUACCAA |
| | bold = 5′ UTR and 3′ | GGCGGCAACGUGGUGAUCACACAGAGACCUCAGUUCAUCACA |
| | UTR; | AGCUGGAGACCCGGCAUCGCCACCGUGACCUGGAAUCAGUG |
| | italics = KOZAK | CAACGGCCCCGAGUUCGCCGACGGCAGCUGGGCCUACUACA |
| | sequence; | GAGAGUACAUCGCCUGGGUGGUGUUCCCCAAGAAGGUGAUG |
| | lowercase = polyA | ACCAAGAACGGCUACCCCCUGUUCAUCGAGGUGCACAACAAG |
| | tail | GGCAGCUGGAGCGAGGAGAACACCGGCGACAGCGACAGCUA |
| | (Amino acid | CUUCUUCCUGAAGGGCUACAAGUGGGACGAGAGAGCCUUCG |
| | SEQ ID NO: 37) | ACGCCGGCAACCUGUGUCAGAAGCCCGGCGAGACCACAAGAC |
| | | UGACCGAGAAGUUCAACGACAUCAUCUUCAAGGUGGCCCUGC |
| | | CCGCCGACCUGCCCCUGGGCGACUACAGCGUGACCAUCCCC |
| | | UACACAAGCGGCAUUCAGAGACACUUCGCUAGCUACCUGGGC |
| | | GCUAGAUUCAAGAUCCCCUACAACGUGGCCAAGACCCUGCCU |
| | | AGAGAGAACGAGAUGCUGUUCCUGUUCAAGAACAUCGGCGGC |
| | | UGCAGACCUAGCGCUCAGAGCCUGGAGAUCAAGCACGGCGA |
| | | CCUGAGCAUCAACAGCGCCAACAACCACUACGCCGCUCAGAC |
| | | CCUGAGCGUGAGCUGCGACGUGCCCGCCAACAUCAGAUUCA |
| | | UGCUGCUGAGAAACACCACCCCCACCUACAGCCACGGCAAGA |
| | | AGUUCAGCGUGGGCCUGGGCCACGGCUGGGACAGCAUCGUG |
| | | AGCGUGAACGGCGUGGACACCGGCGAGACUACCAUGAGAUG |
| | | GUACAAGGCCGGCACACAGAGCCUGACCAUCGGCAGCAGACU |
| | | GUACGGUGAAAGCAGCAAGAUCCAACCGGGCGUGCUGAGUG |
| | | GCUCGGCAACCCUGCUGAUGAUCCUGCCCGGAGGCAGCAGC |
| | | GGCGGCGCCGACGUGCAGAUCAACAUCAGAGGCAACGUGUA |
| | | CAUCCCCCCCUGAUGAGCUCGCUUUCUUGCUGUCCAAUUUC |
| | | UAUUAAAGGUUCCUUUGUUCCCUAAGUCCAACUACUAAACU |
| | | GGGGGAUAUUAUGAAGGGCCUUGAGCAUCUGGAUUCUGCCU |
| | | AAUAAAAAACAUUUAUUUUCAUUGCAAaaaaaaaaaaaaaaaaaaa |
| | | aaaaaaaaaaaaaaaaaaaaaaaaaaaaaaaaaaaaaaaaaaaaaaaaaaaaaa |
| | | aaaaaaa |
| 38 | PapG_{DSF} N96S | METDTLLLWVLLLWVPGSTGWNNIVFYSLGNVNSYQGGNVVITQR |
| | N242S N286S DAF | PQFITSWRPGIATVTWNQCNGPEFADGSWAYYREYIAWVVFPKK |
| | Ser-Gly GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGCRPSAQSLEI |
| | Interdomain linker | KHGDLSINSANNHYAAQTLSVSCDVPANIRFMLLRSTTPTYSHGKK |
| | (underlined); | FSVGLGHGWDSIVSVNGVDTGETTMRWYKAGTQSLTIGSRLYGE |
| | Stop codons | SSKIQPGVLSGSATLLMILPGGSSGGADVQINIRGNVYIPPSSGGG |
| | (asterisks) | GSSGSGSSSGTTRLLSGHTCFTLTGLLGTLVTMGLLT** |
| 59 | BMD2 PapG_{DSF} | AGGAAAUAAGAGAGGAUAAGACGACUAAGGAGACAUACAGA |
| | N96S N242S N286S | AUAAGAGGCAGCCACCAUGGAGACCGACACCCUCCUGCUGU |
| | DAF Ser-Gly GPI_ | GGGUGCUGUUACUGUGGGUGCCCGGUAGCACCGGCUGGAAC |
| | modRNA | AACAUCGUGUUCUACAGCCUGGGCAACGUGAACAGCUACCAA |
| | Underline = 5′ cap; | GGCGGCAACGUGGUGAUCACACAGAGACCUCAGUUCAUCACA |
| | bold = 5′ UTR and 3′ | AGCUGGAGACCCGGCAUCGCCACCGUGACCUGGAAUCAGUG |
| | UTR; | CAACGGCCCCGAGUUCGCCGACGGCAGCUGGGCCUACUACA |
| | italics = KOZAK | GAGAGUACAUCGCCUGGGUGGUGUUCCCCAAGAAGGUGAUG |
| | sequence; | ACCAAGAACGGCUACCCCCUGUUCAUCGAGGUGCACAACAAG |
| | lowercase = polyA | GGCAGCUGGAGCGAGGAGAACACCGGCGACAGCGACAGCUA |
| | tail | CUUCUUCCUGAAGGGCUACAAGUGGGACGAGAGAGCCUUCG |
| | (Amino acid | ACGCCGGCAACCUGUGUCAGAAGCCGGGGGAGACGACCCGG |
| | SEQ ID NO: 38) | CUUACAGAAAAGUUCAACGACAUCAUCUUCAAGGUGGCCCUG |
| | | CCCGCCGACCUGCCCCUGGGCGACUACAGCGUGACCAUCCC |
| | | CUACACAAGCGGCAUUCAGAGACACUUCGCUAGCUACCUGGG |
| | | CGCUAGAUUCAAGAUCCCCUACAACGUGGCCAAGACCCUGCC |
| | | UAGAGAGAACGAGAUGCUGUUCCUGUUCAAGAACAUCGGCGG |
| | | CUGCAGACCUAGCGCUCAGAGCCUGGAGAUCAAGCACGGCG |
| | | ACCUGAGCAUCAACAGCGCCAACAACCACUACGCCGCUCAGA |
| | | CCCUGAGCGUGAGCUGCGACGUGCCCGCCAACAUCAGAUUC |
| | | AUGCUGCUGAGAUCAACCACCCCCACCUACAGCCACGGCAAG |
| | | AAGUUCAGCGUGGGCCUGGGCCACGGCUGGGACAGCAUCGU |
| | | GAGCGUGAACGGCGUGGACACGGGCGAGACCACUAUGCGUU |
| | | GGUACAAGGCCGGCACACAGAGCCUGACCAUCGGCAGCAGAC |
| | | UGUACGGCGAGAGCAGCAAGAUUCAACCGGGCGUGCUUAGU |
| | | GGUUCCGCCACCCUGCUGAUGAUUCUGCCCGGCGGAAGCAG |
| | | CGGCGGCGCCGACGUGCAGAUCAACAUCAGAGGCAACGUGU |
| | | ACAUACCCCCUUCGAGCGGAGGCGGCGGAUCGUCCGGCUCC |
| | | GGGAGCAGCAGCGGCACCACACGGCUGCUGAGCGGCCACAC |
| | | CUGCUUCACCCUGACCGGCCUGCUGGGCACCCUGGUGACCA |
| | | UGGGCCUGCUGACCUGAUGAGCUCGCUUUCUUGCUGUCCAA |
| | | UUUCUAUUAAAGGUUCCUUUGUUCCCUAAGUCCAACUACUA |
| | | AACUGGGGGAUAUUAUGAAGGGCCUUGAGCAUCUGGAUUCU |
| | | GCCUAAUAAAAAACAUUUAUUUUCAUUGCAAaaaaaaaaaaaaaa |
| | | aaaaaaaaaaaaaaaaaaaaaaaaaaaaaaaaaaaaaaaaaaaaaaaaaaaaaa |
| | | aaaaaaaaaaaa |
| 60 | BMD562 PapG_{DSF} | AGGAAAUAAGAGAAAGAGGAUAAGACGACUAAGGAGACAUA |
| | N96S N242S N286S | CAGAAUAAGAGGCAGCCACCAUGGAGACCGACACCCUCCUG |
| | DAF Ser-Gly GPI_ | CUGUGGGUGCUGUUACUGUGGGUGCCCGGUAGCACCGGCUG |
| | modRNA | GAACAACAUCGUGUUCUACAGCCUGGGCAACGUGAACAGCUA |
| | Underline = 5′ cap; | CCAAGGCGGCAACGUGGUGAUCACACAGAGACCUCAGUUCAU |
| | bold = 5′ UTR and 3′ | CACAAGCUGGAGACCCGGCAUCGCCACCGUGACCUGGAAUCA |
| | UTR; | GUGCAACGGCCCCGAGUUCGCCGACGGCAGCUGGGCCUACU |
| | italics = KOZAK | ACAGAGAGUACAUCGCCUGGGUGGUGUUCCCCAAGAAGGUG |
| | sequence; | AUGACCAAGAACGGCUACCCCCUGUUCAUCGAGGUGCACAAC |
| | lowercase = polyA | AAGGGCAGCUGGAGCGAGGAGAACACCGGCGACAGCGACAG |
| | tail | CUACUUCUUCCUGAAGGGCUACAAGUGGGACGAGAGAGCCU |
| | (Amino acid | UCGACGCCGGCAACCUGUGUCAGAAGCCGGGGGAGACGACC |
| | SEQ ID NO: 38) | CGGCUUACAGAAAAGUUCAACGACAUCAUCUUCAAGGUGGCC |
| | | CUGCCCGCCGACCUGCCCCUGGGCGACUACAGCGUGACCAU |
| | | CCCCUACACAAGCGGCAUUCAGAGACACUUCGCUAGCUACCU |
| | | GGGCGCUAGAUUCAAGAUCCCCUACAACGUGGCCAAGACCCU |
| | | GCCUAGAGAGAACGAGAUGCUGUUCCUGUUCAAGAACAUCGG |
| | | CGGCUGCAGACCUAGCGCUCAGAGCCUGGAGAUCAAGCACG |
| | | GCGACCUGAGCAUCAACAGCGCCAACAACCACUACGCCGCUC |
| | | AGACCCUGAGCGUGAGCUGCGACGUGCCCGCCAACAUCAGA |
| | | UUCAUGCUGCUGAGAUCAACCACCCCCACCUACAGCCACGGC |
| | | AAGAAGUUCAGCGUGGGCCUGGGCCACGGCUGGGACAGCAU |
| | | CGUGAGCGUGAACGGCGUGGACACGGGCGAGACCACUAUGC |
| | | GUUGGUACAAGGCCGGCACACAGAGCCUGACCAUCGGCAGC |
| | | AGACUGUACGGCGAGAGCAGCAAGAUUCAACCGGGCGUGCU |
| | | UAGUGGUUCCGCCACCCUGCUGAUGAUUCUGCCCGGCGGAA |
| | | GCAGCGGCGGCGCCGACGUGCAGAUCAACAUCAGAGGCAAC |
| | | GUGUACAUACCCCCUUCGAGCGGAGGCGGCGGAUCGUCCGG |
| | | CUCCGGGAGCAGCAGCGGCACCACACGGCUGCUGAGCGGCC |
| | | ACACCUGCUUCACCCUGACCGGCCUGCUGGGCACCCUGGUG |
| | | ACCAUGGGCCUGCUGACCUGAUGAGCUCGCUUUCUUGCUGU |
| | | CCAAUUUCUAUUAAAGGUUCCUUUGUUCCCUAAGUCCAACU |
| | | ACUAAACUGGGGGAUAUUAUGAAGGGCCUUGAGCAUCUGGA |
| | | UUCUGCCUAAUAAAAAACAUUUAUUUUCAUUGCAAaaaaaaaaa |
| | | aaaaaaaaaaaaaaaaaaaaaaaaaaaaaaaaaaaaaaaaaaaaaaaaaaaaaa |
| | | aaaaaaaaaaaaaaaaa |
| 61 | BMD576 PapG_{DSF} | AGGAGGACUGGUCGAACCUGCAUAGUGAUCAUAAGGUCAGC |
| | N96S N242S N286S | AUAGCCACCAUGGAGACCGACACCCUCCUGCUGUGGGUGCU |
| | DAF Ser-Gly GPI_ | GUUACUGUGGGUGCCCGGUAGCACCGGCUGGAACAACAUCG |
| | modRNA | UGUUCUACAGCCUGGGCAACGUGAACAGCUACCAAGGCGGCA |
| | Underline = 5′ cap; | ACGUGGUGAUCACACAGAGACCUCAGUUCAUCACAAGCUGGA |
| | bold = 5′ UTR and 3′ | GACCCGGCAUCGCCACCGUGACCUGGAAUCAGUGCAACGGC |
| | UTR; | CCCGAGUUCGCCGACGGCAGCUGGGCCUACUACAGAGAGUA |
| | italics = KOZAK | CAUCGCCUGGGUGGUGUUCCCCAAGAAGGUGAUGACCAAGA |
| | sequence; | ACGGCUACCCCCUGUUCAUCGAGGUGCACAACAAGGGCAGCU |
| | lowercase = polyA | GGAGCGAGGAGAACACCGGCGACAGCGACAGCUACUUCUUC |
| | tail | CUGAAGGGCUACAAGUGGGACGAGAGAGCCUUCGACGCCGG |
| | (Amino acid | CAACCUGUGUCAGAAGCCGGGGGAGACGACCCGGCUUACAG |
| | SEQ ID NO: 38) | AAAAGUUCAACGACAUCAUCUUCAAGGUGGCCCUGCCCGCCG |
| | | ACCUGCCCCUGGGCGACUACAGCGUGACCAUCCCCUACACAA |
| | | GCGGCAUUCAGAGACACUUCGCUAGCUACCUGGGCGCUAGA |
| | | UUCAAGAUCCCCUACAACGUGGCCAAGACCCUGCCUAGAGAG |
| | | AACGAGAUGCUGUUCCUGUUCAAGAACAUCGGCGGCUGCAGA |
| | | CCUAGCGCUCAGAGCCUGGAGAUCAAGCACGGCGACCUGAG |
| | | CAUCAACAGCGCCAACAACCACUACGCCGCUCAGACCCUGAG |
| | | CGUGAGCUGCGACGUGCCCGCCAACAUCAGAUUCAUGCUGC |
| | | UGAGAUCAACCACCCCCACCUACAGCCACGGCAAGAAGUUCA |
| | | GCGUGGGCCUGGGCCACGGCUGGGACAGCAUCGUGAGCGUG |
| | | AACGGCGUGGACACGGGCGAGACCACUAUGCGUUGGUACAA |
| | | GGCCGGCACACAGAGCCUGACCAUCGGCAGCAGACUGUACG |
| | | GCGAGAGCAGCAAGAUUCAACCGGGCGUGCUUAGUGGUUCC |
| | | GCCACCCUGCUGAUGAUUCUGCCCGGCGGAAGCAGCGGCGG |
| | | CGCCGACGUGCAGAUCAACAUCAGAGGCAACGUGUACAUACC |
| | | CCCUUCGAGCGGAGGCGGCGGAUCGUCCGGCUCCGGGAGCA |
| | | GCAGCGGCACCACACGGCUGCUGAGCGGCCACACCUGCUUC |
| | | ACCCUGACCGGCCUGCUGGGCACCCUGGUGACCAUGGGCCU |
| | | GCUGACCUGAUGAGCUCGCUUUCUUGCUGUCCAAUUUCUAU |
| | | UAAAGGUUCCUUUGUUCCCUAAGUCCAACUACUAAACUGGG |
| | | GGAUAUUAUGAAGGGCCUUGAGCAUCUGGAUUCUGCCUAAU |
| | | AAAAAACAUUUAUUUUCAUUGCAAaaaaaaaaaaaaaaaaaaaaaaa |
| | | aaaaaaaaaaaaaaaaaaaaaaaaaaaaaaaaaaaaaaaaaaaaaaaaaaaaaa |
| | | aaa |
| 39 | PapG_{DSF} N96S | METDTLLLWVLLLWVPGSTGWNNIVFYSLGNVNSYQGGNVVITQR |
| | N242S N286S | PQFITSWRPGIATVTWNQCNGPEFADGSWAYYREYIAWVVFPKK |
| | K172A DAF Ser- | VMTKNGYPLFIEVHNKGSWSEENTGDSDSYFFLKGYKWDERAFD |
| | Gly GPI | AGNLCQKPGETTRLTEKFNDIIFKVALPADLPLGDYSVTIPYTSGIQ |
| | Mouse IgK signal | RHFASYLGARFAIPYNVAKTLPRENEMLFLFKNIGGCRPSAQSLEI |
| | peptide (italics); | KHGDLSINSANNHYAAQTLSVSCDVPANIRFMLLRSTTPTYSHGKK |
| | Interdomain linker | FSVGLGHGWDSIVSVNGVDTGETTMRWYKAGTQSLTIGSRLYGE |
| | (underlined); | SSKIQPGVLSGSATLLMILPGGSSGGADVQINIRGNVYIPPSSGGG |
| | Stop codons | GSSGSGSSSGTTRLLSGHTCFTLTGLLGTLVTMGLLT** |
| | (asterisks) | |
| 62 | BMD2 PapG_{DSF} | AGGAAAUAAGAGAGGAUAAGACGACUAAGGAGACAUACAGA |
| | N96S N242S N286S | AUAAGAGGCAGCCACCAUGGAGACCGACACCCUCCUGCUGU |
| | K172A DAF Ser-Gly | GGGUGCUGUUACUGUGGGUGCCCGGUAGCACCGGCUGGAAC |
| | GPI_modRNA | AACAUCGUGUUCUACAGCCUGGGCAACGUGAACAGCUACCAA |
| | Underline = 5′ cap; | GGCGGCAACGUGGUGAUCACACAGAGACCUCAGUUCAUCACA |
| | bold = 5′ UTR and 3′ | AGCUGGAGACCCGGCAUCGCCACCGUGACCUGGAAUCAGUG |
| | UTR; | CAACGGCCCCGAGUUCGCCGACGGCAGCUGGGCCUACUACA |
| | italics = KOZAK | GAGAGUACAUCGCCUGGGUGGUGUUCCCCAAGAAGGUGAUG |
| | sequence; | ACCAAGAACGGCUACCCCCUGUUCAUCGAGGUGCACAACAAG |
| | lowercase = polyA | GGCAGCUGGAGCGAGGAGAACACCGGCGACAGCGACAGCUA |
| | tail | CUUCUUCCUGAAGGGCUACAAGUGGGACGAGAGAGCCUUCG |
| | (Amino acid | ACGCCGGCAACCUGUGUCAGAAGCCGGGGGAGACGACCCGG |
| | SEQ ID NO: 39) | CUUACAGAAAAGUUCAACGACAUCAUCUUCAAGGUGGCCCUG |
| | | CCCGCCGACCUGCCCCUGGGCGACUACAGCGUGACCAUCCC |
| | | CUACACAAGCGGCAUUCAGAGACACUUCGCUAGCUACCUGGG |
| | | CGCUAGAUUCGCCAUCCCCUACAACGUGGCCAAGACCCUGCC |
| | | UAGAGAGAACGAGAUGCUGUUCCUGUUCAAGAACAUCGGCGG |
| | | CUGCAGACCUAGCGCUCAGAGCCUGGAGAUCAAGCACGGCG |
| | | ACCUGAGCAUCAACAGCGCCAACAACCACUACGCCGCUCAGA |
| | | CCCUGAGCGUGAGCUGCGACGUGCCCGCCAACAUCAGAUUC |
| | | AUGCUGCUGAGAUCAACCACCCCCACCUACAGCCACGGCAAG |
| | | AAGUUCAGCGUGGGCCUGGGCCACGGCUGGGACAGCAUCGU |
| | | GAGCGUGAACGGCGUGGACACGGGCGAGACCACUAUGCGUU |
| | | GGUACAAGGCCGGCACACAGAGCCUGACCAUCGGCAGCAGAC |
| | | UGUACGGCGAGAGCAGCAAGAUUCAACCGGGCGUGCUUAGU |
| | | GGUUCCGCCACCCUGCUGAUGAUUCUGCCCGGCGGAAGCAG |
| | | CGGCGGCGCCGACGUGCAGAUCAACAUCAGAGGCAACGUGU |
| | | ACAUACCCCCUUCGAGCGGAGGCGGCGGAUCGUCCGGCUCC |
| | | GGGAGCAGCAGCGGCACCACACGGCUGCUGAGCGGCCACAC |
| | | CUGCUUCACCCUGACCGGCCUGCUGGGCACCCUGGUGACCA |
| | | UGGGCCUGCUGACCUGAUGAGCUCGCUUUCUUGCUGUCCAA |
| | | UUUCUAUUAAAGGUUCCUUUGUUCCCUAAGUCCAACUACUA |
| | | AACUGGGGGAUAUUAUGAAGGGCCUUGAGCAUCUGGAUUCU |
| | | GCCUAAUAAAAAACAUUUAUUUUCAUUGCAAaaaaaaaaaaaaaa |
| | | aaaaaaaaaaaaaaaaaaaaaaaaaaaaaaaaaaaaaaaaaaaaaaaaaaaaaa |
| | | aaaaaaaaaaaa |
| 63 | BMD562 PapG_{DSF} | AGGAAAUAAGAGAAAGAGGAUAAGACGACUAAGGAGACAUA |
| | N96S N242S N286S | CAGAAUAAGAGGCAGCCACCAUGGAGACCGACACCCUCCUG |
| | K172A DAF Ser-Gly | CUGUGGGUGCUGUUACUGUGGGUGCCCGGUAGCACCGGCUG |
| | GPI_modRNA | GAACAACAUCGUGUUCUACAGCCUGGGCAACGUGAACAGCUA |
| | Underline = 5′ cap; | CCAAGGCGGCAACGUGGUGAUCACACAGAGACCUCAGUUCAU |
| | bold = 5′ UTR and 3′ | CACAAGCUGGAGACCCGGCAUCGCCACCGUGACCUGGAAUCA |
| | UTR; | GUGCAACGGCCCCGAGUUCGCCGACGGCAGCUGGGCCUACU |
| | italics = KOZAK | ACAGAGAGUACAUCGCCUGGGUGGUGUUCCCCAAGAAGGUG |
| | sequence; | AUGACCAAGAACGGCUACCCCCUGUUCAUCGAGGUGCACAAC |
| | lowercase = polyA | AAGGGCAGCUGGAGCGAGGAGAACACCGGCGACAGCGACAG |
| | tail | CUACUUCUUCCUGAAGGGCUACAAGUGGGACGAGAGAGCCU |
| | (Amino acid | UCGACGCCGGCAACCUGUGUCAGAAGCCGGGGGAGACGACC |
| | SEQ ID NO: 39) | CGGCUUACAGAAAAGUUCAACGACAUCAUCUUCAAGGUGGCC |
| | | CUGCCCGCCGACCUGCCCCUGGGCGACUACAGCGUGACCAU |
| | | CCCCUACACAAGCGGCAUUCAGAGACACUUCGCUAGCUACCU |
| | | GGGCGCUAGAUUCGCCAUCCCCUACAACGUGGCCAAGACCCU |
| | | GCCUAGAGAGAACGAGAUGCUGUUCCUGUUCAAGAACAUCGG |
| | | CGGCUGCAGACCUAGCGCUCAGAGCCUGGAGAUCAAGCACG |
| | | GCGACCUGAGCAUCAACAGCGCCAACAACCACUACGCCGCUC |
| | | AGACCCUGAGCGUGAGCUGCGACGUGCCCGCCAACAUCAGA |
| | | UUCAUGCUGCUGAGAUCAACCACCCCCACCUACAGCCACGGC |
| | | AAGAAGUUCAGCGUGGGCCUGGGCCACGGCUGGGACAGCAU |
| | | CGUGAGCGUGAACGGCGUGGACACGGGCGAGACCACUAUGC |
| | | GUUGGUACAAGGCCGGCACACAGAGCCUGACCAUCGGCAGC |
| | | AGACUGUACGGCGAGAGCAGCAAGAUUCAACCGGGCGUGCU |
| | | UAGUGGUUCCGCCACCCUGCUGAUGAUUCUGCCCGGCGGAA |
| | | GCAGCGGCGGCGCCGACGUGCAGAUCAACAUCAGAGGCAAC |
| | | GUGUACAUACCCCCUUCGAGCGGAGGCGGCGGAUCGUCCGG |
| | | CUCCGGGAGCAGCAGCGGCACCACACGGCUGCUGAGCGGCC |
| | | ACACCUGCUUCACCCUGACCGGCCUGCUGGGCACCCUGGUG |
| | | ACCAUGGGCCUGCUGACCUGAUGAGCUCGCUUUCUUGCUGU |
| | | CCAAUUUCUAUUAAAGGUUCCUUUGUUCCCUAAGUCCAACU |
| | | ACUAAACUGGGGGAUAUUAUGAAGGGCCUUGAGCAUCUGGA |
| | | UUCUGCCUAAUAAAAAACAUUUAUUUUCAUUGCAAaaaaaaaaa |
| | | aaaaaaaaaaaaaaaaaaaaaaaaaaaaaaaaaaaaaaaaaaaaaaaaaaaaaa |
| | | aaaaaaaaaaaaaaaaa |
| 64 | BMD576 PapG_{DSF} | AGGAGGACUGGUCGAACCUGCAUAGUGAUCAUAAGGUCAGC |
| | N96S N242S N286S | AUAGCCACCAUGGAGACCGACACCCUCCUGCUGUGGGUGCU |
| | K172A DAF Ser-Gly | GUUACUGUGGGUGCCCGGUAGCACCGGCUGGAACAACAUCG |
| | GPI_modRNA | UGUUCUACAGCCUGGGCAACGUGAACAGCUACCAAGGCGGCA |
| | Underline = 5′ cap; | ACGUGGUGAUCACACAGAGACCUCAGUUCAUCACAAGCUGGA |
| | bold = 5′ UTR and 3′ | GACCCGGCAUCGCCACCGUGACCUGGAAUCAGUGCAACGGC |
| | UTR; | CCCGAGUUCGCCGACGGCAGCUGGGCCUACUACAGAGAGUA |
| | italics = KOZAK | CAUCGCCUGGGUGGUGUUCCCCAAGAAGGUGAUGACCAAGA |
| | sequence; | ACGGCUACCCCCUGUUCAUCGAGGUGCACAACAAGGGCAGCU |
| | lowercase = polyA | GGAGCGAGGAGAACACCGGCGACAGCGACAGCUACUUCUUC |
| | tail | CUGAAGGGCUACAAGUGGGACGAGAGAGCCUUCGACGCCGG |
| | (Amino acid | CAACCUGUGUCAGAAGCCGGGGGAGACGACCCGGCUUACAG |
| | SEQ ID NO: 39) | AAAAGUUCAACGACAUCAUCUUCAAGGUGGCCCUGCCCGCCG |
| | | ACCUGCCCCUGGGCGACUACAGCGUGACCAUCCCCUACACAA |
| | | GCGGCAUUCAGAGACACUUCGCUAGCUACCUGGGCGCUAGA |
| | | UUCGCCAUCCCCUACAACGUGGCCAAGACCCUGCCUAGAGAG |
| | | AACGAGAUGCUGUUCCUGUUCAAGAACAUCGGCGGCUGCAGA |
| | | CCUAGCGCUCAGAGCCUGGAGAUCAAGCACGGCGACCUGAG |
| | | CAUCAACAGCGCCAACAACCACUACGCCGCUCAGACCCUGAG |
| | | CGUGAGCUGCGACGUGCCCGCCAACAUCAGAUUCAUGCUGC |
| | | UGAGAUCAACCACCCCCACCUACAGCCACGGCAAGAAGUUCA |
| | | GCGUGGGCCUGGGCCACGGCUGGGACAGCAUCGUGAGCGUG |
| | | AACGGCGUGGACACGGGCGAGACCACUAUGCGUUGGUACAA |
| | | GGCCGGCACACAGAGCCUGACCAUCGGCAGCAGACUGUACG |
| | | GCGAGAGCAGCAAGAUUCAACCGGGCGUGCUUAGUGGUUCC |
| | | GCCACCCUGCUGAUGAUUCUGCCCGGCGGAAGCAGCGGCGG |
| | | CGCCGACGUGCAGAUCAACAUCAGAGGCAACGUGUACAUACC |
| | | CCCUUCGAGCGGAGGCGGCGGAUCGUCCGGCUCCGGGAGCA |
| | | GCAGCGGCACCACACGGCUGCUGAGCGGCCACACCUGCUUC |
| | | ACCCUGACCGGCCUGCUGGGCACCCUGGUGACCAUGGGCCU |
| | | GCUGACCUGAUGAGCUCGCUUUCUUGCUGUCCAAUUUCUAU |
| | | UAAAGGUUCCUUUGUUCCCUAAGUCCAACUACUAAACUGGG |
| | | GGAUAUUAUGAAGGGCCUUGAGCAUCUGGAUUCUGCCUAAU |
| | | AAAAAACAUUUAUUUUCAUUGCAAaaaaaaaaaaaaaaaaaaaaaaa |
| | | aaaaaaaaaaaaaaaaaaaaaaaaaaaaaaaaaaaaaaaaaaaaaaaaaaaaaa |
| | | aaa |
| 40 | PapG_{DSF} N96S | METDTLLLWVLLLWVPGSTGWNNIVFYSLGNVNSYQGGNVVITQR |
| | N242S N286S Thy1 | PQFITSWRPGIATVTWNQCNGPEFADGSWAYYREYIAWVVFPKK |
| | Ser-Gly GPI | VMTKNGYPLFIEVHNKGSWSEENTGDSDSYFFLKGYKWDERAFD |
| | Mouse IgK signal | AGNLCQKPGETTRLTEKFNDIIFKVALPADLPLGDYSVTIPYTSGIQ |
| | peptide (italics); | RHFASYLGARFKIPYNVAKTLPRENEMLFLFKNIGGCRPSAQSLEI |
| | Interdomain linker | KHGDLSINSANNHYAAQTLSVSCDVPANIRFMLLRSTTPTYSHGKK |
| | (underlined); | FSVGLGHGWDSIVSVNGVDTGETTMRWYKAGTQSLTIGSRLYGE |
| | Stop codons | SSKIQPGVLSGSATLLMILPGGSSGGADVQINIRGNVYIPPSSGGG |
| | (asterisks) | GSSGSGSSCEGISLLAQNTSWLLLLLLSLSLLQATDFMSL** |
| 65 | BMD576 PapG_{DSF} | AGGAGGACUGGUCGAACCUGCAUAGUGAUCAUAAGGUCAGC |
| | N96S N242S N286S | AUAGCCACCAUGGAGACCGACACCCUCCUGCUGUGGGUGCU |
| | Thy1 Ser-Gly GPI_ | GUUACUGUGGGUGCCCGGUAGCACCGGCUGGAACAACAUCG |
| | modRNA | UGUUCUACAGCCUGGGCAACGUGAACAGCUACCAAGGCGGCA |
| | Underline = 5′ cap; | ACGUGGUGAUCACACAGAGACCUCAGUUCAUCACAAGCUGGA |
| | bold = 5′ UTR and 3′ | GACCCGGCAUCGCCACCGUGACCUGGAAUCAGUGCAACGGC |
| | UTR; | CCCGAGUUCGCCGACGGCAGCUGGGCCUACUACAGAGAGUA |
| | italics = KOZAK | CAUCGCCUGGGUGGUGUUCCCCAAGAAGGUGAUGACCAAGA |
| | sequence; | ACGGCUACCCCCUGUUCAUCGAGGUGCACAACAAGGGCAGCU |
| | lowercase = polyA | GGAGCGAGGAGAACACCGGCGACAGCGACAGCUACUUCUUC |
| | tail | CUGAAGGGCUACAAGUGGGACGAGAGAGCCUUCGACGCCGG |
| | (Amino acid | CAACCUGUGUCAGAAGCCGGGGGAGACCACAAGACUUACCGA |
| | SEQ ID NO: 40) | GAAGUUCAACGACAUCAUCUUCAAGGUGGCCCUGCCCGCCGA |
| | | CCUGCCCCUGGGCGACUACAGCGUGACCAUCCCCUACACAAG |
| | | CGGCAUUCAGAGACACUUCGCUAGCUACCUGGGCGCUAGAU |
| | | UCAAGAUCCCCUACAACGUGGCCAAGACCCUGCCUAGAGAGA |
| | | ACGAGAUGCUGUUCCUGUUCAAGAACAUCGGCGGCUGCAGA |
| | | CCUAGCGCUCAGAGCCUGGAGAUCAAGCACGGCGACCUGAG |
| | | CAUCAACAGCGCCAACAACCACUACGCCGCUCAGACCCUGAG |
| | | CGUGAGCUGCGACGUGCCCGCCAACAUCAGAUUCAUGCUGC |
| | | UGAGAUCAACCACCCCCACCUACAGCCACGGCAAGAAGUUCA |
| | | GCGUGGGCCUGGGCCACGGCUGGGACAGCAUCGUGAGCGUG |
| | | AACGGCGUGGACACGGGCGAGACCACUAUGCGUUGGUACAA |
| | | GGCCGGCACACAGAGCCUGACCAUCGGCAGCAGACUGUAUG |
| | | GCGAAAGCUCCAAAAUCCAACCCGGCGUGCUGUCGGGGAGC |
| | | GCCACCCUGCUGAUGAUACUGCCCGGAGGCAGCAGCGGCGG |
| | | CGCCGACGUGCAGAUCAACAUCAGAGGCAACGUGUAUAUUCC |
| | | GCCUAGCAGUGGUGGUGGGGGUAGCUCCGGCUCCGGAAGCA |
| | | GCUGCGAGGGCAUCAGCCUGCUGGCUCAGAACACAAGCUGG |
| | | CUGCUGCUGCUGCUGCUGAGCCUGAGCCUGCUGCAAGCCAC |
| | | CGACUUCAUGAGCCUGUGAUGAGCUCGCUUUCUUGCUGUCC |
| | | AAUUUCUAUUAAAGGUUCCUUUGUUCCCUAAGUCCAACUAC |
| | | UAAACUGGGGGAUAUUAUGAAGGGCCUUGAGCAUCUGGAUU |
| | | CUGCCUAAUAAAAAACAUUUAUUUUCAUUGCAAaaaaaaaaaaa |
| | | aaaaaaaaaaaaaaaaaaaaaaaaaaaaaaaaaaaaaaaaaaaaaaaaaaaaaa |
| | | aaaaaaaaaaaaaaa |
| 41 | PapG_{DSF} N96S | METDTLLLWVLLLWVPGSTGWNNIVFYSLGNVNSYQGGNVVITQR |
| | N242S N286S | PQFITSWRPGIATVTWNQCNGPEFADGSWAYYREYIAWVVFPKK |
| | K172A Thy1 Ser- | VMTKNGYPLFIEVHNKGSWSEENTGDSDSYFFLKGYKWDERAFD |
| | Gly GPI | AGNLCQKPGETTRLTEKFNDIIFKVALPADLPLGDYSVTIPYTSGIQ |
| | Mouse IgK signal | RHFASYLGARFAIPYNVAKTLPRENEMLFLFKNIGGCRPSAQSLEI |
| | peptide (italics); | KHGDLSINSANNHYAAQTLSVSCDVPANIRFMLLRSTTPTYSHGKK |
| | Interdomain linker | FSVGLGHGWDSIVSVNGVDTGETTMRWYKAGTQSLTIGSRLYGE |
| | (underlined); | SSKIQPGVLSGSATLLMILPGGSSGGADVQINIRGNVYIPPSSGGG |
| | Stop codons | GSSGSGSSCEGISLLAQNTSWLLLLLLSLSLLQATDFMSL** |
| | (asterisks) | |
| 66 | BMD576 PapG_{DSF} | AGGAGGACUGGUCGAACCUGCAUAGUGAUCAUAAGGUCAGC |
| | N96S N242S N286S | AUAGCCACCAUGGAGACCGACACCCUCCUGCUGUGGGUGCU |
| | K172A Thy1 Ser- | GUUACUGUGGGUGCCCGGUAGCACCGGCUGGAACAACAUCG |
| | Gly GPI_modRNA | UGUUCUACAGCCUGGGCAACGUGAACAGCUACCAAGGCGGCA |
| | Underline = 5′ cap; | ACGUGGUGAUCACACAGAGACCUCAGUUCAUCACAAGCUGGA |
| | bold = 5′ UTR and 3′ | GACCCGGCAUCGCCACCGUGACCUGGAAUCAGUGCAACGGC |
| | UTR; | CCCGAGUUCGCCGACGGCAGCUGGGCCUACUACAGAGAGUA |
| | italics = KOZAK | CAUCGCCUGGGUGGUGUUCCCCAAGAAGGUGAUGACCAAGA |
| | sequence; | ACGGCUACCCCCUGUUCAUCGAGGUGCACAACAAGGGCAGCU |
| | lowercase = polyA | GGAGCGAGGAGAACACCGGCGACAGCGACAGCUACUUCUUC |
| | tail | CUGAAGGGCUACAAGUGGGACGAGAGAGCCUUCGACGCCGG |
| | (Amino acid | CAACCUGUGUCAGAAGCCGGGGGAGACCACAAGACUUACCGA |
| | SEQ ID NO: 41) | GAAGUUCAACGACAUCAUCUUCAAGGUGGCCCUGCCCGCCGA |
| | | CCUGCCCCUGGGCGACUACAGCGUGACCAUCCCCUACACAAG |
| | | CGGCAUUCAGAGACACUUCGCUAGCUACCUGGGCGCUAGAU |
| | | UCGCCAUCCCCUACAACGUGGCCAAGACCCUGCCUAGAGAGA |
| | | ACGAGAUGCUGUUCCUGUUCAAGAACAUCGGCGGCUGCAGA |
| | | CCUAGCGCUCAGAGCCUGGAGAUCAAGCACGGCGACCUGAG |
| | | CAUCAACAGCGCCAACAACCACUACGCCGCUCAGACCCUGAG |
| | | CGUGAGCUGCGACGUGCCCGCCAACAUCAGAUUCAUGCUGC |
| | | UGAGAUCAACCACCCCCACCUACAGCCACGGCAAGAAGUUCA |
| | | GCGUGGGCCUGGGCCACGGCUGGGACAGCAUCGUGAGCGUG |
| | | AACGGCGUGGACACGGGCGAGACCACUAUGCGUUGGUACAA |
| | | GGCCGGCACACAGAGCCUGACCAUCGGCAGCAGACUGUAUG |
| | | GCGAAAGCUCCAAAAUCCAACCCGGCGUGCUGUCGGGGAGC |
| | | GCCACCCUGCUGAUGAUACUGCCCGGAGGCAGCAGCGGCGG |
| | | CGCCGACGUGCAGAUCAACAUCAGAGGCAACGUGUAUAUUCC |
| | | GCCUAGCAGUGGUGGUGGGGGUAGCUCCGGCUCCGGAAGCA |
| | | GCUGCGAGGGCAUCAGCCUGCUGGCUCAGAACACAAGCUGG |
| | | CUGCUGCUGCUGCUGCUGAGCCUGAGCCUGCUGCAAGCCAC |
| | | CGACUUCAUGAGCCUGUGAUGAGCUCGCUUUCUUGCUGUCC |
| | | AAUUUCUAUUAAAGGUUCCUUUGUUCCCUAAGUCCAACUAC |
| | | UAAACUGGGGGAUAUUAUGAAGGGCCUUGAGCAUCUGGAUU |
| | | CUGCCUAAUAAAAAACAUUUAUUUUCAUUGCAAaaaaaaaaaaa |
| | | aaaaaaaaaaaaaaaaaaaaaaaaaaaaaaaaaaaaaaaaaaaaaaaaaaaaaa |
| | | aaaaaaaaaaaaaaa |

In Table 11, in the PapG_{DSF} construct sequences, the underlined amino acids are Gly-Ser linkers separating full-length PapG from the stabilizing donor strand F-peptide (DSF) as well as Gly-Ser linkers separating the DSF from the C-terminal GPI membrane anchoring signal of the human DAF protein or Thyl. PapG_{DSF} contains alleles N96S, N242S, N286S (fully aglycosylated) (numbering based on processed polypeptide starting with the proximal phenylalanine residue).

TABLE 12
Additional sequences

| SEQ ID | |
|---|---|
| NO: | Description and sequence |
| 67 | >mouse IgGκ signal peptide: |
| | METDTLLLWVLLLWVPGSTG |
| 68 | > PapG_{LD} V1 polypeptide: |
| | WNNIVFYSLGNVNSYQGGNVVITQRPQFITSWRPGIATVTWNQCNGPEFADG |
| | SWAYYREYIAWVVFPKKVMTKNGYPLFIEVHNKGSWSEENTGDNDSYFFLKG |
| | YKWDERAFDAGNLCQKPGETTRLTEKFNDIIFKVALPADLPLGDYSVTIPYTSGI |
| | QRHFASYLGARFKIPYNVAKTLPRENEMLFLFKNIGG |
| 69 | > PapG_{LD} V2 polypeptide: |
| | WNNIVFYSLGNVNSYQGGNVVITQRPQFITSWRPGIATVTWNQCNGPEFADG |
| | SWAYYREYIAWVVFPKKVMTQNGYPLFIEVHNKGSWSEENTGDNDSYFFLKG |
| | YKWDERAFDAGNLCQKPGETTRLTEKFDDIIFKVALPADLPLGDYSVKIPYTSG |
| | MQRHFASYLGARFKIPYNVAKTLPRENEMLFLFKNIGG |
| 70 | >PapG_{DSF} polypeptide (the underlined amino acids are Gly-Ser linkers |
| | separating full-length PapG from the stabilizing donor strand F-peptide |
| | (DSF)): |
| | WNNIVFYSLGNVNSYQGGNVVITQRPQFITSWRPGIATVTWNQCNGPEFADG |
| | SWAYYREYIAWVVFPKKVMTKNGYPLFIEVHNKGSWSEENTGDNDSYFFLKG |
| | YKWDERAFDAGNLCQKPGETTRLTEKFNDIIFKVALPADLPLGDYSVTIPYTSGI |
| | QRHFASYLGARFKIPYNVAKTLPRENEMLFLFKNIGGCRPSAQSLEIKHGDLSI |
| | NSANNHYAAQTLSVSCDVPANIRFMLLRNTTPTYSHGKKFSVGLGHGWDSIVS |
| | VNGVDTGETTMRWYKAGTQNLTIGSRLYGESSKIQPGVLSGSATLLMILPGGS |
| | SGGADVQINIRGNVYIPP |
| 71 | >Linker |
| | GGSSGGGGSSGSGSSSG |
| 72 | > Linker |
| | SSGGGGSSGSGSSSG |
| 73 | >Linker |
| | SSGGGGSSGSGSS |
| 74 | >Linker |
| | SSGGG |
| 75 | >polyA tail: |
| | AAAAAAAAAAAAAAAAAAAAAAAAAAAAAAAAAAAAAAAAAAAAAAAAAAAAA |
| | AAAAAAAAAAAAAAAAAAAAAAAAAAAAA |

TABLE 13
Polypeptide Sequences of WT PapG and ATLAS collection of E. coli UPEC
isolates:

>PapGI WT_P13720 (SEQ ID NO: 119)
MKKWFPAFLFLSLSGGNDALAGWHNVMFYAFNDYLTTNAGNVKVIDQPQLYIPWNTGSATA
TYYSCSGPEFASGVYFQEYLAWMVVPKHVYTNEGFNIFLDVQSKYGWSMENENDKDFYFF
VNGYEWDTWTNNGARICFYPGNMKQLNNKFNDLVFRVLLPVDLPKGHYNFPVRYIRGIQHH
YYDLWQDHYKMPYDQIKQLPATNTLMLSFDNVGGCQPSTQVLNIDHGSIVIDRANGNIASQT
LSIYCDVPVSVKISLLRNTPPIYNNNKFSVGLGNGWDSIISLDGVEQSEEILRWYTAGSKTVKI
ESRLYGEEGKRKPGELSGSMTMVLSFP
>PapGII_1_WT CFT073 reference strain (SEQ ID NO: 120)
MKKWFPALLFSLCVSGESSAWNNIVFYSLGNVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDAGNLCQKPGETTRLTEKFNDIIFKVALPADLPLGDYSVTIPYTSGIQRHFA
SYLGARFKIPYNVAKTLPRENEMLFLFKNIGGCRPSAQSLEIKHGDLSINSANNHYAAQTLSV
SCDVPANIRFMLLRNTTPTYSHGKKFSVGLGHGWDSIVSVNGVDTGETTMRWYKAGTQNL
TIGSRLYGESSKIQPGVLSGSATLLMILP
>PapGII_2 WT_CFT073 reference strain (SEQ ID NO: 121)
MKKWFPALLFSLCVSGESSAWNNIVFYSLGNVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDAGNLCQKPGETTRLTEKFNDIIFKVALPADLPLGDYSVTIPYTSGIQRHFA
SYLGARFKIPYNVAKTLPRENEMLFLFKNIGGCRPSAQSLEIKHGDLSINSANNHYAAQTLSV
SCDVPANIRFMLLRNTTPTYSHGKKFSVGLGHGWDSIVSVNGVDTGETTMRWYKAGTQNL
TIGSRLYGESSKIQPGVLSGSATLLMILP
>PapGIII WT_P42188 (SEQ ID NO: 122)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEANTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTNPAYSHGQQFSVGLGHGWDSIISINGVDTGETTMRWYRAGTQNLTT
GSRLYGESSKIQPGVLSGSATLLMILP
>2134-PapGIII (SEQ ID NO: 123)
MKKWLPAFLFLSLSGCNDALAANQSTMFYSFNDNIYRPQLSVKVTDIVQFIVDINSASSTATL
NYVACNGFTWTHGLYWSEYFAWLVVPKHVSYNGYNIYLELQSRGSFSLDAEDNDNYYLTK
GFAWDEANTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTAPAYSHGQQFSVGLGHGWDSIVSVNGVDKGETTMRWYRAGTQNLTI
GSRLYGESSKIQPGVLSGSATLLMILP
>2204-PapGIII (SEQ ID NO: 124)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEANTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSVNGVDKGETTMRWYSAGTQNLTI
GSRLYGESSKIQPGVLSGSATLLMILP
>2216-PapGIII (SEQ ID NO: 125)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEVNTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSVNGVDKGETTMRWYSAGTQNLTI
GSRLYGESSKIQPGVLSGSATLLMILP
>2223-PapGIII (SEQ ID NO: 126)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEANTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTAPAYSHGQQFSVGLGHGWDSIVSVNGVDKGETTMRWYRAGTQNLTI
GSRLYGESSKIQPGVLSGSATLLMILP
>2098-PapGIII (SEQ ID NO: 127)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEANTSGQTCFNIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTNPAYSHGQQFSVGLGHGWDSIISINGVDTGETTMRWYRAGTQNLTIG
SRLYGESSKIQPGVLSGSATLLMILP
>2094-PapGIII (SEQ ID NO: 128)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSKGGFSLDAEDNDNYYLTK
GFAWDEVNSSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSVNGVDKGETTMRWYSAGTQNLTI
GSRLYGESSKIQPGVLSGSATLLMILP
>2192-PapGIII (SEQ ID NO: 129)
MKKWLPAFLFLSLSGCNDALAANQSTMFYSFNDNIYRPQLSVKVADIVQFIVDINSASSTATL
SYVACNGFTWTHGLYWSEYFAWLVVPKHVSYNGYNIYLELQSKGGFSLDAEDNDNYYLTK
GFAWDEVNSSGRVCFNIGEKRSLAWSFGGVTLNARFPVDLPKGDYTFPVKFLRGIQRNNYD
YIGGRYKIPSSLMKTFPFNGTLNFPIKNTGGCRPSAQSLEINHGDLSINSANNHYAAQTLSVS
CDVPTNIRFFLLSNTAPAYSHGQQFSVGLGHGWDSIVSVNGVDKGETTMRWYRAGTQNLTI
GSRLYGESSKIQPGVLSGSATLLMILP
>2091-PapGIII (SEQ ID NO: 130)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSKGGFSLDAEDNDNYYLTK
GFAWDEVNSSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSINGVDTGETTMRWYRAGTQNLTI
GSRLYGESSKIQPGVLSGSATLLMILP
>2108-PapGIII (SEQ ID NO: 131)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQYKGGFSLDAEDNDNYYLTK
GFAWDEVNSSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSINGVDTGETTMRWYRAGTQNLTI
GSRLYGESSKIQPGVLSGSATLLIILP
>2099-PapGIII (SEQ ID NO: 132)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGGFSLDAEDNDNYYLTK
GFAWDEVNTSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSINGVDTGETTMRWYRAGTQNLTI
GSRLYGESSKIQPGVLSGSATLLMILP
>2089-PapGIII (SEQ ID NO: 133)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEVNSSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSINGVDTGETTMRWYRAGTQNLTI
GSRLYGESSKIQPGVLSGSATLLMILP
>2100-PapGIII (SEQ ID NO: 134)
MKKWLPAFLFLSLSGCNDALAANQSTMFYSFNDNIYRPQLSVKVTDIVQFIVDINSASSTATL
SYVACNGFTWTHGLYWSEYFAWLVVPKHVSYNGYNIYLELQSRGSFSLDAEDNDNYYLTK
GFAWDEVNSSGRVCFDIGEKRSLAWSFGGVTLNARLPVDLPKGDYTFPVKFLRGIQRNNYD
YIGGRYKIPSSLMKTFPFNGTLNFSIKNTGGCRPSAQSLEINHGDLSINSANNHYAAQTLSVS
CDVPTNIRFFLLSNTTPAYSHGQQFSVGLGHGWDSIVSINGVDTGETTMRWYRAGTQNLTI
GSRLYGESSKIQPGVLSGSATLLMILP
>2116-PapGII (SEQ ID NO: 135)
MKKWFPALLFSLCVSGESSAWNNIVFYSLGDVNSYQGGNVVITQRPQFITSWRPGIATVTW
NQCNGPEFADGFWAYYREYIAWVVFPKKVMTQNGYPLFIEVHNKGNWSEENTGDNDSYFF
LKGDKWDERAFDAANLCQKLGETTRLTEQFNDIIFKVALPADLPLGDYSVTIPYTSGIQRHFA
NYFGARFKIPYNVAKTLPRENKMLFLFKNTGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGDLSGSATLLMILP
>2221-PapGII (SEQ ID NO: 136)
MKKWFPALLFSLCVSGESSAWNNIVFYSLGDVNSYQGGNVVITQRPQFITSWRPAIATVTW
NQCNGPEFADGSWAYYREYIAWVVFPKKVMTKNGYPLFIEVHNKGSWSEENTGDNDSYFF
LKGYKWDERAFDTANLCQKPGETTRLTEKFDDIIFKVALPADLPLGDYSVTIPYTSGIQRHFA
SYLGARFKIPYNVAKTLPRENEMLFLFKNIGGCRPSAQSLEIKHGDLSINSANNHYAAQTLSV
SCDVPANIRFMLLRNTTPTYSHGKKFSVGLGHGWDSIVSVNGVDTGETTMRWYKAGTQNL
TIGSRLYGESSKIQPGVLSGSATLLMILP
>2210-PapGII (SEQ ID NO: 137)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2196-PapGII (SEQ ID NO: 138)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2186-PapGII (SEQ ID NO: 139)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2179-PapGII (SEQ ID NO: 140)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2124-PapGII (SEQ ID NO: 141)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2085-PapGII (SEQ ID NO: 142)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2079-PapGII (SEQ ID NO: 143)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGDNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2212-PapGII (SEQ ID NO: 144)
MKKWFPALLFSLCVSGESSAWNNIVFYSLGDVNSYQGGNVVITQRPQFITSWRPGIATVTW
NQCNGPEFADGSWAYYREYIAWVVFPKKVMTKNGYPLFIEVHNKGSWSEENTGYNDSYFF
LKGYKWDERAFDTANLCQKPGETIRLTEKFDDIIFKVALPADLPLGDYSVTIPYTSGIQRHFAS
YLGARFKIPYNVAKTLPRENEMLFLFKNIGGCRPSAQSLEIKHGDLSINSANNHYAAQTLSVS
CDVPANIRFMLLRNTTPTYSHGKKFSVGLGHGWDSIVSVNGVDTGETTMRWYKAGTQNLTI
GSRLYGESSKIQPGVLSGSATLLMILP
>2130-PapGII (SEQ ID NO: 145)
MKKWFPALLFSLCVSGESSAWNNIVFYSLGDVNSYQGGNVVITQRPQFITSWRPGIATVTW
NQCNGPGFVDGSWAYYREYIAWVVFPKKVMTQNGYPLFIEVHNKGSWSEENTGDNDSYFF
LKGYKWDERAFDTANLCQKPGETTRLTEKFDDIIFKVALPADLPLGDYSVTIPYTSGIQRHFA
SYLGARFKIPYNVAKTLPRENEMLFLFKNIGGCRPSAQSLEIKHGDLSINSANNHYAAQTLSV
SCDVPANIRFMLLRNTTPTYSHGKKFSVGLGHGWDSIVSVNGVDTGETTMRWYKAGTQNL
TIGSRLYGESSKIQPGVLSGSATLLMILP
>2181-PapGII (SEQ ID NO: 146)
MKKWFPALLFSLCVSGESSAWNNIVFYSLGDVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2102-PapGII (SEQ ID NO: 147)
MKKWFPALLFSLCVSGESSAWNNIVFYSLGDVNSYQGGNVVITQRPQFITSWRPGIATVTW
NQCNGPGFADGYWAYYREYIAWVVFPKKVMTKNGYPLFIEVHNKGSWSEENTGDNDSYFF
LKGYKWDERAFDTANLCQKPGEKNRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2169-PapGII (SEQ ID NO: 148)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2161-PapGII (SEQ ID NO: 149)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2150-PapGII (SEQ ID NO: 150)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2142-PapGII (SEQ ID NO: 151)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2082-PapGII (SEQ ID NO: 152)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2114-PapGII (SEQ ID NO: 153)
MKKWFPALLFSLCVSGESSAWNNIVFYSLGDVNSYQGGNVVITQRPQFITSWRPGIATVTW
NQCNGPGFADGFWAYYREYIAWVVFPKKVMTQNGYPLFIEVHNKGSWSEENTGDNDSYFF
LKGYKWDERAFDTANLCQKPGEKTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTAPTYSHGKKFSVGLGHGWDSIVSVNGVDTGETTMRWYKAGTQN
LTIGSRLYGESSKIQPGVLSGSATLLMILP
>2129-PapGII (SEQ ID NO: 154)
MKKWFPALLFSLCVSGESSAWNNIVFYSLGDVNSYQGGNVVITQRPQFITSWRPGIATVTW
NQCNGPGFADGSWAYYREYIAWVVFPKKVMTQNGYPLFIEVHNKGSWSEENTGDNDSYFF
LKGYKWDERAFDAGNLCQKPGETTRLTEKFDDIIFKVALPADLPLGDYSVKIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2101-PapGII (SEQ ID NO: 155)
MKKWFPALLFSLCVSGESSAWNNIVFYSLGDVNSYQGGNVVITQRPQFITSWRPGIATVTW
NQCNGPGFADGSWAYYREYIAWVVFPKKVMTQNGYPLFIEVHNKGNWSEENSGDNDSYF
FLKGYKWDERAFDAGNLCQKPGETTRLTEKFDDIIFKVALPADLPLGDYSVKIPYTSGMQRH
FASYLGARFKIPYNVAKTLPRENEMLFLFKNIGGCRPSAQSLEIKHGDLSINSANNHYAAQTL
SVSCDVPANIRFMLLRNTTPTYSHGKKFSVGLGHGWDSIVSVNGVDTGETTMRWYKAGTQ
NLTIGSRLYGESSKIQPGVLSGSATLLMILP
>2222-PapGII (SEQ ID NO: 156)
MKKWFPALLFSLCVSGESSAWNNIVFYSLGDVNSYQGGNVVITQRPQFITSWRPGIATVTW
NQCNGPGFTDGSWAYYREYIAWVVFPKKVMTQNGYPLFIEVHNKGNWSEENSGDNDSYFF
LKGYKWDERAFDAGNLCQKPGETTRLTEKFDDIIFKVALPADLPLGDYSVKIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2119-PapGII (SEQ ID NO: 157)
MKKWFPALLFSLCVSGESSAWNNIVFYSLGNVNSYQGGNVVITQRPQFITSWRPGIATVTW
NQCNGPGFADGYWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMFFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2206-PapGII (SEQ ID NO: 158)
MKKWFPALLFSLCVSGESSAWNNIVFYSLGNVNSYQGGNVVITQRPQFITSWRPGIATVTW
NQCNGPGFVGGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2131-PapGII (SEQ ID NO: 159)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2110-PapGII (SEQ ID NO: 160)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2123-PapGII (SEQ ID NO: 161)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2132-PapGII (SEQ ID NO: 162)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2148-PapGII (SEQ ID NO: 163)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2167-PapGII (SEQ ID NO: 164)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP
>2184-PapGII (SEQ ID NO: 165)
MKKWFPALLFSLCVSGESSAWNNIVFYSLGNVNSYQGGNVVITQRPQFITSWRPGIATVTW
NQCNGPGFADGSWAYYREYIAWVVFPKKVMTKNGYPLFIEVHNKGSWSEENTGDNDSYFF
LKGYKWDERAFDAGNLCQKPGETTRLTEKFDDIIFKVALPADLPLGDYSVTIPYTSGMQRHF
ASYLGARFKIPYNVAKTLPRENEMLFLFKNIGGCRPSAQSLEIKHGDLSINSANNHYAAQTLS
VSCDVPANIRFMLLRNTTPTYSHGKKFSVGLGHGWDSIVSVNGVDTGETTMRWYKAGTQN
LTIGSRLYGESSKIQPGVLSGSATLLMILP

TABLE 14
Nanoparticle sequences

SEQ ID NO: 76
MEGMDPLAVLAESRLLPLLTVRGGEDLAGLATVLELMGVGALEITLRTEKGLEALKALRKSGL
LLGAGTVRSPKEAEAALEAGAAFLVSPGLLEEVAALAQARGVPYLPGVLTPTEVERALALGL
SALKFFPAEPFQGVRVLRAYAEVFPEVRFLPTGGIKEEHLPHYAALPNLLAVGGSWLLQGDL
AAVMKKVKAAKALLSPQAPG
SEQ ID NO: 77
MTKKVGIVDT TFARVDMAEA AIRTLKALSP NIKIIRKTVP GIKDLPVACK
KLLEEEGCDIVMALGMPGKA EKDKVCAHEA SLGLMLAQLM TNKHIIEVFV HEDEAKDDDE
LDILALVRAIEHAANVYYLL FKPEYLTRMA GKGLRQGRED AGPARE
SEQ ID NO: 78
MTKKVGIVDT TFARVDMASA AILTLKMESP NIKIIRKTVP GIKDLPVACK
KLLEEEGCDIVMALGMPGKA EKDKVCAHEA SLGLMLAQLM TNKHIIEVFV HEDEAKDDAE
LKILAARRAIEHALNVYYLL FKPEYLTRMA GKGLRQGFED AGPARE
SEQ ID NO: 79
MSTINNQLKALKVIPVIAIDNAEDIIPLGKVLAENGLPAAEITFRSSAAV
KAIMLLRSAQPEMLIGAGTI LNGVQALAAK EAGATFVVSP GFNPNTVRAC QIIGIDIVPG
VNNPSTVEAALEMGLTTLKF FPAEASGGIS MVKSLVGPYGDIRLMPTGGI TPSNIDNYLA
IPQVLACGGTWMVDKKLVTN GEWDEIARLT REIVEQVNP
SEQ ID NO: 80
MPIFTLNTNI KATDVPSDFL SLTSRLVGLI LSKPGSYVAV HINTDQQLSF
GGSTNPAAFGTLMSIGGIEP SKNRDHSAVL FDHLNAMLGI PKNRMYIHFV NLNGDDVGWN
GTTF
SEQ ID NO: 81
MNQHSHKDYE TVRIAVVRAR WHADIVDACV EAFEIAMAAI GGDRFAVDVF
DVPGAYEIPLHARTLAETGR YGAVLGTAFV VNGGIYRHEF VASAVIDGMM NVQLSTGVPV
LSAVLTPHRYRDSAEHHRFF AAHFAVKGVE AARACIEILA AREKIAA
SEQ ID NO: 82
MKMEELFKKH KIVAVLRANS VEEAIEKAVA VFAGGVHLIE ITFTVPDADT
VIKALSVLKEKGAIIGAGTV TSVEQCRKAV ESGAEFIVSP HLDEEISQFC KEKGVFYMPG
VMTPTELVKAMKLGHTILKL FPGEVVGPQF VKAMKGPFPN VKFVPTGGVN
LDNVCEWFKA GVLAVGVGSALVKGTPDEVR EKAKAFVEKI RGCTE
SEQ ID NO: 83
MNQHSHKDYE TVRIAVVRAR WHAEIVDACV SAFEAAMADI GGDRFAVDVF
DVPGAYEIPLHARTLAETGR YGAVLGTAFV VNGGIYRHEF VASAVIDGMM NVQLSTGVPV
LSAVLTPHRYRDSDAHTLLF LALFAVKGME AARACVEILA AREKIAA
SEQ ID NO: 84
MFTKSGDDGN TNVINKRVGK DSPLVNFLGD LDELNSFIGF AISKIPWEDM
KKDLERVQVELFEIGEDLST QSSKKKIDES YVLWLLAATA IYRIESGPVK LFVIPGGSEE
ASVLHVTRSVARRVERNAVK YTKELPEINR MIIVYLNRLS SLLFAMALVA NKRRNQSEKI
YEIGKSW
SEQ ID NO: 85
MNQHSHKDYE TVRIAVVRAR WHADIVDQCV RAFEEAMADA GGDRFAVDVF
DVPGAYEIPLHARTLAETGR YGAVLGTAFV VNGGIYRHEF VASAVIDGMM NVQLSTGVPV
LSAVLTPHRYRSSREHHEFF REHFMVKGVE AAAACITILA AREKIAA
SEQ ID NO: 86
MGHTKGPTPQ QHDGSALRIG IVHARWNKTI IMPLLIGTIA KLLECGVKAS
NIVVQSVPGSWELPIAVQRL YSASQLQTPS SGPSLSAGDL LGSSTTDLTA LPTTTASSTG
PFDALIAIGVLIKGETMHFE YIADSVSHGL MRVQLDTGVP VIFGVLTVLT DDQAKARAGV
IEGSHNHGEDWGLAAVEMGV RRRDWAAGKT E
SEQ ID NO: 87
MYEVDHADVY DLFYLGRGKD YAAEASDIAD LVRSRTPEAS SLLDVACGTG
THLEHFTKEFGDTAGLELSE DMLTHARKRL PDATLHQGDM RDFQLGRKFS
AVVSMFSSVG YLKTVAELGAAVASFAEHLE PGGVVVVEPW WFPETFADGW
VSADVVRRDG RTVARVSHSV REGNATRMEVHFTVADPGKG VRHFSDVHLI
TLFHQREYEA AFMAAGLRVE YLEGGPSGRG LFVGVPA
SEQ ID NO: 88
MGMKEKFVLI ITHGDFGKGL LSGAEVIIGK QENVHTVGLN LGDNIEKVAK
EVMRIIIAKLAEDKEIIIVV DLFGGSPFNI ALEMMKTFDV KVITGINMPM LVELLTSINV
YDTTELLENISKIGKDGIKV IEKSSLKM
SEQ ID NO: 89
MKYDGSKLRI GILHARWNLE IIAALVAGAI KRLQEFGVKA ENIIIETVPG
SFELPYGSKLFVEKQKRLGK PLDAIIPIGV LIKGSTMHFE YICDSTTHQL MKLNFELGIP
VIFGVLTCLTDEQAEARAGL IEGKMHNHGE DWGAAAVEMA TKFN
SEQ ID NO: 90
MAVKGLGEVD QKYDGSKLRI GILHARWNRK IILALVAGAV LRLLEFGVKA
ENIIIETVPGSFELPYGSKL FVEKQKRLGK PLDAIIPIGV LIKGSTMHFE YICDSTTHQL
MKLNFELGIPVIFGVLTCLT DEQAEARAGL IEGKMHNHGE DWGAAAVEMA TKFN
SEQ ID NO: 91
MGANWYLDNE SSRLSFTSTK NADIAEVHRF LVLHGKVDPK GLAEVEVETE
SISTGIPLRDMLLRVLVFQV SKFPVAQINA QLDMRPINNL APGAQLELRL PLTVSLRGKS
HSYNAELLATRLDERRFQVV TLEPLVIHAQ DFDMVRAFNA LRLVAGLSAV SLSVPVGAVL
IFTAR
SEQ ID NO: 92
MTDYIRDGSA IKALSFAIIL AEADLRHIPQ DLQRLAVRVI HACGMVDVAN
DLAFSEGAGKAGRNALLAGA PILCDARMVA EGITRSRLPA DNRVIYTLSD PSVPELAKKI
GNTRSAAALDLWLPHIEGSI VAIGNAPTAL FRLFELLDAG APKPALIIGM PVGFVGAAES
KDELAANSRGVPYVIVRGRR GGSAMTAAAV NALASERE
SEQ ID NO: 93
MITVFGLKSK LAPRREKLAE VIYSSLHLGL DIPKGKHAIR FLCLEKEDFY
YPFDRSDDYTVIEINLMAGR SEETKMLLIF LLFIALERKL GIRAHDVEIT IKEQPAHCWG
FRGRTGDSARDLDYDIYV
SEQ ID NO: 94
MGSDLQKLQR FSTCDISDGL LNVYNIPTGG YFPNLTAISP PQNSSIVGTA
YTVLFAPIDDPRPAVNYIDS VPPNSILVLA LEPHLQSQFH PFIKITQAMY GGLMSTRAQY
LKSNGTVVFGRIRDVDEHRT LNHPVFAYGV GSCAPKAVVK AVGTNVQLKI LTSDGVTQTI
CPGDYIAGDNNGIVRIPVQE TDISKLVTYI EKSIEVDRLV SEAIKNGLPA KAAQTARRMV
LKDYI
SEQ ID NO: 95
MSGMRVYLGA DHAGYELKQA IIAFLKMTGH EPIDCGALRY DADDDYPAFC
IAAATRTVADPGSLGIVLGG SGNGEQIAAN KVPGARCALA WSVQTAALAR EHNNAQLIGI
GGRMHTLEEALRIVKAFVTT PWSKAQRHQR RIDILAEYER THEAPPVPGA PA
SEQ ID NO: 96
MGDDARIAAI GDVDELNSQI GVLLAEPLPD DVRAALSAIQ HDLFDLGGEL
CIPGHAAITEDHLLRLALWL VHYNGQLPPL EEFILPGGAR GAALAHVCRT VCRRAERSIK
ALGASEPLNIAPAAYVNLLS DLLFVLARVL NRAAGGADVL WDRTRAH
SEQ ID NO: 97
MILSAEQSFT LRHPHGQAAA LAFVREPAAA LAGVQRLRGL DSDGEQVWGE
LLVRVPLLGEVDLPFRSEIV RTPQGAELRP LTLTGERAWV AVSGQATAAE GGEMAFAFQF
QAHLATPEAEGEGGAAFEVM VQAAAGVTLL LVAMALPQGL AAGLPPA
SEQ ID NO: 98
MTKKVGIVDT TFARVDMASA AILTLKMESP NIKIIRKTVP GIKDLPVACK
KLLEEEGCDIVMALGMPGKK EKDKVCAHEA SLGLMLAQLM TNKHIIEVFV HEDEAKDDAE
LKILAARRAIEHALNVYYLL FKPEYLTRMA GKGLRQGFED AGPARE
SEQ ID NO: 99
MDDINNQLKR LKVIPVIAID NAEDIIPLGK VLAENGLPAA EITFRSSAAV
KAIMLLRSAQPEMLIGAGTI LNGVQALAAK EAGADFVVSP GFNPNTVRAC QIIGIDIVPG
VNNPSTVEQALEMGLTTLKF FPAEASGGIS MVKSLVGPYG DIRLMPTGGI TPDNIDNYLA
IPQVLACGGTWMVDKKLVRN GEWDEIARLT REIVEQVNP
SEQ ID NO: 100
MPIFTLNTNI KADDVPSDFL SLTSRLVGLI LSKPGSYVAV HINTDQQLSF
GGSTNPAAFGTLMSIGGIEP DKNRDHSAVL FDHLNAMLGI PKNRMYIHFV NLNGDDVGWN
GTTF
SEQ ID NO: 101
MPIFTLNTNI KADDVPSDFL SLTSRLVGLI LSEPGSYVAV HINTDQQLSF
GGSTNPAAFGTLMSIGGIEP DKNEDHSAVL FDHLNAMLGI PKNRMYIHFV DLDGDDVGWN
GTTF
SEQ ID NO: 102
MNQHSHKDHE TVRIAVVRAR WHADIVDACV EAFEIAMAAI GGDRFAVDVF
DVPGAYEIPLHARTLAETGR YGAVLGTAFV VNGGIYRHEF VASAVIDGMM NVQLDTGVPV
LSAVLTPHRYRDSDEHHRFF AAHFAVKGVE AARACIEILN AREKIAA
SEQ ID NO: 103
MNQHSHKDHE TVRIAVVRAR WHADIVDACV EAFEIAMAAI GGDRFAVDVF
DVPGAYEIPLHARTLAETGR YGAVLGTAFV VDGGIYDHEF VASAVIDGMM NVQLDTGVPV
LSAVLTPHEYEDSDEDHEFF AAHFAVKGVE AARACIEILN AREKIAA
SEQ ID NO: 104
MKMEELFKKH KIVAVLRANS VEEAIEKAVA VFAGGVHLIE ITFTVPDADT
VIKALSVLKEKGAIIGAGTV TSVEQCRKAV ESGAEFIVSP HLDEEISQFC KEKGVFYMPG
VMTPTELVKAMKLGHDILKL FPGEVVGPQF VKAMKGPFPN VKFVPTGGVN
LDNVCEWFKA GVLAVGVGDALVKGDPDEVR EKAKKFVEKI RGCTE
SEQ ID NO: 105
MKMEELFKKH KIVAVLRANS VEEAIEKAVA VFAGGVHLIE ITFTVPDADT
VIKALSVLKEKGAIIGAGTV TSVEQCRKAV ESGAEFIVSP HLDEEISQFC KEKGVFYMPG
VMTPTELVKAMKLGHDILKL FPGEVVGPEF VEAMKGPFPN VKFVPTGGVD
LDDVCEWFDA GVLAVGVGDALVEGDPDEVR EDAKEFVEEI RGCTE
SEQ ID NO: 106
MKMEELFKKH KIVAVLRANS VEEAIEKAVA VFAGGVHLIE ITFTVPDADT
VIKALSVLKEKGAIIGAGTV TSVEQCRKAV ESGAEFIVSP HLDEEISQFC KEKGVFYMPG
VMTPTELVKAMKLGHDILKL FPGEVVGPQF VKAMKGPFPN VKFVPTGGVN
LDNVCKWFKA GVLAVGVGKALVKGKPDEVR EKAKKFVKKI RGCTE
SEQ ID NO: 107
MNQHSHKDHE TVRIAVVRAR WHAEIVDACV SAFEAAMRDI GGDRFAVDVF
DVPGAYEIPLHARTLAETGR YGAVLGTAFV VNGGIYRHEF VASAVIDGMM NVQLDTGVPV
LSAVLTPHRYRDSDAHTLLF LALFAVKGME AARACVEILA AREKIAA
SEQ ID NO: 108
MNQHSHKDHE TVRIAVVRAR WHAEIVDACV SAFEAAMRDI GGDRFAVDVF
DVPGAYEIPLHARTLAETGR YGAVLGTAFV VDGGIYDHEF VASAVIDGMM NVQLDTGVPV
LSAVLTPHEYEDSDADTLLF LALFAVKGME AARACVEILA AREKIAA
SEQ ID NO: 109
MNQHSHKDHE TVRIAVVRAR WHAEIVDACV SAFEAAMRDI GGDRFAVDVF
DVPGAYEIPLHARTLAETGR YGAVLGTAFV VNGGIYRHEF VASAVINGMM NVQLNTGVPV
LSAVLTPHNYDKSKAHTLLF LALFAVKGME AARACVEILA AREKIAA
SEQ ID NO: 110
MTKKVGIVDT TFARVDMASA AILTLKMESP NIKIIRKTVP GIKDLPVACK
KLLEEEGCDIVMALGMPGKX EKDKVCAHEA SLGLMLAQLM TNKHIIEVFV HEDEAKDDAE
LKILAARRAIEHALNVYYLL FKPEYLTRMA GKGLRQGFED AGPARE
SEQ ID NO: 111
MXXINNQLKX LKVIPVIAID NAEDIIPLGK VLAENGLPAA EITFRSSAAV
KAIMLLRSAQPEMLIGAGTI LNGVQALAAK EAGAXFVVSP GFNPNTVRAC QIIGIDIVPG
VNNPSTVEXALEMGLTTLKF FPAEASGGIS MVKSLVGPYG DIRLMPTGGI TPXNIDNYLA
IPQVLACGGTWMVDKKLVXN GEWDEIARLT REIVEQVNP
SEQ ID NO: 112
MPIFTLNTNI KAXDVPSDFL SLTSRLVGLI LSXPGSYVAV HINTDQQLSF
GGSTNPAAFGTLMSIGGIEP XKNXDHSAVL FDHLNAMLGI PKNRMYIHFV XLXGDDVGWN
GTTF
SEQ ID NO: 113
MNQHSHKDXE TVRIAVVRAR WHADIVDACV EAFEIAMAAI GGDRFAVDVF
DVPGAYEIPLHARTLAETGR YGAVLGTAFV VXGGIYXHEF VASAVIDGMM NVQLXTGVPV
LSAVLTPHXYXDSXEXHXFFAAHFAVKGVE AARACIEILX AREKIAA
SEQ ID NO: 114
MKMEELFKKH KIVAVLRANS VEEAIEKAVA VFAGGVHLIE ITFTVPDADT
VIKALSVLKEKGAIIGAGTV TSVEQCRKAV ESGAEFIVSP HLDEEISQFC KEKGVFYMPG
VMTPTELVKAMKLGHXILKL FPGEVVGPXF VXAMKGPFPN VKFVPTGGVX
LDXVCXWFXA GVLAVGVGXALVXGXPDEVR EXAKXFVXXI RGCTE
SEQ ID NO: 115
MNQHSHKDXE TVRIAVVRAR WHAEIVDACV SAFEAAMXDI GGDRFAVDVF
DVPGAYEIPLHARTLAETGR YGAVLGTAFV VXGGIYXHEF VASAVIXGMM NVQLXTGVPV
LSAVLTPHXYXXSXAXTLLF LALFAVKGME AARACVEILA AREKIAA
SEQ ID NO: 116
taatgcttaa gtcgaacaga aagtaatcgt attgtacacg gccgcataat cgaaattaatacgactcact ataggggaat
tgtgagcgga taacaattcc ccatcttagt atattagttaagtataagaa ggagatatac tt
SEQ ID NO: 117
taaagaagga gatatcat
SEQ ID NO: 118
tgagaaggag atatcat

### CITATIONS

1. Danne, C. and S. Dramsi, Pili of gram-positive bacteria: roles in host colonization. Res Microbiol, 2012. 163 (9-10): p. 645-58.
2. Nielubowicz, G. R. and H.L. Mobley, Host-pathogen interactions in urinary tract infection. Nat Rev Urol, 2010. 7 (8): p. 430-41.
3. Snyder, J. A., et al., Coordinate Expression of Fimbriae in Uropathogenic<i>Escherichia coli</i>. Infection and Immunity, 2005. 73 (11): p. 7588-7596.
4. Connell, I., et al., Type 1 fimbrial expression enhances Escherichia coli virulence for the urinary tract. Proc Natl Acad Sci USA, 1996. 93 (18): p. 9827-32.
5. Johnson, J. R., Virulence factors in Escherichia coli urinary tract infection. Clin Microbiol Rev, 1991. 4 (1): p. 80-128.
6. Gharbi, M., et al., Antibiotic management of urinary tract infection in elderly patients in primary care and its association with bloodstream infections and all cause mortality: population based cohort study. BMJ, 2019. 364: p. 1525.
7. Martin, G. S., D.M. Mannino, and M. Moss, The effect of age on the development and outcome of adult sepsis. Crit Care Med, 2006. 34 (1): p. 15-21.
8. Ambite, I., et al., Molecular determinants of disease severity in urinary tract infection. Nature Reviews Urology, 2021. 18 (8): p. 468-486.
9. Kuehn, M. J., et al., P pili in uropathogenic E. coli are composite fibres with distinct fibrillar adhesive tips. Nature, 1992. 356 (6366): p. 252-255.
10. Ford, B., et al., The structure of the PapD-PapGII pilin complex reveals an open and flexible P5 pocket. J Bacteriol, 2012. 194 (23): p. 6390-7.
11. Kisiela, D. I., et al., Toggle switch residues control allosteric transitions in bacterial adhesins by participating in a concerted repacking of the protein core. PLoS pathogens, 2021. 17 (4): p. e1009440-e1009440.
12. Du, M., et al., Processive dynamics of the usher assembly platform during uropathogenic Escherichia coli P pilus biogenesis. Nat Commun, 2021. 12 (1): p. 5207.
13. Lee, Y. M., K. W. Dodson, and S. J. Hultgren, Adaptor function of PapF depends on donor strand exchange in P-pilus biogenesis of Escherichia coli. J Bacteriol, 2007. 189 (14): p. 5276-83.
14. Geibel, S. and G. Waksman, The molecular dissection of the chaperone-usher pathway. Biochim Biophys Acta, 2014. 1843 (8): p. 1559-67.
15. Hultgren, S. J., et al., The PapG adhesin of uropathogenic Escherichia coli contains separate regions for receptor binding and for the incorporation into the pilus. Proc Natl Acad Sci USA, 1989. 86 (12): p. 4357-61.
16. Barnhart, M. M., et al., Chaperone-subunit-usher interactions required for donor strand exchange during bacterial pilus assembly. J Bacteriol, 2003. 185 (9): p. 2723-30.
17. Johnson, J. R., J. J. Brown, and J. N. Maslow, Clonal distribution of the three alleles of the Gal(alpha1-4) Gal-specific adhesin gene papG among Escherichia coli strains from patients with bacteremia. J Infect Dis, 1998. 177 (3): p. 651-61.
18. Strömberg, N., et al., Saccharide orientation at the cell surface affects glycolipid receptor function. Proceedings of the National Academy of Sciences, 1991. 88 (20): p. 9340-9344.
19. Johnson, J. R., et al., Evidence of Commonality between Canine and Human Extraintestinal Pathogenic<i>Escherichia coli</i>Strains That Express <i>papG</i>Allele III. Infection and Immunity, 2000. 68 (6): p. 3327-3336.
20. Biggel, M., et al., Horizontally acquired papGII-containing pathogenicity islands underlie the emergence of invasive uropathogenic Escherichia coli lineages. Nature Communications, 2020. 11 (1).
21. Johnson, J. R., et al., Bacterial Characteristics in Relation to Clinical Source of <i>Escherichia coli</i>Isolates from Women with Acute Cystitis or Pyelonephritis and Uninfected Women. Journal of Clinical Microbiology, 2005. 43 (12): p. 6064-6072.
22. Lanne, B., et al., Glycoconjugate receptors for P-fimbriated Escherichia coli in the mouse. An animal model of urinary tract infection. J Biol Chem, 1995. 270 (15): p. 9017-25.
23. Roberts, J. A., et al., The Gal(alpha 1-4) Gal-specific tip adhesin of Escherichia coli P-fimbriae is needed for pyelonephritis to occur in the normal urinary tract. Proceedings of the National Academy of Sciences, 1994. 91 (25): p. 11889-11893.
24. Roberts, J. A., et al., Antibody responses and protection from pyelonephritis following vaccination with purified Escherichia coli PapDG protein. J Urol, 2004. 171 (4): p. 1682-5.
25. Pecha, B., D. Low, and P. O'Hanley, Gal-Gal pili vaccines prevent pyelonephritis by piliated Escherichia coli in a murine model. Single-component Gal-Gal pili vaccines prevent pyelonephritis by homologous and heterologous piliated E. coli strains. Journal of Clinical Investigation, 1989. 83 (6): p. 2102-2108.
26. O'Hanley, P., et al., Molecular basis of Escherichia coli colonization of the upper urinary tract in BALB/c mice. Gal-Gal pili immunization prevents Escherichia coli pyelonephritis in the BALB/c mouse model of human pyelonephritis. Journal of Clinical Investigation, 1985. 75 (2): p. 347-360.
27. Dodson, K. W., et al., Structural basis of the interaction of the pyelonephritic E. coli adhesin to its human kidney receptor. Cell, 2001. 105 (6): p. 733-43.
28. Conover, M. S., et al., Inflammation-Induced Adhesin-Receptor Interaction Provides a Fitness Advantage to Uropathogenic E. coli during Chronic Infection. Cell Host Microbe, 2016. 20 (4): p. 482-492.
29. Welch, R. A., et al., Extensive mosaic structure revealed by the complete genome sequence of uropathogenic <i>Escherichia coli</i>. Proceedings of the National Academy of Sciences, 2002. 99 (26): p. 17020-17024.
30. Rasko, D. A., et al., Identification of DNA sequences from a second pathogenicity island of uropathogenic Escherichia coli CFT073: probes specific for uropathogenic populations. J Infect Dis, 2001. 184 (8): p. 1041-9.
31. Mobley, H. L. T., et al., Isogenic P-fimbrial deletion mutants of pyelonephritogenic Escherichia coli: the role of? Gal(1?4)? Gal binding in virulence of a wild-type strain. Molecular Microbiology, 1993. 10 (1): p. 143-155.
32. Cao, L., et al., Global site-specific analysis of glycoprotein N-glycan processing. Nat Protoc, 2018. 13 (6): p. 1196-1212.
33. Gupta, R. and S. Brunak, Prediction of glycosylation across the human proteome and the correlation to protein function. Pac Symp Biocomput, 2002: p. 310-22.
34. Bock, K., et al., Specificity of binding of a strain of uropathogenic Escherichia coli to Gal alpha 1—4Gal-containing glycosphingolipids. J Biol Chem, 1985. 260 (14): p. 8545-51.
35. Ohlsson, J., et al., Discovery of potent inhibitors of PapG adhesins from uropathogenic Escherichia coli through synthesis and evaluation of galabiose derivatives. Chembiochem, 2002. 3 (8): p. 772-9.
36. Jacob-Dubuisson, F., et al., Initiation of assembly and association of the structural elements of a bacterial pilus depend on two specialized tip proteins. EMBO J, 1993. 12 (3): p. 837-47.
37. Uhlin, B. E., et al., Adhesion to human cells by Escherichia coli lacking the major subunit of a digalactoside-specific pilus-adhesin. Proc Natl Acad Sci USA, 1985. 82 (6): p. 1800-4
38. Miyagawa-Yamaguchi, A., N. Kotani, and K. Honke, Each GPI-anchored protein species forms a specific lipid raft depending on its GPI attachment signal. Glycoconj J, 2015. 32 (7): p. 531-40.
39. Gislason, M. H., et al., Prediction of GPI-anchored proteins with pointer neural networks. Current Research in Biotechnology, 2021. 3: p. 6-13.
40. Moran, P. and I. W. Caras, A nonfunctional sequence converted to a signal for glycophosphatidylinositol membrane anchor attachment. J Cell Biol, 1991. 115 (2): p. 329-36.
41. Galian, C., et al., Efficient glycosylphosphatidylinositol (GPI) modification of membrane proteins requires a C-terminal anchoring signal of marginal hydrophobicity. J Biol Chem, 2012. 287 (20): p. 16399-409.
42. Lisanti, M. P., et al., A glycophospholipid membrane anchor acts as an apical targeting signal in polarized epithelial cells. J Cell Biol, 1989. 109 (5): p. 2145-56.
43. Bradley, J. E., J. M. Chan, and J. S. Hagood, Effect of the GPI anchor of human Thy-1 on antibody recognition and function. Lab Invest, 2013. 93 (3): p. 365-74.
44. Miyagawa-Yamaguchi, A., N. Kotani, and K. Honke, Expressed glycosylphosphatidylinositol-anchored horseradish peroxidase identifies co-clustering molecules in individual lipid raft domains. PLoS One, 2014. 9 (3): p. e93054.
45. Cocchi, F., et al., The soluble ectodomain of herpes simplex virus gD contains a membrane-proximal pro-fusion domain and suffices to mediate virus entry. Proc Natl Acad Sci USA, 2004. 101 (19): p. 7445-50.
46. Källenius G, Möllby R, Svenson S B, Winberg J, Lundblad A, et al. The Pk antigen as receptor for the haemagglutinin of pyelonephritic Escherichia coli. 1980. FEMS Microbiology Letters 7:297-302.
47. Patton JP, Nash DB, Abrutyn E. Urinary Tract Infection: Economic Considerations. 1991. Medical Clinics of North America 75:495-513.
48. Svanborg C, Godaly G. Bacterial virulence in urinary tract infection. 1997. Infectious disease clinics of North America 11:513-29.
49. Leffler H, Edén C S. Chemical identification of a glycosphingolipid receptor for Escherichia coli attaching to human urinary tract epithelial cells and agglutinating human erythrocytes. 1980. FEMS Microbiology Letters 8:127-34.

1. A mutant of a wild-type PapG polypeptide, wherein the mutant comprises at least one amino acid mutation relative to the amino acid sequence of the wild type PapG polypeptide, wherein the mutation is selected from the group consisting of: G18, G75, G86, S89, N96, G104, W107, G122, G147, G168, R170, K172, N24S, and N286, or any combination thereof, and wherein the amino acid positions are numbered according to SEQ ID NO:120.

2. The mutant PapG polypeptide according to claim 1, comprising at least one mutation selected from the group consisting of: G18A, G75A, G86A, S89T, N96S, G104A, W107A, G122A, G147A, G168A, R170A, K172A, N242S, and N286S, or any combination thereof.

3. The mutant PapG polypeptide according to claim 2, comprising the mutation N96S.

4. The mutant PapG polypeptide according to claim 2, comprising the mutations N96S and G86A.

5. The mutant PapG polypeptide according to claim 2, comprising the mutations N96S and S89T.

6. The mutant PapG polypeptide according to claim 2, comprising the mutations N96S and G104A.

7. The mutant PapG polypeptide according to claim 2, comprising the mutations N96S and G168A.

8. The mutant PapG polypeptide according to claim 2, comprising the mutations N96S and G18A.

9. The mutant PapG polypeptide according to claim 2, comprising the mutations N96S and G75A.

10. The mutant PapG polypeptide according to claim 2, comprising the mutations N96S and G122A.

11. The mutant PapG polypeptide according to claim 2, comprising the mutations N96S and G147A.

12. The mutant PapG polypeptide according to claim 2, comprising the mutations N96S and W107A.

13. The mutant PapG polypeptide according to claim 2, comprising the mutations N96S and R170A.

14. The mutant PapG polypeptide according to claim 2, comprising the mutations N96S and K172A.

15. The mutant PapG polypeptide according to claim 2, comprising the mutations N96S and N286S.

16. The mutant PapG polypeptide according to claim 2, comprising the mutations N96S, N242S and N286S.

17. The mutant PapG polypeptide according to claim 2, comprising the mutations N96S, N242S and K172A.

18. The mutant PapG polypeptide according to claim 2, comprising the mutations N96S, N242S, N286S and K172A.

19. The mutant PapG polypeptide according to claim 2, comprising the amino acid sequence set forth in any one of SEQ ID NO: 11-41.

20. The mutant PapG polypeptide of claim 19, wherein the mutant PapG polypeptide comprises an amino acid sequence having at least 80% identity to the amino acid sequence set forth in any one of SEQ ID NO: 11-41.

21. The mutant PapG polypeptide of claim 20, wherein the PapG polypeptide has at least 90%, 95, 96%, 97%, 98% or 99% identity to the amino acid sequence set forth in any one of SEQ ID NO: 11-41.

22. The mutant PapG polypeptide according to any of claims 1-21, wherein the polypeptide is isolated.

23. A pharmaceutical composition comprising (i) the mutant PapG polypeptide according to any one of claims 1-21 and (ii) a pharmaceutically acceptable carrier.

24. An immunogenic composition comprising the mutant PapG polypeptide according to any one of claims 1-21.

25. An immunogenic composition according to claim 24, further comprising at least one additional antigen.

26. An immunogenic composition according to claim 25, wherein the at least one additional antigen is a saccharide, or a polysaccharide, or a glycoconjugate, or a protein.

27. An immunogenic composition according to claim 24, further comprising at least one adjuvant.

28. A nucleic acid molecule comprising a nucleotide sequence that encodes an amino acid sequence of the mutant PapG polypeptide according to any one of claims 1-21.

29. the mutant PapG polypeptide according to any of claims 1-22, wherein the polypeptide is immunogenic.

30. A recombinant mammalian cell, comprising a polynucleotide encoding the mutant PapG polypeptide according to any one of claims 1-21.

31. A culture comprising the recombinant cell of claim 30, wherein said culture is at least 5 liters in size.

32. A method for producing the mutant PapG polypeptide according to any one of claims 1-21, comprising culturing a recombinant mammalian cell according to claim 30 under suitable conditions, thereby expressing the polypeptide; and harvesting the polypeptide.

33. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of a composition according to any one of claims 23-27.

34. A method according to claim 33, wherein the subject is at risk of developing a urinary tract infection.

35. A method according to claim 33, wherein the subject is at risk of developing bacteremia.

36. A method according to claim 33, wherein the subject is at risk of developing sepsis or urosepsis, cystitis and/or pyelonephritis.

37. A method of eliciting an immune response against E. coli in a mammal, comprising administering to the mammal an effective amount of a composition according to any one of claims 23-29.

38. A method according to claim 37, wherein the immune response comprises opsonophagocytic and/or neutralizing antibodies against E. coli.

39. A method according to claim 37, wherein the immune response protects the mammal from an E. coli infection.

40. A method of preventing, treating or ameliorating a bacterial infection, disease or condition in a subject, comprising administering to the subject an immunologically effective amount of a composition according to any one of claims 23-28.

41. A ribonucleic acid polynucleotide (RNA) molecule comprising at least one open reading frame (ORF) encoding a PapG antigenic polypeptide.

42. The RNA molecule of claim 41, wherein the PapG antigenic polypeptide is a full-length, truncated, fragment or variant thereof.

43. The RNA molecule of any one of claims 41 to 42, wherein the PapG antigenic polypeptide comprises at least one mutation relative to the amino acid sequence of a wild type PapG polypeptide.

44. The RNA molecule of any one of claims 41 to 43, wherein the mutation is selected from the group consisting of: G18, G75, G86, S89, N96, G104, W107, G122, G147, G168, R170, K172, N24S, and N286, or any combination thereof, and wherein the amino acid positions are numbered according to SEQ ID NO:120.

45. The RNA molecule of claim 44, comprising at least one mutation selected from the group 20) consisting of: G18A, G75A, G86A, S89T, N96S, G104A, W107A, G122A, G147A, G168A, R170A, K172A, N242S, and N286S, or any combination thereof.

46. The RNA molecule of any one of embodiments 41-45, wherein the PapG antigenic polypeptide comprises PapG_{DSF} (SEQ ID NO: 69), PapG_{DSF} mutant, PapG_{LD} mutant, or an immunogenic fragment thereof.

47. The RNA molecule of any one of claims 41 to 46, wherein the PapG antigenic polypeptide comprises amino acids having an amino acid sequence as set forth in SEQ ID NO: 11 to 41.

48. The RNA molecule of claim 47, wherein the PapG antigenic polypeptide comprises amino acids having an amino acid sequence as set forth in SEQ ID NO: 27 to 41.

49. The RNA molecule of any one of claims 41 to 48, wherein the PapG polypeptide comprises an amino acid sequence having at least 80% identity to the amino acid sequence set forth in any one of SEQ ID NO: 11 to 41.

50. The RNA molecule of any one of claims 41 to 49, wherein the PapG polypeptide comprises an amino acid sequence having at least 90%, 95, 96%, 97%, 98% or 99% identity to the amino acid sequence set forth in any one of SEQ ID NO: 11 to 41.

51. The RNA molecule of any one of claims 41-50, wherein the RNA is fused to a C-terminal membrane targeting domain.

52. The RNA molecule of claim 51, wherein the RNA molecule and the C-terminal membrane targeting domain are separated by a linker.

53. The RNA molecule of claim 52, wherein the linker has the amino acid sequence
(SEQ ID NO: 71)
GGSSGGGGSSGSGSSSG,
(SEQ ID NO: 72)
SSGGGGSSGSGSSSG,
(SEQ ID NO: 73)
SSGGGGSSGSGSS,
or
(SEQ ID NO: 74)
SSGGG.

54. The RNA molecule of any of claims 51-53, wherein the C-terminal membrane targeting domain is derived from a viral glycoprotein.

55. The RNA molecule of claim 54, wherein the viral glycoprotein is HSV gD (TMD).

56. The RNA molecule of any of claims 51-53, wherein the C-terminal membrane targeting domain is human Thyl-GPI or human DAF-GPI.

57. The RNA molecule of any of claims 41-56, wherein the open reading frame is codon-optimized.

58. The RNA molecule of claim 57, wherein the PapG antigenic polypeptide comprises amino acids having an amino acid sequence as set forth in SEQ ID NO: 11-41.

59. The RNA molecule of any one of claims 41 to 58, further comprising a 5′ untranslated region (5′ UTR).

60. The RNA molecule of claim 59, wherein the 5′ UTR comprises nucleotides having SEQ ID NO: 1, 3, 5 or 7.

61. The RNA molecule any one of claims 41 to 60, further comprising a 3′ untranslated region (3′ UTR).

62. The RNA molecule of claim 61, wherein the 3′ UTR comprises nucleotides having SEQ ID NO: 2, 4, 6, 8 or 10.

63. The RNA molecule of any one of claims 41 to 62, further comprising a 3′ poly-A tail.

64. The RNA of claim 63, wherein the poly A tail comprises a sequence having SEQ ID NO: 75.

65. The RNA molecule of any one of claims 41 to 64, wherein the RNA molecule comprises a 5′ UTR and a 3′ UTR.

66. The RNA molecule of any one of claims 41 to 65, wherein the RNA molecule comprises a 5′ UTR, 3′ UTR, and poly-A tail.

67. The RNA molecule of any one of claims 41 to 66, wherein the nucleic acid comprises nucleotides having a sequence as set forth in any one of SEQ ID NO: 42-66.

68. The RNA molecule of any one of claims 41 to 67, wherein the RNA molecule further comprises a 5′ cap moiety.

69. The RNA molecule of claim 68, wherein the 5′ cap moiety is m⁷G (5′)ppp(5′)(2′OMeA)pG.

70. The RNA molecule of any of claims 41 to 69, wherein the RNA molecule comprises stabilized RNA.

71. The RNA molecule of any one of claims 41 to 70, wherein the RNA comprises at least one modified nucleotide.

72. The RNA molecule of claim 71, wherein the modified nucleotide is pseudouridine, 1-methyl-3′-pseudouridylyl, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, or 5-methoxyuridine OR 2′-O-methyl uridine.

73. The RNA molecule of claim 72, wherein the modified nucleotide is 1-methyl-3′-pseudouridylyl.

74. The RNA molecule of any one of claims 41 to 73, wherein the RNA is mRNA.

75. A composition comprising the RNA molecule of any one of claims 41 to 74, wherein the RNA molecule is formulated in a lipid nanoparticle (RNA-LNP).

76. The composition of claim 75, wherein lipid nanoparticle comprises at least one of a cationic lipid, a PEG-lipid, a neutral lipid, and a steroid or steroid analog.

77. The composition of claim 76, wherein the lipid nanoparticle comprises a cationic lipid.

78. The composition of claim 77, wherein the cationic lipid is (4-hydroxybutyl) azanediyl)bis(hexane-6,1-diyl)bis(2-hexyldecanoate) (ALC-0315).

79. The composition of any one of claims 41 to 78, wherein the lipid nanoparticle comprises a PEG-lipid.

80. The composition of claim 79, wherein the PEG-lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]—N,N-ditetradecylacetamide, glycol-lipids including PEG-c-DOMG, PEG-c-DMA, PEG-s-DMG,N-[(methoxy polyethylene glycol) 2000) carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy) propyl-1-O-((o-methoxy(polyethoxy)ethyl) butanedioate (PEG-S-DMG), a PEGylated ceramide (PEG-cer), or a PEG dialkoxypropylcarbamate such as co-methoxy(polyethoxy)ethyl-N-(2,3di(tetradecanoxy) propyl) carbamate or 2,3-di(tetradecanoxy) propyl-N-(u>-methoxy(polyethoxy)ethyl) carbamate.

81. The composition of claim 80, wherein the PEG-lipid is 2-[(polyethylene glycol)-2000]—N,N-ditetradecylacetamide (ALC-0159).

82. The composition of any one of claims 75-81, wherein the lipid nanoparticle comprises a neutral lipid.

83. The composition of claim 82, wherein the neutral lipid is distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 30) 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).

84. The composition of claim 83, wherein the neutral lipid is 1,2-distearoyl-sn-glycero-3-phosphocholine (DSPC).

85. The composition of any one of claims 75 to 84, wherein the lipid nanoparticle comprises a steroid or steroid analog.

86. The composition of claim 85, wherein the steroid or steroid analog is cholesterol.

87. The composition of any one of claims 75 to 86, wherein lipid nanoparticle wherein has a mean diameter of about 1 to about 500 nm.

88. The composition of any one of claims 75 to 87, wherein the composition is a vaccine.

89. The composition of any one of claims 75 to 88, wherein the lipid nanoparticle size is at least 40 nm.

90. The composition of any one of claims 75 to 89, wherein the lipid nanoparticle size is at most 180 nm.

91. The composition of any one of claims 75 to 90, wherein the composition is a vaccine.

92. A method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of the RNA molecule, RNA-LNP and/or vaccine of any one of claims 41 to 91.

93. The method of claim 92, wherein the subject is at risk of developing a urinary tract infection.

94. The method of claim 92, wherein the subject is at risk of developing bacteremia.

95. The method of claim 92, wherein the subject is at risk of developing sepsis or urosepsis.

96. The method of claim 92, wherein the subject is at risk of developing cystitis.

97. The method of claim 92, wherein the subject is at risk of developing pyelonephritis.

98. Use of the RNA molecule, RNA-LNP and/or composition of any one of claims 41 to 91 in the manufacture of a medicament for use in (i) inducing an immune response in a subject against extra-intestinal pathogenic E. coli, or (ii) inducing the production of opsonophagocytic and/or neutralizing antibodies in a subject that are specific to extra-intestinal pathogenic E. coli.

99. The use of claim 98, wherein the infection, disease or condition is a urinary tract infection.

100. The use of claim 98, wherein the subject is at risk of developing bacteremia.

101. The use of claim 98, wherein the subject is at risk of developing sepsis or urosepsis.

102. The use of claim 98, wherein the subject is at risk of developing cystitis.

103. The use of claim 98, wherein the subject is at risk of developing pyelonephritis.

104. The method or use of any one of claims 92 to 103, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older, from about 18 years of age to 49 years of age, or from 50 years of age to 64 years of age.

105. The method or use of claim 104, wherein the subject is about 50 years of age or older.

106. The method or use of claim 104, wherein the subject is a pregnant woman.

107. The method or use of any one of claims 104 to 106, wherein the RNA molecule or composition is administered as a vaccine.

108. The method or use of any one of claims 104 to 107, wherein the RNA molecule or composition is administered by intradermal or intramuscular injection.


SEQUENCE LISTING (165 sequences)

SEQ ID NO: 1 - RNA, 51 nt, Synthetic construct
  source 1..51: mol_type = other RNA
aggaaataagagaggataagacgactaaggagacatacagaataagaggca

SEQ ID NO: 2 - RNA, 134 nt, Synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 3 - RNA, 44 nt, Synthetic construct
  source 1..44: mol_type = other RNA
aggaggactggtcgaacctgcatagtgatcataaggtcagcata

SEQ ID NO: 4 - RNA, 134 nt, Synthetic construct; identical to SEQ ID NO: 2
  source 1..134: mol_type = other RNA

SEQ ID NO: 5 - RNA, 55 nt, Synthetic construct
  source 1..55: mol_type = other RNA
aggaaataagagaggataagacgactaaggagacatacagaataagaaacaggca

SEQ ID NO: 6 - RNA, 134 nt, Synthetic construct; identical to SEQ ID NO: 2
  source 1..134: mol_type = other RNA

SEQ ID NO: 7 - RNA, 55 nt, Synthetic construct
  source 1..55: mol_type = other RNA
aggaaataagagaaagaggataagacgactaaggagacatacagaataagaggca

SEQ ID NO: 8 - RNA, 134 nt, Synthetic construct; identical to SEQ ID NO: 2
  source 1..134: mol_type = other RNA

SEQ ID NO: 9 - RNA, 53 nt, Synthetic construct
  source 1..53: mol_type = other RNA
aggacatttgcttctgacacaactgtgttcactagcaacctcaaacagccacc

SEQ ID NO: 10 - RNA, 133 nt, Synthetic construct
  source 1..133: mol_type = other RNA
gctcgctttcttgctgtccaatttctattaaaggttcctttgttccctaagtccaactactaaactgggggatattatgaagggccttgagcatctggattctgcctaataaaaaacatttattttcattgca

SEQ ID NO: 11 - protein, 216 aa, Escherichia coli
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 12 - protein, 216 aa, Escherichia coli
METDTLLLWVLLLWVPGSTGWNNIVFYSLGNVNSYQGGNVVITQRPQFITSWRPGIATVTWNQCNGPEFADGSWAYYREYIAWVVFPKKVMTQNGYPLFIEVHNKGSWSEENTGDNDSYFFLKGYKWDERAFDAGNLCQKPGETTRLTEKFDDIIFKVALPADLPLGDYSVKIPYTSGMQRHFASYLGARFKIPYNVAKTLPRENEMLFLFKNIGG

SEQ ID NO: 13 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 14 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKASWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 15 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWTEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 16 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKAYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 17 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLAARFKIPYNVAKTLPRENEMLFLFKNIGG

SEQ ID NO: 18 - protein, 216 aa, Synthetic construct
METDTLLLWVLLLWVPGSTGWNNIVFYSLGNVNSYQGANVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 19 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNAYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 20 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPAETTRLTEKFNDIIFKVALPADLPLGDYSVTIPYTSGIQRHFASYLGARFKIPYNVAKTLPRENEMLFLFKNIGG

SEQ ID NO: 21 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLADYSVTIPYTSGIQRHFASYLGARFKIPYNVAKTLPRENEMLFLFKNIGG

SEQ ID NO: 22 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKADERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 23 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGAAFKIPYNVAKTLPRENEMLFLFKNIGG

SEQ ID NO: 24 - protein, 216 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFAIPYNVAKTLPRENEMLFLFKNIGG

SEQ ID NO: 25 - skipped

SEQ ID NO: 26 - protein, 357 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFAIPYNVAKTLPRENEMLFLFKNIGGCRPSAQSLEIKHGDLSINSANNHYAAQTLSVSCDVPANIRFMLLRSTTPTYSHGKKFSVGLGHGWDSIVSVNGVDTGETTMRWYKAGTQSLTIGSRLYGESSKIQPGVLSGSATLLMILPGGSSGGADVQINIRGNVYIPP

SEQ ID NO: 27 - protein, 258 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGSSGGGGSSGSGSSSGTTRLLSGHTCFTLTGLLGTLVTMGLLT

SEQ ID NO: 28 - protein, 261 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGSSGGGGSSGSGSSCEGISLLAQNTSWLLLLLLSLSLLQATDFMSL

SEQ ID NO: 29 - protein, 281 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGSSGGGATPNNMGLIAGAVGGSLLAALVICGIVYWMRRHTQKAPKRIRLPHIREDDQPSSHQPLFY

SEQ ID NO: 30 - protein, 258 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGAAFKIPYNVAKTLPRENEMLFLFKNIGGSSGGGGSSGSGSSSGTTRLLSGHTCFTLTGLLGTLVTMGLLT

SEQ ID NO: 31 - protein, 258 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFAIPYNVAKTLPRENEMLFLFKNIGGSSGGGGSSGSGSSSGTTRLLSGHTCFTLTGLLGTLVTMGLLT

SEQ ID NO: 32 - protein, 258 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKAYKWDERAFDAGNLCQKPGETTRLTEKFNDIIFKVALPADLPLGDYSVTIPYTSGIQRHFASYLGARFKIPYNVAKTLPRENEMLFLFKNIGGSSGGGGSSGSGSSSGTTRLLSGHTCFTLTGLLGTLVTMGLLT

SEQ ID NO: 33 - protein, 258 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKADERAFDAGNLCQKPGETTRLTEKFNDIIFKVALPADLPLGDYSVTIPYTSGIQRHFASYLGARFKIPYNVAKTLPRENEMLFLFKNIGGSSGGGGSSGSGSSSGTTRLLSGHTCFTLTGLLGTLVTMGLLT

SEQ ID NO: 34 - protein, 399 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSSGTTRLLSGHTCFTLTGLLGTLVTMGLLT

SEQ ID NO: 35 - protein, 402 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSCEGISLLAQNTSWLLLLLLSLSLLQATDFMSL

SEQ ID NO: 36 - protein, 422 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGATPNNMGLIAGAVGGSLLAALVICGIVYWMRRHTQKAPKRIRLPHIREDDQPSSHQPLFY

SEQ ID NO: 37 - protein, 357 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQSLTIGSRLYGESSKIQPGVLSGSATLLMILPGGSSGGADVQINIRGNVYIPP

SEQ ID NO: 38 - protein, 399 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRSTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSSGTTRLLSGHTCFTLTGLLGTLVTMGLLT

SEQ ID NO: 39 - protein, 399 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFAIPYNVAKTLPRENEMLFLFKNIGGCRPSAQSLEIKHGDLSINSANNHYAAQTLSVSCDVPANIRFMLLRSTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSSGTTRLLSGHTCFTLTGLLGTLVTMGLLT

SEQ ID NO: 40 - protein, 402 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRSTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSCEGISLLAQNTSWLLLLLLSLSLLQATDFMSL

SEQ ID NO: 41 - protein, 402 aa, Synthetic construct
METDTLLLWVLLLWVPGSTGWNNIVFYSLGNVNSYQGGNVVITQRPQFITSWRPGIATVTWNQCNGPEFADGSWAYYREYIAWVVFPKKVMTKNGYPLFIEVHNKGSWSEENTGDSDSYFFLKGYKWDERAFDAGNLCQKPGETTRLTEKFNDIIFKVALPADLPLGDYSVTIPYTSGIQRHFASYLGARFAIPYNVAKTLPRENEMLFLFKNIGGCRPSAQSLEIKHGDLSINSANNHYAAQTLSVSCDVPANIRFMLLRSTTPTYSHGKKFSVGLGHGWDSIVSVNGVDTGETTMRWYKAGTQSLTIGSRLYGESSKIQPGVLSGSATLLMILPGGSSGGADVQINIRGNVYIPPSSGGGGSSGSGSSCEGISLLAQNTSWLLLLLLSLSLLQATDFMSL

SEQ ID NO: 42 - RNA, 1051 nt, Synthetic construct
  source 1..1051: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - RNA, 1055 nt, Synthetic construct
  source 1..1055: mol_type = other RNA
aggaaataagagaaagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - RNA, 1054 nt, Synthetic construct
  source 1..1054: mol_type = other RNA
aggaaataagagaggataagacgactaaggagacatacagaataagaaacaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - RNA, 1044 nt, Synthetic construct
  source 1..1044: mol_type = other RNA
aggaggactggtcgaacctgcatagtgatcataaggtcagcatagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - RNA, 1060 nt, Synthetic construct
  source 1..1060: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcatgtgaagggatcagtctcctcgcccaaaacacttcatggctgttgctcctcctgttgtccctttctcttttgcaggcaacagattttatgagcttgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - RNA, 1120 nt, Synthetic construct
  source 1..1120: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtgcgacccccaacaacatggggctgatagccggtgccgtaggcggcagcctgctggccgccctggtgatctgcggcatcgtgtactggatgagaagacacacacagaaggcccccaagagaatcagactgccccacatcagagaggacgatcagcctagcagccatcagcccctgttctactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - RNA, 925 nt, Synthetic construct
  source 1..925: mol_type = other RNA
aggaaataagagaggataagacgactaaggagacatacagaataagaggcagccaccatggagaccgacacactgctgctgtgggtgctgctgctgtgggtgcccggctccaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - RNA, 1051 nt, Synthetic construct
  source 1..1051: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctgccttcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - RNA, 1051 nt, Synthetic construct
  source 1..1051: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - RNA, 1055 nt, Synthetic construct
  source 1..1055: mol_type = other RNA
aggaaataagagaaagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - RNA, 1044 nt, Synthetic construct
  source 1..1044: mol_type = other RNA
aggaggactggtcgaacctgcatagtgatcataaggtcagcatagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - RNA, 1051 nt, Synthetic construct
  source 1..1051: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaaggcctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - RNA, 1051 nt, Synthetic construct
  source 1..1051: mol_type = other RNA
aggaaataagagaggataagacgactaaggagacatacagaataagaggcagccaccatggagaccgacacactgctgctgtgggtgcttttgttgtgggtgcccggttctaccggatggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaaggccgacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggcagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 55 - RNA, 1474 nt, Synthetic construct
  source 1..1474: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagaaacaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 56 - RNA, 1483 nt, Synthetic construct
  source 1..1483: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagaccacaagacttaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagaaacaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtatggcgaaagctccaaaatccaacccggcgtgctgtcggggagcgccaccctgctgatgatactgcccggaggcagcagcggcggcgccgacgtgcagatcaacatcagaggcaacgtgtatattccgcctagcagtggtggtgggggtagctccggctccggaagcagctgcgagggcatcagcctgctggctcagaacacaagctggctgctgctgctgctgctgagcctgagcctgctgcaagccaccgacttcatgagcctgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 57 - RNA, 1543 nt, Synthetic construct
  source 1..1543: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagaaacaccacccccacctacagccacggcaagaagttcagcgtgggcctgggccacggctgggacagcatcgtgagcgtgaacggcgtggacaccggcgagactaccatgagatggtacaaggccggcacacagagcctgaccatcggcagcagactgtatggcgaaagcagcaagattcagcccggcgtgttaagcggctctgcgaccctcctgatgatactgcccggtggaagcagtgggggcgccgacgtgcagatcaacatcagaggcaatgtctacatcccccctagctctggtggcggcgcaacccccaacaacatgggccttattgcgggcgccgttggtggttccctgctggccgccctggtgatctgcggcatcgtgtactggatgagaagacacacacagaaggcccccaagagaatcagactgccccacatcagagaggacgatcagcctagcagccatcagcccctgttctactgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 58 - RNA, 1348 nt, Synthetic construct
  source 1..1348: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagcccggcgagaccacaagactgaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagaaacaccacccccacctacagccacggcaagaagttcagcgtgggcctgggccacggctgggacagcatcgtgagcgtgaacggcgtggacaccggcgagactaccatgagatggtacaaggccggcacacagagcctgaccatcggcagcagactgtacggtgaaagcagcaagatccaaccgggcgtgctgagtggctcggcaaccctgctgatgatcctgcccggaggcagcagcggcggcgccgacgtgcagatcaacatcagaggcaacgtgtacatccccccctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 59 - RNA, 1474 nt, Synthetic construct
  source 1..1474: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - RNA, 1478 nt, Synthetic construct
  source 1..1478: mol_type = other RNA
aggaaataagagaaagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - RNA, 1467 nt, Synthetic construct
  source 1..1467: mol_type = other RNA
aggaggactggtcgaacctgcatagtgatcataaggtcagcatagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - RNA, 1474 nt, Synthetic construct
  source 1..1474: mol_type = other RNA
aggaaataagagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 1478 nt, Synthetic construct
  source 1..1478: mol_type = other RNA
aggaaataagagaaagaggataagacgactaaggagacatacagaataagaggcagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - RNA, 1467 nt, Synthetic construct
  source 1..1467: mol_type = other RNA
aggaggactggtcgaacctgcatagtgatcataaggtcagcatagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagacgacccggcttacagaaaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtacggcgagagcagcaagattcaaccgggcgtgcttagtggttccgccaccctgctgatgattctgcccggcggaagcagcggcggcgccgacgtgcagatcaacatcagaggcaacgtgtacatacccccttcgagcggaggcggcggatcgtccggctccgggagcagcagcggcaccacacggctgctgagcggccacacctgcttcaccctgaccggcctgctgggcaccctggtgaccatgggcctgctgacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 1476 nt, Synthetic construct
  source 1..1476: mol_type = other RNA
aggaggactggtcgaacctgcatagtgatcataaggtcagcatagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagaccacaagacttaccgagaagttcaacgacatcatcttcaaggtggccctgcccgccgacctgcccctgggcgactacagcgtgaccatcccctacacaagcggcattcagagacacttcgctagctacctgggcgctagattcaagatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtatggcgaaagctccaaaatccaacccggcgtgctgtcggggagcgccaccctgctgatgatactgcccggaggcagcagcggcggcgccgacgtgcagatcaacatcagaggcaacgtgtatattccgcctagcagtggtggtgggggtagctccggctccggaagcagctgcgagggcatcagcctgctggctcagaacacaagctggctgctgctgctgctgctgagcctgagcctgctgcaagccaccgacttcatgagcctgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 66 - RNA, 1476 nt, Synthetic construct
  source 1..1476: mol_type = other RNA
aggaggactggtcgaacctgcatagtgatcataaggtcagcatagccaccatggagaccgacaccctcctgctgtgggtgctgttactgtgggtgcccggtagcaccggctggaacaacatcgtgttctacagcctgggcaacgtgaacagctaccaaggcggcaacgtggtgatcacacagagacctcagttcatcacaagctggagacccggcatcgccaccgtgacctggaatcagtgcaacggccccgagttcgccgacggcagctgggcctactacagagagtacatcgcctgggtggtgttccccaagaaggtgatgaccaagaacggctaccccctgttcatcgaggtgcacaacaagggcagctggagcgaggagaacaccggcgacagcgacagctacttcttcctgaagggctacaagtgggacgagagagccttcgacgccggcaacctgtgtcagaagccgggggagaccacaagacttaccgagaagttcaacgacatcatcttcaaggtggccctgcccgccgacctgcccctgggcgactacagcgtgaccatcccctacacaagcggcattcagagacacttcgctagctacctgggcgctagattcgccatcccctacaacgtggccaagaccctgcctagagagaacgagatgctgttcctgttcaagaacatcggcggctgcagacctagcgctcagagcctggagatcaagcacggcgacctgagcatcaacagcgccaacaaccactacgccgctcagaccctgagcgtgagctgcgacgtgcccgccaacatcagattcatgctgctgagatcaaccacccccacctacagccacggcaagaagttcagcgtgggcctgggccacggctgggacagcatcgtgagcgtgaacggcgtggacacgggcgagaccactatgcgttggtacaaggccggcacacagagcctgaccatcggcagcagactgtatggcgaaagctccaaaatccaacccggcgtgctgtcggggagcgccaccctgctgatgatactgcccggaggcagcagcggcggcgccgacgtgcagatcaacatcagaggcaacgtgtatattccgcctagcagtggtggtgggggtagctccggctccggaagcagctgcgagggcatcagcctgctggctcagaacacaagctggctgctgctgctgctgctgagcctgagcctgctgcaagccaccgacttcatgagcctgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - protein, 20 aa, Mus musculus
METDTLLLWVLLLWVPGSTG

SEQ ID NO: 68 - protein, 196 aa, Escherichia coli
WNNIVFYSLGNVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGG

SEQ ID NO: 69 - protein, 196 aa, Escherichia coli
WNNIVFYSLGNVNSYQGGNVVITQRPQFITSWRPGIATVTWNQCNGPEFADGSWAYYREYIAWVVFPKKVMTQNGYPLFIEVHNKGSWSEENTGDNDSYFFLKGYKWDERAFDAGNLCQKPGETTRLTEKFDDIIFKVALPADLPLGDYSVKIPYTSGMQRHFASYLGARFKIPYNVAKTLPRENEMLFLFKNIGG

SEQ ID NO: 70 - protein, 337 aa, Synthetic construct
WNNIVFYSLGNVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILPGGSSGGADVQINIRGNVYIPP

SEQ ID NO: 71 - protein, 17 aa, Synthetic construct
GGSSGGGGSSGSGSSSG

SEQ ID NO: 72 - protein, 15 aa, Synthetic construct
SSGGGGSSGSGSSSG

SEQ ID NO: 73 - protein, 13 aa, Synthetic construct
SSGGGGSSGSGSS

SEQ ID NO: 74 - protein, 5 aa, Synthetic construct
SSGGG

SEQ ID NO: 75 - protein, 82 aa, Synthetic construct
AAAAAAAAAAAAAAAAAAAAAAAAAAAAAAAAAAAAAAAAAAAAAAAAAAAAAAAAAAAAAAAAAAAAAAAAAAAAAAAAAA

SEQ ID NO: 76 - protein, 207 aa, Synthetic construct
MEGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 77 - protein, 156 aa, Synthetic construct
MTKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 78 - protein, 156 aa, Synthetic construct
MTKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 79 - protein, 209 aa, Synthetic construct
MSTINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 80 - protein, 114 aa, Synthetic construct
MPIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 81 - protein, 157 aa, Synthetic construct
MNQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 82 - protein, 205 aa, Synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 83 - protein, 157 aa, Synthetic construct
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 84 - protein, 177 aa, Synthetic construct
MFTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 85 - protein, 157 aa, Synthetic construct
MNQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 86 - protein, 201 aa, Synthetic construct
MGHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 87 - protein, 237 aa, Synthetic construct
MYEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 88 - protein, 138 aa, Synthetic construct
MGMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 89 - protein, 154 aa, Synthetic construct
MKYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 90 - protein, 164 aa, Synthetic construct
MAVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 91 - protein, 175 aa, Synthetic construct
MGANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 92 - protein, 208 aa, Synthetic construct
MTDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 93 - protein, 128 aa, Synthetic construct
MITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 94 - protein, 235 aa, Synthetic construct
MGSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 95 - protein, 162 aa, Synthetic construct
MSGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 96 - protein, 157 aa, Synthetic construct
MGDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 97 - protein, 157 aa, Synthetic construct
MILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 98 - protein, 156 aa, Synthetic construct
MTKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 99 - protein, 209 aa, Synthetic construct
MDDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 100 - protein, 114 aa, Synthetic construct
MPIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 101 - protein, 114 aa, Synthetic construct
MPIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 102 - protein, 157 aa, Synthetic construct
MNQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 103 - protein, 157 aa, Synthetic construct
MNQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 104 - protein, 205 aa, Synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 105 - protein, 205 aa, Synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 106 - protein, 205 aa, Synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 107 - protein, 157 aa, Synthetic construct
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 108 - protein, 157 aa, Synthetic construct
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 109 - protein, 157 aa, Synthetic construct
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 110 - protein, 156 aa, Synthetic construct
MTKKVGIVDTTFARVDMASAAILTLKMESPNIKIIRKTVPGIKDLPVACKKLLEEEGCDIVMALGMPGKXEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 111 - protein, 209 aa, Synthetic construct
MXXINNQLKXLKVIPVIAIDNAEDIIPLGKVLAENGLPAAEITFRSSAAVKAIMLLRSAQPEMLIGAGTILNGVQALAAKEAGAXFVVSPGFNPNTVRACQIIGIDIVPGVNNPSTVEXALEMGLTTLKFFPAEASGGISMVKSLVGPYGDIRLMPTGGITPXNIDNYLAIPQVLACGGTWMVDKKLVXNGEWDEIARLTREIVEQVNP

SEQ ID NO: 112 - protein, 114 aa, Synthetic construct
MPIFTLNTNIKAXDVPSDFLSLTSRLVGLILSXPGSYVAVHINTDQQLSFGGSTNPAAFGTLMSIGGIEPXKNXDHSAVLFDHLNAMLGIPKNRMYIHFVXLXGDDVGWNGTTF

SEQ ID NO: 113 - protein, 157 aa, Synthetic construct
MNQHSHKDXETVRIAVVRARWHADIVDACVEAFEIAMAAIGGDRFAVDVFDVPGAYEIPLHARTLAETGRYGAVLGTAFVVXGGIYXHEFVASAVIDGMMNVQLXTGVPVLSAVLTPHXYXDSXEXHXFFAAHFAVKGVEAARACIEILXAREKIAA

SEQ ID NO: 114 - protein, 205 aa, Synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHXILKLFPGEVVGPXFVXAMKGPFPNVKFVPTGGVXLDXVCXWFXAGVLAVGVGXALVXGXPDEVREXAKXFVXXIRGCTE

SEQ ID NO: 115 - protein, 157 aa, Synthetic construct
MNQHSHKDXETVRIAVVRARWHAEIVDACVSAFEAAMXDIGGDRFAVDVFDVPGAYEIPLHARTLAETGRYGAVLGTAFVVXGGIYXHEFVASAVIXGMMNVQLXTGVPVLSAVLTPHXYXXSXAXTLLFLALFAVKGMEAARACVEILAAREKIAA

SEQ ID NO: 116 - DNA, 142 bp, Synthetic construct
  source 1..142: mol_type = other DNA
taatgcttaagtcgaacagaaagtaatcgtattgtacacggccgcataatcgaaattaatacgactcactataggggaattgtgagcggataacaattccccatcttagtatattagttaagtataagaaggagatatactt

SEQ ID NO: 117 - DNA, 18 bp, Synthetic construct
  source 1..18: mol_type = other DNA
taaagaaggagatatcat

SEQ ID NO: 118 - DNA, 17 bp, Synthetic construct
  source 1..17: mol_type = other DNA
tgagaaggagatatcat

SEQ ID NO: 119 - protein, 335 aa, Escherichia coli
MKKWFPAFLFLSLSGGNDALAGWHNVMFYAFNDYLTTNAGNVKVIDQPQLYIPWNTGSATATYYSCSGPEFASGVYFQEYLAWMVVPKHVYTNEGFNIFLDVQSKYGWSMENENDKDFYFFVNGYEWDTWTNNGARICFYPGNMKQLNNKFNDLVFRVLLPVDLPKGHYNFPVRYIRGIQHHYYDLWQDHYKMPYDQIKQLPATNTLMLSFDNVGGCQPSTQVLNIDHGSIVIDRANGNIASQTLSIYCDVPVSVKISLLRNTPPIYNNNKFSVGLGNGWDSIISLDGVEQSEEILRWYTAGSKTVKIESRLYGEEGKRKPGELSGSMTMVLSFP

SEQ ID NO: 120 - protein, 336 aa, Escherichia coli
MKKWFPALLFSLCVSGESSAWNNIVFYSLGNVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDAGNLCQKPGETTRLTEKFNDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 121 - protein, 336 aa, Escherichia coli; identical to SEQ ID NO: 120

SEQ ID NO: 122 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEANTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTNPAYSHGQQFSVGLGHGWDSIISINGVDTGETTMRWYRAGTQNLTTGSRLYGESSKIQPGVLSGSATLLMILP

SEQ ID NO: 123 - protein, 335 aa, Escherichia coli
MKKWLPAFLFLSLSGCNDALAANQSTMFYSFNDNIYRPQLSVKVTDIVQFIVDINSASSTATLNYVACNGFTWTHGLYWSEYFAWLVVPKHVSYNGYNIYLELQSRGSFSLDAEDNDNYYLTKGFAWDEANTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTAPAYSHGQQFSVGLGHGWDSIVSVNGVDKGETTMRWYRAGTQNLTIGSRLYGESSKIQPGVLSGSATLLMILP

SEQ ID NO: 124 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEANTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSVNGVDKGETTMRWYSAGTQNLTIGSRLYGESSKIQPGVLSGSATLLMILP

SEQ ID NO: 125 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEVNTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSVNGVDKGETTMRWYSAGTQNLTIGSRLYGESSKIQPGVLSGSATLLMILP

SEQ ID NO: 126 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEANTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTAPAYSHGQQFSVGLGHGWDSIVSVNGVDKGETTMRWYRAGTQNLTIGSRLYGESSKIQPGVLSGSATLLMILP

SEQ ID NO: 127 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEANTSGQTCFNIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTNPAYSHGQQFSVGLGHGWDSIISINGVDTGETTMRWYRAGTQNLTIGSRLYGESSKIQPGVLSGSATLLMILP

SEQ ID NO: 128 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSKGGFSLDAEDNDNYYLTKGFAWDEVNSSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSVNGVDKGETTMRWYSAGTQNLTIGSRLYGESSKIQPGVLSGSATLLMILP

SEQ ID NO: 129 - protein, 335 aa, Escherichia coli
MKKWLPAFLFLSLSGCNDALAANQSTMFYSFNDNIYRPQLSVKVADIVQFIVDINSASSTATLSYVACNGFTWTHGLYWSEYFAWLVVPKHVSYNGYNIYLELQSKGGFSLDAEDNDNYYLTKGFAWDEVNSSGRVCFNIGEKRSLAWSFGGVTLNARFPVDLPKGDYTFPVKFLRGIQRNNYDYIGGRYKIPSSLMKTFPFNGTLNFPIKNTGGCRPSAQSLEINHGDLSINSANNHYAAQTLSVSCDVPTNIRFFLLSNTAPAYSHGQQFSVGLGHGWDSIVSVNGVDKGETTMRWYRAGTQNLTIGSRLYGESSKIQPGVLSGSATLLMILP

SEQ ID NO: 130 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSKGGFSLDAEDNDNYYLTKGFAWDEVNSSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSINGVDTGETTMRWYRAGTQNLTIGSRLYGESSKIQPGVLSGSATLLMILP

SEQ ID NO: 131 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQYKGGFSLDAEDNDNYYLTKGFAWDEVNSSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSINGVDTGETTMRWYRAGTQNLTIGSRLYGESSKIQPGVLSGSATLLIILP

SEQ ID NO: 132 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGGFSLDAEDNDNYYLTKGFAWDEVNTSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSINGVDTGETTMRWYRAGTQNLTIGSRLYGESSKIQPGVLSGSATLLMILP

SEQ ID NO: 133 - protein, 335 aa, Escherichia coli
MKKWLPAFLFLSLSGCNDALAANQSTMFYSFNDNIYRPQLSVKVTDIVQFIVDINSASSTATLSYVACNGFTWTHGLYWSEYFAWLVVPKHVSYNGYNIYLELQSRGSFSLDAEDNDNYYLTKGFAWDEVNSSGRVCFDIGEKRSLAWSFGGVTLNARLPVDLPKGDYTFPVKFLRGIQRNNYDYIGGRYKIPSSLMKTFPFNGTLNFSIKNTGGCRPSAQSLEINHGDLSINSANNHYAAQTLSVSCDVPTNIRFFLLSNTTPAYSHGQQFSVGLGHGWDSIVSINGVDTGETTMRWYRAGTQNLTIGSRLYGESSKIQPGVLSGSATLLMILP

SEQ ID NO: 134 - protein, 335 aa, Escherichia coli; identical to SEQ ID NO: 133

SEQ ID NO: 135 - protein, 336 aa, Escherichia coli
MKKWFPALLFSLCVSGESSAWNNIVFYSLGDVNSYQGGNVVITQRPQFITSWRPGIATVTWNQCNGPEFADGFWAYYREYIAWVVFPKKVMTQNGYPLFIEVHNKGNWSEENTGDNDSYFFLKGDKWDERAFDAANLCQKLGETTRLTEQFNDIIFKVALPADLPLGDYSVTIPYTSGIQRHFANYFGARFKIPYNVAKTLPRENKMLFLFKNTGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGDLSGSATLLMILP

SEQ ID NO: 136 - protein, 336 aa, Escherichia coli
MKKWFPALLFSLCVSGESSAWNNIVFYSLGDVNSYQGGNVVITQRPQFITSWRPAIATVTWNQCNGPEFADGSWAYYREYIAWVVFPKKVMTKNGYPLFIEVHNKGSWSEENTGDNDSYFFLKGYKWDERAFDTANLCQKPGETTRLTEKFDDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 137 - protein, 336 aa, Escherichia coli
MKKWFPALLFSLCVSGESSAWNNIVFYSLGDVNSYQGGNVVITQRPQFITSWRPGIATVTWNQCNGPEFADGSWAYYREYIAWVVFPKKVMTKNGYPLFIEVHNKGSWSEENTGDNDSYFFLKGYKWDERAFDTANLCQKPGETIRLTEKFDDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 138 - protein, 336 aa, Escherichia coli; identical to SEQ ID NO: 137

SEQ ID NO: 139 - protein, 336 aa, Escherichia coli; identical to SEQ ID NO: 137

SEQ ID NO: 140 - protein, 336 aa, Escherichia coli; identical to SEQ ID NO: 137

SEQ ID NO: 141 - protein, 336 aa, Escherichia coli; identical to SEQ ID NO: 137

SEQ ID NO: 142 - protein, 336 aa, Escherichia coli; identical to SEQ ID NO: 137

SEQ ID NO: 143 - protein, 336 aa, Escherichia coli; identical to SEQ ID NO: 137

SEQ ID NO: 144 - protein, 336 aa, Escherichia coli
MKKWFPALLFSLCVSGESSAWNNIVFYSLGDVNSYQGGNVVITQRPQFITSWRPGIATVTWNQCNGPEFADGSWAYYREYIAWVVFPKKVMTKNGYPLFIEVHNKGSWSEENTGYNDSYFFLKGYKWDERAFDTANLCQKPGETIRLTEKFDDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 145 - protein, 336 aa, Escherichia coli
MKKWFPALLFSLCVSGESSAWNNIVFYSLGDVNSYQGGNVVITQRPQFITSWRPGIATVTWNQCNGPGFVDGSWAYYREYIAWVVFPKKVMTQNGYPLFIEVHNKGSWSEENTGDNDSYFFLKGYKWDERAFDTANLCQKPGETTRLTEKFDDIIFKVALPADLPLGDYSVTIPYTSGIQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 146 - protein, 336 aa, Escherichia coli
MKKWFPALLFSLCVSGESSAWNNIVFYSLGDVNSYQGGNVVITQRPQFITSWRPGIATVTWNQCNGPGFADGSWAYYREYIAWVVFPKKVMTKNGYPLFIEVHNKGSWSEENTGDNDSYFFLKGYKWDERAFDTANLCQKPGEKTRLTEKFDDIIFKVALPADLPLGDYSVTIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 147 - protein, 336 aa, Escherichia coli
MKKWFPALLFSLCVSGESSAWNNIVFYSLGDVNSYQGGNVVITQRPQFITSWRPGIATVTWNQCNGPGFADGYWAYYREYIAWVVFPKKVMTKNGYPLFIEVHNKGSWSEENTGDNDSYFFLKGYKWDERAFDTANLCQKPGEKNRLTEKFDDIIFKVALPADLPLGDYSVTIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 148 - protein, 336 aa, Escherichia coli
MKKWFPALLFSLCVSGESSAWNNIVFYSLGDVNSYQGGNVVITQRPQFITSWRPGIATVTWNQCNGPGFADGFWAYYREYIAWVVFPKKVMTQNGYPLFIEVHNKGSWSEENTGDNDSYFFLKGYKWDERAFDTANLCQKPGEKTRLTEKFDDIIFKVALPADLPLGDYSVTIPYTSGMQRHFASYLGARFKIPYNVAKTLPRENEMLFLFKNIGGCRPSAQSLEIKHGDLSINSANNHYAAQTLSVSCDVPANIRFMLLRNTAPTYSHGKKFSVGLGHGWDSIVSVNGVDTGETTMRWYKAGTQNLTIGSRLYGESSKIQPGVLSGSATLLMILP

SEQ ID NO: 149 - protein, 336 aa, Escherichia coli; identical to SEQ ID NO: 148

SEQ ID NO: 150 - protein, 336 aa, Escherichia coli; identical to SEQ ID NO: 148

SEQ ID NO: 151 - protein, 336 aa, Escherichia coli; identical to SEQ ID NO: 148

SEQ ID NO: 152 - protein, 336 aa, Escherichia coli; identical to SEQ ID NO: 148

SEQ ID NO: 153 - protein, 336 aa, Escherichia coli; identical to SEQ ID NO: 148

SEQ ID NO: 154 - protein, 336 aa, Escherichia coli
MKKWFPALLFSLCVSGESSAWNNIVFYSLGDVNSYQGGNVVITQRPQFITSWRPGIATVTWNQCNGPGFADGSWAYYREYIAWVVFPKKVMTQNGYPLFIEVHNKGSWSEENTGDNDSYFFLKGYKWDERAFDAGNLCQKPGETTRLTEKFDDIIFKVALPADLPLGDYSVKIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 155 - protein, 336 aa, Escherichia coli
MKKWFPALLFSLCVSGESSAWNNIVFYSLGDVNSYQGGNVVITQRPQFITSWRPGIATVTWNQCNGPGFADGSWAYYREYIAWVVFPKKVMTQNGYPLFIEVHNKGNWSEENSGDNDSYFFLKGYKWDERAFDAGNLCQKPGETTRLTEKFDDIIFKVALPADLPLGDYSVKIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 156 - protein, 336 aa, Escherichia coli
MKKWFPALLFSLCVSGESSAWNNIVFYSLGDVNSYQGGNVVITQRPQFITSWRPGIATVTWNQCNGPGFTDGSWAYYREYIAWVVFPKKVMTQNGYPLFIEVHNKGNWSEENSGDNDSYFFLKGYKWDERAFDAGNLCQKPGETTRLTEKFDDIIFKVALPADLPLGDYSVKIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 157 - protein, 336 aa, Escherichia coli
MKKWFPALLFSLCVSGESSAWNNIVFYSLGNVNSYQGGNVVITQRPQFITSWRPGIATVTWNQCNGPGFADGYWAYYREYIAWVVFPKKVMTKNGYPLFIEVHNKGSWSEENTGDNDSYFFLKGYKWDERAFDAGNLCQKPGETTRLTEKFDDIIFKVALPADLPLGDYSVTIPYTSGMQRHFASYLGARFKIPYNVAKTLPRENEMFFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 158 - protein, 336 aa, Escherichia coli
MKKWFPALLFSLCVSGESSAWNNIVFYSLGNVNSYQGGNVVITQRPQFITSWRPGIATVTWNQCNGPGFVGGSWAYYREYIAWVVFPKKVMTKNGYPLFIEVHNKGSWSEENTGDNDSYFFLKGYKWDERAFDAGNLCQKPGETTRLTEKFDDIIFKVALPADLPLGDYSVTIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 159 - protein, 336 aa, Escherichia coli
MKKWFPALLFSLCVSGESSAWNNIVFYSLGNVNSYQGGNVVITQRPQFITSWRPGIATVTWNQCNGPGFADGSWAYYREYIAWVVFPKKVMTKNGYPLFIEVHNKGSWSEENTGDNDSYFFLKGYKWDERAFDAGNLCQKPGETTRLTEKFDDIIFKVALPADLPLGDYSVTIPYTSGMQRHFASYLGARFKIPYNVAKTLPRENEMLFLFKNIGGCRPSAQSLEIKHGDLSINSANNHYAAQTLSVSCDVPANIRFMLLRNTTPTYSHGKKFSVGLGHGWDSIVSVNGVDTGETTMRWYKAGTQNLTIGSRLYGESSKIQPGVLSGSATLLMILP

SEQ ID NO: 160 - protein, 336 aa, Escherichia coli; identical to SEQ ID NO: 159

SEQ ID NO: 161 - protein, 336 aa, Escherichia coli; identical to SEQ ID NO: 159

SEQ ID NO: 162 - protein, 336 aa, Escherichia coli; identical to SEQ ID NO: 159

SEQ ID NO: 163 - protein, 336 aa, Escherichia coli; identical to SEQ ID NO: 159

SEQ ID NO: 164 - protein, 336 aa, Escherichia coli; identical to SEQ ID NO: 159

SEQ ID NO: 165 - protein, 336 aa, Escherichia coli; identical to SEQ ID NO: 159